(19) United States
(12) Patent Application Publication
(10) Pub. No.: US 2023/0027888 A1
(43) Pub. Date: Jan. 26, 2023

(54) ORGANIC ELECTROLUMINESCENT DEVICE, ORGANIC ELECTROLUMINESCENCE DISPLAY DEVICE, ELECTRONIC DEVICE AND COMPOUND

(71) Applicant: IDEMITSU KOSAN CO.,LTD., Tokyo (JP)

(72) Inventors: Hisato MATSUMOTO, Tokyo (JP); Toshinari OGIWARA, Tokyo (JP)

(73) Assignee: IDEMITSU KOSAN CO.,LTD., Tokyo (JP)

(21) Appl. No.: 17/808,015

(22) Filed: Jun. 21, 2022

(30) Foreign Application Priority Data
Jun. 25, 2021 (JP) 2021-106074

(51) Int. Cl. H01L 51/00; C07D 409/12; C09K 11/06

(52) U.S. Cl. CPC H01L 51/0061; C07D 409/12; C09K 11/06; H01L 2251/558; H01L 51/5012

(57) ABSTRACT

There is provided an organic EL device, including: an anode; a cathode; an emitting layer provided between the anode and the cathode; a first layer provided between the anode and the emitting layer; and a second layer provided between the anode and the first layer, in which the emitting layer contains a delayed fluorescent compound, the first layer contains a first compound, the second layer contains a second compound, an ionization potential Ip(HT1) of the first compound satisfies Numerical Formula 1, a hole mobility μh(HT1) of the first compound satisfies Numerical Formula 2, an ionization potential Ip(HT2) of the second compound satisfies Numerical Formula 3, and the first layer has a film thickness of 15 nm or more,

Ip(HT1)≥5.69 eV (Numerical Formula 1)

μh(HT1)≥1.00×10⁻⁵ cm²/Vs (Numerical Formula 2)

Ip(HT2)≥5.60 eV (Numerical Formula 3).

The entire disclosure of Japanese Patent Application No. 2021-106074, filed Jun. 25, 2021 is expressly incorporated by reference herein.

### TECHNICAL FIELD

The present invention relates to an organic electroluminescence device, an organic electroluminescence display device, an electronic device, and a compound.

### BACKGROUND ART

When voltage is applied to an organic electroluminescence device (hereinafter, occasionally referred to as an organic EL device), holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Studies have thus been made to improve performance of the organic EL device.

For instance, the organic EL device is expected to emit light more efficiently using triplet excitons in addition to singlet excitons. In view of the above, a highly-efficient fluorescent organic EL device using thermally activated delayed fluorescence (hereinafter simply referred to as “delayed fluorescence” in some cases) has been proposed and studied.

A Thermally Activated Delayed Fluorescence (TADF) mechanism uses such a phenomenon that inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI Chihaya, published by Kodansha, issued on Apr. 1, 2012, on pages 261-268).

For instance, Literatures 1, 2, and 3 describe an organic electroluminescence device using a delayed fluorescent compound.

In order to improve performance of an electronic device such as a display, there is a demand for further improvement in performance of an organic electroluminescence device.

### SUMMARY OF THE INVENTION

An object of the invention is to provide an organic electroluminescence device and an organic electroluminescence display device capable of achieving higher performance (especially, a decrease in voltage), specifically, both luminous efficiency and voltage suitable for practical use, an electronic device including the organic electroluminescence device, and an electronic device including the organic electroluminescence display device.

Another object of the invention is to provide a compound with which an organic electroluminescence device, an organic electroluminescence display device, and an electronic device can achieve higher performance (especially, a decrease in voltage), specifically, both luminous efficiency and voltage suitable for practical use. According to an aspect of the invention, there is provided an organic electroluminescence device, including: an anode; a cathode; an emitting layer provided between the anode and the cathode; a first layer provided between the anode and the emitting layer; and a second layer provided between the anode and the first layer, in which the emitting layer contains a delayed fluorescent compound; the first layer contains a first compound; the second layer contains a second compound; an ionization potential Ip(HT1) of the first compound satisfies a numerical formula (Numerical Formula 1) below; a hole mobility μh(HT1) of the first compound satisfies a numerical formula (Numerical Formula 2) below; an ionization potential Ip(HT2) of the second compound satisfies a numerical formula (Numerical Formula 3) below; and the first layer has a film thickness of 15 nm or more.

Ip(HT1)≥5.69 eV (Numerical Formula 1)

μh(HT1)≥1.00×10⁻⁵ cm²/Vs (Numerical Formula 2)

Ip(HT2)≥5.60 eV (Numerical Formula 3)

According to another aspect of the invention, there is provided an electronic device including the organic electroluminescence device according to the above aspect of the invention.

According to still another aspect of the invention, there is provided an organic electroluminescence display device, including: an anode and a cathode arranged to face each other; a blue-emitting organic EL device as a blue pixel; a green-emitting organic EL device as a green pixel; and a red-emitting organic EL device as a red pixel, in which the red pixel includes the organic electroluminescence device according to the aspect of the invention as the red-emitting organic EL device; the red-emitting organic EL device includes: a red emitting layer as the emitting layer; the first layer provided between the red emitting layer and the anode; and the second layer provided between the first layer and the anode; the blue-emitting organic EL device includes a blue emitting layer provided between the anode and the cathode; the green-emitting organic EL device includes a green emitting layer provided between the anode and the cathode; and the second layer is provided between the anode and each of the blue emitting layer, the green emitting layer, and the first layer in a shared manner across the blue-emitting organic EL device, the green-emitting organic EL device, and the red-emitting organic EL device.

According to a further aspect of the invention, there is provided an electronic device including the organic electroluminescence display device according to the above aspect of the invention.

According to a still further aspect of the invention, there is provided a compound represented by a formula (10) below.

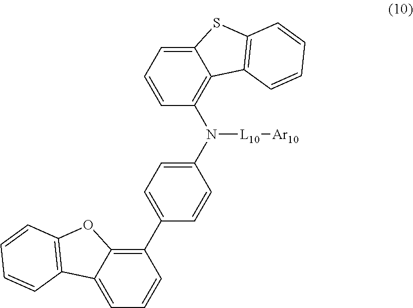

In the formula (10):

L₁₀ is a single bond, or a substituted or unsubstituted arylene group having 6 to 12 ring carbon atoms;

a substituent, if present, for L₁₀ is an unsubstituted phenyl group;

Ar₁₀ is a substituted or unsubstituted aryl group having 6 to 18 ring carbon atoms; and

a substituent, if present, for Ar₁₀ is an unsubstituted phenyl group or an unsubstituted naphthyl group.

According to the aspects of the invention, there are provided an organic electroluminescence device and an organic electroluminescence display device capable of achieving higher performance (especially, a decrease in voltage), specifically, both luminous efficiency and voltage suitable for practical use, an electronic device including the organic electroluminescence device, and an electronic device including the organic electroluminescence display device.

According to the aspect of the invention, there is provided a compound with which an organic electroluminescence device, an organic electroluminescence display device, and an electronic device can achieve higher performance (especially, a decrease in voltage), specifically, both luminous efficiency and voltage suitable for practical use.

### BRIEF DESCRIPTION OF DRAWING(S)

FIG. 1 schematically shows an exemplary arrangement of an organic electroluminescence device according to a first exemplary embodiment of the invention.

FIG. 2 schematically shows a device for measuring transient PL.

FIG. 3 shows an example of decay curves of the transient PL.

FIG. 4 schematically shows a relationship in energy level and energy transfer between a compound M1 and a compound M2 in an emitting layer of an exemplary organic electroluminescence device according to the first exemplary embodiment of the invention.

FIG. 5 schematically shows a relationship in energy level and energy transfer between the compound M1, the compound M2 and a compound M3 in an emitting layer of an exemplary organic electroluminescence device according to a second exemplary embodiment of the invention.

FIG. 6 schematically shows a relationship in energy level and energy transfer between the compound M2 and a compound M4 in an emitting layer of an exemplary organic electroluminescence device according to a third exemplary embodiment of the invention.

FIG. 7 schematically shows an exemplary arrangement of an organic electroluminescence display device according to a fourth exemplary embodiment of the invention.

FIG. 8 schematically shows an exemplary arrangement of an organic electroluminescence display device according to a fifth exemplary embodiment of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). Herein, an unsubstituted aryl group refers to an “unsubstituted aryl group” in a “substituted or unsubstituted aryl group,” and a substituted aryl group refers to a “substituted aryl group” in a “substituted or unsubstituted aryl group.” A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and a monovalent aryl group derived by removing one hydrogen atom from cyclic structures represented by formulae (TEMP-1) to (TEMP-15) below.

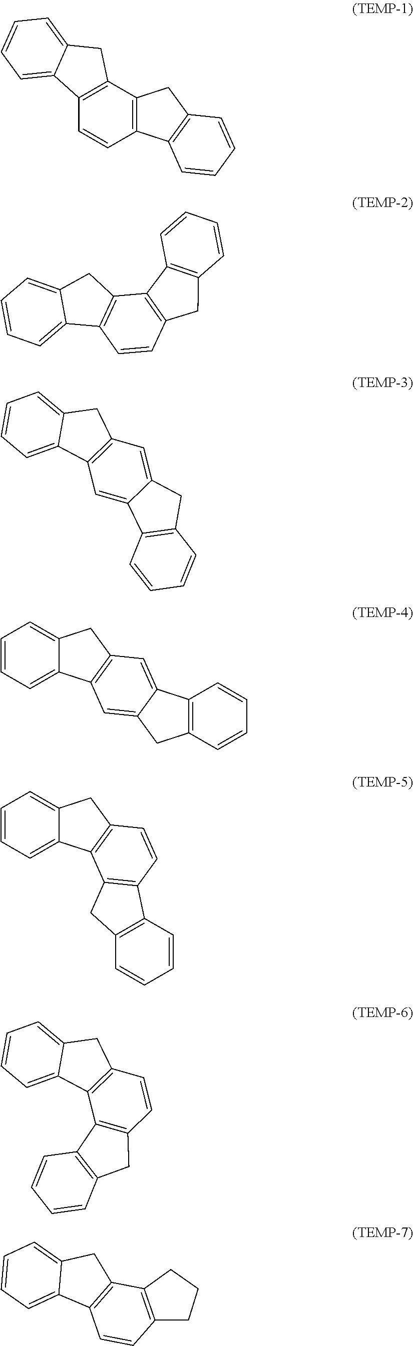

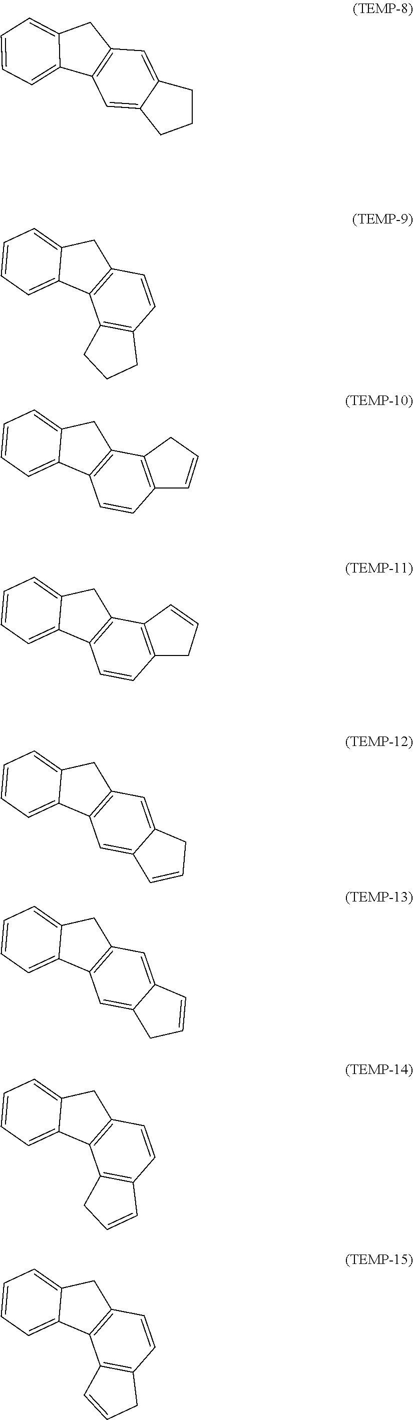

### Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.” A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).
Monovalent Heterocyclic Groups Derived by Removing One Hydrogen Atom from Cyclic Structures Represented by Formulae (TEMP-16) to (TEMP-33) (Specific Example Group G2A4):

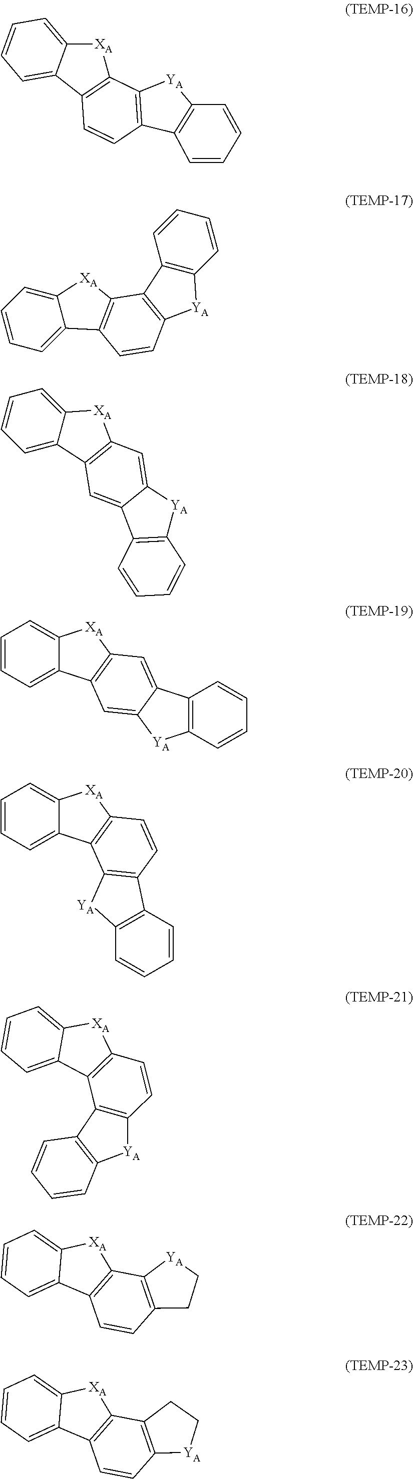

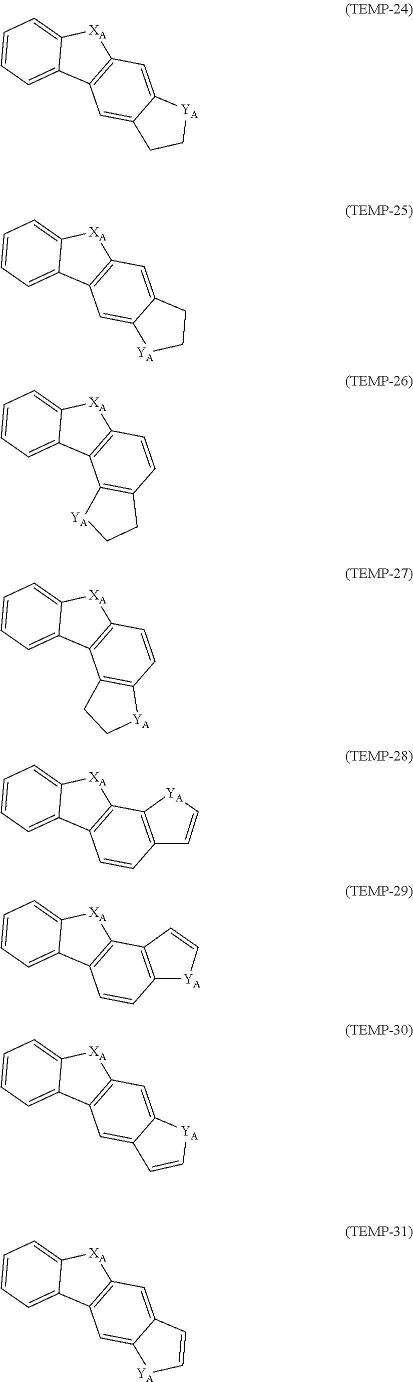

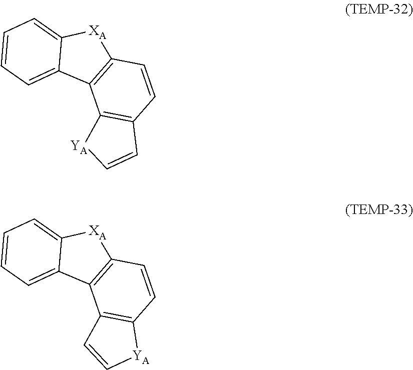

In the formulae (TEMP-16) to (TEMP-33), X_{A} and Y_{A} are each independently an oxygen atom, a sulfur atom, NH or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

phenyldibenzothiophenyl group, methyldibenzothiophenyl group, t-butyldibenzothiophenyl group, and monovalent residue of spiro[9H-thioxanthene-9,9′-[9H]fluorene].
Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of XA or YA in a form of NH, and a hydrogen atom of one of XA and YA in a form of a methylene group (CH2).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.” A simply termed “alkyl group” herein includes both of an “unsubstituted alkyl group” and a “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

methyl group, ethyl group, n-propyl group, isopropyl group, n-butyl group, isobutyl group, s-butyl group, and t-butyl group.

### Substituted Alkyl Group (Specific Example Group G3B):

heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.” A simply termed “alkenyl group” herein includes both of an “unsubstituted alkenyl group” and a “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

vinyl group, allyl group, 1-butenyl group, 2-butenyl group, and 3-butenyl group.

### Substituted Alkenyl Group (Specific Example Group G4B):

1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. Herein, an unsubstituted alkynyl group refers to an “unsubstituted alkynyl group” in a “substituted or unsubstituted alkynyl group.” A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A):

ethynyl group

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.” A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

4-methylcyclohexyl group.
Group Represented by —Si(R₉₀₁)(R₉₀₂)(R₉₀₃)
Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6); where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —Si(G1)(G1)(G1) are mutually the same or different;

a plurality of G2 in —Si(G1)(G2)(G2) are mutually the same or different;

a plurality of G1 in —Si(G1)(G1)(G2) are mutually the same or different;

a plurality of G2 in —Si(G2)(G2)(G2) are mutually the same or different;

a plurality of G3 in —Si(G3)(G3)(G3) are mutually the same or different; and

a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6);

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6);

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6),

where:

G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3;

G6 represents a “substituted or unsubstituted cycloalkyl group” in the specific example group G6;

a plurality of G1 in —N(G1)(G1) are mutually the same or different;

a plurality of G2 in —N(G2)(G2) are mutually the same or different;

a plurality of G3 in —N(G3)(G3) are mutually the same or different; and

a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “substituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

Substituted or Unsubstituted Alkylthio Group Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

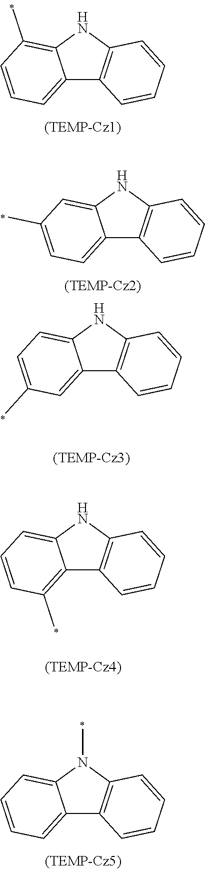

The (9-phenyl)carbazolyl group mentioned herein is, unless otherwise specified herein, specifically a group represented by one of formulae below.

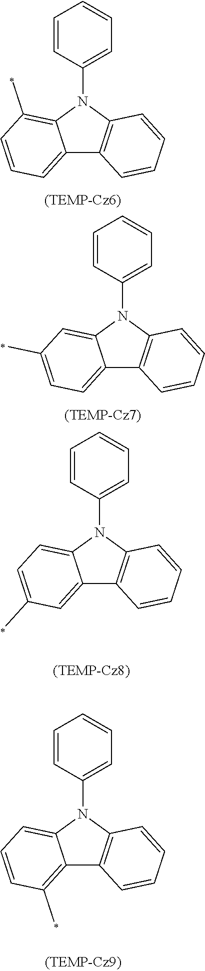

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

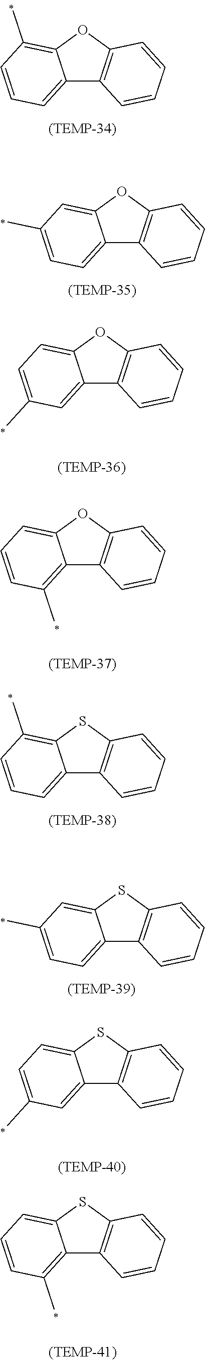

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

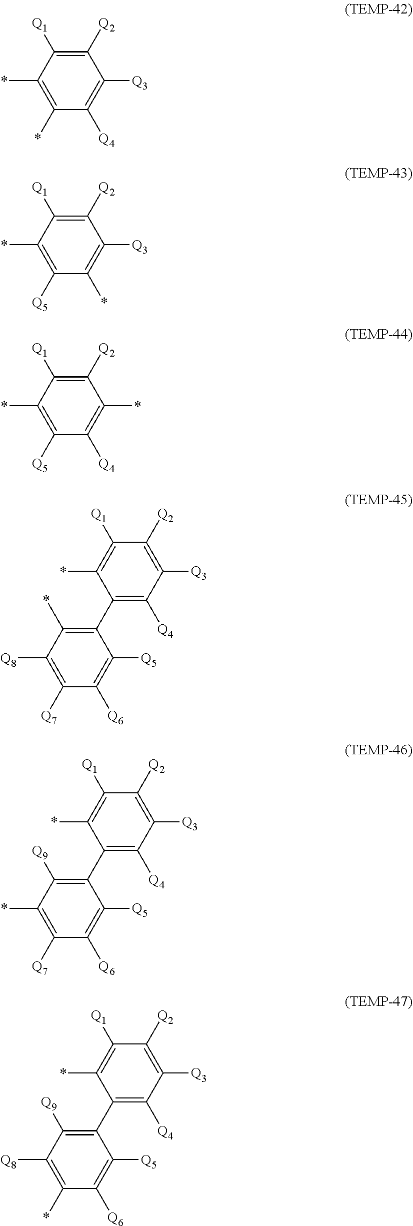

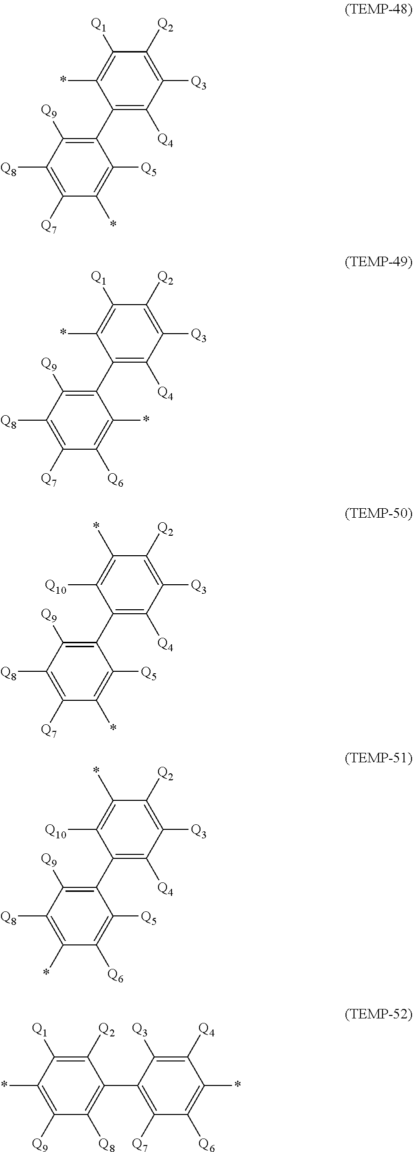

In the formulae (TEMP-42) to (TEMP-52), Q₁ to Q₁₀ are each independently a hydrogen atom or a substituent.

In the formulae (TEMP-42) to (TEMP-52), * represents a bonding position.

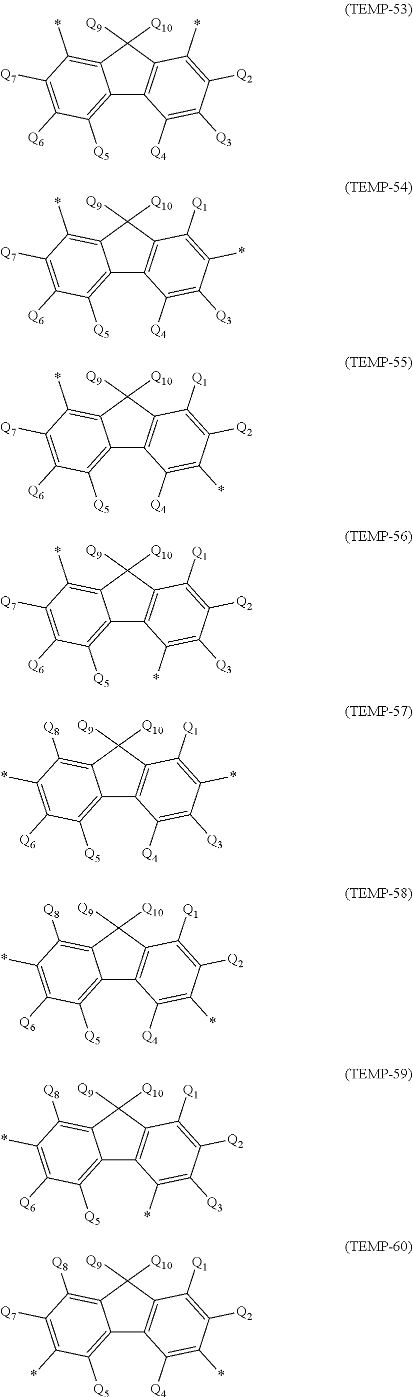

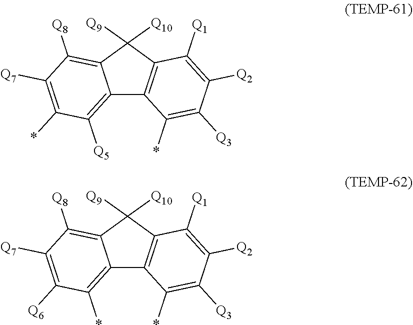

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

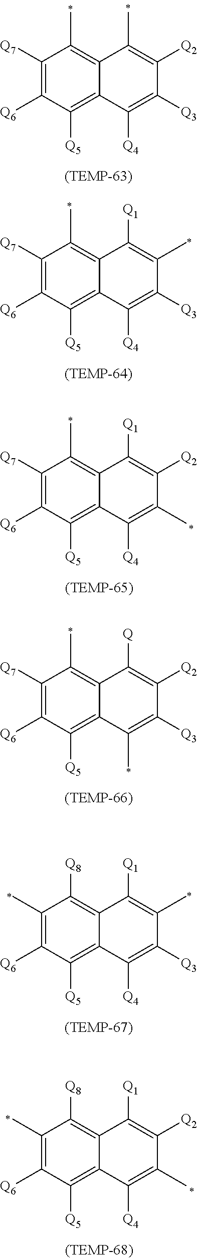

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

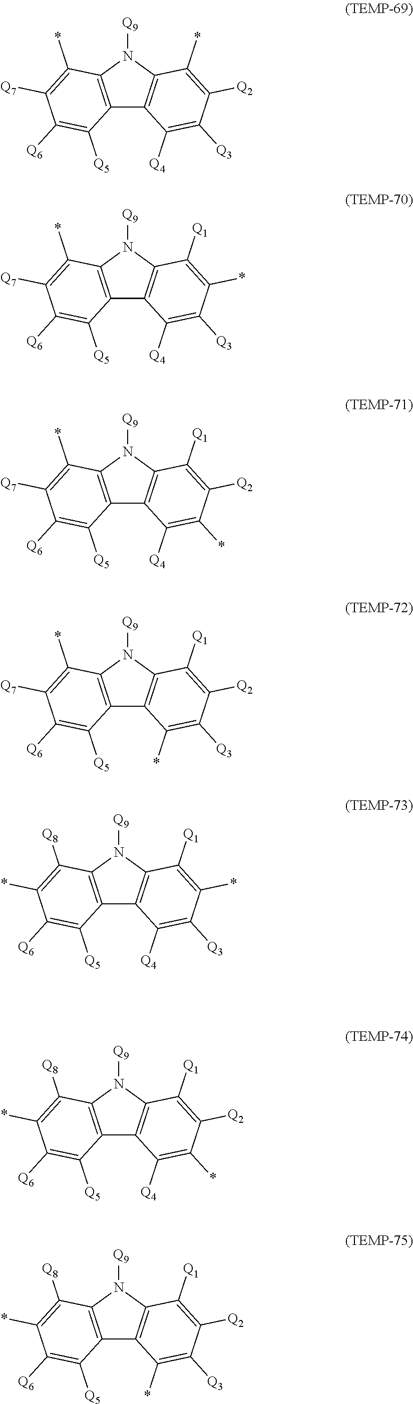

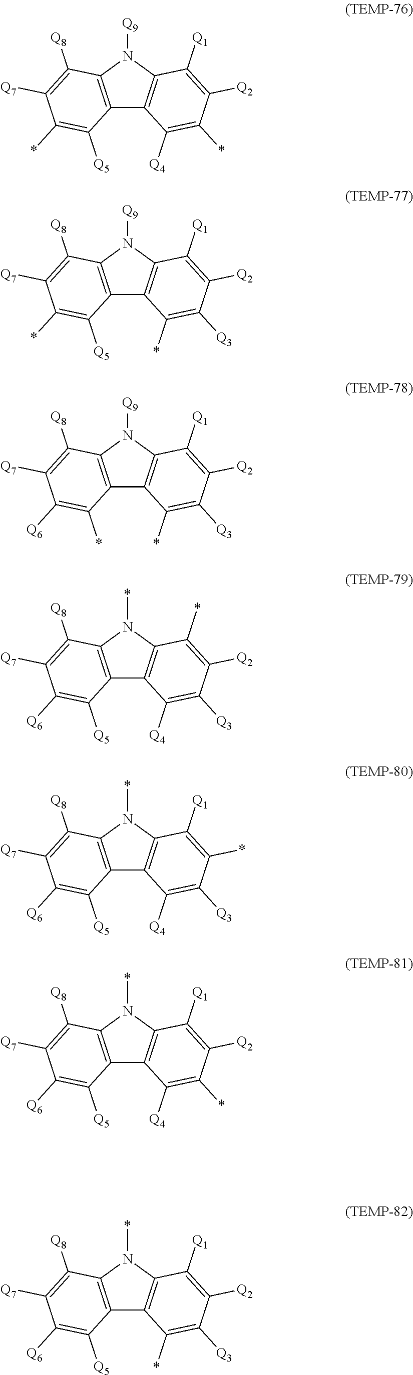

In the formulae (TEMP-69) to (TEMP-82), Q₁ to Q₉ are each independently a hydrogen atom or a substituent.

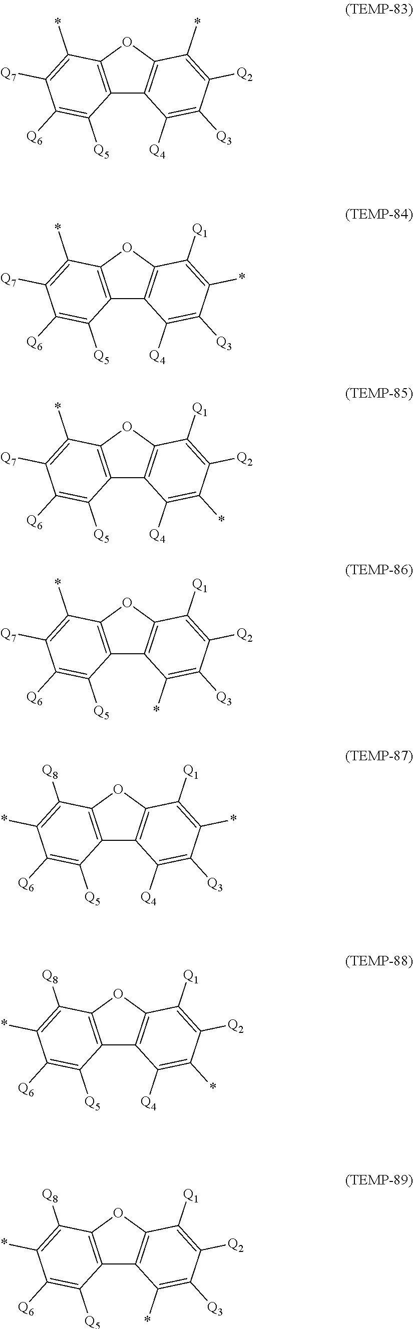

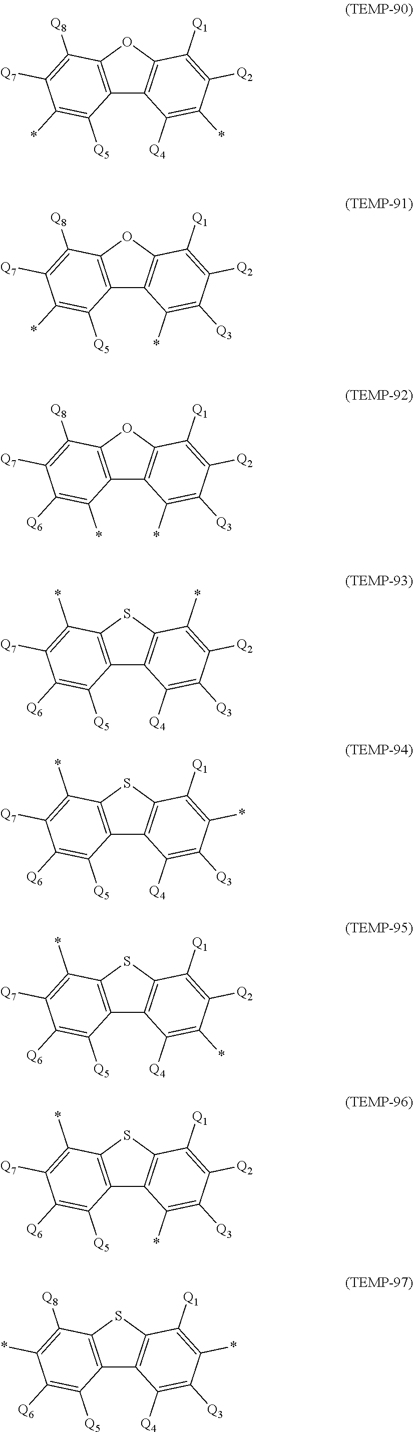

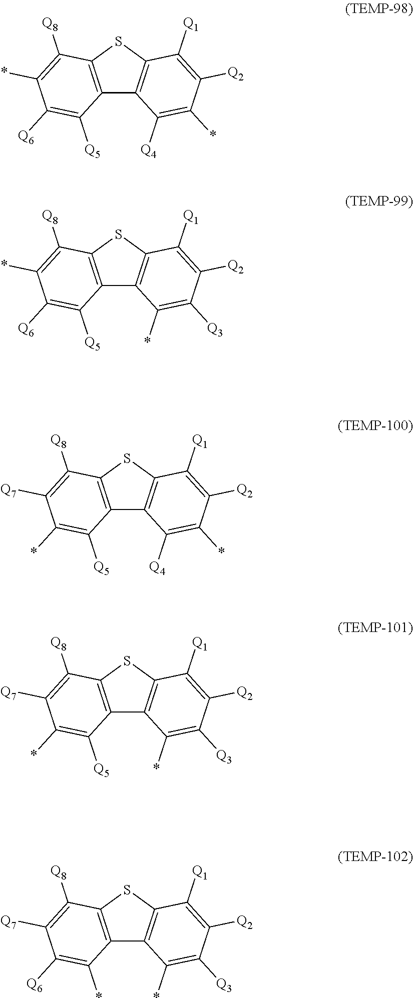

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

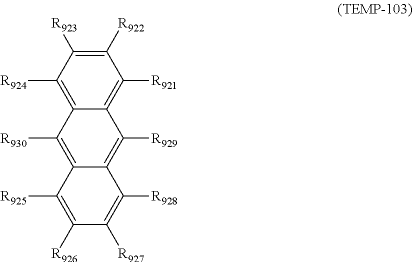

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

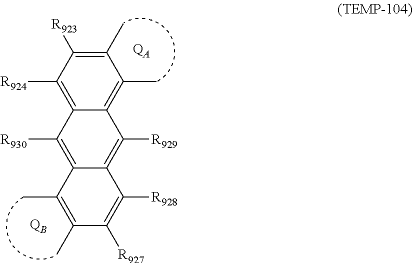

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

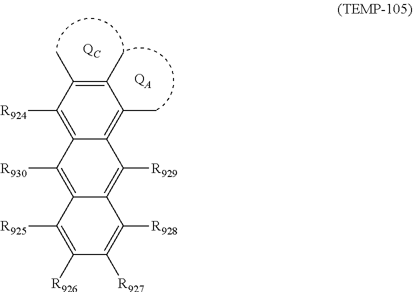

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, a substituent for the substituted or unsubstituted group (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms;

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when two or more R₉₀₁ are present, the two or more R₉₀₁ are mutually the same or different;

when two or more R₉₀₂ are present, the two or more R₉₀₂ are mutually the same or different;

when two or more R₉₀₃ are present, the two or more R₉₀₃ are mutually the same or different;

when two or more R₉₀₄ are present, the two or more R₉₀₄ are mutually the same or different;

when two or more R₉₀₅ are present, the two or more R₉₀₅ are mutually the same or different;

when two or more R₉₀₆ are present, the two or more R₉₀₆ are mutually the same or different; and

when two or more R₉₀₇ are present, the two or more R₉₀₇ are mutually the same or different.

In an exemplary embodiment, the substituent for the substituted or unsubstituted group is selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

Herein, a numerical formula represented by “A≥B” means that the value A is equal to the value B, or the value A is larger than the value B.

Herein, a numerical formula represented by “A≤B” means that the value A is equal to the value B, or the value A is smaller than the value B.

### First Exemplary Embodiment

An arrangement of an organic EL device according to a first exemplary embodiment of the invention will be described below.

The organic EL device according to the exemplary embodiment includes an organic layer between both electrodes of an anode and a cathode. The organic layer includes at least one layer formed from an organic compound. Alternatively, the organic layer is provided by laminating a plurality of layers each formed from an organic compound. The organic layer may further contain an inorganic compound.

In the exemplary embodiment, at least three of the organic layers are an emitting layer provided between the anode and the cathode, a first layer provided between the emitting layer and the anode, and a second layer provided between the first layer and the cathode. For instance, the organic layer may be provided by the first layer and the second layer, or may further include at least one layer usable for an organic EL device. Examples of the layer usable in the organic EL device, which are not particularly limited, include at least one layer selected from the group consisting of a hole injecting layer, hole transporting layer, electron blocking layer, electron injecting layer, electron transporting layer, and hole blocking layer.

An organic EL device according to the exemplary embodiment includes: an anode; a cathode; an emitting layer provided between the anode and the cathode; a first layer provided between the anode and the emitting layer; and a second layer provided between the anode and the first layer, in which the emitting layer contains a delayed fluorescent compound, the first layer contains a first compound, the second layer contains a second compound, an ionization potential Ip(HT1) of the first compound satisfies a numerical formula (Numerical Formula 1) below, a hole mobility μh(HT1) of the first compound satisfies a numerical formula (Numerical Formula 2) below, an ionization potential Ip(HT2) of the second compound satisfies a numerical formula (Numerical Formula 3) below, and the first layer has a film thickness of 15 nm or more.

Ip(HT1)≥5.69 eV (Numerical Formula 1)

μh(HT1)≥1.00×10⁻⁵ cm²/Vs (Numerical Formula 2)

Ip(HT2)≥5.60 eV (Numerical Formula 3)

Herein, a zone disposed between the anode and the emitting layer and formed by a plurality of organic layers is occasionally referred to as a hole transporting zone. Herein, a layer provided in a shared manner across a plurality of devices is occasionally referred to as a common layer, and a layer not provided in a shared manner across a plurality of devices is occasionally referred to as a non-common layer.

In an organic EL device using a TADF mechanism, there is a demand for an increase in total film thickness of the hole transporting zone according to how the organic EL device is used. The reason thereof is explained below.

When organic EL devices are provided, as a red pixel, green pixel, and blue pixel (RGB pixels), in an organic EL display device, in order to improve mass productivity and reduce production costs, the hole transporting layer is typically formed, as the common layer, in a shared manner across the RGB pixels. The hole transporting layer is formed from the same material to have the same film thickness.

On the other hand, since cavity adjustment is performed for each pixel in the organic EL display device including the RGB pixels, the total film thickness of the hole transporting zone needs to be optimized for each pixel according to the emission wavelength. Specifically, the total film thickness of the hole transporting zone needs to be larger, as the emission wavelength of the pixel is longer. Here, the film thickness of the hole transporting layer as the common layer is determined according to the pixel of which wavelength is not the longest among the RGB pixels (in a case of RGB pixels only, B pixel having the shortest wavelength). Thus, the film thickness of the hole transporting layer as the common layer is insufficient (excessively thin) for the rest of the pixels. To increase the total film thickness of the hole transporting layer for the rest of the pixels, the film thickness of the non-common layer (e.g., electron blocking layer) in the hole transporting zone needs to be increased. However, when the film thickness of the non-common layer (e.g., electron blocking layer) in the hole transporting zone of any of the RGB pixels is simply increased in the above arrangement where the film thickness of the hole transporting layer as the common layer is determined according to the pixel of which wavelength is not the longest among the RGB pixels, the device performance is decreased (especially, an increase in voltage). Measures are thus needed to solve the problem.

In conventional techniques, an increase in film thickness while keeping low voltage has been performed as follows: in a case where the pixel of which film thickness needs to increase is a phosphorescent pixel, the non-common layer (e.g., electron blocking layer) with an increased film thickness is provided in the hole transporting zone, and a material having a small absolute value of the ionization potential Ip is contained in the hole transporting layer as the common layer. However, in a case where the pixel of which film thickness needs to increase is a pixel that emits light using the TADF mechanism, studies for increasing the film thickness of the non-common layer have not been performed.

Inventors of the invention have studied for inhibiting a decrease in device performance (especially, an increase in voltage) even when the film thickness of the non-common layer (e.g., electron blocking layer) is increased in an arrangement using the TADF emitting layer.

The inventors of the invention have found out that the arrangement using the TADF emitting layer has the following problem: unlike a case where a phosphorescent emitting layer is used, when the non-common layer with an increased film thickness is simply provided and a material having a small absolute value of the ionization potential Ip is contained in the hole transporting layer as the common layer, voltage in the device is increased, making it impossible to practically use the device. The reason thereof is considered that the absolute value of the ionization potential Ip of the TADF emitting layer is larger than that of the phosphorescent emitting layer.

In order to solve the problem in which high voltage in the device makes it impossible to practically use the device, the inventors have found out that both luminous efficiency and voltage suitable for practical use can be achieved by increasing the film thickness of the first layer (e.g., electron blocking layer) disposed between the emitting layer and the anode to 15 nm or more and containing the first compound (Numerical Formula 2), in which the absolute value of the ionization potential Ip is large (Numerical Formula 1) and the hole mobility μh(HT1) is high, in the first layer having an increased film thickness, and containing the second compound (Numerical Formula 3), in which the absolute value of the ionization potential Ip is large, in the second layer (e.g., hole transporting layer) disposed between the first layer and the anode.

Using the second compound in which the absolute value of the ionization potential Ip is large can improve hole injection from the second layer to the first layer. The second compound satisfying Numerical Formula 3 thus contributes to a decrease in voltage in the device.

According to the organic EL device of the exemplary embodiment, higher performance (especially, a decrease in voltage), specifically, both luminous efficiency and voltage suitable for practical use can be achieved, even when the first layer has an increased film thickness.

Further, when the organic EL device according to the exemplary embodiment is provided in an organic EL display device in which at least one of the RGB pixels emits light using the TADF mechanism, cavity adjustment can be easily performed by simply increasing the film thickness of the first layer. Furthermore, the organic EL display device can be improved in mass productivity.

Literature 1 (WO 2020/241580), Literature 2 (WO 2019/013063), and Literature 3 (U.S. Patent Application Publication No. 2020/0203621) describe an organic EL device using a TADF emitting layer. However, in the organic EL device described in Literatures 1 and 2, the film thickness of the first layer (hole transporting layer or electron blocking layer close to the emitting layer) is not increased.

In the organic EL device described in Literature 3, the film thickness of the first layer (hole transporting layer or electron blocking layer close to the emitting layer) is increased. However, the ionization potential Ip of the compound used for the first layer fails to satisfy Numerical Formula 1. Further, Literature 3 does not describe a problem that may be caused when the film thickness of the first layer is increased.

Accordingly, none of Literatures 1 to 3 pay attention to the fact that a decrease in device performance is inhibited in the device having the first layer with an increased film thickness. Further, none of Literatures 1 to 3 pay attention to parameters (ionization potential Ip and hole mobility μh) of the compound used for the first layer and parameters (ionization potential Ip) of the compound used for the second layer (e.g., hole transporting layer close to the anode).

In the organic EL device according to the exemplary embodiment, the ionization potential Ip(HT1) of the first compound and the ionization potential Ip(HT2) of the second compound preferably satisfy a numerical formula (Numerical Formula 10) below. This facilitates a decrease in voltage in the device.

Ip(HT1)>Ip(HT2) (Numerical Formula 10)

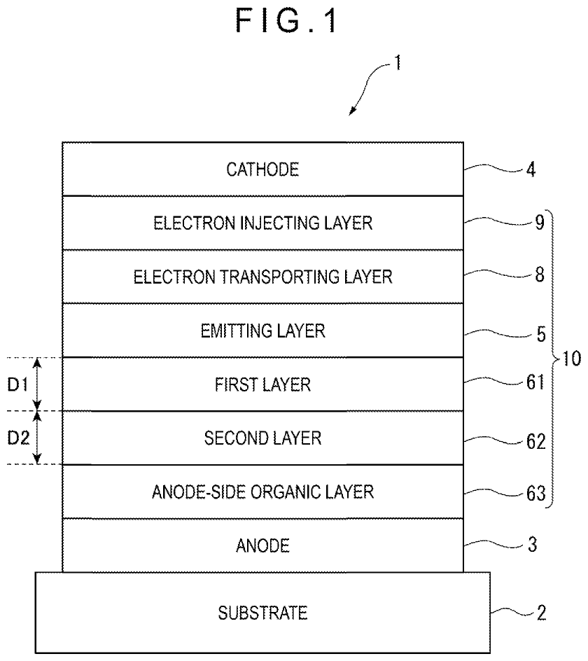

FIG. 1 schematically shows an exemplary arrangement of an organic EL device according to the exemplary embodiment.

An organic EL device 1 includes a light-transmissive substrate 2, an anode 3, a cathode 4, and an organic layer 10 provided between the anode 3 and the cathode 4. The organic layer 10 is provided by layering, on the anode 3, an anode-side organic layer 63, a second layer 62, a first layer 61, an emitting layer 5, an electron transporting layer 8, and an electron injecting layer 9 in this order. In FIG. 1, D1 represents a film thickness of the first layer 61. D1 (the film thickness of the first layer 61) is 15 nm or more. D2 represents a film thickness of the second layer 62. D2 (the film thickness of the second layer 62) is preferably in a range from 80 nm to 140 nm.

In the organic EL device 1 of FIG. 1, the hole transporting zone includes the anode-side organic layer 61, the first layer 61, and the second layer 62.

The first layer 61 is preferably in direct contact with the emitting layer 5.

The first layer 61 is also preferably in direct contact with the second layer 62.

The first layer 61 is preferably a hole transporting layer or electron blocking layer, more preferably an electron blocking layer.

The second layer 62 is preferably in direct contact with the first layer 61.

The second layer 62 is also preferably in direct contact with the anode-side organic layer 63.

The second layer 62 is preferably a hole transporting layer.

The anode-side organic layer 63 is also preferably in direct contact with the second layer 62.

The anode-side organic layer 63 is also preferably in direct contact with the anode 3.

The anode-side organic layer 63 is preferably a hole injecting layer or hole transporting layer, more preferably a hole injecting layer.

The anode-side organic layer 63 can be provided by using, for instance, materials for the hole injecting layer and hole transporting layer described in after-described Arrangement of Organic EL Device.

The emitting layer 5 preferably contains no phosphorescent material (dopant material).

The emitting layer 5 preferably contains no phosphorescent metal complex.

The emitting layer 5 preferably contains no heavy metal complex. Examples of the heavy metal complex include an iridium complex, osmium complex, and platinum complex.

The emitting layer 5 preferably contains no phosphorescent rare-earth metal complex.

The emitting layer 5 may contain a metal complex, but preferably contains no metal complex.

In an exemplary arrangement of the exemplary embodiment, the first layer has a film thickness of 25 nm or more.

In another exemplary arrangement of the exemplary embodiment, the first layer has a film thickness of 35 nm or more.

In still another exemplary arrangement of the exemplary embodiment, the first layer has a film thickness of 45 nm or more.

In a further exemplary arrangement of the exemplary embodiment, the first layer has a film thickness of 55 nm or more.

In a still further exemplary arrangement of the exemplary embodiment, the first layer has a film thickness of 110 nm or less.

In a still further exemplary arrangement of the exemplary embodiment, the first layer has a film thickness of 100 nm or less.

In a still further exemplary arrangement of the exemplary embodiment, the first layer has a film thickness of 90 nm or less.

In an exemplary arrangement of the exemplary embodiment, the second layer has a film thickness of 80 nm or more.

In another exemplary arrangement of the exemplary embodiment, the second layer has a film thickness of 90 nm or more.

In still another exemplary arrangement of the exemplary embodiment, the second layer has a film thickness of 100 nm or more.

In a further exemplary arrangement of the exemplary embodiment, the second layer has a film thickness of 140 nm or less.

In a still further exemplary arrangement of the exemplary embodiment, the second layer has a film thickness of 130 nm or less.

In a still further exemplary arrangement of the exemplary embodiment, the second layer has a film thickness of 120 nm or less.

### First Layer

The first layer contains the first compound.

The first compound may be any compound having an ionization potential Ip(HT1) of 5.69 eV or more (Numerical Formula 1) and a hole mobility μh(HT1) of 1.00×10⁻⁵ cm²/Vs or more (Numerical Formula 2).

The first compound is preferably an amine compound.

As the first compound, for instance, a compound having an ionization potential Ip(HT1) of 5.69 eV or more and a hole mobility μh(HT1) of 1.00×10⁻⁵ cm²/Vs or more can be selected for use from compounds represented by formulae (31) to (33) and a formula (X) below.

### Compounds Represented by Formulae (31) to (33)

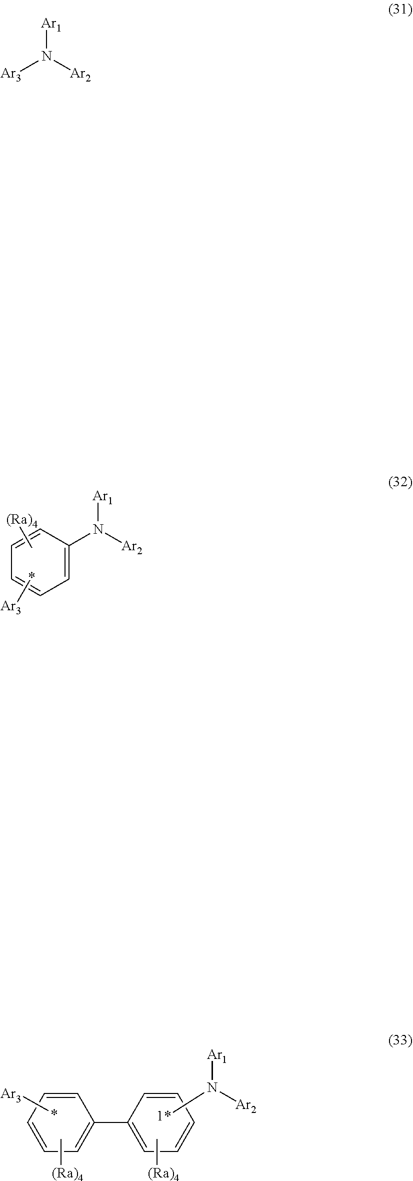

In the formulae (31) to (33):

Ar₁ and Ar₂ are each independently a substituted or unsubstituted aryl group having 6 to 50 ring carbon atoms, or a substituted or unsubstituted heterocyclic group having 5 to 50 ring atoms;

each Ar₃ is independently a group represented by a formula (3A) or (3B); in the formula (32), * represents a bonding position to a carbon atom in a six-membered ring having Ra; in the formula (33), * represents a bonding position to a carbon atom in a six-membered ring having Ra; and in the formula (33), 1* represents a bonding position to a carbon atom in a six-membered ring having Ra;

at least one combination of adjacent two or more of a plurality of Ra are mutually bonded to form a substituted or unsubstituted monocyclic ring, mutually bonded to form a substituted or unsubstituted fused ring, or not mutually bonded;

Ra forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms; and the plurality of Ra are mutually the same or different.

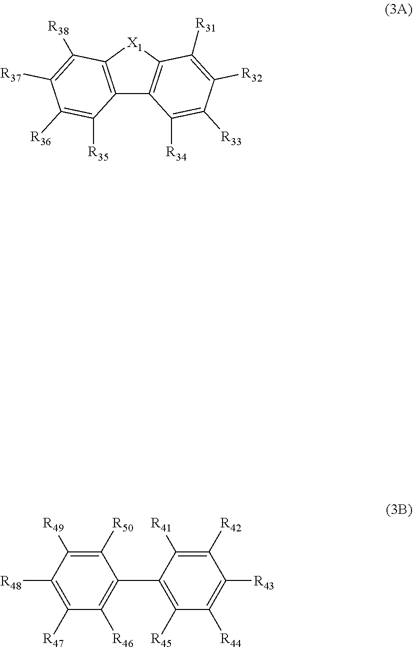

In the formulae (3A) and (3B):

X₁ is an oxygen atom, sulfur atom, CR₃₀₁R₃₀₂, or NR₃₀₃;

a combination of R₃₀₁ and R₃₀₂ are mutually bonded to form a substituted or unsubstituted monocyclic ring, mutually bonded to form a substituted or unsubstituted fused ring, or not mutually bonded;

at least one combination of adjacent two or more of R₃₁ to R₃₄ are mutually bonded to form a substituted or unsubstituted monocyclic ring, mutually bonded to form a substituted or unsubstituted fused ring, or not mutually bonded;

at least one combination of adjacent two or more of R₃₅ to R₃₈ are mutually bonded to form a substituted or unsubstituted monocyclic ring, mutually bonded to form a substituted or unsubstituted fused ring, or not mutually bonded;

at least one combination of adjacent two or more of R₄₁ to R₅₀ are mutually bonded to form a substituted or unsubstituted monocyclic ring, mutually bonded to form a substituted or unsubstituted fused ring, or not mutually bonded;

R₃₀₃, and R₃₀₁, R₃₀₂, R₃₁ to R₃₈ and R₄₁ to R₅₀ forming neither the substituted or unsubstituted monocyclic ring nor the substituted or unsubstituted fused ring each independently represent the same as Ra in the formula (32); and any one of R₃₀₁ to R₃₀₃ and R₃₁ to R₃₈ in the formula (3A) is a single bond bonded to a nitrogen atom in the formula (31), a single bond bonded to a carbon atom in a six-membered ring in the formula (32), or a single bond bonded to a carbon atom in a six-membered ring in the formula (33); and any one of R₄₁ to R₅₀ in the formula (3B) is a single bond bonded to a nitrogen atom in the formula (31), a single bond bonded to a carbon atom in a six-membered ring in the formula (32), or a single bond bonded to a carbon atom in a six-membered ring in the formula (33).

In the first compound, R₉₀₁, R₉₀₂, R₉₀₃, R₉₀₄, R₉₀₅, R₉₀₆, R₉₀₇, R₉₀₈, R₉₀₉, R₉₃₁, R₉₃₂, R₉₃₃, R₉₃₄, R₉₃₅, R₉₃₆, and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₉₀₈ are present, the plurality of R₉₀₈ are mutually the same or different;

when a plurality of R₉₀₉ are present, the plurality of R₉₀₉ are mutually the same or different;

when a plurality of R₉₃₁ are present, the plurality of R₉₃₁ are mutually the same or different;

when a plurality of R₉₃₂ are present, the plurality of R₉₃₂ are mutually the same or different;

when a plurality of R₉₃₃ are present, the plurality of R₉₃₃ are mutually the same or different;

when a plurality of R₉₃₄ are present, the plurality of R₉₃₄ are mutually the same or different;

when a plurality of R₉₃₅ are present, the plurality of R₉₃₅ are mutually the same or different;

when a plurality of R₉₃₆ are present, the plurality of R₉₃₆ are mutually the same or different; and

when a plurality of R₉₃₇ are present, the plurality of R₉₃₇ are mutually the same or different.

The first compound is also preferably a compound represented by a formula (X) below.

### Compound Represented by Formula (X)

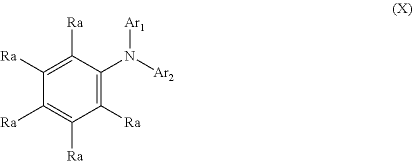

In the formula (X):

Ar₁ and Ar₂ each independently represent the same as Ar₁ and Ar₂ in the formula (32);

at least one combination of adjacent two or more of a plurality of Ra are mutually bonded to form a substituted or unsubstituted monocyclic ring, mutually bonded to form a substituted or unsubstituted fused ring, or not mutually bonded;

Ra forming neither the substituted or unsubstituted monocyclic ring nor the substituted or unsubstituted fused ring each independently represent the same as Ra forming neither the substituted or unsubstituted monocyclic ring nor the substituted or unsubstituted fused ring in the formula (32); and the plurality of Ra are mutually the same or different.

When the first compound is a compound represented by the formula (31), it is preferable that each Ar₃ is independently a group represented by any of formulae (30A) to (30G) below.

When the first compound is a compound represented by the formula (32) or (33), it is preferable that each Ar₃ is independently a group represented by any of formulae (30A) to (30H) below.

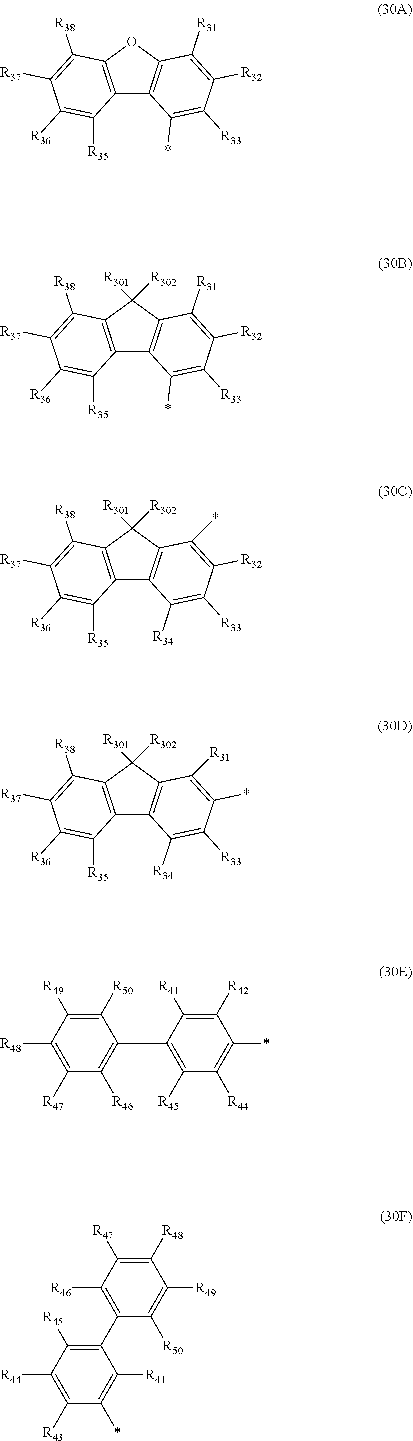

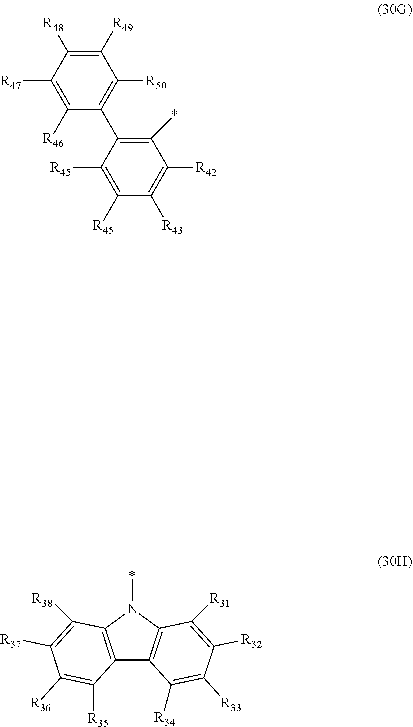

In the formulae (30A) to (30D), R₃₀₁, R₃₀₂, and R₃₁ to R₃₈ each independently represent the same as R₃₀₁, R₃₀₂, and R₃₁ to R₃₈ in the formula (3A); in the formulae (30E) to (30G), R₄₁ to R₅₀ each independently represent the same as R₄₁ to R₅₀ in the formula (3B); and in the formula (30H), R₃₁ to R₃₈ each independently represent the same as R₃₁ to R₃₈ in the formula (3A); and each * represents a bonding position.

In the first compound, it is preferable that Ar₁ and Ar₂ are each independently a substituted or unsubstituted phenyl group, a substituted or unsubstituted biphenyl group, a substituted or unsubstituted terphenyl group, a substituted or unsubstituted dibenzofuranyl group, a substituted or unsubstituted dibenzothienyl group, a substituted or unsubstituted fluorenyl group, a substituted or unsubstituted carbazolyl group, a substituted or unsubstituted naphthyl group, or a substituted or unsubstituted phenanthrenyl group.

In the first compound, it is more preferable that Ar₁ and Ar₂ are each independently an unsubstituted phenyl group, an unsubstituted biphenyl group, an unsubstituted terphenyl group, an unsubstituted dibenzofuranyl group, an unsubstituted dibenzothienyl group, a substituted or unsubstituted fluorenyl group, a substituted or unsubstituted carbazolyl group, an unsubstituted naphthyl group, or an unsubstituted phenanthrenyl group.

The first compound is preferably a compound represented by any of formulae (301) to (310) below.

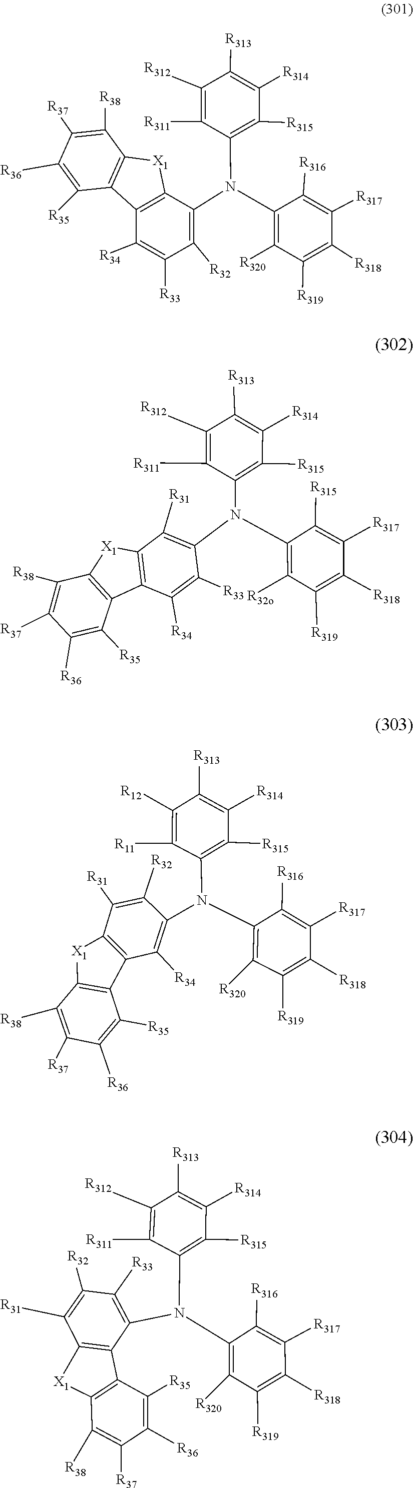

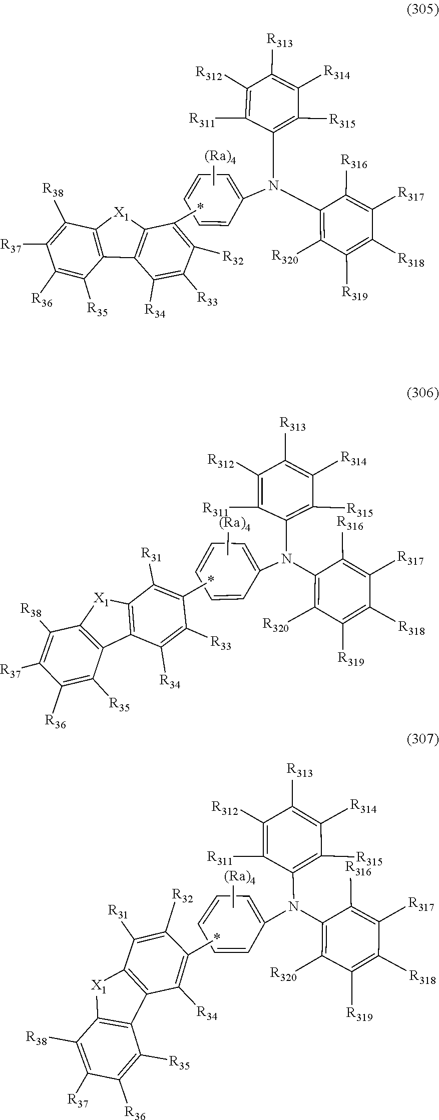

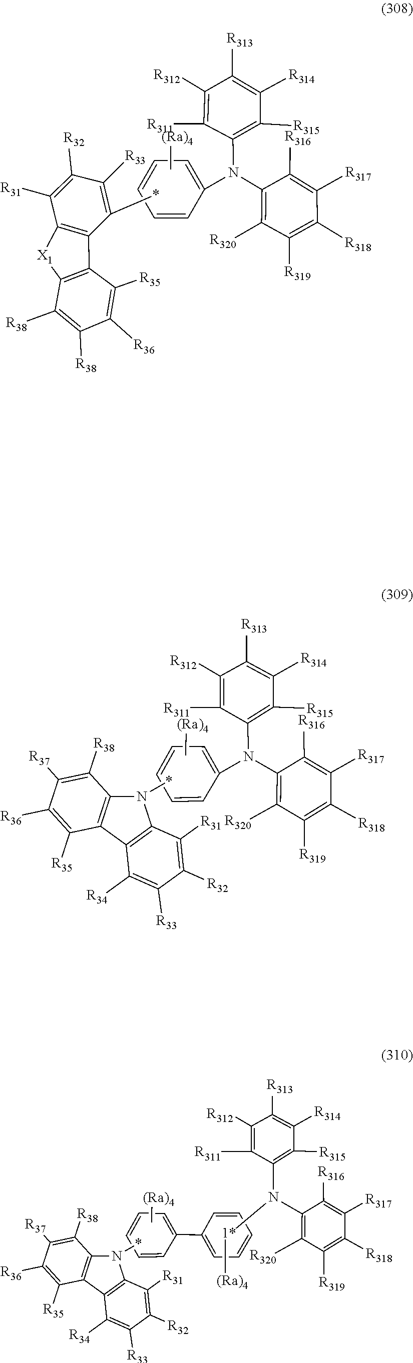

In the formulae (301) to (310):

X₁ and R₃₁ to R₃₈ each independently represent the same as X₁ and R₃₁ to R₃₈ in the formula (3A); each Ra independently represents the same as Ra in the formula (32);

at least one combination of adjacent two or more of R₃₁₁ to R₃₁₅ are mutually bonded to form a substituted or unsubstituted monocyclic ring, mutually bonded to form a substituted or unsubstituted fused ring, or not mutually bonded;

at least one combination of adjacent two or more of R₃₁₆ to R₃₂₀ are mutually bonded to form a substituted or unsubstituted monocyclic ring, mutually bonded to form a substituted or unsubstituted fused ring, or not mutually bonded;

R₃₁₁ to R₃₂₀ forming neither the substituted or unsubstituted monocyclic ring nor the substituted or unsubstituted fused ring each independently represent the same as Ra in the formula (32);

each * represents a bonding position to a carbon atom in a six-membered ring having Ra; and

1* represents a bonding position to a carbon atom in a six-membered ring having Ra.

In the first compound, it is preferable that R₃₁ to R₃₈, R₄₁ to R₅₀, R₃₀₁ to R₃₀₃ and Ra are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the first compound, it is preferable that R₃₁ to R₃₈ and R₄₁ to R₅₀ are each independently a hydrogen atom, or a substituted or unsubstituted phenyl group.

In the first compound, it is preferable that R₃₁ to R₃₈ and R₄₁ to R₅₀ are each a hydrogen atom.

In the first compound, it is preferable that R₃₀₁ to R₃₀₃ are each independently a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the first compound, it is preferable that R₃₀₁ and R₃₀₂ are each independently a substituted or unsubstituted alkyl group having 1 to 50 carbon atoms, or a substituted or unsubstituted aryl group having 6 to 50 ring carbon atoms.

In the first compound, it is more preferable that R₃₀₁ and R₃₀₂ are each independently a methyl group, or a substituted or unsubstituted phenyl group.

In the first compound, it is also preferable that a combination of R₃₀₁ and R₃₀₂ are mutually bonded to form a substituted or unsubstituted monocyclic ring, or mutually bonded to form a substituted or unsubstituted fused ring.

In the first compound: it is preferable that each Ra is independently a hydrogen atom, an unsubstituted alkyl group having 1 to 50 carbon atoms, an unsubstituted aryl group having 6 to 50 ring carbon atoms, or an unsubstituted heterocyclic group having 5 to 50 ring atoms; and it is more preferable that each Ra is independently a hydrogen atom.

In the first compound, it is preferable that R₃₁₁ to R₃₂₀ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the first compound, it is also preferable that at least one combination of adjacent two or more of R₃₁₁ to R₃₁₅ are mutually bonded to form a substituted or unsubstituted monocyclic ring, or mutually bonded to form a substituted or unsubstituted fused ring.

In the first compound, it is also preferable that at least one combination of adjacent two or more of R₃₁₆ to R₃₂₀ are mutually bonded to form a substituted or unsubstituted monocyclic ring, or mutually bonded to form a substituted or unsubstituted fused ring.

It is preferable that each substituent for the “substituted or unsubstituted” group for Ar₁ and Ar₂ is independently a substituted or unsubstituted phenyl group, a substituted or unsubstituted biphenyl group, a substituted or unsubstituted terphenyl group, a substituted or unsubstituted dibenzofuranyl group, a substituted or unsubstituted dibenzothienyl group, a substituted or unsubstituted fluorenyl group, a substituted or unsubstituted carbazolyl group, a substituted or unsubstituted naphthyl group, or a substituted or unsubstituted phenanthrenyl group.

It is more preferable that each substituent for the “substituted or unsubstituted” group for Ar₁ and Ar₂ is independently an unsubstituted phenyl group, an unsubstituted biphenyl group, an unsubstituted dibenzofuranyl group, an unsubstituted dibenzothienyl group, a substituted or unsubstituted fluorenyl group, a substituted or unsubstituted carbazolyl group, an unsubstituted naphthyl group, or an unsubstituted phenanthrenyl group.

The first compound is also preferably a compound represented by a formula (10) below.

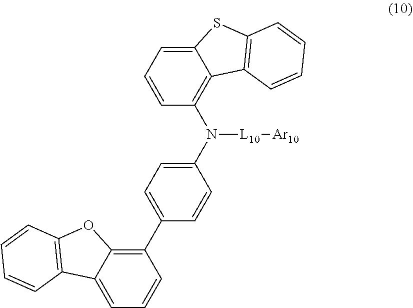

In the formula (10):

L₁₀ is a single bond, or a substituted or unsubstituted arylene group having 6 to 12 ring carbon atoms;

a substituent, if present, for L₁₀ is an unsubstituted phenyl group;

Ar₁₀ is a substituted or unsubstituted aryl group having 6 to 18 ring carbon atoms; and a substituent, if present, for Ar₁₀ is an unsubstituted phenyl group or an unsubstituted naphthyl group.

The compound represented by the formula (10) represents the same as a compound according to a sixth exemplary embodiment, and a preferable range thereof is also the same as that of the compound according to the sixth exemplary embodiment.

In the formula (10), L₁₀ is preferably a single bond, or a substituted or unsubstituted phenylene group.

In the formula (10), Ar₁₀ is preferably a group represented by any of formulae (10a) to (27a) below, and more preferably a group represented by any of the formulae (10a) to (14a), (17a), (18a), and (26a).

In the formula (10), Ar₁₀ is further preferably a group represented by any of formulae (10a) to (13a), (14a-1) to (18a-1), (20a), (21a), and (26a-1) below.

Further, the compound represented by the formula (10) is also an exemplary arrangement of a compound represented by the formula (305).

In the first compound, it is preferable that each substituent for the “substituted or unsubstituted” group is independently the same as a substituent for the “substituted or unsubstituted” group in Ar₁ and Ar₂.

### Ionization Potential Ip(HT1) of First Compound

The ionization potential Ip(HT1) of the first compound satisfies the numerical formula (Numerical Formula 1) below.

The ionization potential Ip(HT1) of the first compound preferably satisfies a numerical formula (Numerical Formula 11) below. A method for measuring the ionization potential Ip is as described in Examples.

Ip(HT1)≥5.69 eV (Numerical Formula 1)

Ip(HT1)≥5.70 eV (Numerical Formula 11)

Hole Mobility μh(HT1) of First Compound

The hole mobility μh(HT1) of the first compound satisfies the numerical formula (Numerical Formula 2) below.

In an exemplary arrangement of the exemplary embodiment, the hole mobility μh(HT1) of the first compound satisfies a numerical formula (Numerical Formula 2A) below.

μh(HT1)≥1.00×10⁻⁵ cm²/Vs (Numerical Formula 2)

μh(HT1)≥1.00×10⁻⁴ cm²/Vs (Numerical Formula 2A)

### Method for Measuring Hole Mobility

Herein, the hole mobility can be measured according to an impedance measurement using a mobility evaluation device manufactured by the following steps. The mobility evaluation device is, for instance, manufactured by the following steps.

A compound HA-2 below is vapor-deposited on a glass substrate having an ITO transparent electrode (anode) so as to cover the transparent electrode, thereby forming a hole injecting layer. A compound HT-A below is vapor-deposited on this formed hole injecting layer to form a hole transporting layer. Subsequently, a compound Target, which is to be measured for a hole mobility, is vapor-deposited to form a measurement target layer. Metal aluminum (Al) is vapor-deposited on this measurement target layer to form a metal cathode.

An arrangement of the mobility evaluation device above is roughly shown as follows.

ITO(130)/HA-2(5)/HT-A(10)/Target(200)/Al(80)

Numerals in parentheses represent a film thickness (nm).

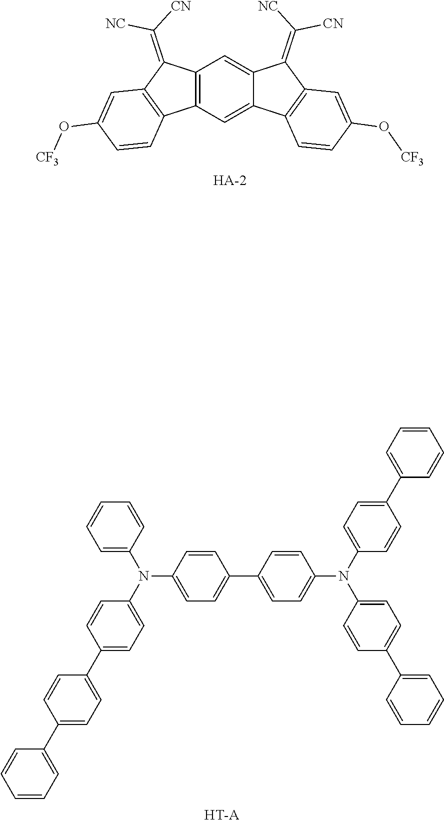 [SMILES: N#CC(C#N)=C1c2cc(OC(F)(F)F)ccc2-c2cc3c(cc21)C(=C(C#N)C#N)c1cc(OC(F)(F)F)ccc1-3 ; "HA-2" ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; "HT-A"]

The mobility evaluation device for the hole mobility is set in an impedance measurement device to perform an impedance measurement. In the impedance measurement, a measurement frequency is swept from 1 Hz to 1 MHz. At this time, an alternating current amplitude of 0.1 V and a direct current voltage V are applied to the device. A modulus M is calculated from a measured impedance Z using the relationship of the calculation formula (C1).

In a bode plot in which an imaginary part of the modulus M is represented by an ordinate axis and the frequency [Hz] is represented by an abscissa axis, an electrical time constant τ of the mobility evaluation device is obtained from a frequency fmax showing a peak using the calculation formula (C2).

A hole mobility μh is calculated from a relationship of a calculation formula (C3-2) below using τ obtained from the calculation formula (C2).

μh=d²/(Vτ) Calculation Formula (C3-2):

d in the calculation formula (C3-2) is a total film thickness of organic thin film(s) forming the device. In a case of the arrangement of the mobility evaluation device for the hole mobility, d=215 [nm] is satisfied.

The hole mobility herein is a value obtained in a case where a square root of an electric field intensity meets E^{1/2}=500 [V^{1/2}/cm^{1/2}]. The square root of the electric field intensity, E^{1/2}, can be calculated from a relationship of a calculation formula (C4) below.

E^{1/2}=V^{1/2}/d^{1/2} Calculation Formula (C4):

For the impedance measurement, a 1260 type by Solartron Analytical is used as the impedance measurement device, and for a higher accuracy, a 1296 type dielectric constant measurement interface by Solartron Analytical can be used together therewith.

### Method for Manufacturing First Compound

The first compound can be manufactured by a known method.

### Specific Examples of First Compound

Specific examples of the first compound include the following compounds.

It should however be noted that the invention is not limited to the specific examples of the compound.

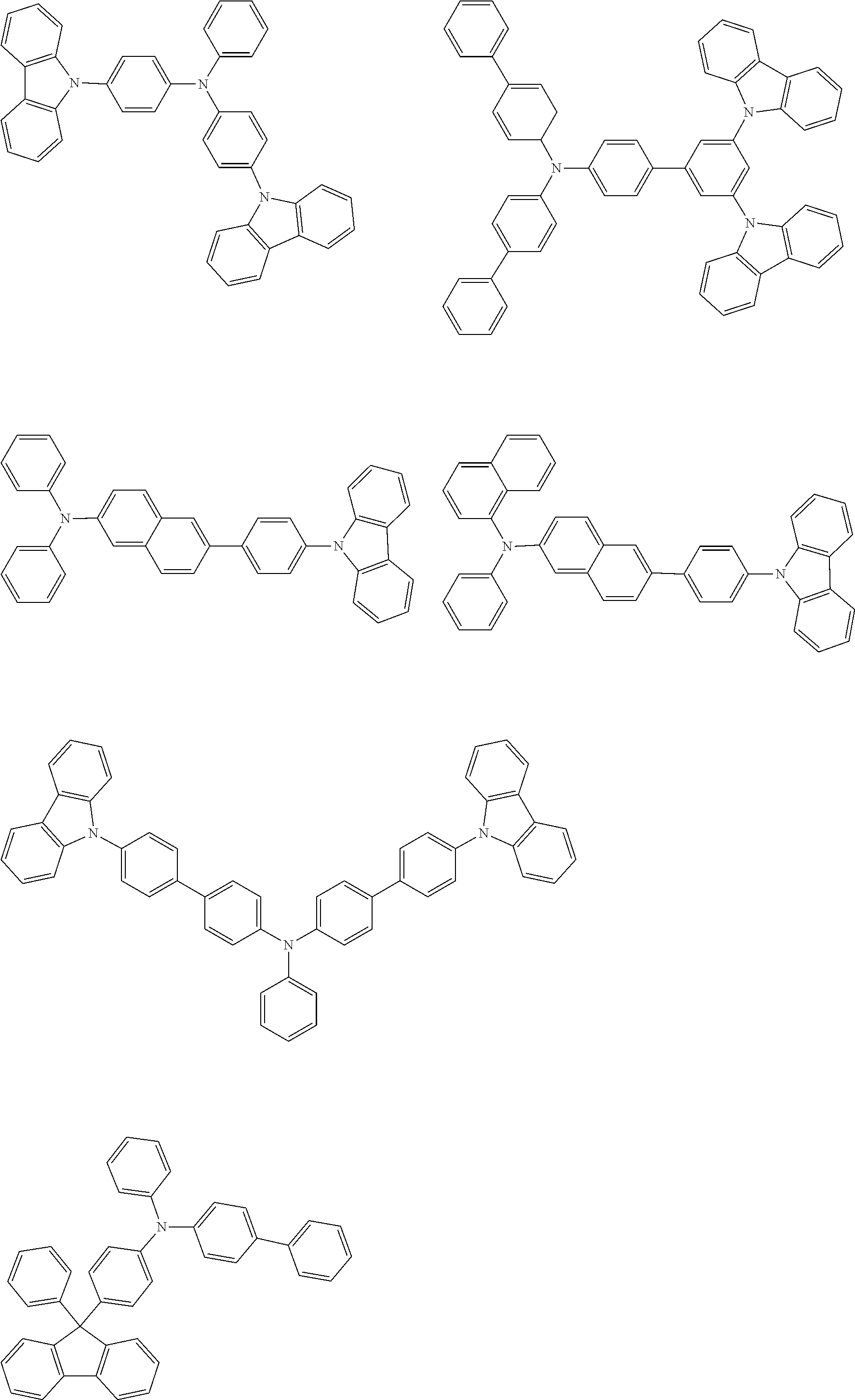

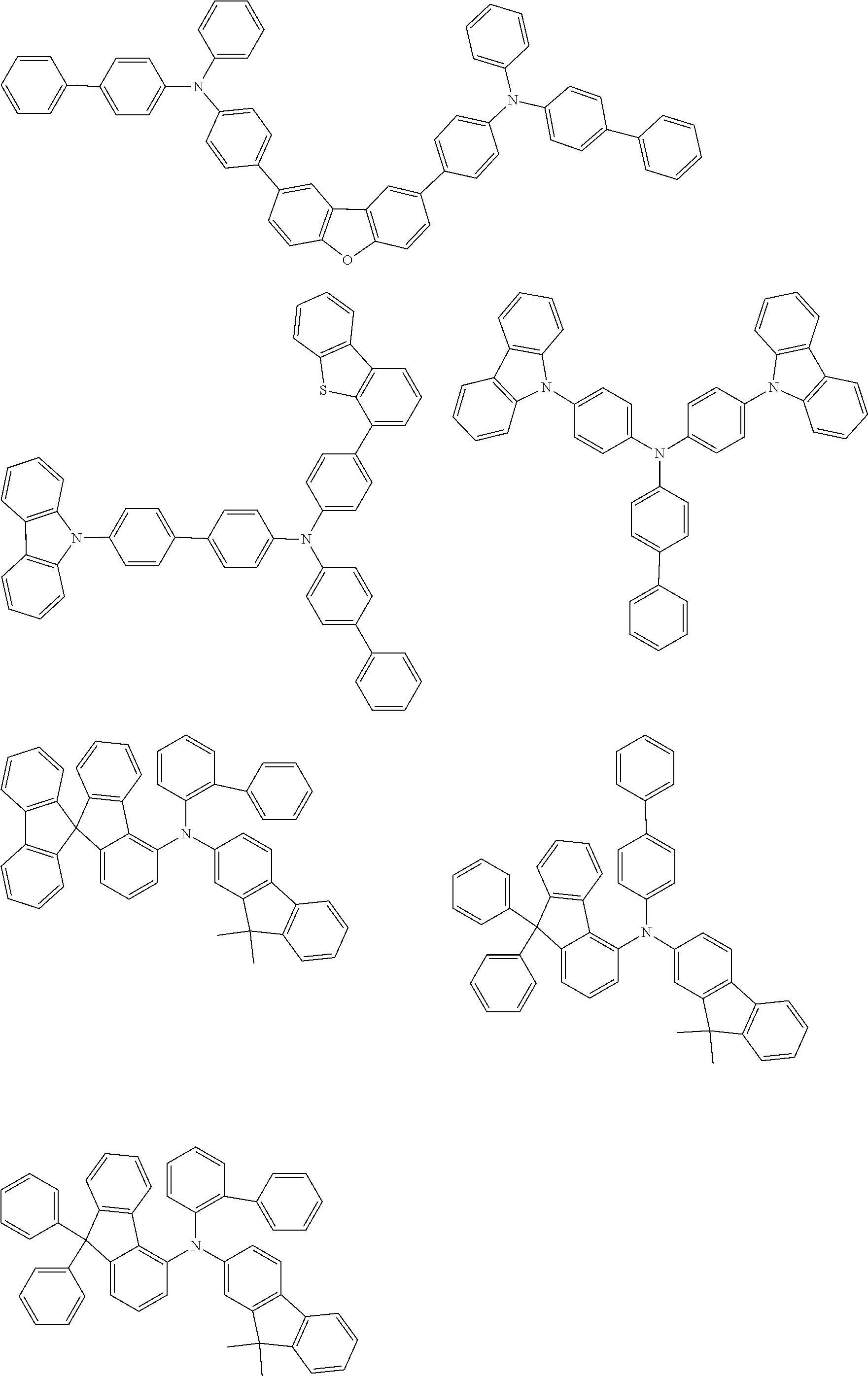

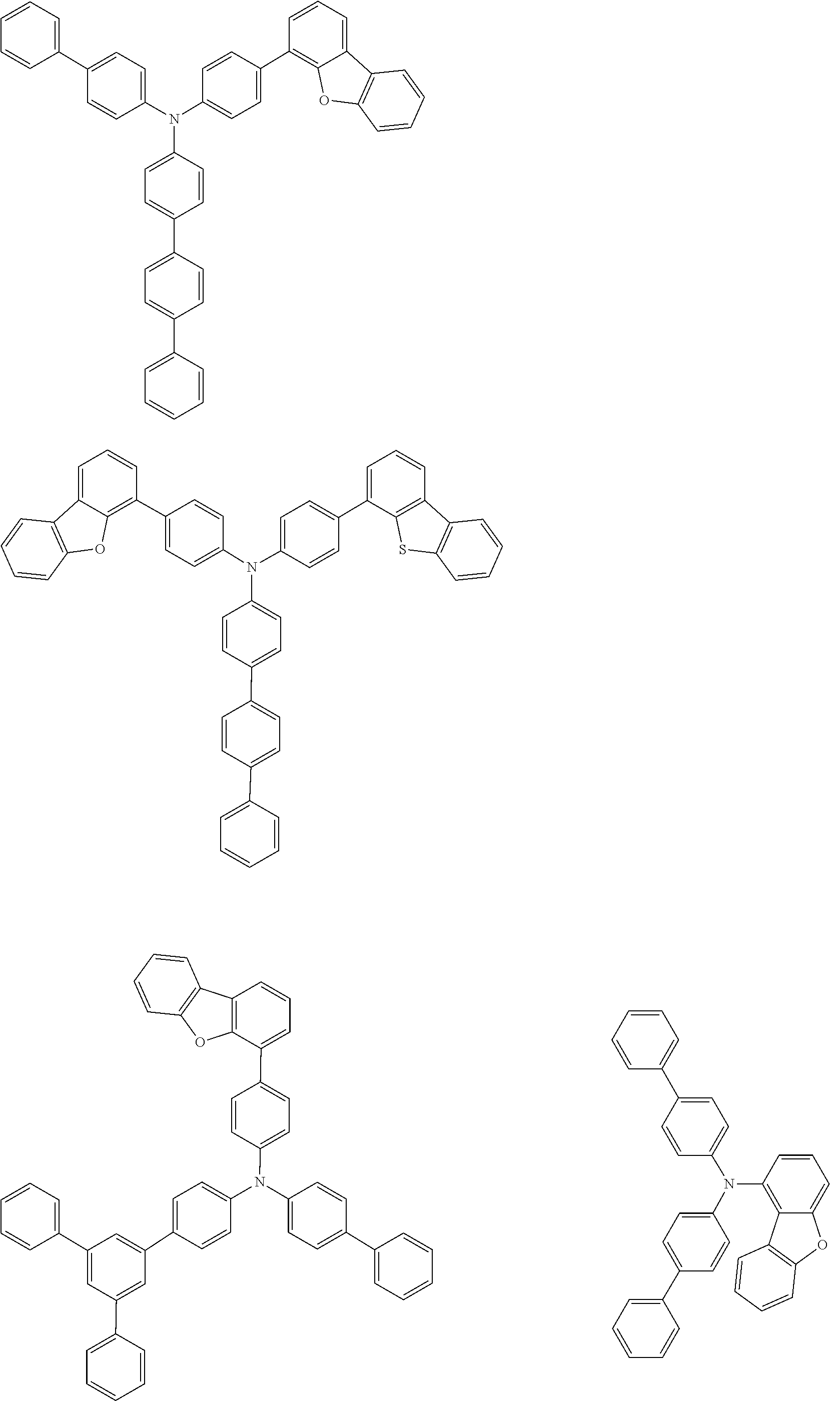 [SMILES: c1ccc(-c2ccc(-c3ccc(N(c4ccc(-c5ccccc5)cc4)c4ccc(-c5cccc6c5oc5ccccc56)cc4)cc3)cc2)cc1 ; c1ccc(-c2ccc(-c3ccc(N(c4ccc(-c5cccc6c5oc5ccccc56)cc4)c4ccc(-c5cccc6c5sc5ccccc56)cc4)cc3)cc2)cc1 ; c1ccc(-c2ccc(N(c3ccc(-c4cc(-c5ccccc5)cc(-c5ccccc5)c4)cc3)c3ccc(-c4cccc5c4oc4ccccc45)cc3)cc2)cc1 ; c1ccc(-c2ccc(N(c3ccc(-c4ccccc4)cc3)c3cccc4oc5ccccc5c34)cc2)cc1]

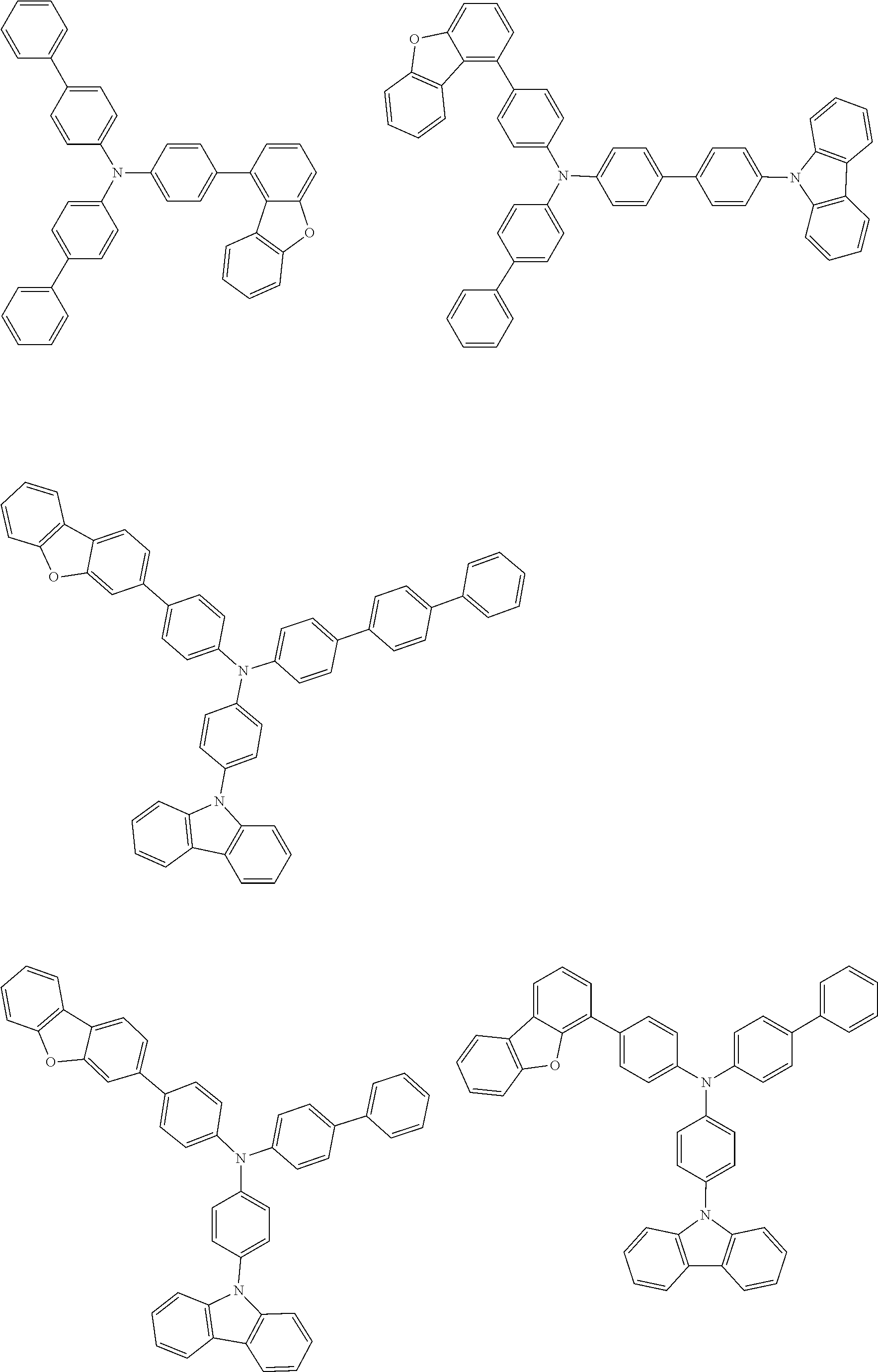

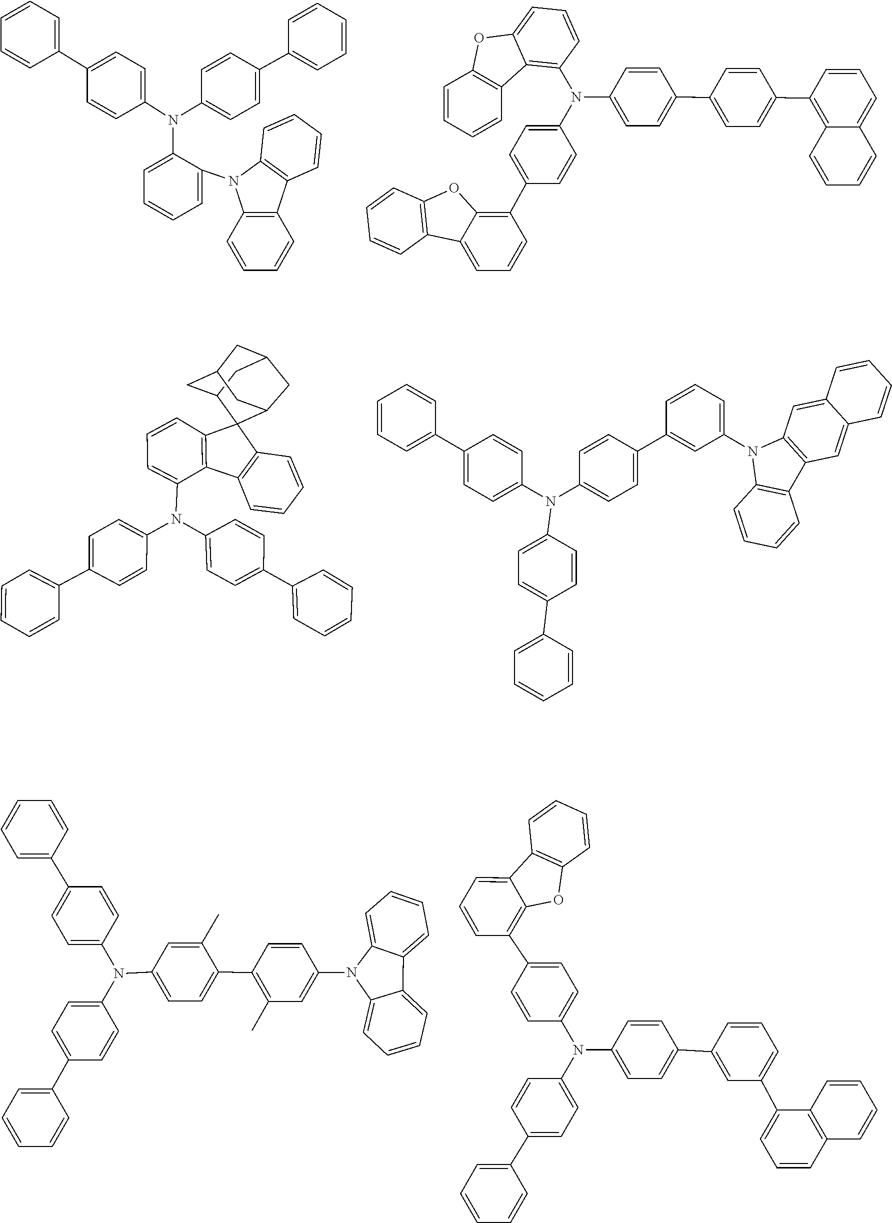

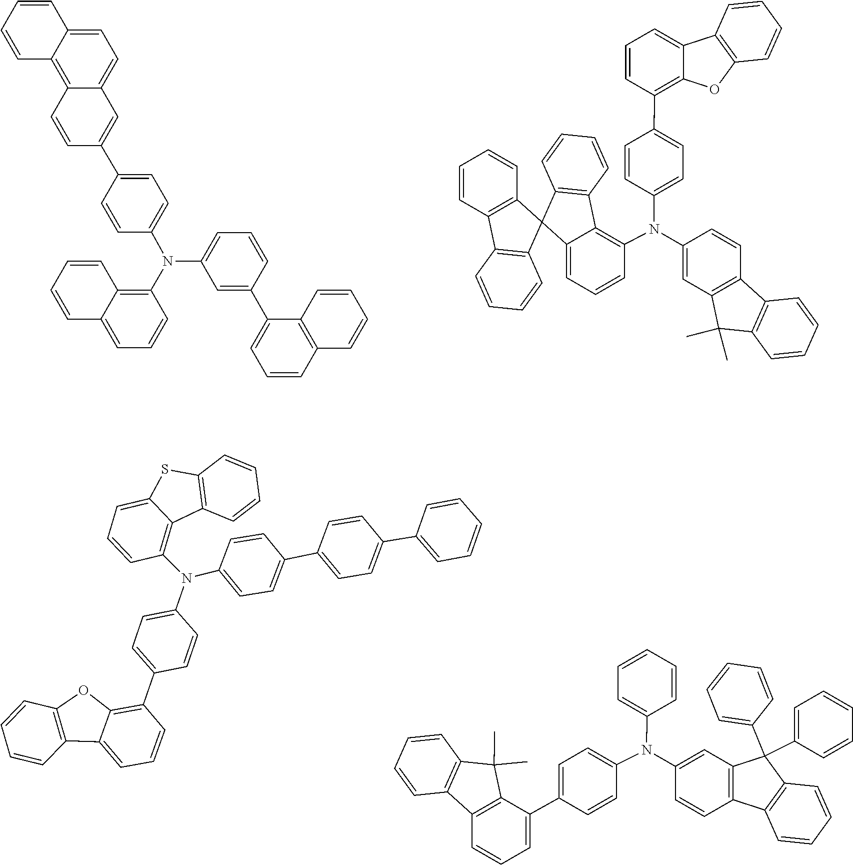

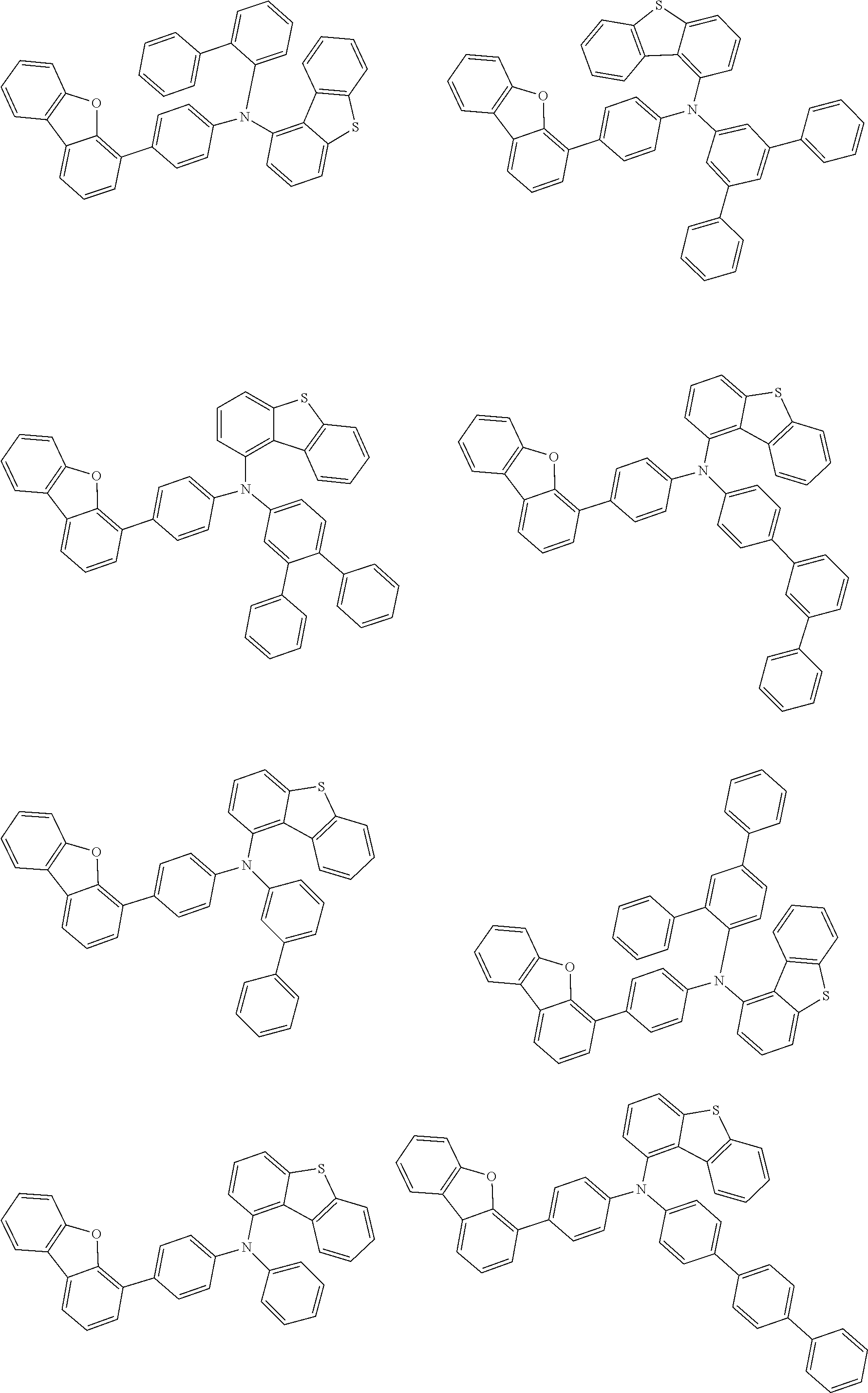

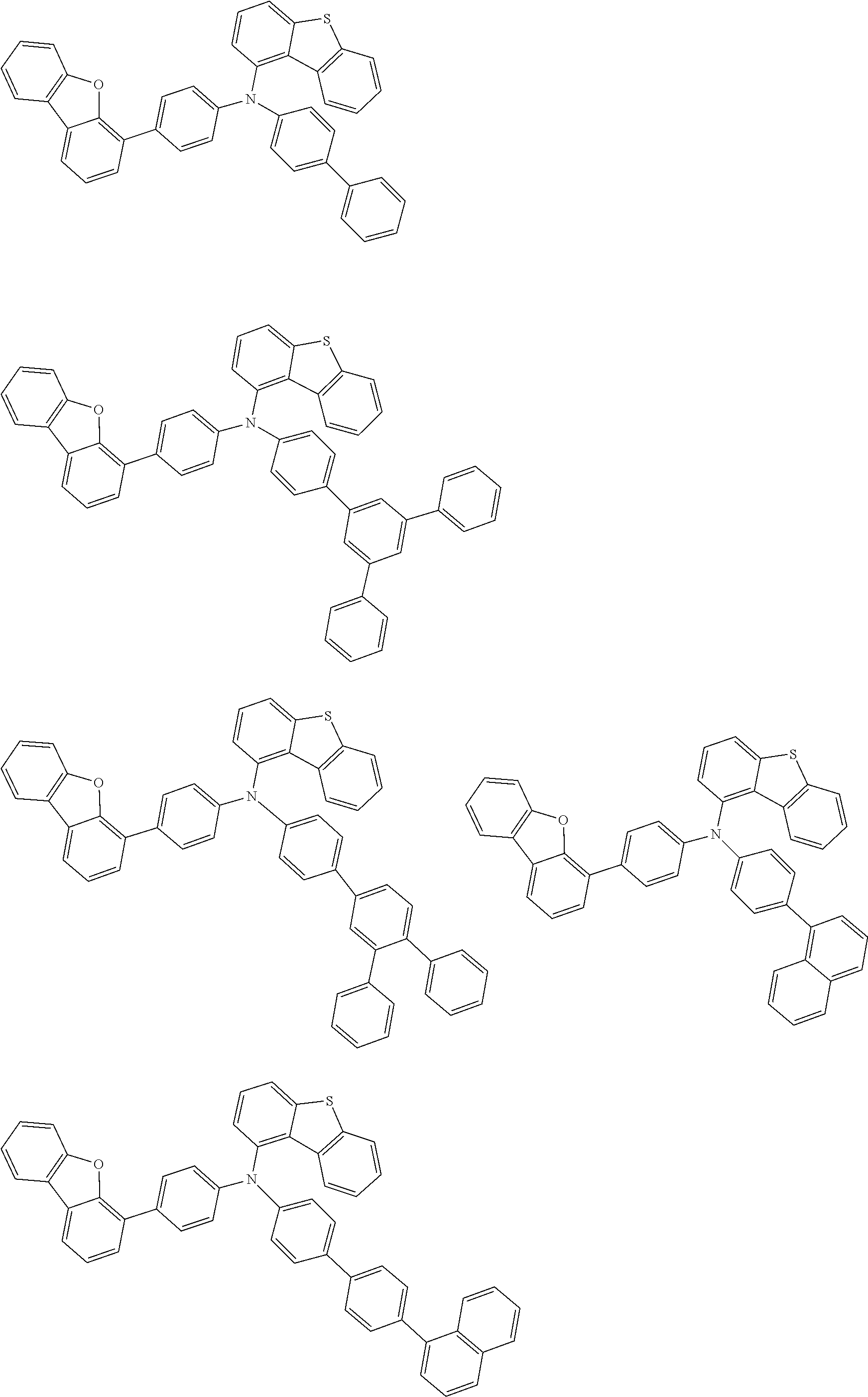 [SMILES: c1ccc(-c2ccc(N(c3ccc(-c4cccc5c4oc4ccccc45)cc3)c3cccc4sc5ccccc5c34)cc2)cc1 ; c1ccc(-c2cc(-c3ccccc3)cc(-c3ccc(N(c4ccc(-c5cccc6c5oc5ccccc56)cc4)c4cccc5sc6ccccc6c45)cc3)c2)cc1 ; c1ccc(-c2ccc(-c3ccc(N(c4ccc(-c5cccc6c5oc5ccccc56)cc4)c4cccc5sc6ccccc6c45)cc3)cc2-c2ccccc2)cc1 ; c1ccc2c(-c3ccc(N(c4ccc(-c5cccc6c5oc5ccccc56)cc4)c4cccc5sc6ccccc6c45)cc3)cccc2c1 ; c1ccc2c(-c3ccc(-c4ccc(N(c5ccc(-c6cccc7c6oc6ccccc67)cc5)c5cccc6sc7ccccc7c56)cc4)cc3)cccc2c1]

### Second Layer

The second layer contains the second compound.

The second compound may be any compound having an ionization potential Ip(HT2) of 5.60 eV or more (Numerical Formula 3).

The second compound is preferably an amine compound.

As the second compound, for instance, a compound having an ionization potential Ip(HT2) of 5.60 eV or more can be selected for use from compounds represented by the formulae (31) to (33) and the formula (X).

The description of the formulae (31) to (33) and the formula (X) for the first compound can be applied to the second compound.

### Ionization Potential Ip(HT2) of Second Compound

The ionization potential Ip(HT2) of the second compound satisfies the numerical formula (Numerical Formula 3) below. The ionization potential Ip(HT2) of the second compound preferably satisfies a numerical formula (Numerical Formula 31) below.

Ip(HT2)≥5.60 eV (Numerical Formula 3)

Ip(HT2)≥5.65 eV (Numerical Formula 31)

Hole Mobility μh(HT2) of Second Compound

In an exemplary arrangement of the exemplary embodiment, the hole mobility μh(HT2) of the second compound satisfies a numerical formula (Numerical Formula 33) below.

In an exemplary arrangement of the exemplary embodiment, the hole mobility μh(HT2) of the second compound satisfies a numerical formula (Numerical Formula 33A) below.

μh(HT2)≥1.00×10⁻⁵ cm²/Vs (Numerical Formula 33)

μh(HT2)≥1.00×10⁻⁴ cm²/Vs (Numerical Formula 33A)

### Method for Manufacturing Second Compound

The second compound can be manufactured by a known method.

Specific Examples of Second Compound Specific examples of the second compound include the following compounds.

It should however be noted that the invention is not limited to the specific examples of the compound.

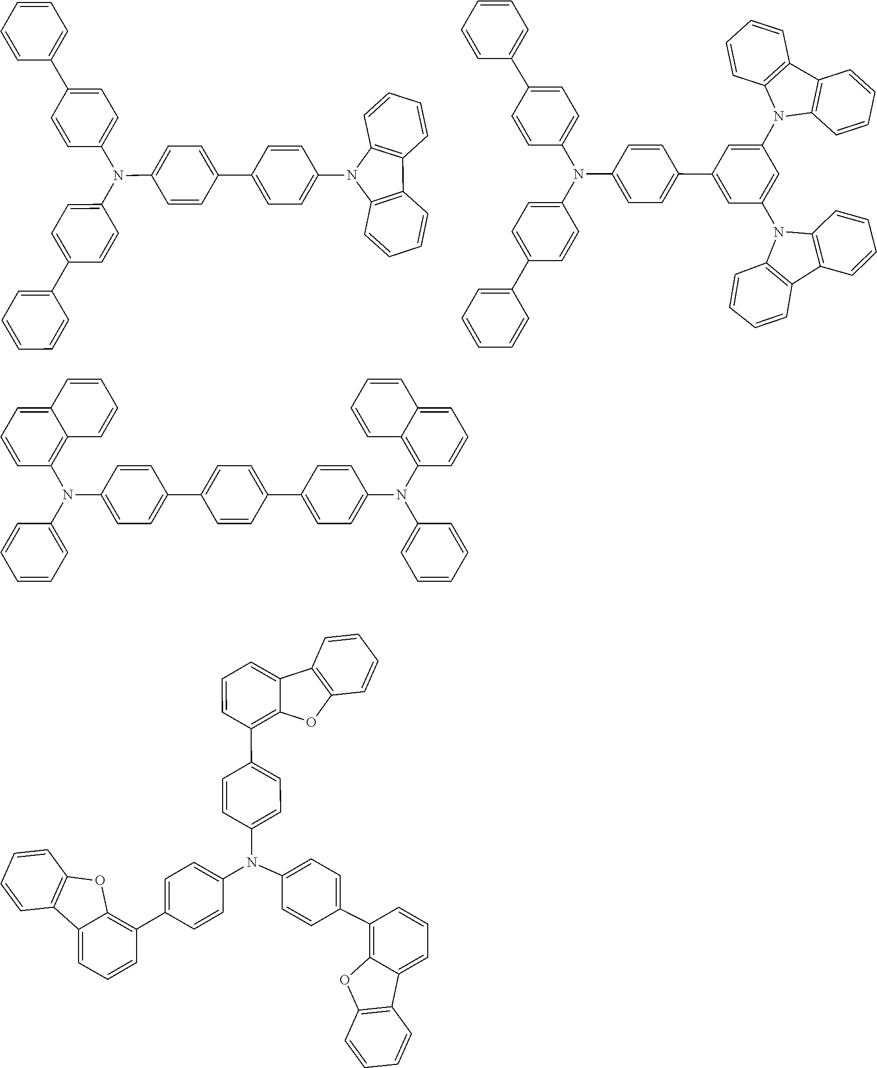

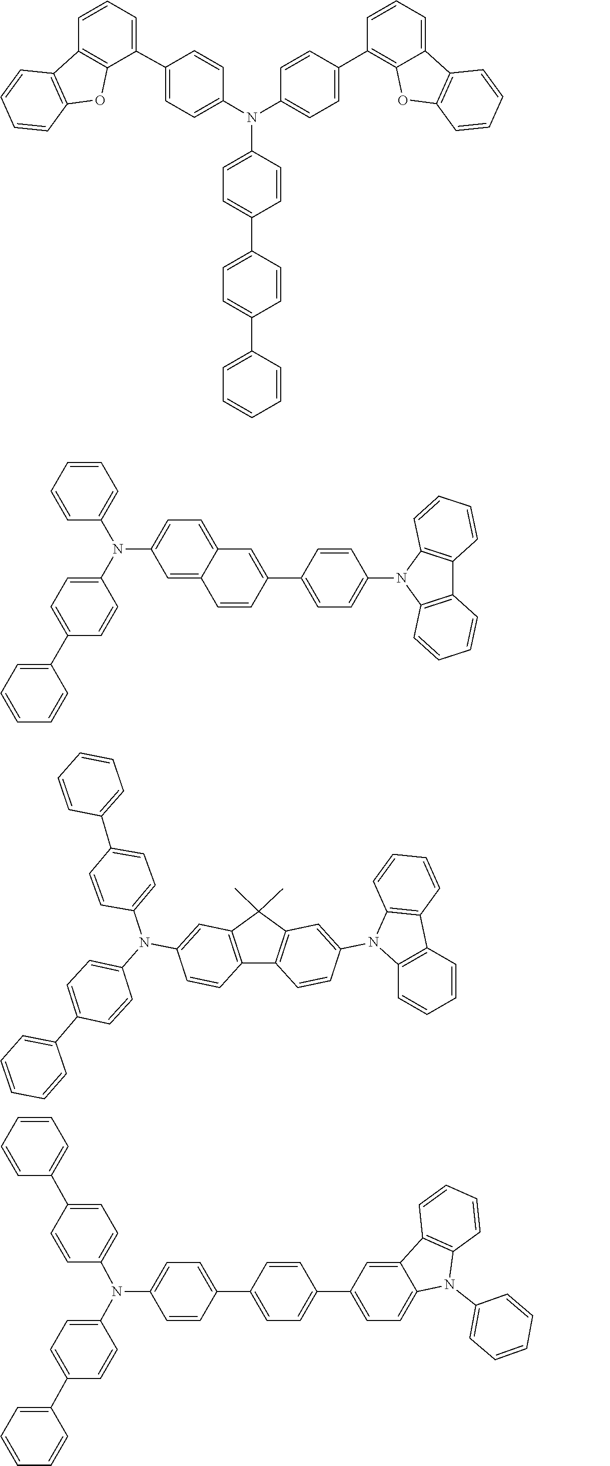

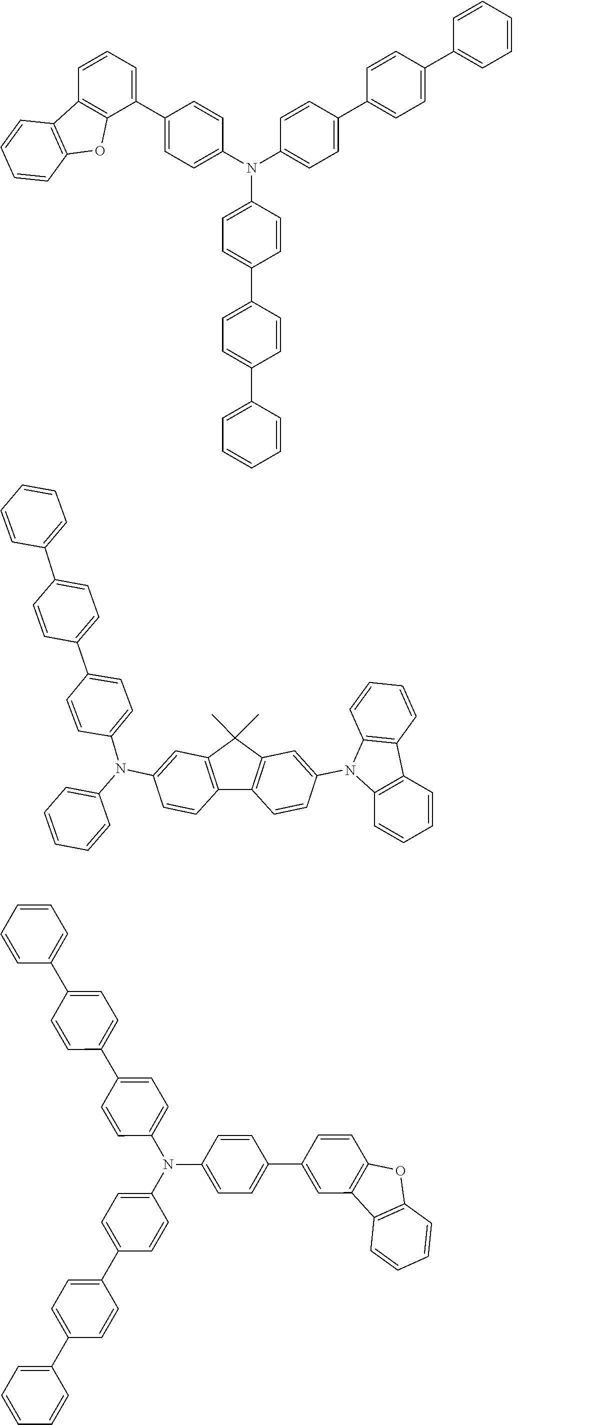 [SMILES: c1ccc(-c2ccc(-c3ccc(N(c4ccc(-c5ccc(-c6ccccc6)cc5)cc4)c4ccc(-c5cccc6c5oc5ccccc56)cc4)cc3)cc2)cc1 ; CC1(C)c2cc(N(c3ccccc3)c3ccc(-c4ccc(-c5ccccc5)cc4)cc3)ccc2-c2ccc(-n3c4ccccc4c4ccccc43)cc21 ; c1ccc(-c2ccc(-c3ccc(N(c4ccc(-c5ccc(-c6ccccc6)cc5)cc4)c4ccc(-c5ccc6oc7ccccc7c6c5)cc4)cc3)cc2)cc1]

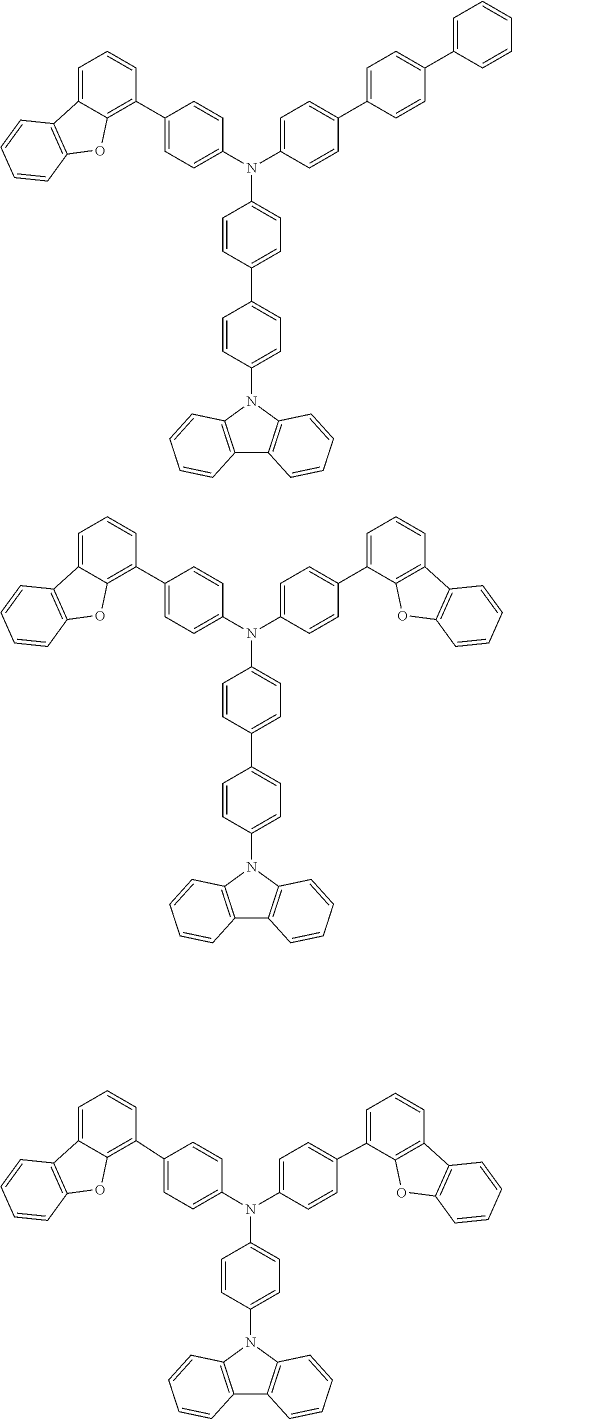 [SMILES: c1ccc(-c2ccc(-c3ccc(N(c4ccc(-c5ccc(-n6c7ccccc7c7ccccc76)cc5)cc4)c4ccc(-c5cccc6c5oc5ccccc56)cc4)cc3)cc2)cc1 ; c1ccc2c(c1)oc1c(-c3ccc(N(c4ccc(-c5ccc(-n6c7ccccc7c7ccccc76)cc5)cc4)c4ccc(-c5cccc6c5oc5ccccc56)cc4)cc3)cccc12 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12]

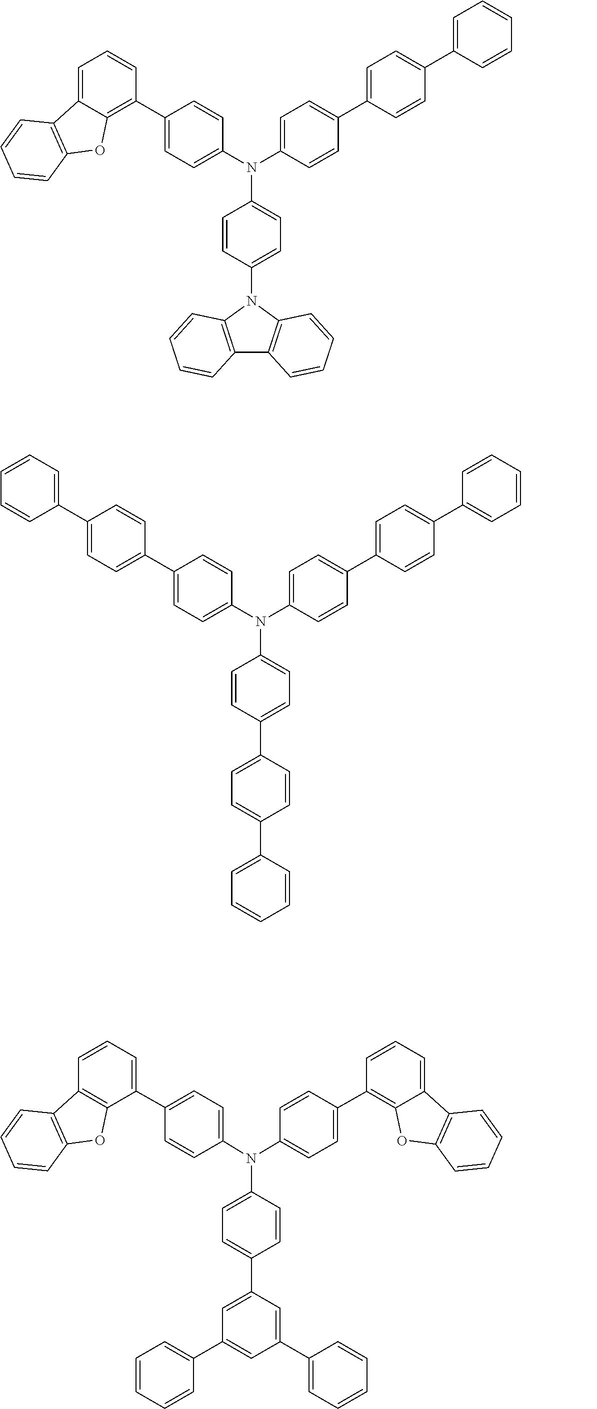 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

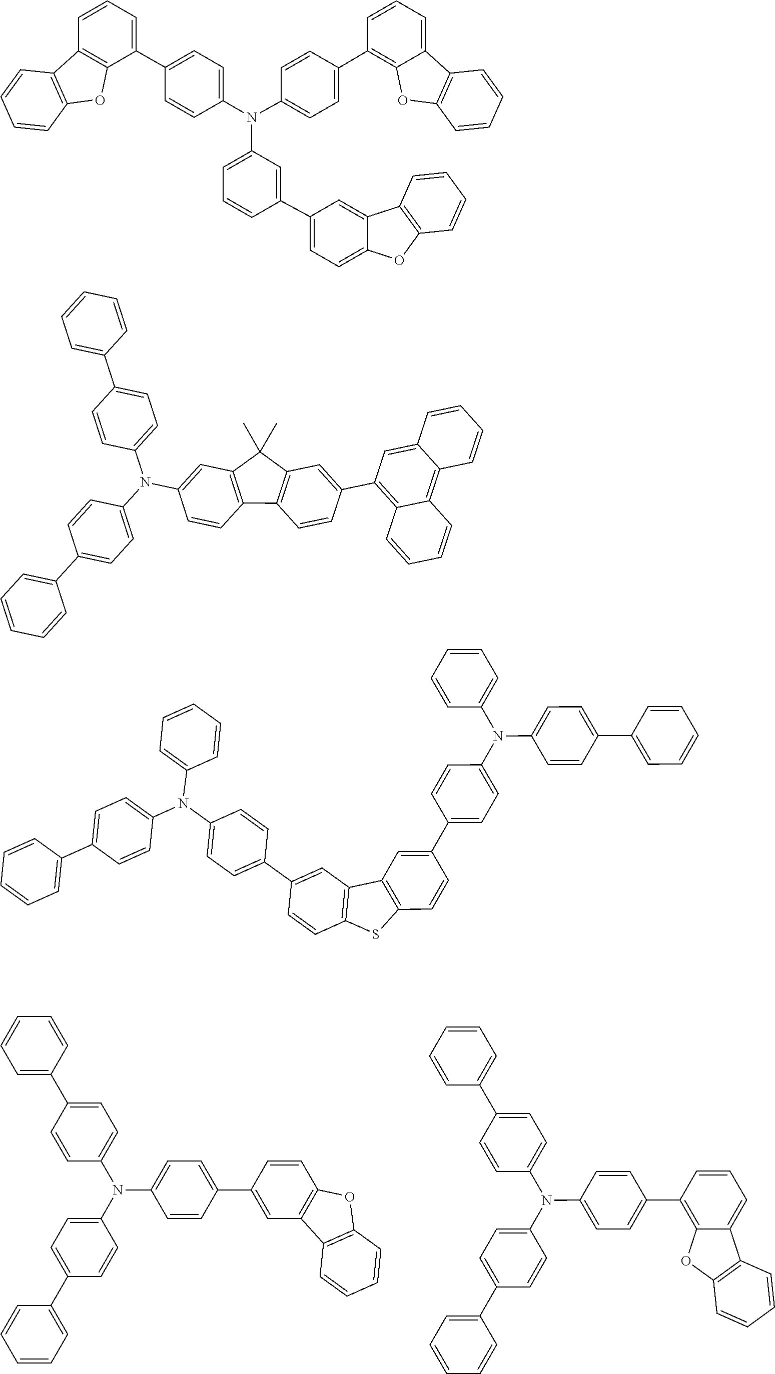 [SMILES: c1cc(-c2ccc3oc4ccccc4c3c2)cc(N(c2ccc(-c3cccc4c3oc3ccccc34)cc2)c2ccc(-c3cccc4c3oc3ccccc34)cc2)c1 ; CC1(C)c2cc(-c3cc4ccccc4c4ccccc34)ccc2-c2ccc(N(c3ccc(-c4ccccc4)cc3)c3ccc(-c4ccccc4)cc3)cc21 ; c1ccc(-c2ccc(N(c3ccccc3)c3ccc(-c4ccc5sc6ccc(-c7ccc(N(c8ccccc8)c8ccc(-c9ccccc9)cc8)cc7)cc6c5c4)cc3)cc2)cc1 ; c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3ccc(-c4cccc5c4oc4ccccc45)cc3)cc2)cc1]

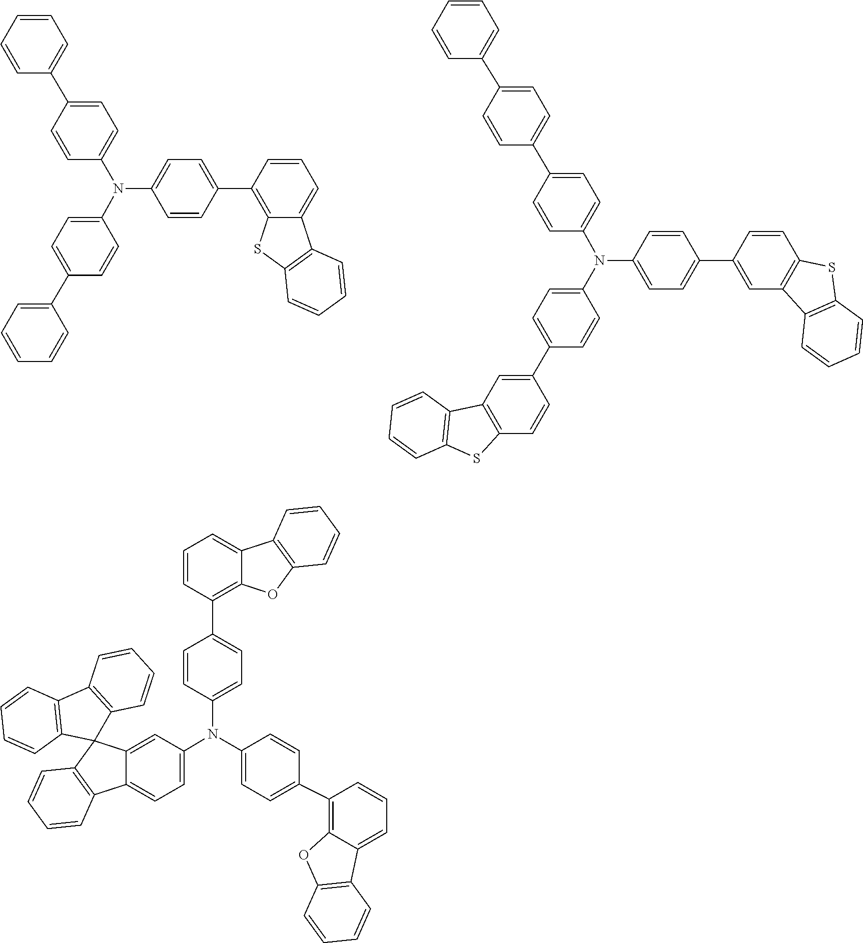 [SMILES: c1ccc(-c2ccc(N(c3ccc(-c4ccccc4)cc3)c3ccc(-c4cccc5c4sc4ccccc45)cc3)cc2)cc1 ; c1ccc(-c2ccc(-c3ccc(N(c4ccc(-c5ccc6sc7ccccc7c6c5)cc4)c4ccc(-c5ccc6sc7ccccc7c6c5)cc4)cc3)cc2)cc1 ; c1ccc2c(c1)-c1ccccc1C21c2ccccc2-c2ccc(N(c3ccc(-c4cccc5c4oc4ccccc45)cc3)c3ccc(-c4cccc5c4oc4ccccc45)cc3)cc21]

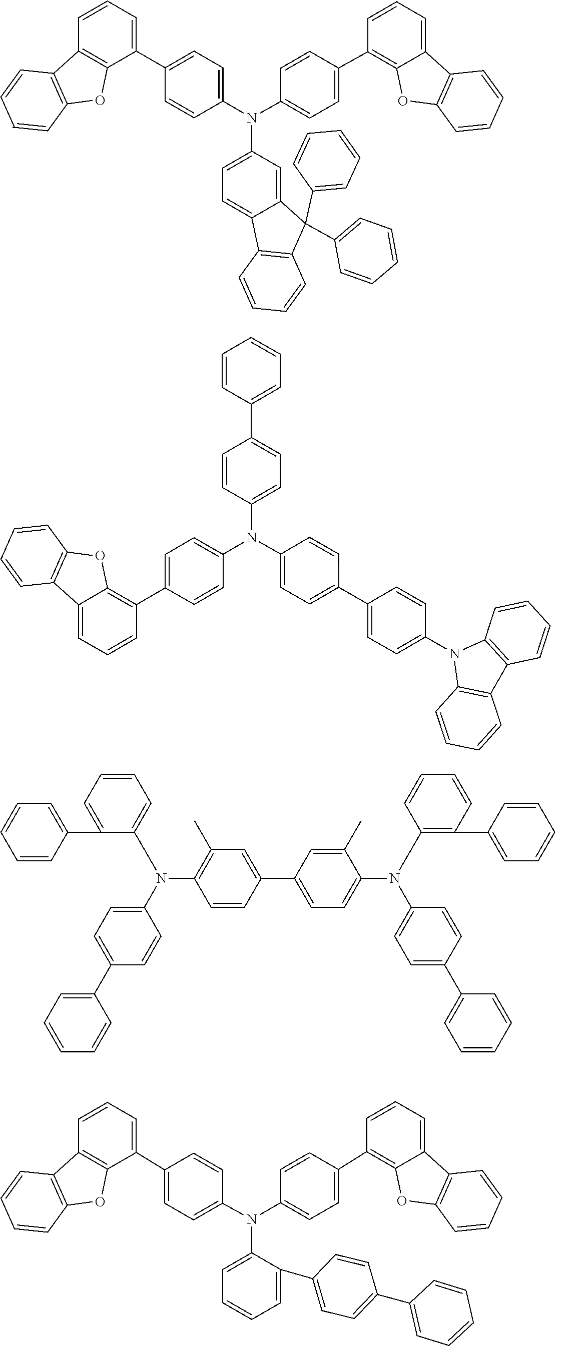

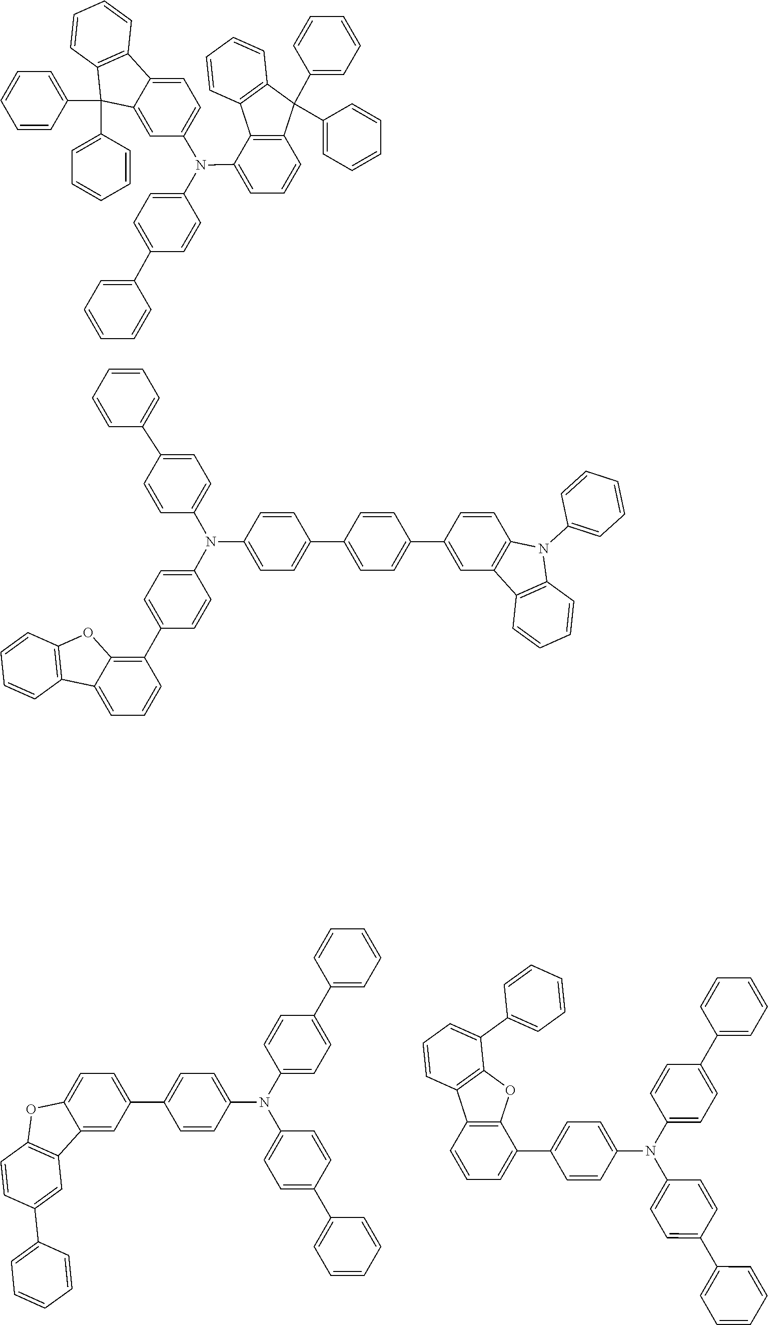

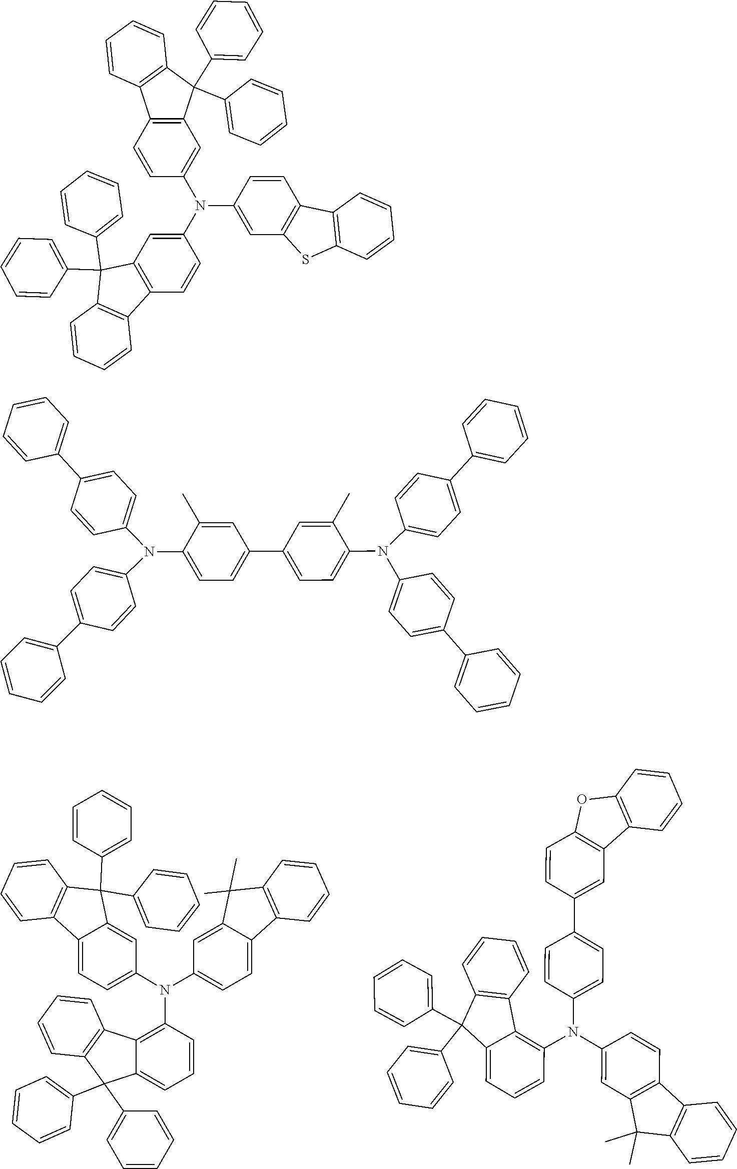 [SMILES: c1ccc(C2(c3ccccc3)c3ccccc3-c3ccc(N(c4ccc5c(c4)C(c4ccccc4)(c4ccccc4)c4ccccc4-5)c4ccc5c(c4)sc4ccccc45)cc32)cc1 ; Cc1cc(-c2ccc(N(c3ccc(-c4ccccc4)cc3)c3ccc(-c4ccccc4)cc3)c(C)c2)ccc1N(c1ccc(-c2ccccc2)cc1)c1ccc(-c2ccccc2)cc1 ; CC1(C)c2ccccc2-c2ccc(N(c3ccc4c(c3)C(c3ccccc3)(c3ccccc3)c3ccccc3-4)c3cccc4c3-c3ccccc3C4(c3ccccc3)c3ccccc3)cc21 ; CC1(C)c2ccccc2-c2ccc(N(c3ccc(-c4ccc5oc6ccccc6c5c4)cc3)c3cccc4c3-c3ccccc3C4(c3ccccc3)c3ccccc3)cc21]

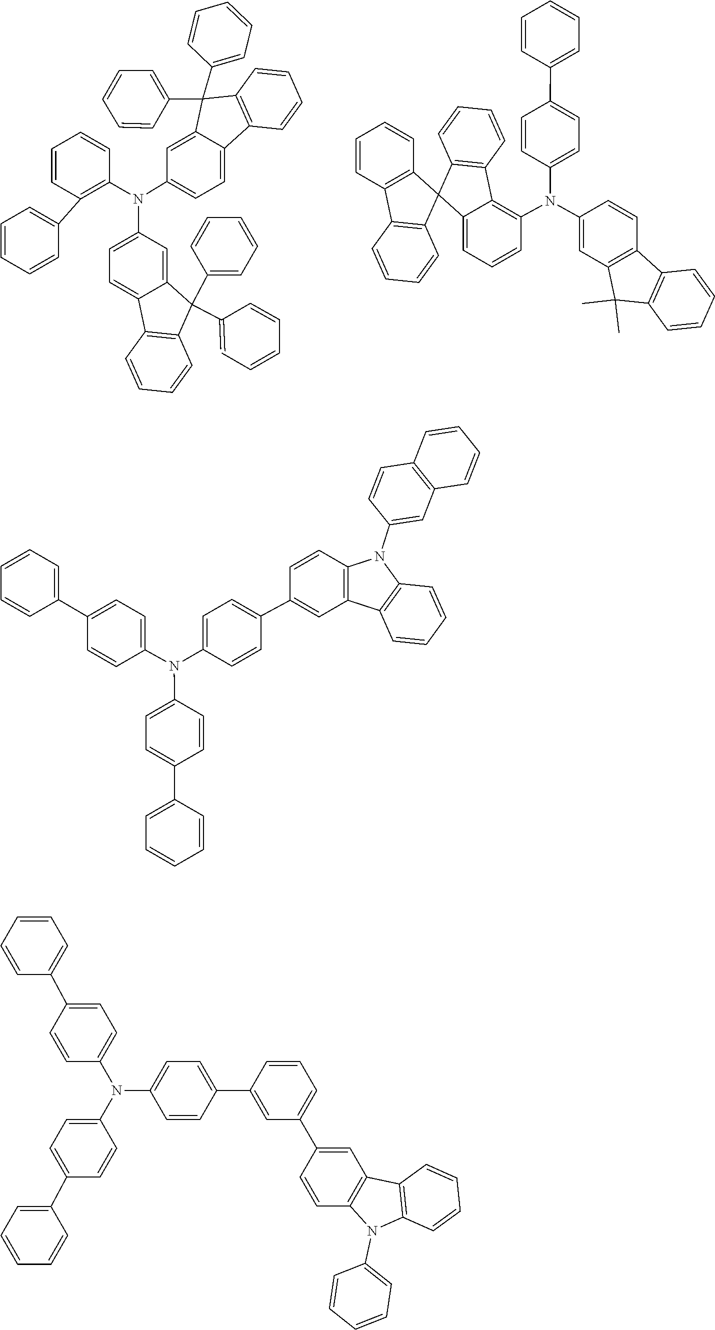 [SMILES: C=C(/C=C\C=C/C)C1(c2ccccc2)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccc(-c4ccccc4)cc3)c3cccc4c3-c3ccccc3C43c4ccccc4-c4ccccc43)cc21 ; c1ccc(-c2ccc(N(c3ccc(-c4ccccc4)cc3)c3ccc(-c4ccc5c(c4)c4ccccc4n5-c4ccc5ccccc5c4)cc3)cc2)cc1 ; c1ccc(-c2ccc(N(c3ccc(-c4ccccc4)cc3)c3ccc(-c4cccc(-c5ccc6c(c5)c5ccccc5n6-c5ccccc5)c4)cc3)cc2)cc1]

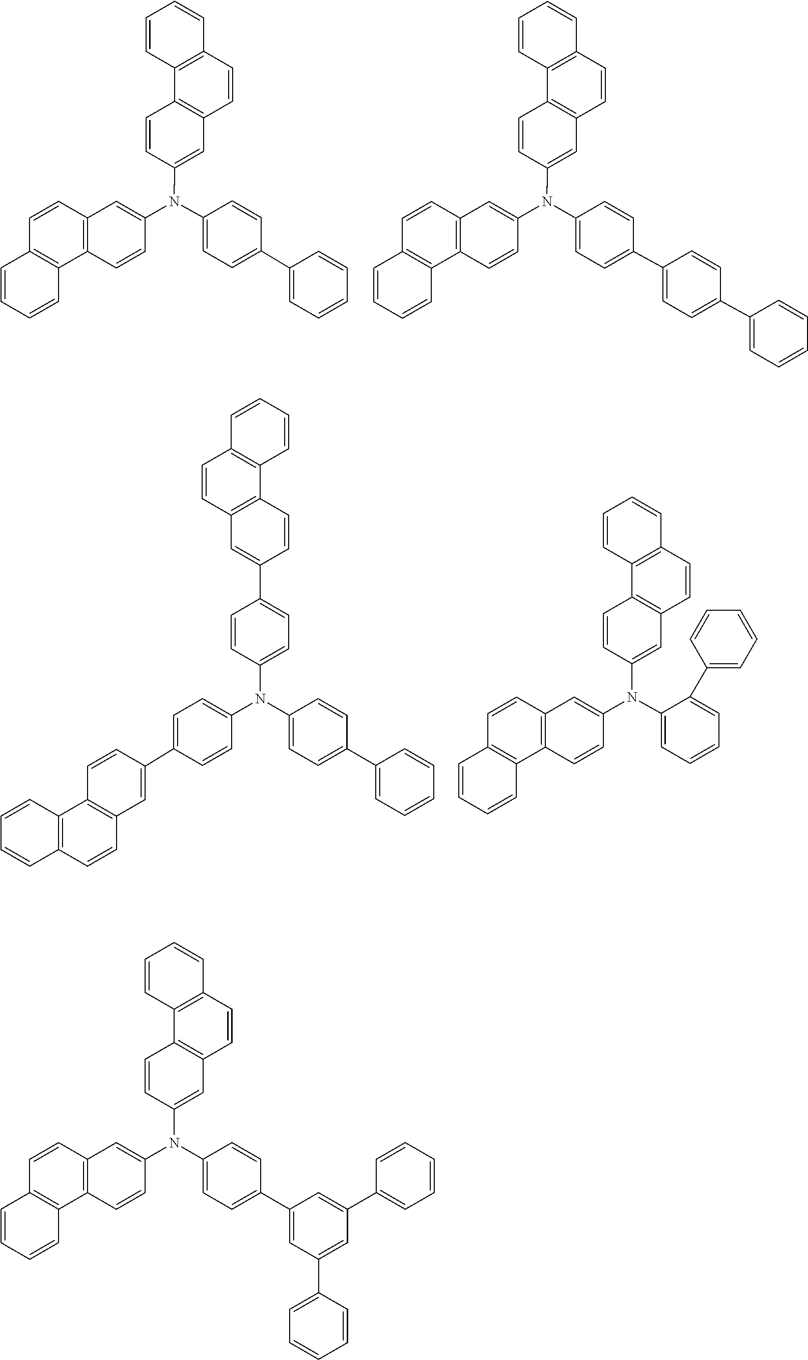

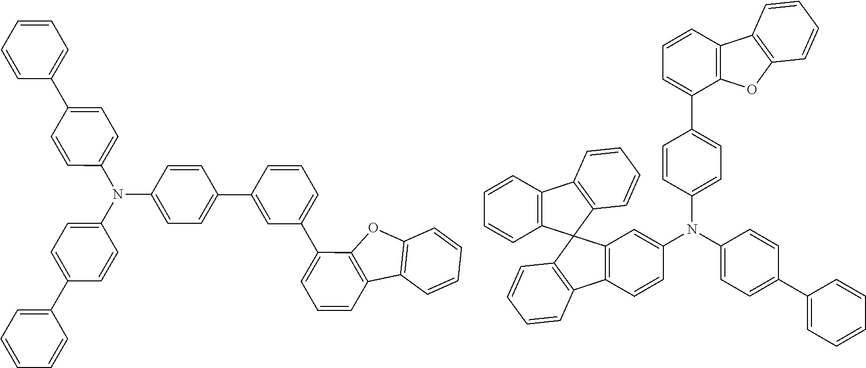

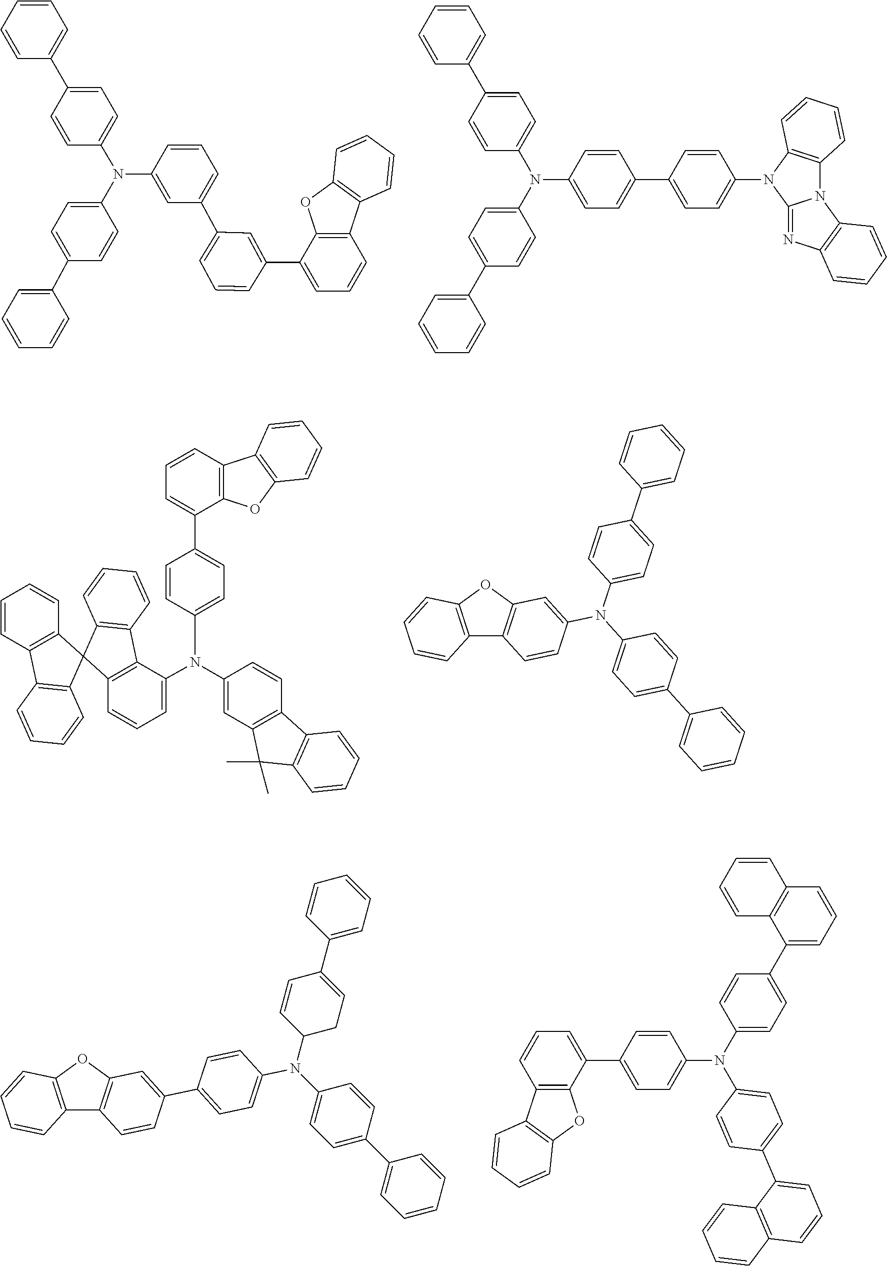

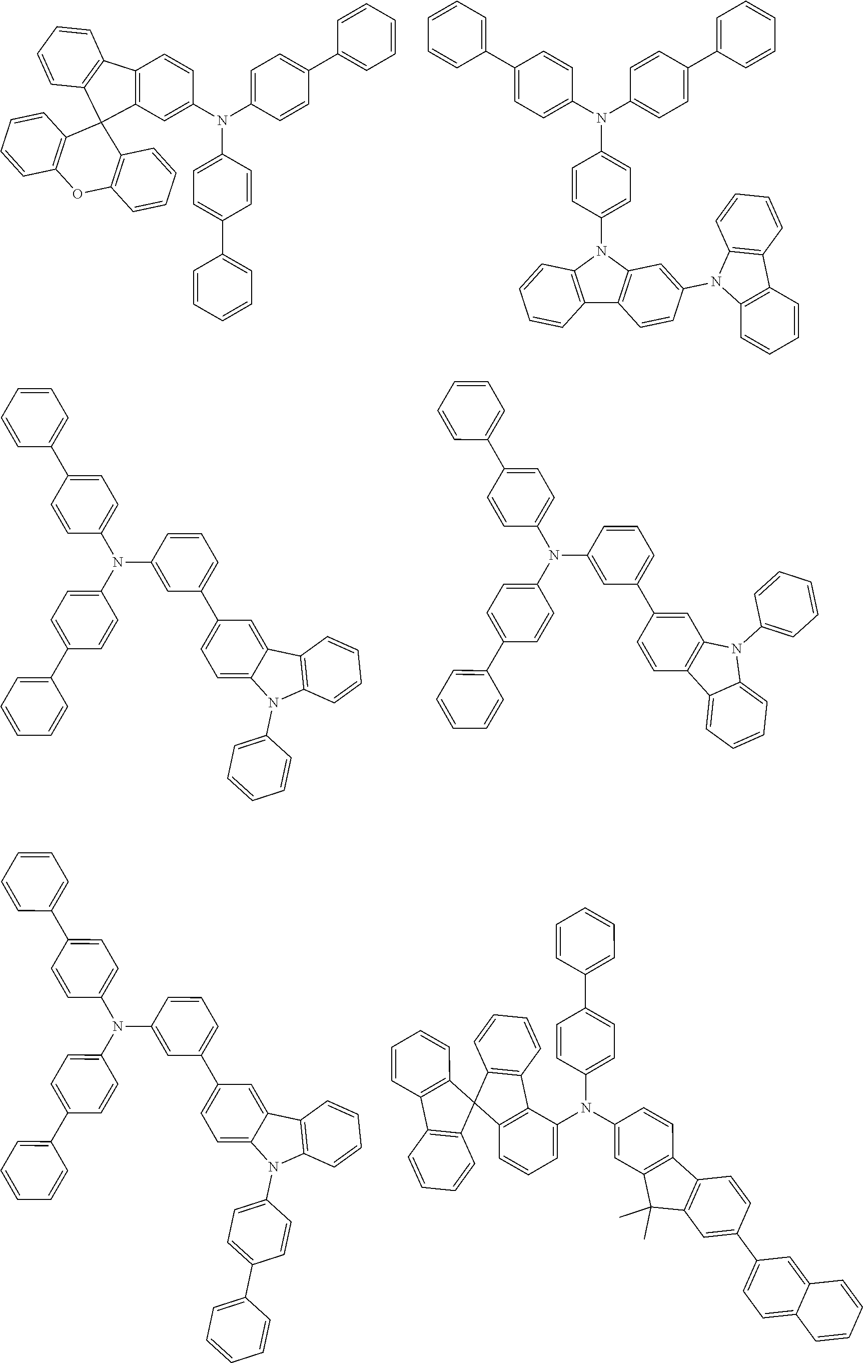

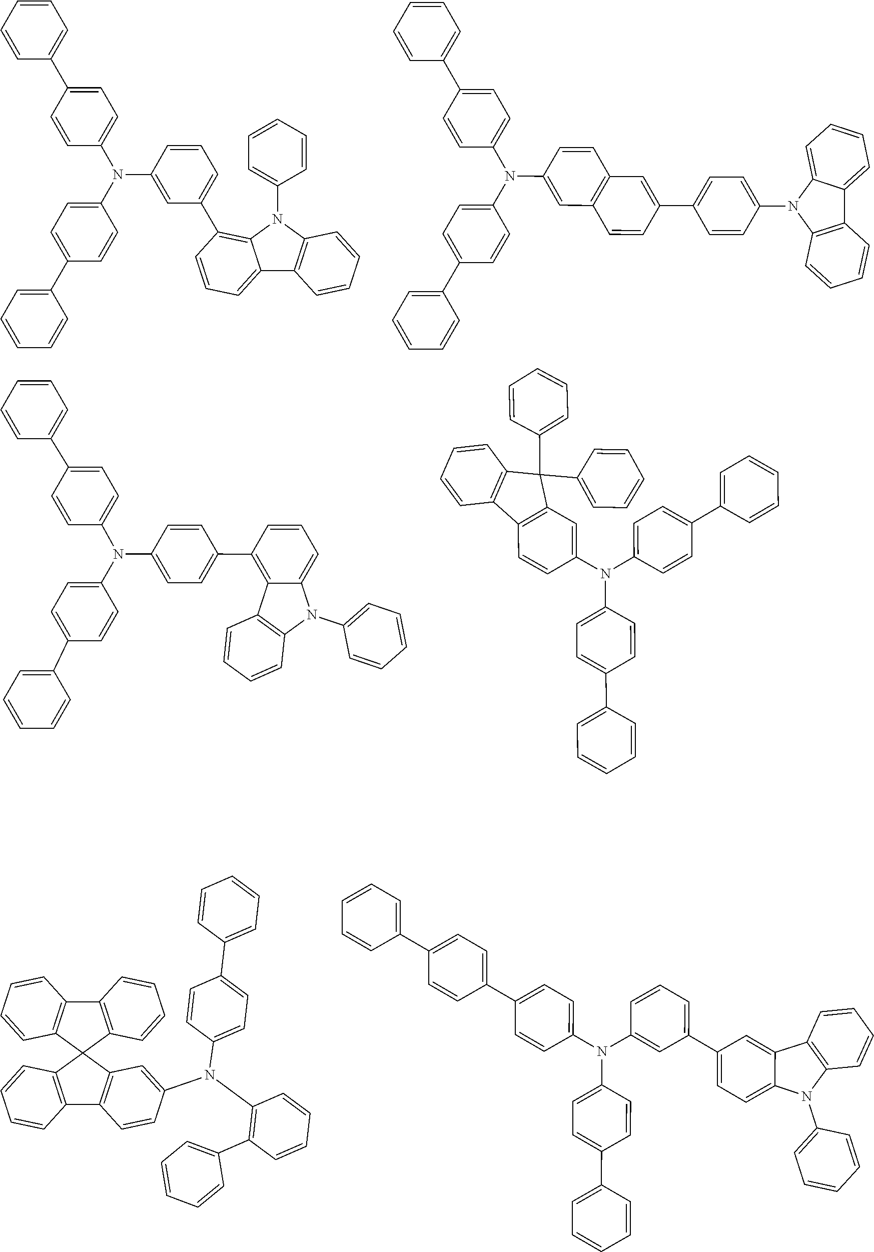

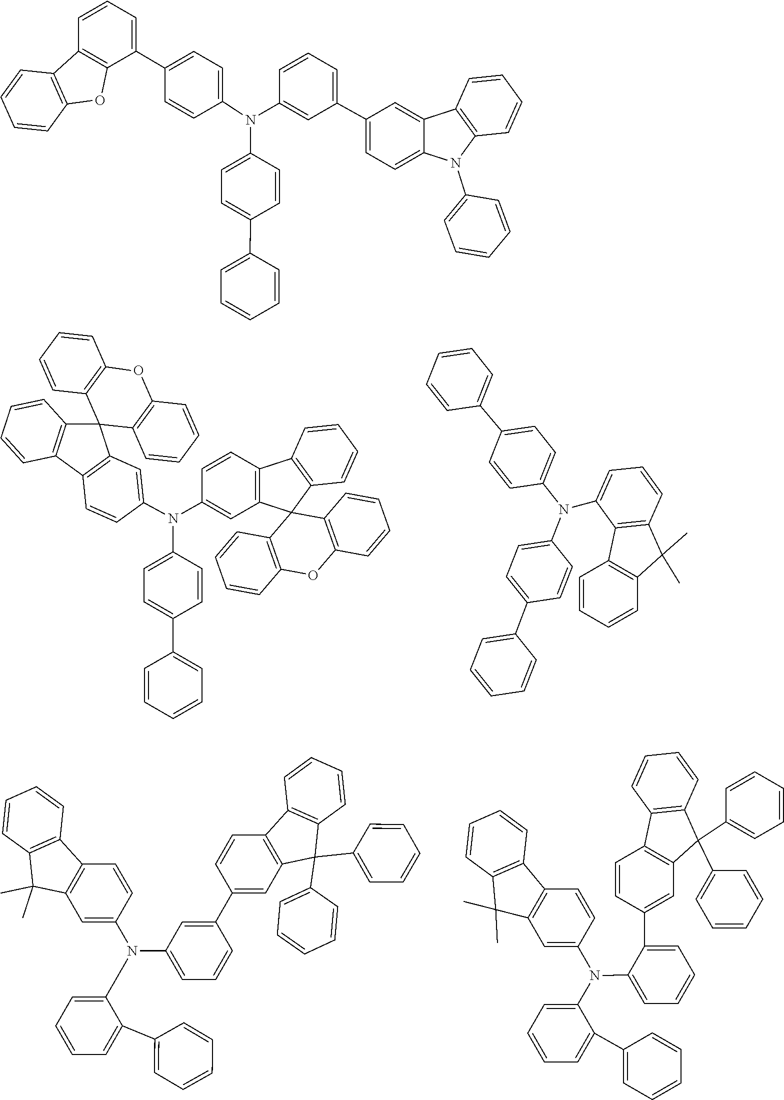

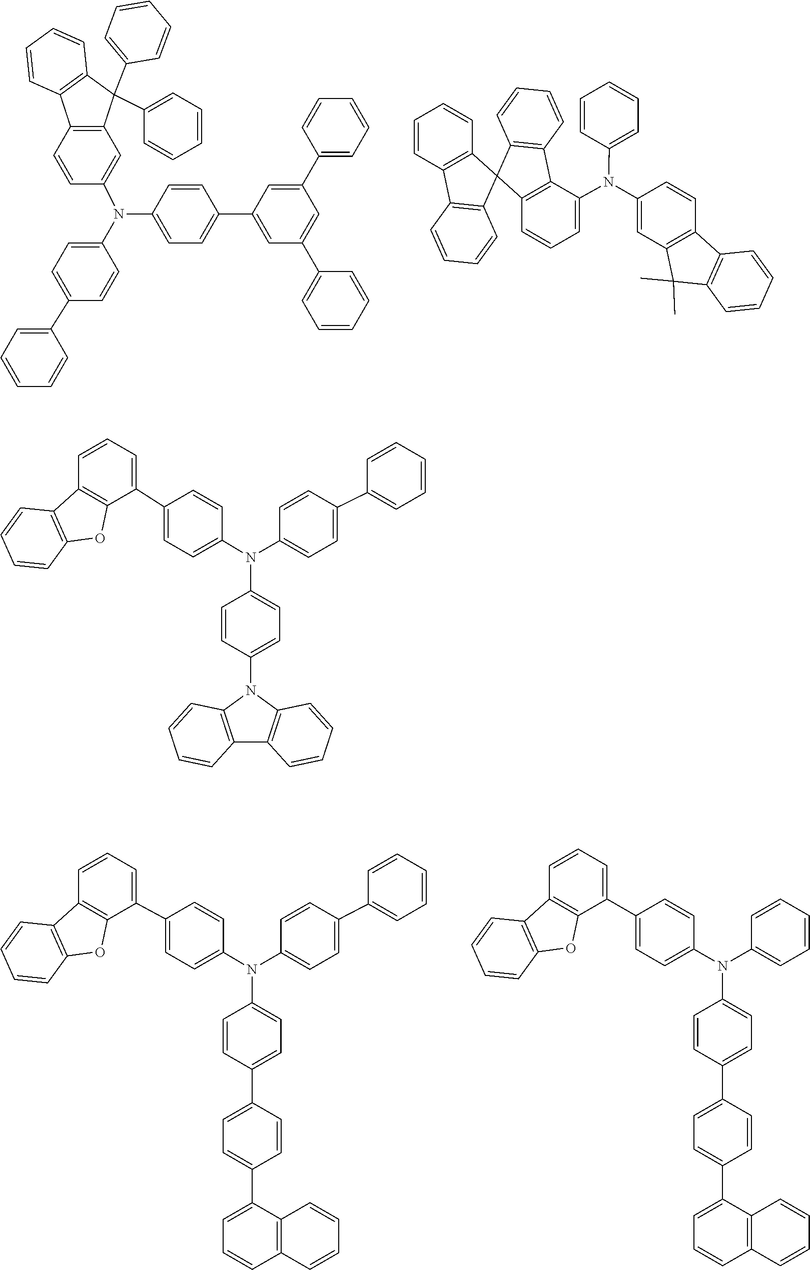

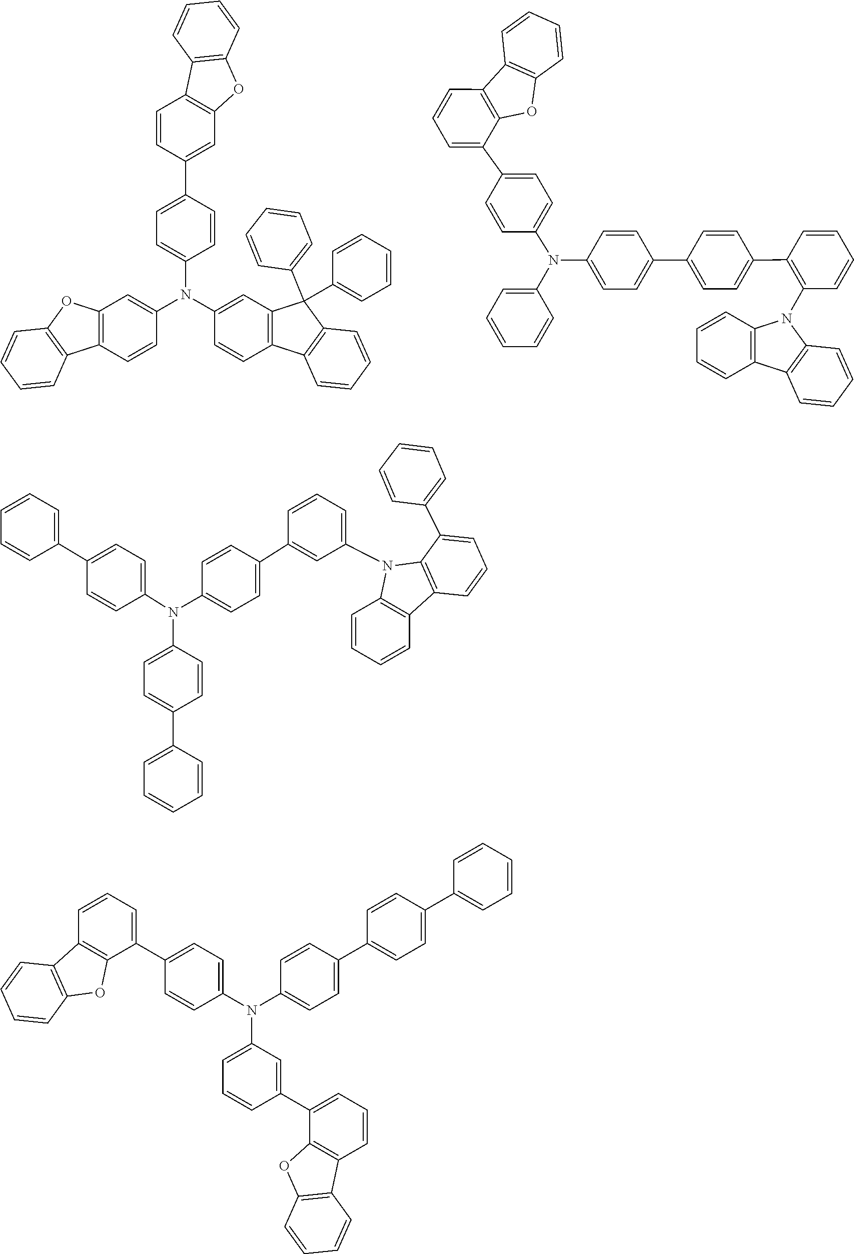 [SMILES: c1ccc(C2(c3ccccc3)c3ccccc3-c3ccc(N(c4ccc(-c5ccc6c(c5)oc5ccccc56)cc4)c4ccc5c(c4)oc4ccccc45)cc32)cc1 ; c1ccc(N(c2ccc(-c3ccc(-c4ccccc4-n4c5ccccc5c5ccccc54)cc3)cc2)c2ccc(-c3cccc4c3oc3ccccc34)cc2)cc1 ; c1ccc(-c2ccc(N(c3ccc(-c4ccccc4)cc3)c3ccc(-c4cccc(-n5c6ccccc6c6cccc(-c7ccccc7)c65)c4)cc3)cc2)cc1 ; c1ccc(-c2ccc(-c3ccc(N(c4ccc(-c5cccc6c5oc5ccccc56)cc4)c4cccc(-c5cccc6c5oc5ccccc56)c4)cc3)cc2)cc1]

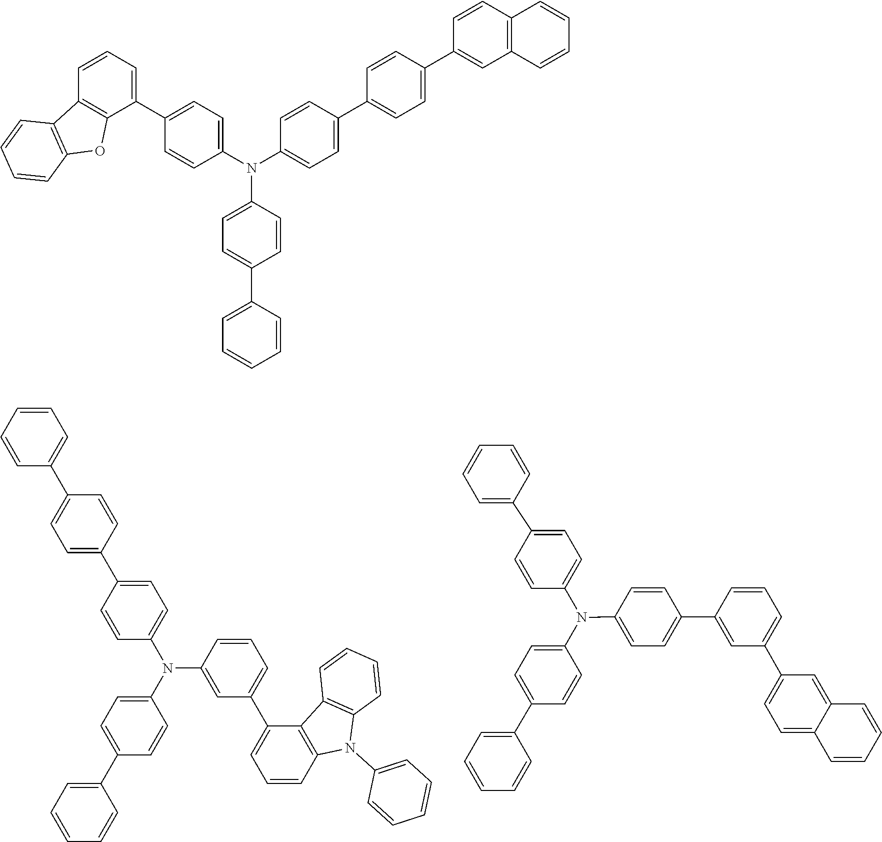

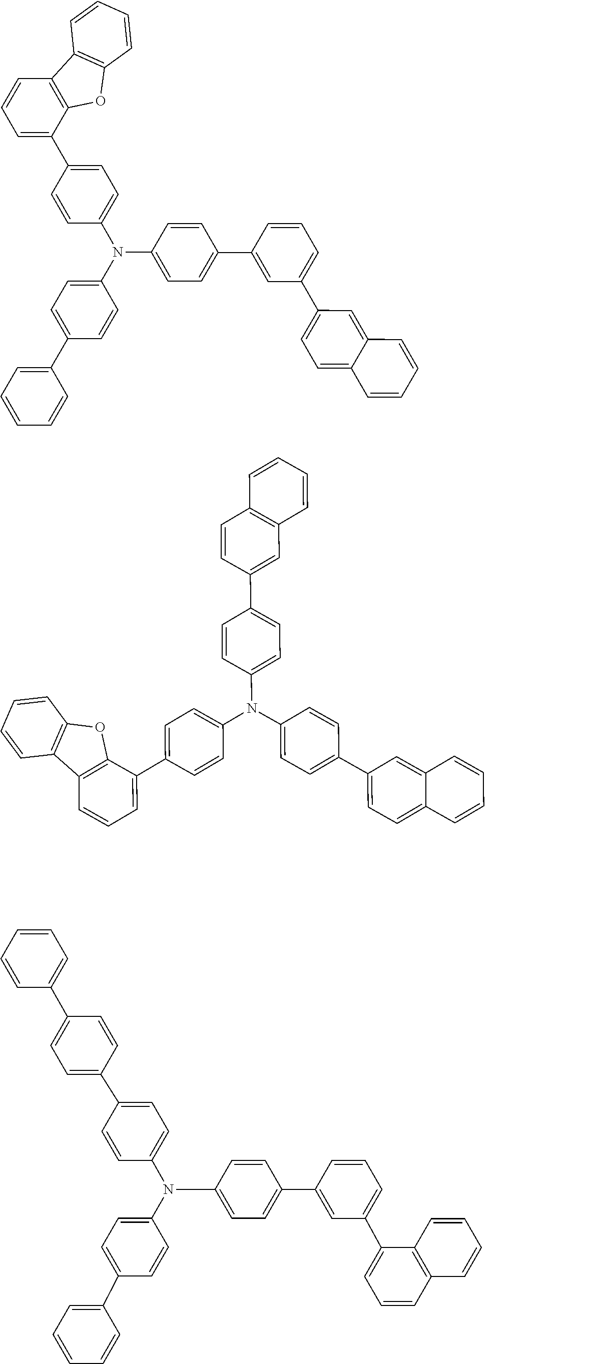 [SMILES: c1ccc(-c2ccc(N(c3ccc(-c4cccc(-c5ccc6ccccc6c5)c4)cc3)c3ccc(-c4cccc5c4oc4ccccc45)cc3)cc2)cc1 ; c1ccc2cc(-c3ccc(N(c4ccc(-c5ccc6ccccc6c5)cc4)c4ccc(-c5cccc6c5oc5ccccc56)cc4)cc3)ccc2c1 ; c1ccc(-c2ccc(-c3ccc(N(c4ccc(-c5ccccc5)cc4)c4ccc(-c5cccc(-c6cccc7ccccc67)c5)cc4)cc3)cc2)cc1]

The emitting layer of the first exemplary embodiment at least contains a delayed fluorescent compound.

An arrangement of the first exemplary embodiment, in which the emitting layer contains a fluorescent compound M1 and a compound M2 as the delayed fluorescent compound, is explained below.

### Emitting Layer

The emitting layer of the organic EL device according to the exemplary embodiment contains the fluorescent compound M1 and the compound M2 as the delayed fluorescent compound.

In this arrangement, the compound M2 is preferably a host material (occasionally also referred to as a matrix material). The compound M1 is preferably a dopant material (occasionally also referred to as a guest material, emitter or luminescent material).

### Compound M2

### Delayed Fluorescence

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a mechanism of generating delayed fluorescence is explained in FIG. 10.38 in the document. The compound M2 of the exemplary embodiment is preferably a compound exhibiting thermally activated delayed fluorescence generated by such a mechanism.

In general, emission of delayed fluorescence can be confirmed by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

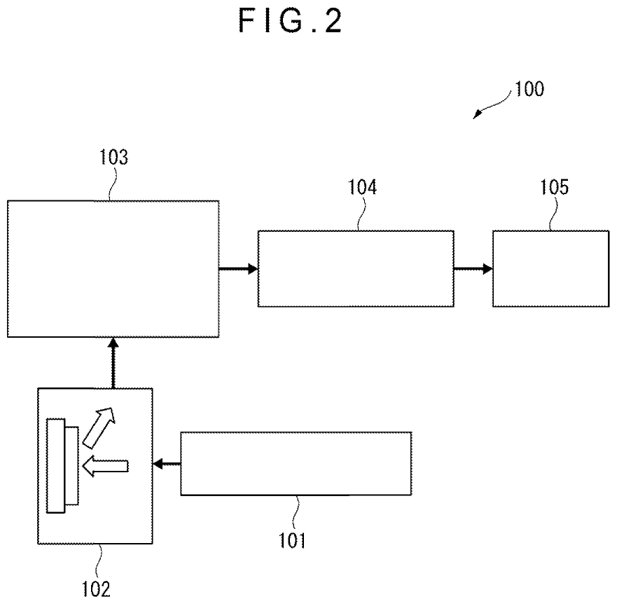

FIG. 2 shows a schematic diagram of an exemplary device for measuring the transient PL. An example of a method of measuring a transient PL using FIG. 2 and an example of behavior analysis of delayed fluorescence will be described.

A transient PL measuring device 100 in FIG. 2 includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. A device for measuring the transient PL is not limited to the device shown in FIG. 2.

The sample housed in the sample chamber 102 is obtained by forming a thin film, in which a matrix material is doped with a doping material at a concentration of 12 mass %, on the quartz substrate.

The thin film sample housed in the sample chamber 102 is irradiated with the pulse laser from the pulse laser 101 to excite the doping material. Emission is extracted in a direction of 90 degrees with respect to a radiation direction of the excited light. The extracted emission is divided by the spectrometer 103 to form a two-dimensional image in the streak camera 104. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was prepared as described above from a reference compound H1 as the matrix material and a reference compound D1 as the doping material and was measured in terms of the transient PL.

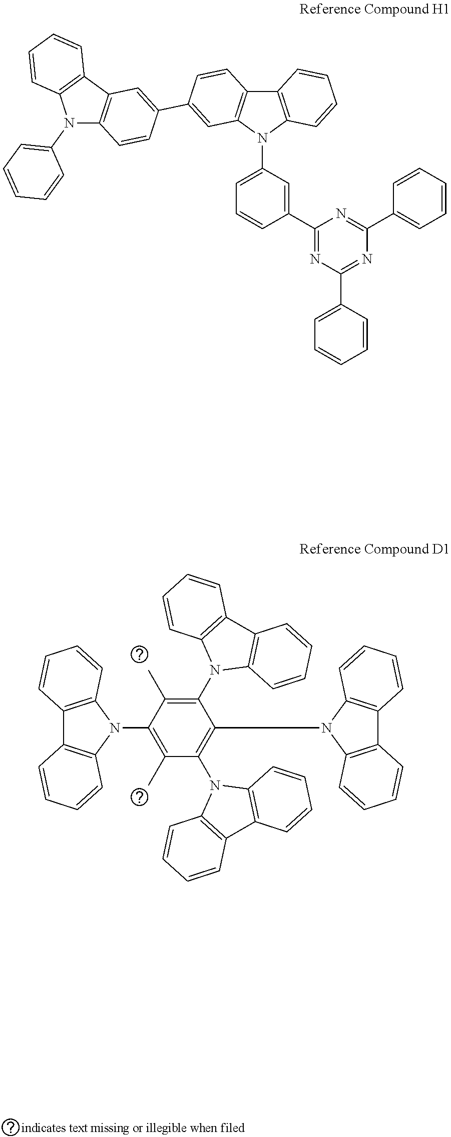 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; Cc1c(-n2c3ccccc3c3ccccc32)c(C)c(-n2c3ccccc3c3ccccc32)c(-n2c3ccccc3c3ccccc32)c1-n1c2ccccc2c2ccccc21]

The decay curve was analyzed with respect to the above thin film sample A and a thin film sample B. The thin film sample B was manufactured in the same manner as described above from a reference compound H2 as the matrix material and the reference compound D1 as the doping material.

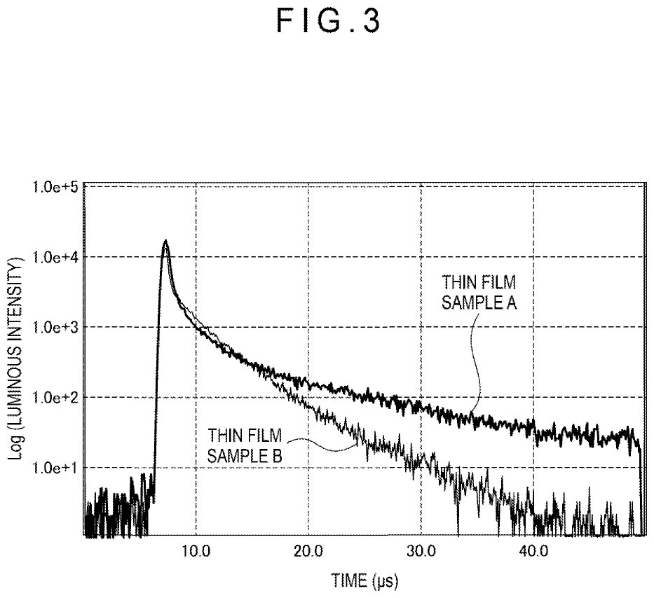

FIG. 3 shows decay curves obtained from transient PL obtained by measuring the thin film samples A and B.

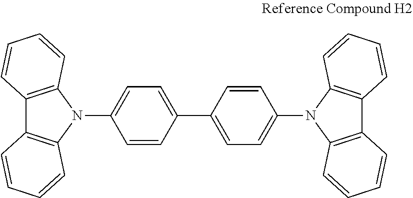 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by inverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

Herein, a sample manufactured by the following method is used for measuring delayed fluorescence of the compound M2. For instance, the compound M2 is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (manufactured by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969.

In the exemplary embodiment, provided that an amount of Prompt emission of a measurement target compound (compound M2) is denoted by X_{P} and the amount of Delay emission is denoted by X_{D}, a value of X_{D}/X_{P} is preferably 0.05 or more.

The amounts of Prompt emission and Delay emission and a ratio of the amounts thereof in compounds other than the compound M2 herein are measured in the same manner as those of the compound M2.

### Compound Represented by Formula (2)

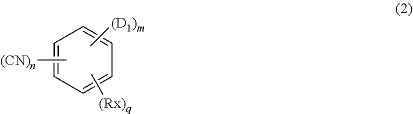

In the formula (2):

n is 1, 2, 3 or 4;

m is 1, 2, 3 or 4;

q is 0, 1, 2, 3 or 4;

m+n+q=6 is satisfied;

CN is a cyano group;

D₁ is a group represented by a formula (2a), (2b) or (2c) below, and when a plurality of D₁ are present, the plurality of D₁ are mutually the same or different; Rx is a hydrogen atom or a substituent, or at least one combination of adjacent ones of Rx are mutually bonded to form a ring, and when a plurality of Rx are present, the plurality of Rx are mutually the same or different;

each Rx as a substituent is independently a halogen atom, a substituted or unsubstituted aryl group having 6 to 30 ring carbon atoms, a substituted or unsubstituted heterocyclic group having 5 to 30 ring atoms, a substituted or unsubstituted amino group, a substituted or unsubstituted carbonyl group, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, or a substituted or unsubstituted arylsilyl group having 6 to 60 ring carbon atoms; and

CN, D₁ and Rx are bonded to respective carbon atoms of a six-membered ring.

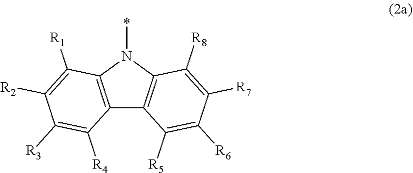

In the formula (2a):

R₁ to R₈ are each independently a hydrogen atom or a substituent, or at least one combination of a combination of R₁ and R₂, a combination of R₂ and R₃, a combination of R₃ and R₄, a combination of R₅ and R₆, a combination of R₆ and R₇, or a combination of R₇ and R₈ are mutually bonded to form a ring;

R₁ to R₈ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, a substituted or unsubstituted arylsilyl group having 6 to 60 ring carbon atoms, a hydroxy group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to a carbon atom in a six-membered ring in the formula (2).

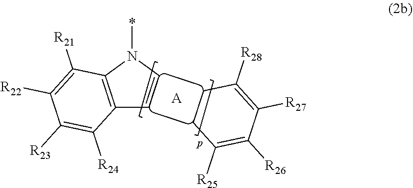

In the formula (2b):

R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;

R₂₁ to R₂₈ as a substituent each independently represent the same as R₁ to R₈ in the formula (2a);

A represents a cyclic structure represented by a formula (211) or (212) below, and the cyclic structure A is fused with adjacent cyclic structure(s) at any position(s);

p is 1, 2, 3 or 4;

when p is 2, 3 or 4, a plurality of cyclic structures A are mutually the same or different; and

* represents a bonding position to a carbon atom in a six-membered ring in the formula (2).

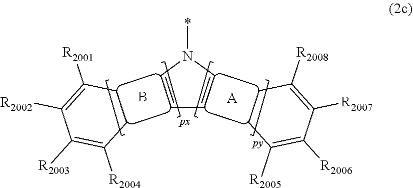

In the formula (2c):

R₂₀₀₁ to R₂₀₀₈ are each independently a hydrogen atom or a substituent, or at least one combination of a combination of R₂₀₀₁ and R₂₀₀₂, a combination of R₂₀₀₂ and R₂₀₀₃, a combination of R₂₀₀₃ and R₂₀₀₄, a combination of R₂₀₀₅ and R₂₀₀₆, a combination of R₂₀₀₆ and R₂₀₀₇, or a combination of R₂₀₀₇ and R₂₀₀₈ are mutually bonded to form a ring;

R₂₀₀₁ to R₂₀₀₈ as a substituent each independently represent the same as R₁ to R₈ as a substituent in the formula (2a);

B represents a cyclic structure represented by the formula (211) or (212), and the cyclic structure B is fused with adjacent cyclic structure(s) at any position(s);

px is 1, 2, 3 or 4;

when px is 2, 3 or 4, a plurality of cyclic structures B are mutually the same or different;

C represents a cyclic structure represented by the formula (211) or (212), and the cyclic structure C is fused with adjacent cyclic structure(s) at any position(s);

py is 1, 2, 3 or 4;

when py is 2, 3 or 4, a plurality of cyclic structures C are mutually the same or different; and

* represents a bonding position to a carbon atom in a six-membered ring in the formula (2).

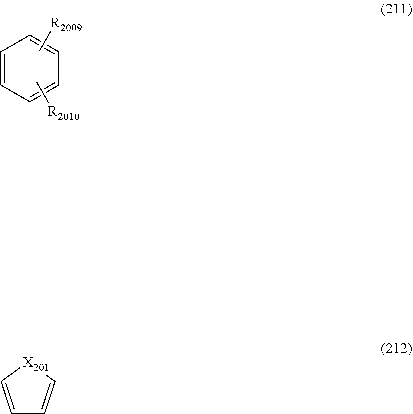

In the formula (211), R₂₀₀₉ and R₂₀₁₀ are each independently a hydrogen atom or a substituent, or bonded to a part of an adjacent cyclic structure to form a ring, or a combination of R₂₀₀₉ and R₂₀₁₀ are mutually bonded to form a ring;

in the formula (212), X₂₀₁ is CR₂₀₁₁R₂₀₁₂, NR₂₀₁₃, a sulfur atom, or an oxygen atom, and R₂₀₁₁, R₂₀₁₂ and R₂₀₁₃ are each independently a hydrogen atom or a substituent, or R₂₀₁₁ and R₂₀₁₂ are mutually bonded to form a ring; and

R₂₀₀₉, R₂₀₁₀, R₂₀₁₁, R₂₀₁₂ and R₂₀₁₃ as a substituent each independently represent the same as R₁ to R₈ as a substituent in the formula (2a).

In the formula (211), R₂₀₀₉ and R₂₀₁₀ are each independently bonded to a part of an adjacent cyclic structure to form a ring, which specifically means any of (I) to (IV) below.

In the formula (211), a combination of R₂₀₀₉ and R₂₀₁₀ are mutually bonded to form a ring, which specifically means (V) below.

(I) When the cyclic structures represented by the formula (211) are adjacent to each other, between the two adjacent rings, at least one combination of the following are mutually bonded to form a ring: R₂₀₀₉ of one of the rings and R₂₀₀₉ of the other of the rings; R₂₀₀₉ of one of the rings and R₂₀₁₀ of the other of the rings; or R₂₀₁₀ of one of the rings and R₂₀₁₀ of the other of the rings.
(II) When the cyclic structure represented by the formula (211) and the benzene ring having R₂₅ to R₂₈ in the formula (2b) are adjacent to each other, between the two adjacent rings, at least one combination of the following are mutually bonded to form a ring: R₂₀₀₉ of one of the rings and R₂₅ of the other of the rings; R₂₀₀₉ of one of the rings and R₂₈ of the other of the rings; R₂₀₁₀ of one of the rings and R₂₅ of the other of the rings; or R₂₀₁₀ of one of the rings and R₂₈ of the other of the rings.
(III) When the cyclic structure represented by the formula (211) and the benzene ring having R₂₀₀₁ to R₂₀₀₄ in the formula (2c) are adjacent to each other, between the two adjacent rings, at least one combination of the following are mutually bonded to form a ring: R₂₀₀₉ of one of the rings and R₂₀₀₁ of the other of the rings; R₂₀₀₉ of one of the rings and R₂₀₀₄ of the other of the rings; R₂₀₁₀ of one of the rings and R₂₀₀₁ of the other of the rings; or R₂₀₁₀ of one of the rings and R₂₀₀₄ of the other of the rings.
(IV) When the cyclic structure represented by the formula (211) and the benzene ring having R₂₀₀₅ to R₂₀₀₈ in the formula (2c) are adjacent to each other, between the two adjacent rings, at least one combination of the following are mutually bonded to form a ring: R₂₀₀₉ of one of the rings and R₂₀₀₅ of the other of the rings; R₂₀₀₉ of one of the rings and R₂₀₀₈ of the other of the rings; R₂₀₁₀ of one of the rings and R₂₀₀₅ of the other of the rings; or R₂₀₁₀ of one of the rings and R₂₀₀₈ of the other of the rings.
(V) The combination of R₂₀₀₉ and R₂₀₁₀ of the cyclic structure represented by the formula (211) are mutually bonded to form a ring. In other words, (V) means that the combination of R₂₀₀₉ and R₂₀₁₀, which are bonded to the same ring, are mutually bonded to form a ring.

In the compound M2 of the exemplary embodiment, it is preferable that:

each Rx is independently a hydrogen atom, an unsubstituted aryl group having 6 to 30 ring carbon atoms, an unsubstituted heterocyclic group having 5 to 30 ring atoms, or an unsubstituted alkyl group having 1 to 30 carbon atoms; and

when Rx is an unsubstituted heterocyclic group having 5 to 30 ring atoms, Rx as the unsubstituted heterocyclic group having 5 to 30 ring atoms is a pyridyl group, pyrimidinyl group, triazinyl group, dibenzofuranyl group, or dibenzothienyl group.

Herein, the triazinyl group refers to a group obtained by excluding one hydrogen atom from 1,3,5-triazine, 1,2,4-triazine, or 1,2,3-triazine.

The triazinyl group is preferably a group obtained by excluding one hydrogen atom from 1,3,5-triazine.

In the compound M2 of the exemplary embodiment, it is more preferable that each Rx is independently a hydrogen atom, an unsubstituted aryl group having 6 to 30 ring carbon atoms, an unsubstituted dibenzofuranyl group, or an unsubstituted dibenzothienyl group.

In the compound M2 of the exemplary embodiment, Rx is further preferably a hydrogen atom.

In the compound M2 of the exemplary embodiment, it is preferable that R₁ to R₈, R₂₁ to R₂₈, R₂₀₀₁ to R₂₀₀₈, R₂₀₀₉ to R₂₀₁₀ and R₂₀₁₁ to R₂₀₁₃ as a substituent are each independently an unsubstituted aryl group having 6 to 30 ring carbon atoms, an unsubstituted heterocyclic group having 5 to 30 ring atoms, or an unsubstituted alkyl group having 1 to 30 carbon atoms.

In the compound M2 of the exemplary embodiment, D₁ is preferably a group represented by one of formulae (D-21) to (D-27) below.

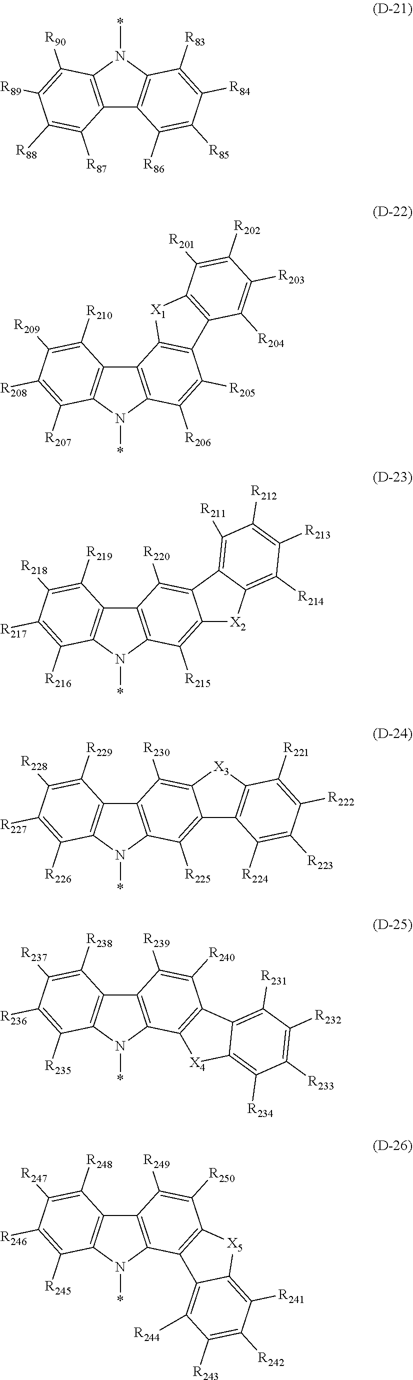

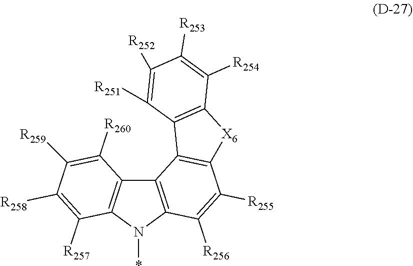

In the formula (D-21), R₈₃ to R₉₀ are each independently a hydrogen atom or a substituent;

in the formulae (D-22) to (D-27): X₁ to X₆ are each independently an oxygen atom, a sulfur atom, or CR₁₅₁R₁₅₂;

R₁₅₁ and R₁₅₂ are each independently a hydrogen atom or a substituent, or R₁₅₁ and R₁₅₂ are bonded to each other to form a ring;

R₂₀₁ to R₂₆₀ are each independently a hydrogen atom or a substituent, or at least one combination of a combination of R₂₀₁ and R₂₀₂, a combination of R₂₀₂ and R₂₀₃, a combination of R₂₀₃ and R₂₀₄, a combination of R₂₀₅ and R₂₀₆, a combination of R₂₀₇ and R₂₀₈, a combination of R₂₀₈ and R₂₀₉, a combination of R₂₀₉ and R₂₁₀, a combination of R₂₁₁ and R₂₁₂, a combination of R₂₁₂ and R₂₁₃, a combination of R₂₁₃ and R₂₁₄, a combination of R₂₁₆ and R₂₁₇, a combination of R₂₁₇ and R₂₁₈, a combination of R₂₁₈ and R₂₁₉, a combination of R₂₂₁ and R₂₂₂, a combination of R₂₂₂ and R₂₂₃, a combination of R₂₂₃ and R₂₂₄, a combination of R₂₂₆ and R₂₂₇, a combination of R₂₂₇ and R₂₂₈, a combination of R₂₂₈ and R₂₂₉, a combination of R₂₃₁ and R₂₃₂, a combination of R₂₃₂ and R₂₃₃, a combination of R₂₃₃ and R₂₃₄, a combination of R₂₃₅ and R₂₃₆, a combination of R₂₃₆ and R₂₃₇, a combination of R₂₃₇ and R₂₃₈, a combination of R₂₃₉ and R₂₄₀, a combination of R₂₄₁ and R₂₄₂, a combination of R₂₄₂ and R₂₄₃, a combination of R₂₄₃ and R₂₄₄, a combination of R₂₄₅ and R₂₄₆, a combination of R₂₄₆ and R₂₄₇, a combination of R₂₄₇ and R₂₄₈, a combination of R₂₄₉ and R₂₅₀, a combination of R₂₅₁ and R₂₅₂, a combination of R₂₅₂ and R₂₅₃, a combination of R₂₅₃ and R₂₅₄, a combination of R₂₅₅ and R₂₅₆, a combination of R₂₅₇ and R₂₅₈, a combination of R₂₅₈ and R₂₅₉, or a combination of R₂₅₉ and R₂₆₀ are bonded to each other to form a ring;

R₈₃ to R₉₀, R₁₅₁, R₁₅₂ and R₂₀₁ to R₂₆₀ as a substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 6 carbon atoms, a substituted or unsubstituted cycloalkyl group having 3 to 8 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and

* represents a bonding position to a carbon atom in a six-membered ring in the formula (2).

In the compound M2 of the exemplary embodiment, D₁ is also more preferably a group represented by formula (D-21), (D-23), (D-24), or (D-25).

In the compound M2 of the exemplary embodiment, D₁ is also further preferably a group represented by formula (D-21), (D-23), or (D-25).

In the compound M2 of the exemplary embodiment, R₈₃ to R₉₀, R₂₀₁ to R₂₆₀, R₁₅₁ and R₁₅₂ are preferably each independently a hydrogen atom, an unsubstituted aryl group having 6 to 14 ring carbon atoms, an unsubstituted heterocyclic group having 5 to 14 ring atoms, or an unsubstituted alkyl group having 1 to 6 carbon atoms.

In the compound M2 of the exemplary embodiment, R₈₃ to R₉₀ and R₂₀₁ to R₂₆₀ are each preferably a hydrogen atom.

In the compound M2 of the exemplary embodiment, R₁₅₁ and R₁₅₂ are preferably each independently an unsubstituted aryl group having 6 to 14 ring carbon atoms, an unsubstituted heterocyclic group having 5 to 14 ring atoms, or an unsubstituted alkyl group having 1 to 6 carbon atoms.

### Compound Represented by Formula (22)

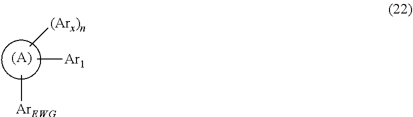

In the formula (22), Ar₁ is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by formulae (1a) to (1j) below.

In the formula (22), Ar_{EWG} is a substituted or unsubstituted heteroaryl group having 5 to 30 ring atoms that includes at least one nitrogen atom in a ring, or an aryl group having 6 to 30 ring carbon atoms that is substituted by at least one cyano group.

In the formula (22), each Arₓ is independently a hydrogen atom or a substituent, and Arₓ as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1j).

In the formula (22), n is 0, 1, 2, 3, 4 or 5, and when n is 2, 3, 4 or 5, a plurality of Arₓ are mutually the same or different.

In the formula (22), a ring (A) is a substituted or unsubstituted aromatic hydrocarbon ring or a substituted or unsubstituted heterocycle, and the ring (A) is a five-membered ring, a six-membered ring, or a seven-membered ring. The ring (A) may be an aromatic hydrocarbon ring or a heterocycle. Ar₁ and Arₓ are bonded to respective ones of elements forming the ring (A).

In the formula (22), at least one of Ar₁ or Arₓ is a group selected from the group consisting of groups represented by the formulae (1a) to (1j).

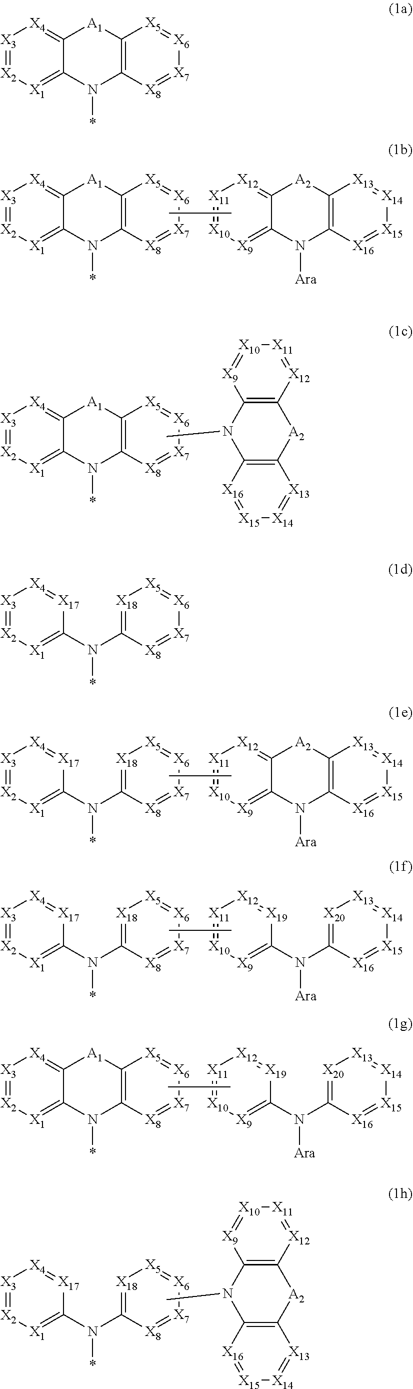

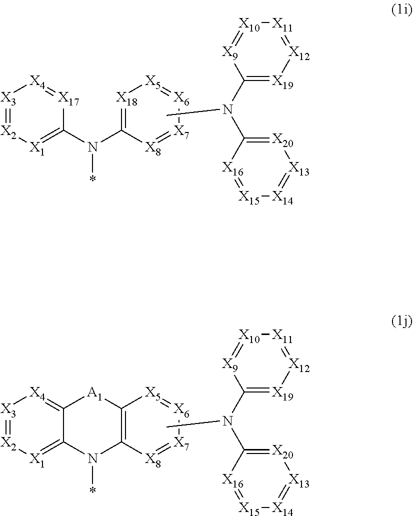

In the formulae (1a) to (1j), X₁ to X₂₀ are each independently a nitrogen atom (N) or a carbon atom bonded with R_{A1} (C—R_{A1})

In the formula (1 b), one of X₅ to X₅ is a carbon atom bonded to one of X₉ to X₁₂, and one of X₉ to X₁₂ is a carbon atom bonded to one of X₅ to X₈.

In the formula (1c), one of X₅ to X₈ is a carbon atom bonded to a nitrogen atom in a ring including A₂.

In the formula (1e), one of X₅ to X₈ and X₁₈ is a carbon atom bonded to one of X₉ to X₁₂, and one of X₉ to X₁₂ is a carbon atom bonded to one of X₅ to X₈ and X₁₈.

In the formula (1f), one of X₅ to X₈ and X₁₈ is a carbon atom bonded to one of X₉ to X₁₂ and X₁₉, and one of X₉ to X₁₂ and X₁₉ is a carbon atom bonded to one of X₅ to X₈ and X₁₈.

In the formula (1g), one of X₅ to X₈ is a carbon atom bonded to one of X₉ to X₁₂ and X₁₉, and one of X₉ to X₁₂ and X₁₉ is a carbon atom bonded to one of X₅ to X₈.

In the formula (1h), one of X₅ to X₈ and X₁₈ is a carbon atom bonded to a nitrogen atom in a ring including A₂.

In the formula (1i), one of X₅ to X₈ and X₁₈ is a carbon atom bonded to a nitrogen atom that links a ring including X₉ to X₁₂ and X₁₉ with a ring including X₁₃ to X₁₆ and X₂₀.

In the formula (1j), one of X₅ to X₈ is a carbon atom bonded to a nitrogen atom that links a ring including X₉ to X₁₂ and X₁₉ with a ring including X₁₃ to X₁₆ and X₂₀.

each R_{A1} is independently a hydrogen atom or a substituent, or at least one combination of combinations of a plurality of R_{A1} are mutually directly bonded to form a ring or bonded via a hetero atom to form a ring; and

R_{A1} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

When a plurality of R_{A1} as a substituent are present, the plurality of R_{A1} are mutually the same or different.

In the formula (1a), when X₁ to X₈ are each a carbon atom bonded with R_{A1} (C—R_{A1}), a plurality of R_{A1} preferably form no ring.

In the formulae (1a) to (1j), * represents a bonding position to the ring (A).

In the formulae (1a) to (1j), A₁ and A₂ are each independently a single bond, an oxygen atom (O), a sulfur atom (S), C(R₂₀₂₁)(R₂₀₂₂), Si(R₂₀₂₃)(R₂₀₂₄), C(═O), S(═O), SO₂ or N(R₂₀₂₅). R₂₀₂₁ to R₂₀₂₅ are each independently a hydrogen atom or a substituent, and R₂₀₂₁ to R₂₀₂₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

In the formulae (1a) to (1j), Ara is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, and a substituted silyl group. Ara is preferably a substituted or unsubstituted aryl group having 6 to 30 ring carbon atoms or a substituted or unsubstituted heteroaryl group having 5 to 30 ring atoms.

The formula (1a) is represented by a formula (1aa) below when A₁ is a single bond, represented by a formula (1ab) below when A₁ is 0, represented by a formula (1ac) below when A₁ is S, represented by a formula (1ad) below when A₁ is C(R₂₀₂₁)(R₂₀₂₂), represented by a formula (1ae) below when A₁ is Si(R₂₀₂₃)(R₂₀₂₄), represented by a formula (1af) below when A₁ is C(═O), represented by a formula (1ag) below when A₁ is S(═O), represented by a formula (1ah) below when A₁ is SO₂, and represented by a formula (1ai) below when A₁ is N(R₂₀₂₅). In the formulae (1aa) to (1ai), X₁ to X₈ and R₂₀₂₁ to R₂₀₂₅ represent the same as described above. Linkages between rings via A₁ and A₂ in the formulae (1b), (1c), (1e) and (1g) to (1j) are the same as those in the formulae (1aa) to (1ai). In the formula (1aa), when X₁ to X₈ are each a carbon atom bonded with R_{A1} (C—R_{A1}), a plurality of R_{A1} as a substituent preferably form no ring.

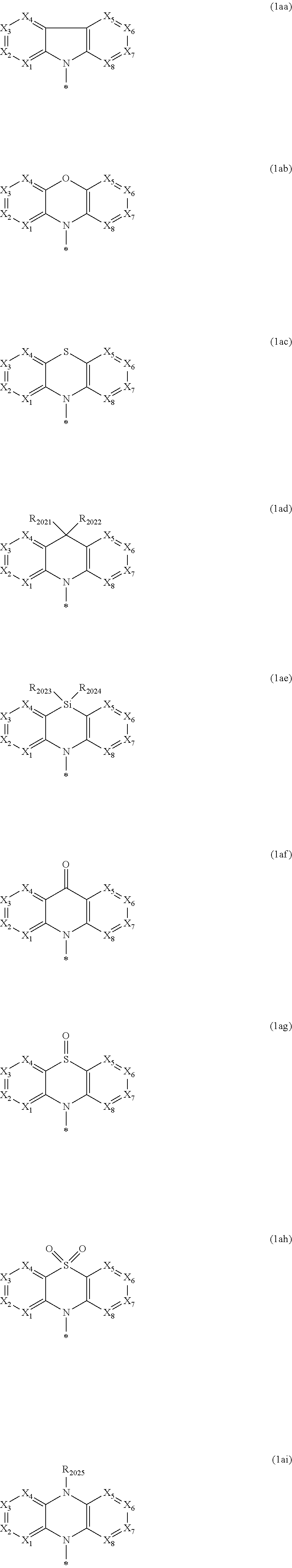

The compound M2 is preferably represented by a formula (221) below.

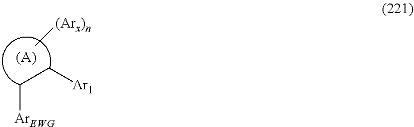

Arₜ Ar_{EWG}, Arₓ, n and a ring (A) in the formula (221) respectively represent the same as Arₜ Ar_{EWG}, Arₓ, n and the ring (A) in the formula (22).

The compound M2 is also preferably represented by a formula (222) below.

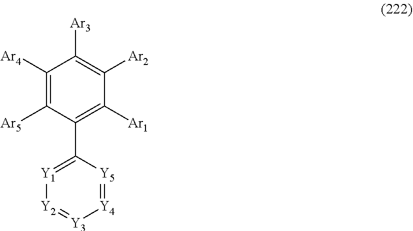

In the formula (222), Y₁ to Y₅ are each independently a nitrogen atom (N), a carbon atom bonded with a cyano group (C—CN), or a carbon atom bonded with R_{A2} (C—R_{A2}), and at least one of Y₁ to Y₅ is N or C—CN. A plurality of R_{A2} are mutually the same or different. R_{A2} are each independently a hydrogen atom or a substituent, R_{A2} as a substituent being a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group; and

a plurality of R_{A2} are mutually the same or different.

In the formula (222), Ar₁ represents the same as Ar₁ in the formula (22).

In the formula (222), Ar₂ to Ar₅ are each independently a hydrogen atom or a substituent, and Ar₂ to Ar₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1c).

In the formula (222), at least one of Ar₁ to Ar₅ is a group selected from the group consisting of groups represented by the formulae (1a) to (1c).

The compound M2 is also preferably a compound represented by a formula (11aa), (11bb) or (11cc) below.

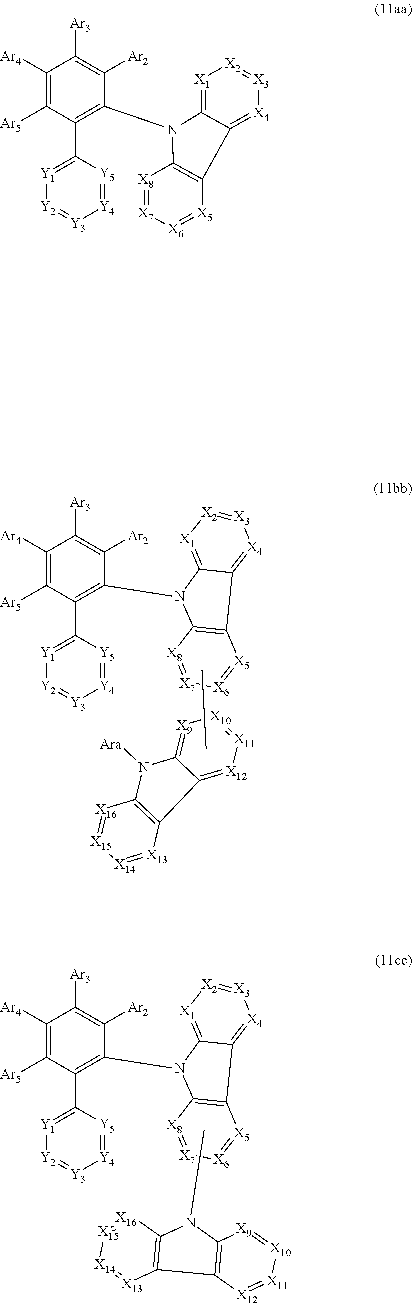

In the formulae (11aa), (11bb) and (11cc), Y₁ to Y₅, R_{A2}, Ar₂ to Ar₅, X₁ to X₁₆, R_{A1} and Ara respectively represent the same as the above-described Y₁ to Y₅, R_{A2}, Ar₂ to Ar_{y}, X₁ to X₁₆, R_{A1} and Ara.

The compound M2 is exemplified by a compound represented by a formula (23) below.

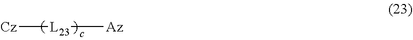

In the formula (23):

Az is a cyclic structure selected from the group consisting of a substituted or unsubstituted pyridine ring, a substituted or unsubstituted pyrimidine ring, a substituted or unsubstituted triazine ring, and a substituted or unsubstituted pyrazine ring;

c is 0, 1, 2, 3, 4 or 5;

when c is 0, Cz and Az are bonded by a single bond;

when c is 1, 2, 3, 4 or 5, L₂₃ is a linking group selected from the group consisting of a substituted or unsubstituted arylene group having 6 to 30 ring carbon atoms, and a substituted or unsubstituted heteroarylene group having 5 to 30 ring atoms;

when c is 2, 3, 4 or 5, a plurality of L₂₃ are mutually the same or different;

the plurality of L₂₃ are mutually bonded to form a ring or not bonded to form no ring; and

Cz is represented by a formula (23a) below.

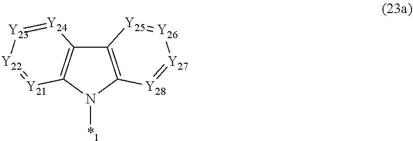

In the formula (23a):

Y₂₁ to Y₂₈ are each independently a nitrogen atom or CR_{A3};

each R_{A3} is independently a hydrogen atom or a substituent, or at least one combination of combinations among a plurality of R_{A3} are mutually bonded to form a ring;

each R_{A3} as a substituent is independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R_{A3} are mutually the same or different; and

*1 represents a bonding position to a carbon atom in a structure of a linking group represented by L₂₃, or a bonding position to a carbon atom in a cyclic structure represented by Az.

Y₂₁ to Y₂₈ are also preferably CR_{A3}.

c in the formula (23) is preferably 0 or 1.

Cz is also preferably represented by a formula (23b), (23c) or (23d) below.

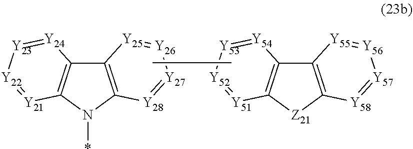

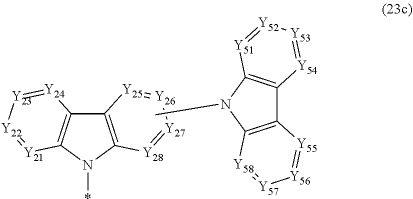

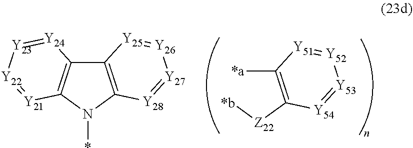

In the formulae (23b), (23c) and (23d), Y₂₁ to Y₂₈ and Y₅₁ to Y₅₈ are each independently a nitrogen atom or CR_{A4};

in the formula (23b), at least one of Y₂₅ to Y₂₈ is a carbon atom bonded to one of Y₅₁ to Y₅₄, and at least one of Y₅₁ to Y₅₄ is a carbon atom bonded to one of Y₂₅ to Y₂₈;

in the formula (23c), at least one of Y₂₅ to Y₂₈ is a carbon atom bonded to a nitrogen atom in a five-membered ring of a nitrogen-containing fused ring including Y₅₁ to Y₅₈;

in the formula (23d), *a and *b each represent a bonding position to one of Y₂₁ to Y₂₈, at least one of Y₂₅ to Y₂₈ is the bonding position represented by *a, and at least one of Y₂₅ to Y₂₈ is the bonding position represented by *b;

n is 1, 2, 3 or 4;

each R_{A4} is independently a hydrogen atom or a substituent, or at least one combination of combinations among a plurality of R_{A4} are mutually bonded to form a ring;

each R_{A4} as a substituent is independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R_{A4} are mutually the same or different;

Z₂₁ and Z₂₂ are each independently any one selected from the group consisting of an oxygen atom, a sulfur atom, NR₄₅ and CR₄₆R₄₇;

R₄₅ is a hydrogen atom or a substituent;

R₄₆ and R₄₇ are each independently a hydrogen atom or a substituent, or a combination of R₄₆ and R₄₇ are mutually bonded to form a ring;

R₄₅, R₄₆ and R₄₇ as a substituent are each independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R₄₅ are mutually the same or different;

a plurality of R₄₆ are mutually the same or different;

a plurality of R₄₇ are mutually the same or different; and

* represents a bonding position to a carbon atom in a structure of a linking group represented by L₂₃, or a bonding position to a carbon atom in a cyclic structure represented by Az.

Z₂₁ is preferably NR₄₅.

When Z₂₁ is NR₄₅, R₄₅ is preferably a substituted or unsubstituted aryl group having 6 to 30 ring carbon atoms.

Z₂₂ is preferably NR₄₅.

When Z₂₂ is NR₄₅, R₄₅ is preferably a substituted or unsubstituted aryl group having 6 to 30 ring carbon atoms.

Y₅₁ to Y₅₈ are preferably CR_{A4}, provided that at least one of Y₅₁ to Y₅₈ is a carbon atom bonded to a cyclic structure represented by the formula (23a).

It is also preferable that Cz is represented by the formula (23d) and n is 1.

Az is preferably a cyclic structure selected from the group consisting of a substituted or unsubstituted pyrimidine group and a substituted or unsubstituted triazine group.

Az is a cyclic structure selected from the group consisting of a substituted pyrimidine ring and a substituted triazine ring, in which a substituent of each of the substituted pyrimidine ring and the substituted triazine ring is more preferably a group selected from the group consisting of a substituted or unsubstituted aryl group having 6 to 30 ring carbon atoms and a substituted or unsubstituted heteroaryl group having 5 to 30 ring atoms, further preferably a substituted or unsubstituted aryl group having 6 to 30 ring carbon atoms.

When the pyrimidine ring and the triazine ring as Az have a substituted or unsubstituted aryl group as a substituent, the aryl group preferably has 6 to 20 ring carbon atoms, more preferably 6 to 14 ring carbon atoms, further preferably 6 to 12 ring carbon atoms.

When Az has a substituted or unsubstituted aryl group as a substituent, the substituent is preferably a group selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a group selected from the group consisting of a substituted or unsubstituted phenyl group, a substituted or unsubstituted biphenyl group, and a substituted or unsubstituted naphthyl group.

When Az has a substituted or unsubstituted heteroaryl group as a substituent, the substituent is preferably a substituent selected from the group consisting of a substituted or unsubstituted carbazolyl group, a substituted or unsubstituted dibenzofuranyl group, and a substituted or unsubstituted dibenzothiophenyl group.

It is preferable that each R_{A4} is independently a hydrogen atom or a substituent, and R_{A4} as a substituent is a substituent selected from the group consisting of a substituted or unsubstituted aryl group having 6 to 30 ring carbon atoms and a substituted or unsubstituted heteroaryl group having 5 to 30 ring atoms.

When R_{A4} as a substituent is a substituted or unsubstituted aryl group having 6 to 30 ring carbon atoms, R_{A4} as a substituent is preferably a substituent selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a substituent selected from the group consisting of a substituted or unsubstituted phenyl group, a substituted or unsubstituted biphenyl group, and a substituted or unsubstituted naphthyl group.

When R_{A4} as a substituent is a substituted or unsubstituted heteroaryl group having 5 to 30 ring atoms, R_{A4} as a substituent is preferably a substituent selected from the group consisting of a substituted or unsubstituted carbazolyl group, a substituted or unsubstituted dibenzofuranyl group, and a substituted or unsubstituted dibenzothiophenyl group.

R₄₅, R₄₆ and R₄₇ as a substituent are preferably each independently a substituent selected from the group consisting of a substituted or unsubstituted aryl group having 6 to 30 ring carbon atoms, a substituted or unsubstituted heteroaryl group having 5 to 30 ring atoms, and a substituted or unsubstituted alkyl group having 1 to 30 carbon atoms.

### Method for Manufacturing Compound M2

The compound M2 can be manufactured by a known method.

Specific examples of the compound M2 include the following compounds. It should however be noted that the invention is not limited to the specific examples of the compound.

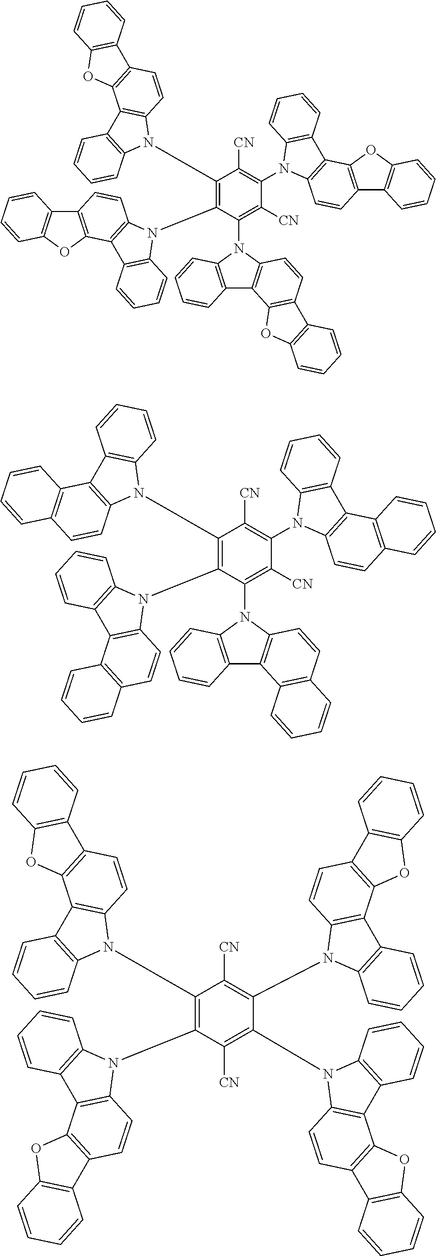 [SMILES: N#Cc1c(-n2c3ccccc3c3c4oc5ccccc5c4ccc32)c(C#N)c(-n2c3ccccc3c3c4oc5ccccc5c4ccc32)c(-n2c3ccccc3c3c4oc5ccccc5c4ccc32)c1-n1c2ccccc2c2c3oc4ccccc4c3ccc21 ; N#Cc1c(-n2c3ccccc3c3c4ccccc4ccc32)c(C#N)c(-n2c3ccccc3c3c4ccccc4ccc32)c(-n2c3ccccc3c3c4ccccc4ccc32)c1-n1c2ccccc2c2c3ccccc3ccc21 ; N#Cc1c(-n2c3ccccc3c3c4oc5ccccc5c4ccc32)c(-n2c3ccccc3c3c4oc5ccccc5c4ccc32)c(C#N)c(-n2c3ccccc3c3c4oc5ccccc5c4ccc32)c1-n1c2ccccc2c2c3oc4ccccc4c3ccc21]

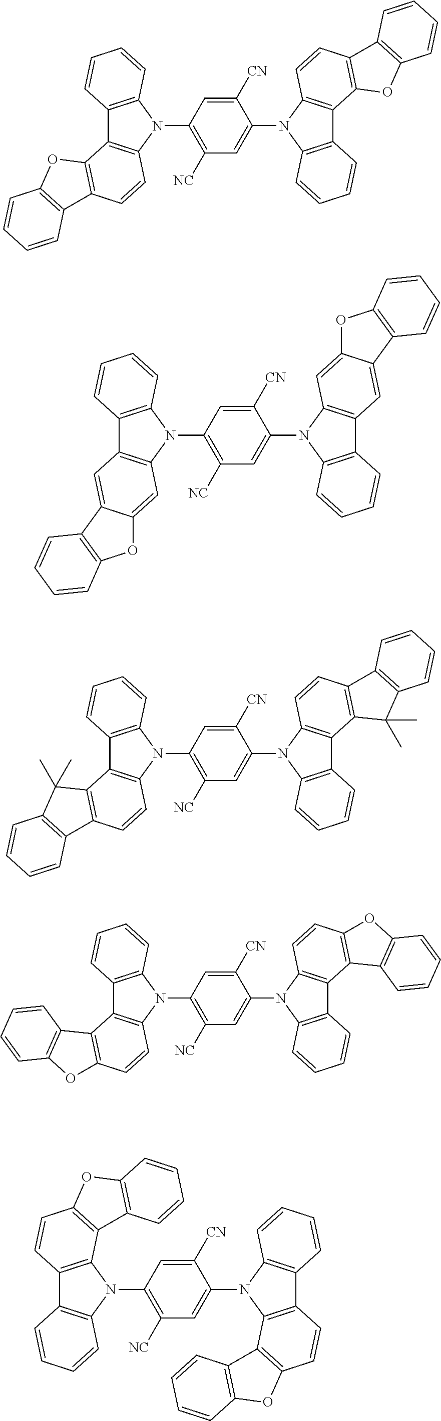 [SMILES: N#Cc1cc(-n2c3ccccc3c3c4oc5ccccc5c4ccc32)c(C#N)cc1-n1c2ccccc2c2c3oc4ccccc4c3ccc21 ; N#Cc1cc(-n2c3ccccc3c3cc4c(cc32)oc2ccccc24)c(C#N)cc1-n1c2ccccc2c2cc3c(cc21)oc1ccccc13 ; CC1(C)c2ccccc2-c2ccc3c(c21)c1ccccc1n3-c1cc(C#N)c(-n2c3ccccc3c3c4c(ccc32)-c2ccccc2C4(C)C)cc1C#N ; N#Cc1cc(-n2c3ccccc3c3c4c(ccc32)oc2ccccc24)c(C#N)cc1-n1c2ccccc2c2c3c(ccc21)oc1ccccc13 ; N#Cc1cc(-n2c3ccccc3c3ccc4oc5ccccc5c4c32)c(C#N)cc1-n1c2ccccc2c2ccc3oc4ccccc4c3c21]

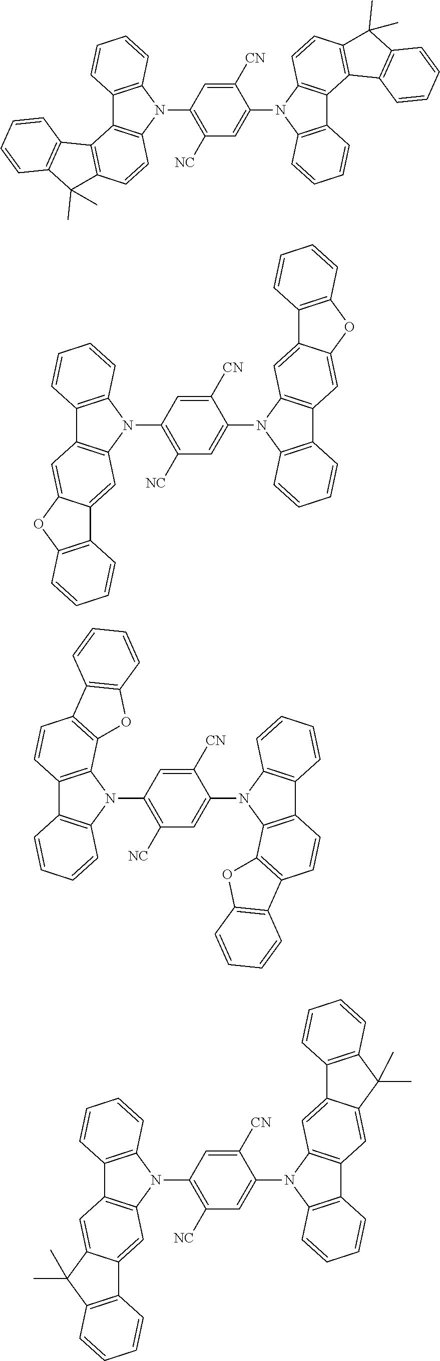

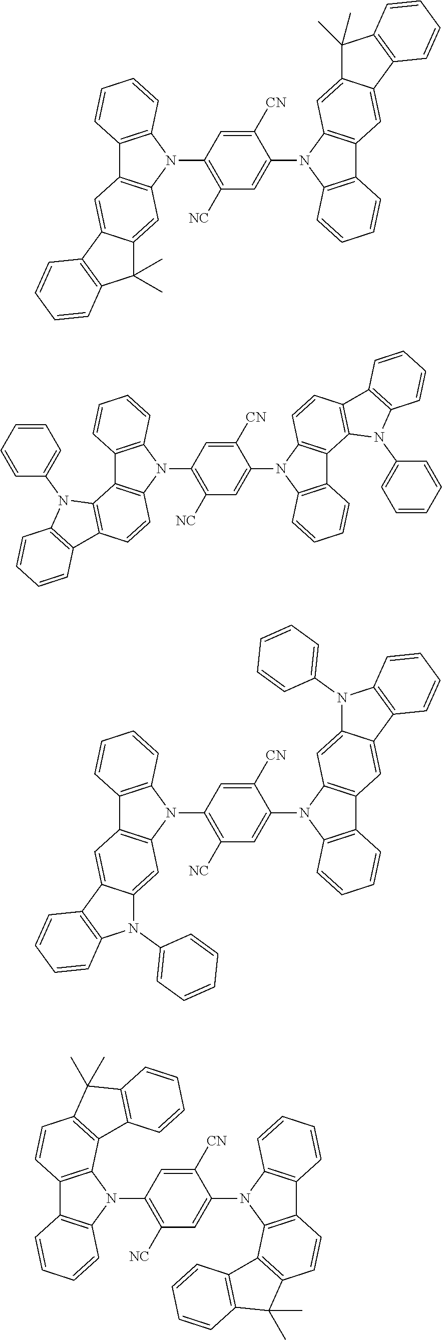 [SMILES: CC1(C)c2ccccc2-c2cc3c4ccccc4n(-c4cc(C#N)c(-n5c6ccccc6c6cc7c(cc65)C(C)(C)c5ccccc5-7)cc4C#N)c3cc21 ; N#Cc1cc(-n2c3ccccc3c3c2ccc2c4ccccc4n(-c4ccccc4)c23)c(C#N)cc1-n1c2ccccc2c2c1ccc1c3ccccc3n(-c3ccccc3)c12 ; N#Cc1cc(-n2c3ccccc3c3cc4c5ccccc5n(-c5ccccc5)c4cc32)c(C#N)cc1-n1c2ccccc2c2cc3c4ccccc4n(-c4ccccc4)c3cc21 ; CC1(C)c2ccccc2-c2c1ccc1c3ccccc3n(-c3cc(C#N)c(-n4c5ccccc5c5ccc6c(c54)-c4ccccc4C6(C)C)cc3C#N)c21]

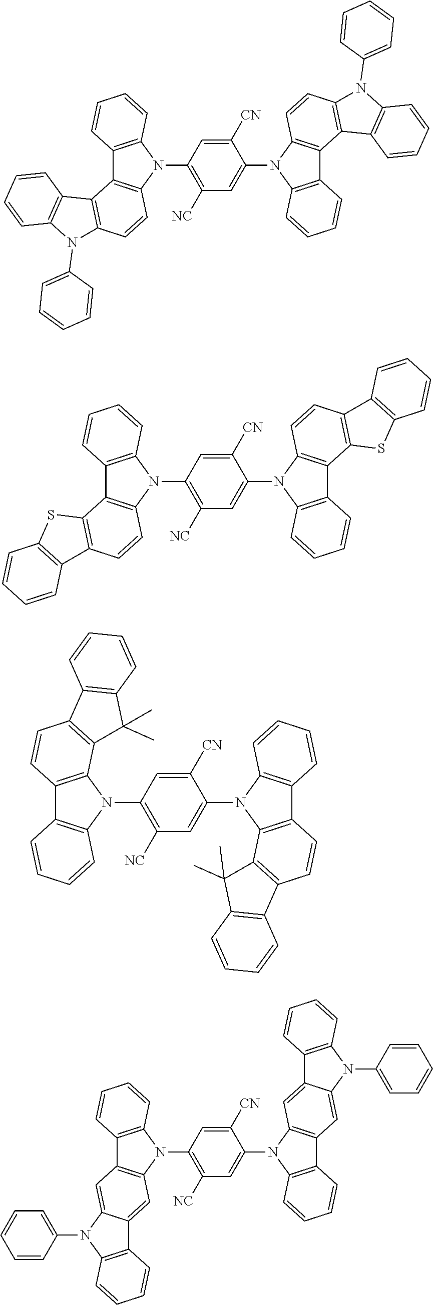 [SMILES: N#Cc1cc(-n2c3ccccc3c3c4c5ccccc5n(-c5ccccc5)c4ccc32)c(C#N)cc1-n1c2ccccc2c2c3c4ccccc4n(-c4ccccc4)c3ccc21 ; N#Cc1cc(-n2c3ccccc3c3c4sc5ccccc5c4ccc32)c(C#N)cc1-n1c2ccccc2c2c3sc4ccccc4c3ccc21 ; CC1(C)c2ccccc2-c2ccc3c4ccccc4n(-c4cc(C#N)c(-n5c6ccccc6c6ccc7c(c65)C(C)(C)c5ccccc5-7)cc4C#N)c3c21 ; N#Cc1cc(-n2c3ccccc3c3cc4c(cc32)c2ccccc2n4-c2ccccc2)c(C#N)cc1-n1c2ccccc2c2cc3c(cc21)c1ccccc1n3-c1ccccc1]

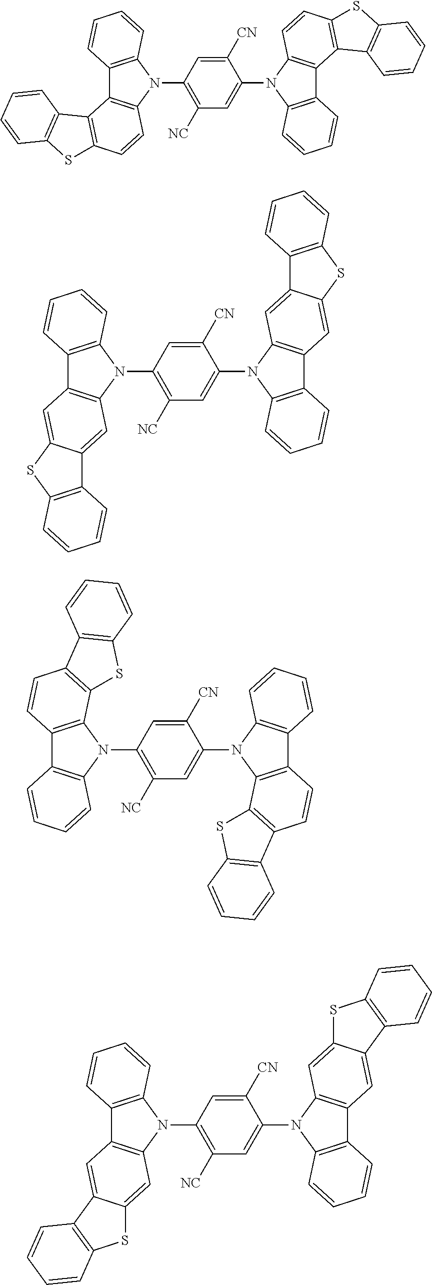

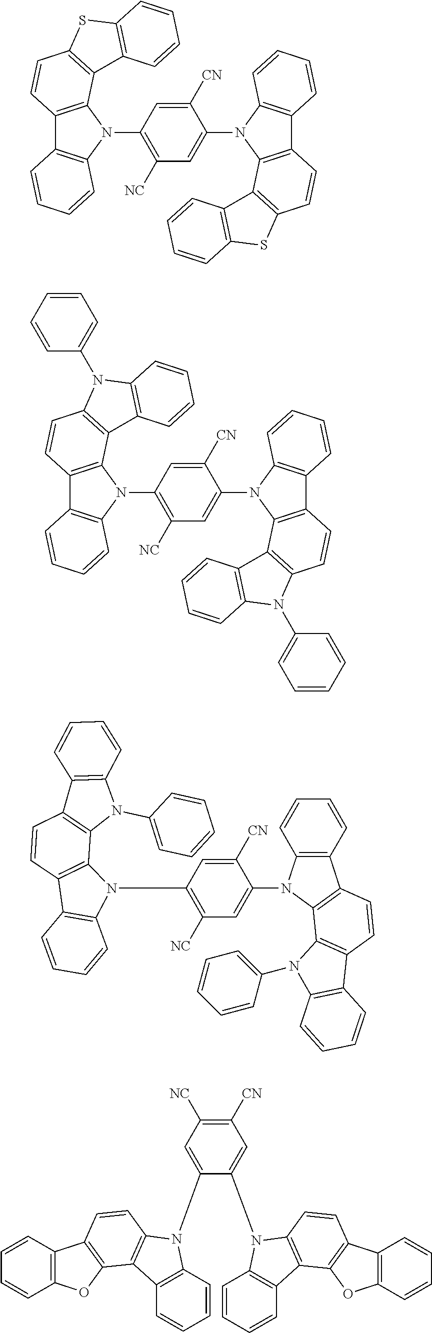 [SMILES: N#Cc1cc(-n2c3ccccc3c3ccc4sc5ccccc5c4c32)c(C#N)cc1-n1c2ccccc2c2ccc3sc4ccccc4c3c21 ; N#Cc1cc(-n2c3ccccc3c3ccc4c(c5ccccc5n4-c4ccccc4)c32)c(C#N)cc1-n1c2ccccc2c2ccc3c(c4ccccc4n3-c3ccccc3)c21 ; N#Cc1cc(-n2c3ccccc3c3ccc4c5ccccc5n(-c5ccccc5)c4c32)c(C#N)cc1-n1c2ccccc2c2ccc3c4ccccc4n(-c4ccccc4)c3c21 ; N#Cc1cc(-n2c3ccccc3c3c4oc5ccccc5c4ccc32)c(-n2c3ccccc3c3c4oc5ccccc5c4ccc32)cc1C#N]

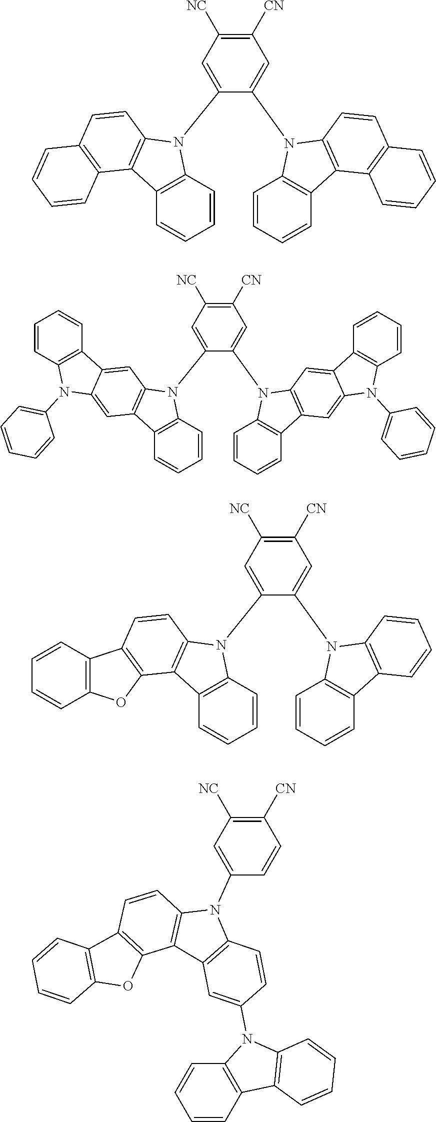

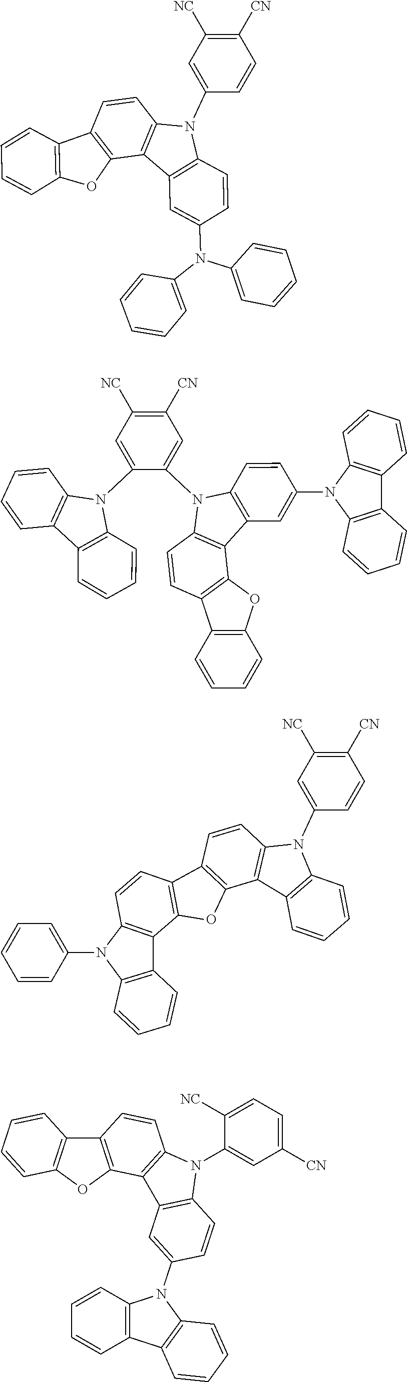 [SMILES: N#Cc1ccc(-n2c3ccc(N(c4ccccc4)c4ccccc4)cc3c3c4oc5ccccc5c4ccc32)cc1C#N ; N#Cc1cc(-n2c3ccccc3c3ccccc32)c(-n2c3ccc(-n4c5ccccc5c5ccccc54)cc3c3c4oc5ccccc5c4ccc32)cc1C#N ; N#Cc1ccc(-n2c3ccccc3c3c4oc5c(ccc6c5c5ccccc5n6-c5ccccc5)c4ccc32)cc1C#N ; N#Cc1ccc(C#N)c(-n2c3ccc(-n4c5ccccc5c5ccccc54)cc3c3c4oc5ccccc5c4ccc32)c1]

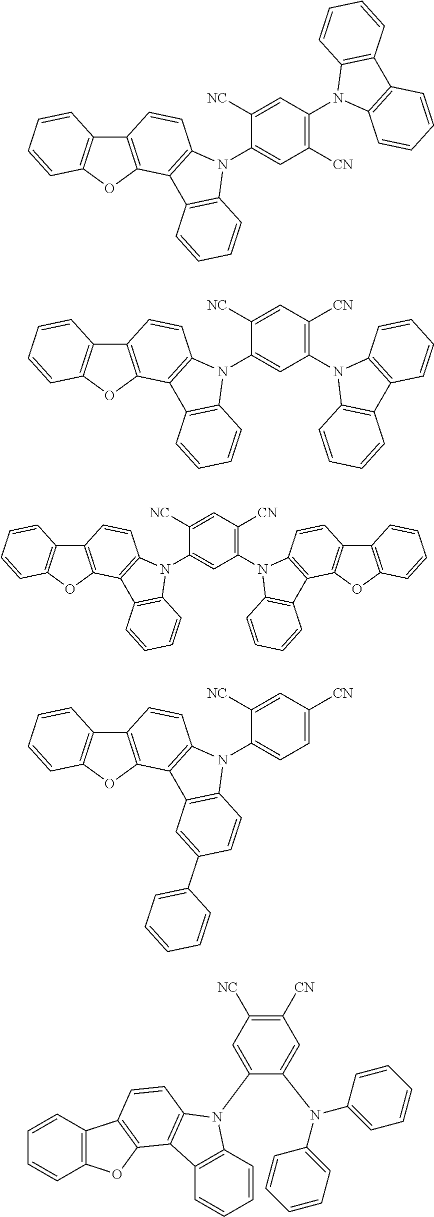

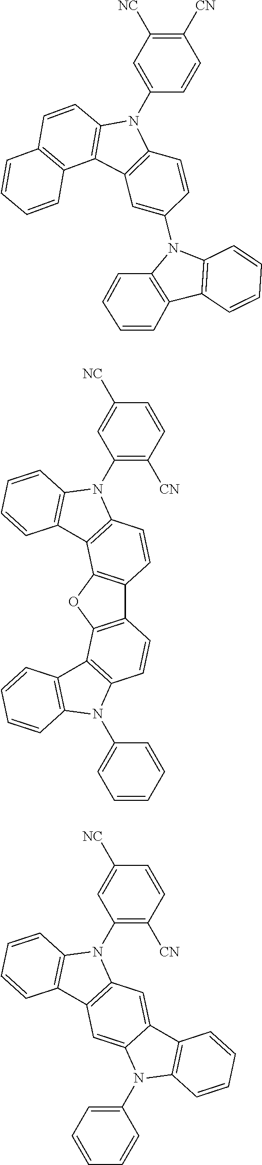

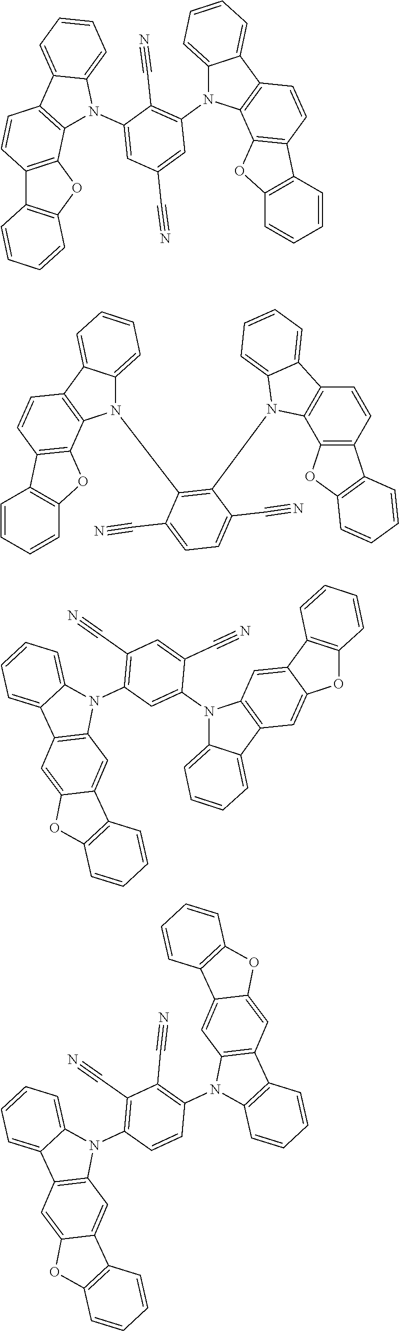 [SMILES: N#Cc1cc(-n2c3ccccc3c3ccc4c5ccccc5oc4c32)c(C#N)c(-n2c3ccccc3c3ccc4c5ccccc5oc4c32)c1 ; N#Cc1ccc(C#N)c(-n2c3ccccc3c3ccc4c5ccccc5oc4c32)c1-n1c2ccccc2c2ccc3c4ccccc4oc3c21 ; N#Cc1cc(C#N)c(-n2c3ccccc3c3cc4oc5ccccc5c4cc32)cc1-n1c2ccccc2c2cc3oc4ccccc4c3cc21 ; N#Cc1c(-n2c3ccccc3c3cc4oc5ccccc5c4cc32)ccc(-n2c3ccccc3c3cc4oc5ccccc5c4cc32)c1C#N]

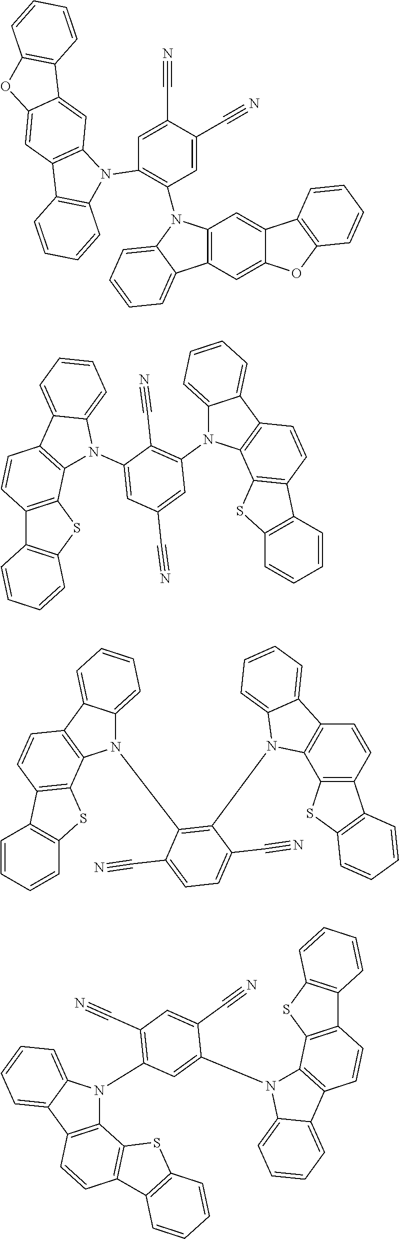 [SMILES: N#Cc1cc(-n2c3ccccc3c3cc4oc5ccccc5c4cc32)c(-n2c3ccccc3c3cc4oc5ccccc5c4cc32)cc1C#N ; N#Cc1cc(-n2c3ccccc3c3ccc4c5ccccc5sc4c32)c(C#N)c(-n2c3ccccc3c3ccc4c5ccccc5sc4c32)c1 ; N#Cc1ccc(C#N)c(-n2c3ccccc3c3ccc4c5ccccc5sc4c32)c1-n1c2ccccc2c2ccc3c4ccccc4sc3c21 ; N#Cc1cc(C#N)c(-n2c3ccccc3c3ccc4c5ccccc5sc4c32)cc1-n1c2ccccc2c2ccc3c4ccccc4sc3c21]

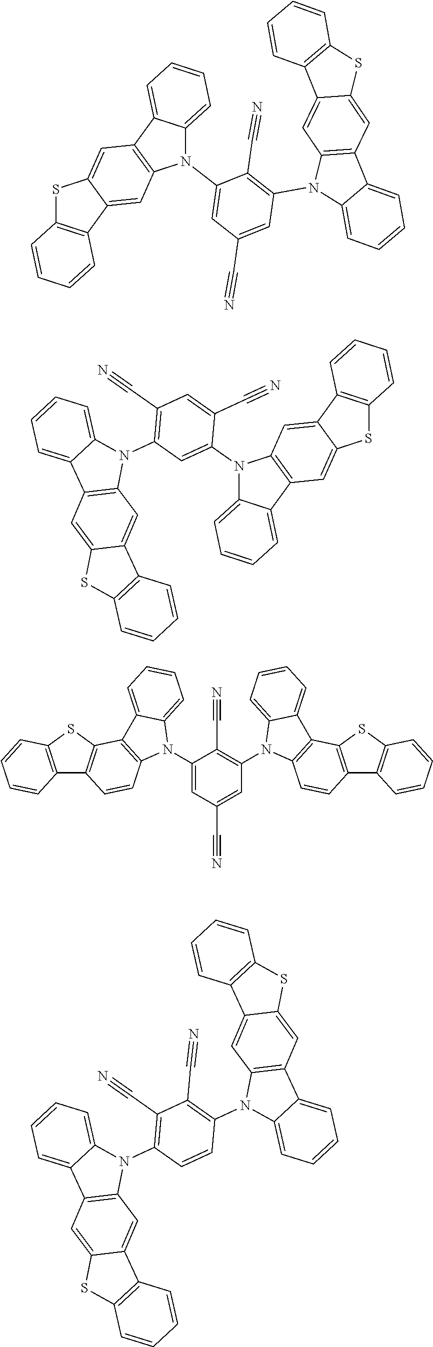

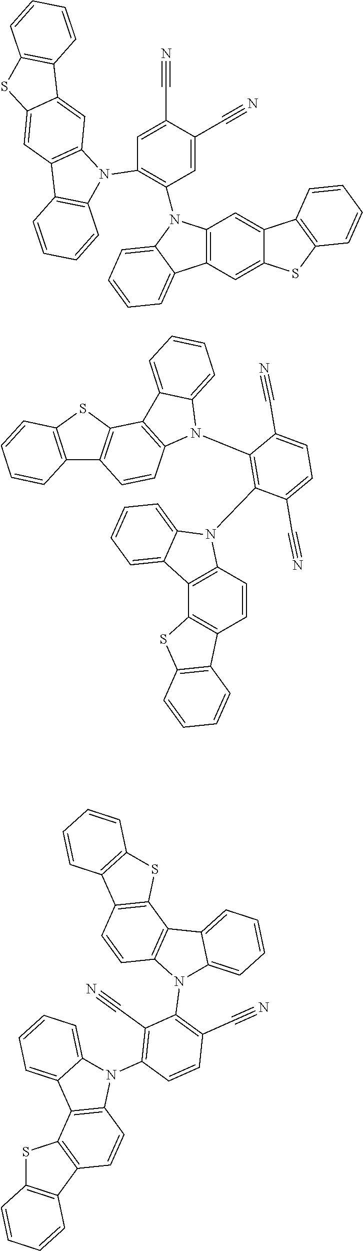

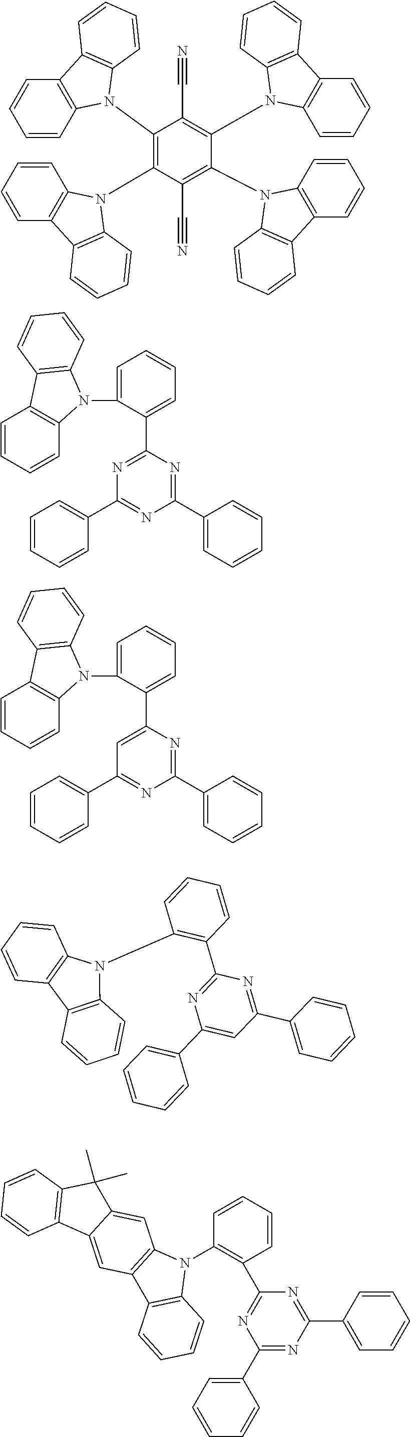

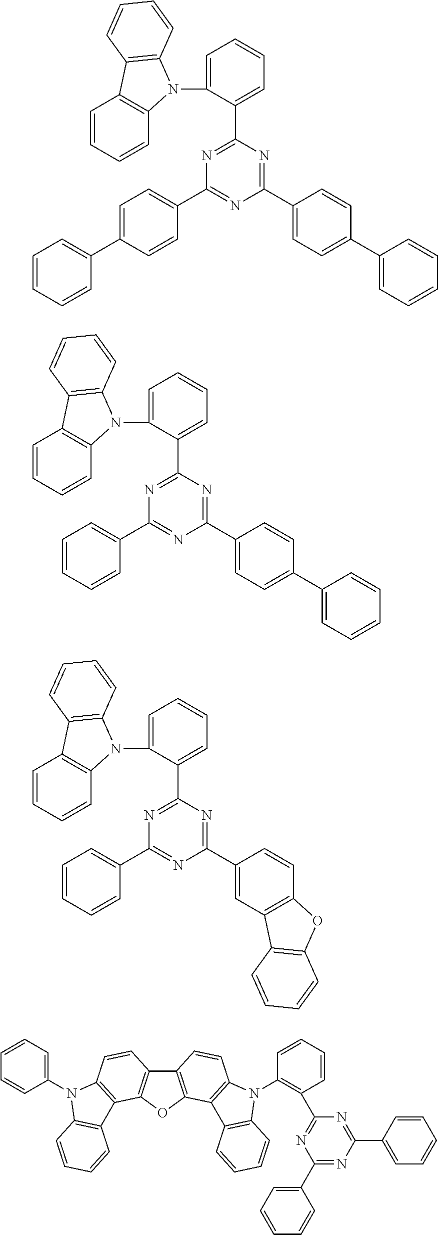

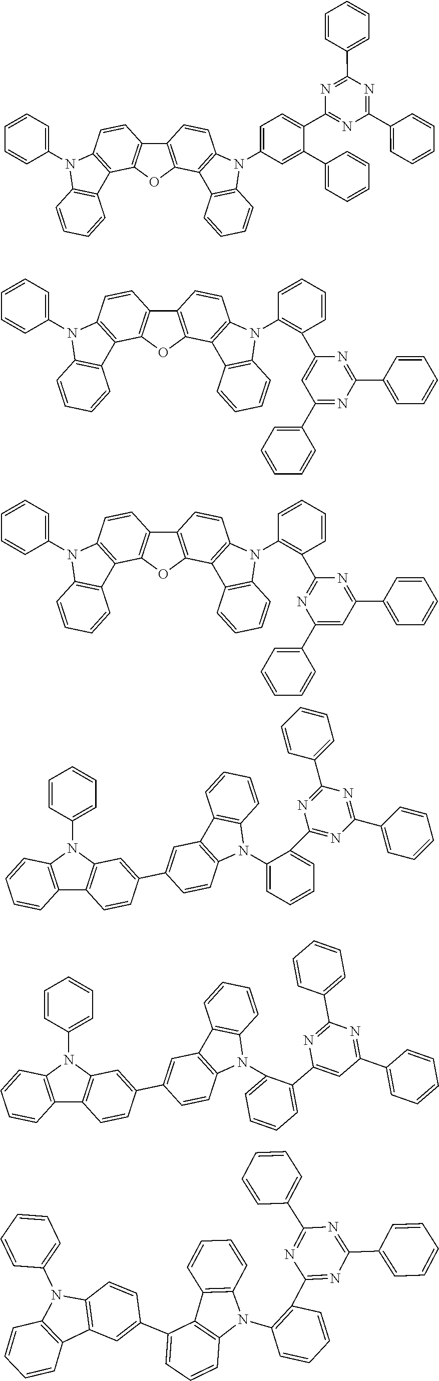

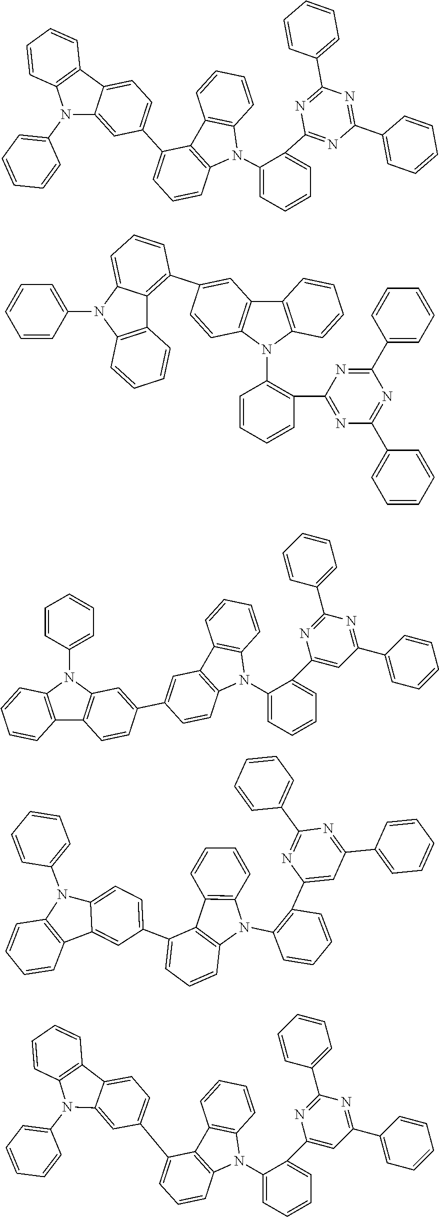

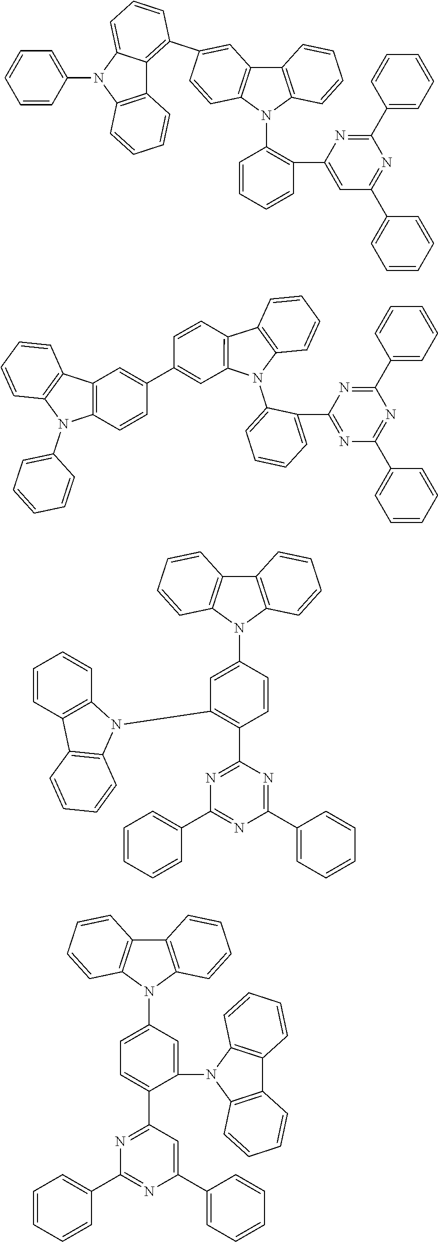

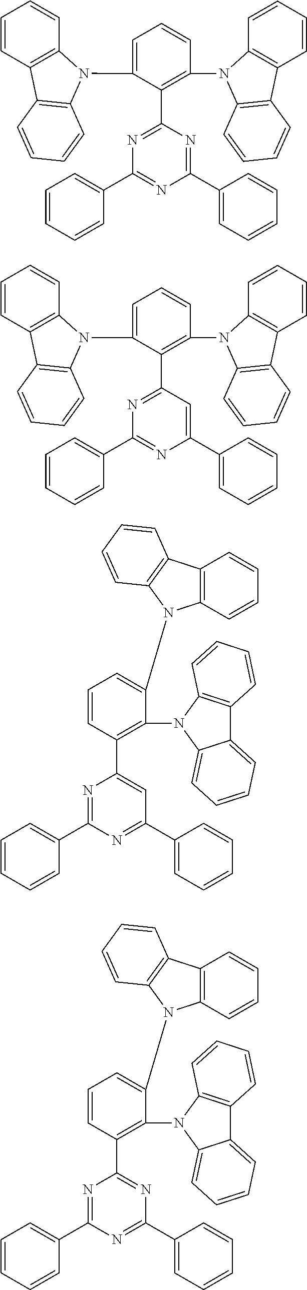

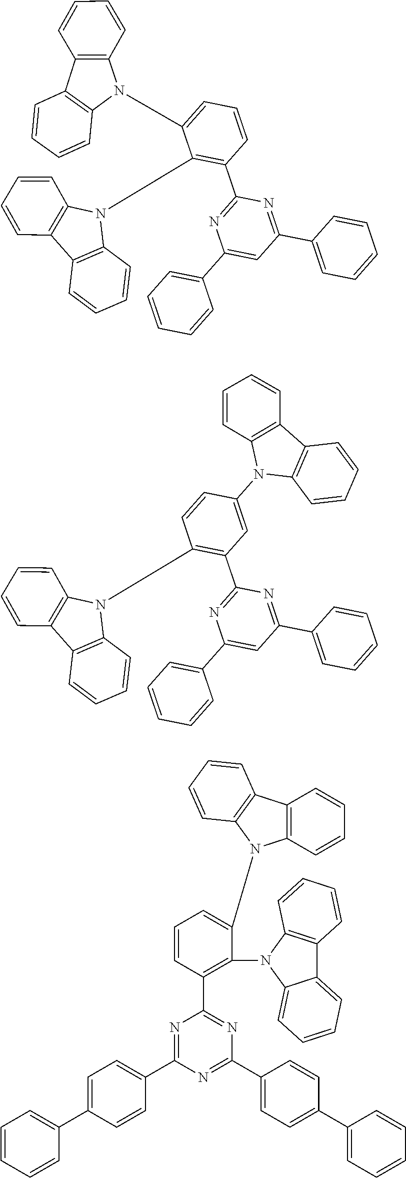

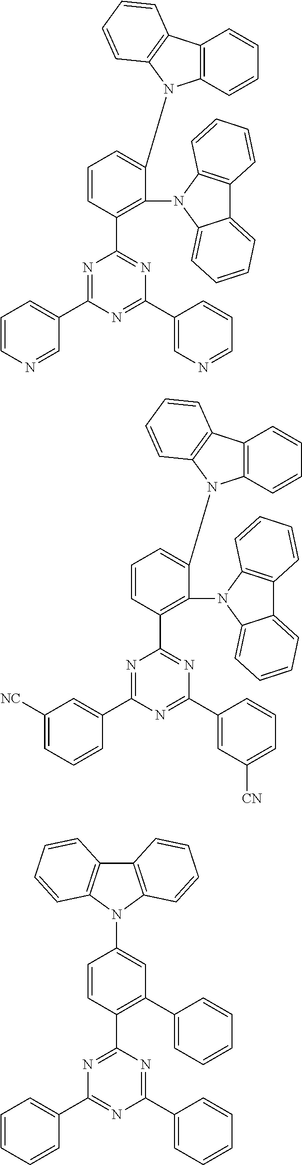

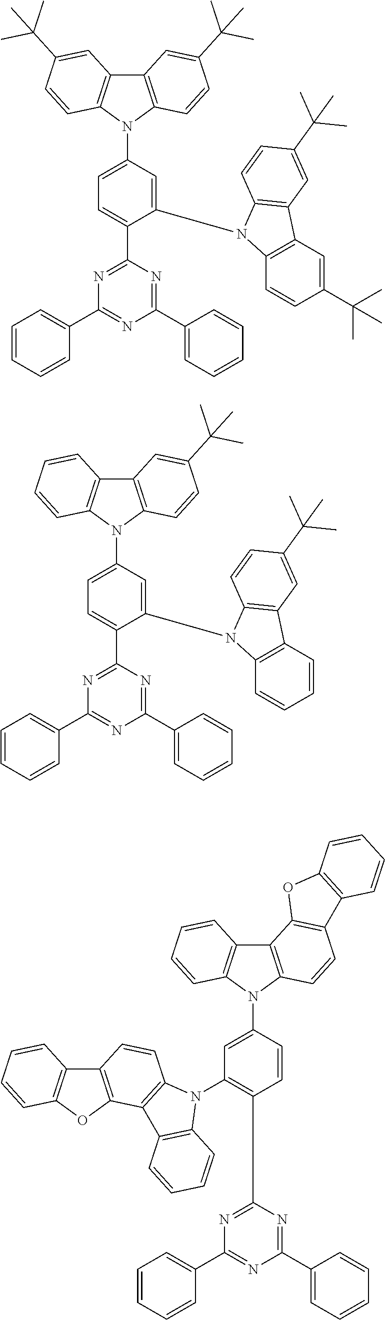

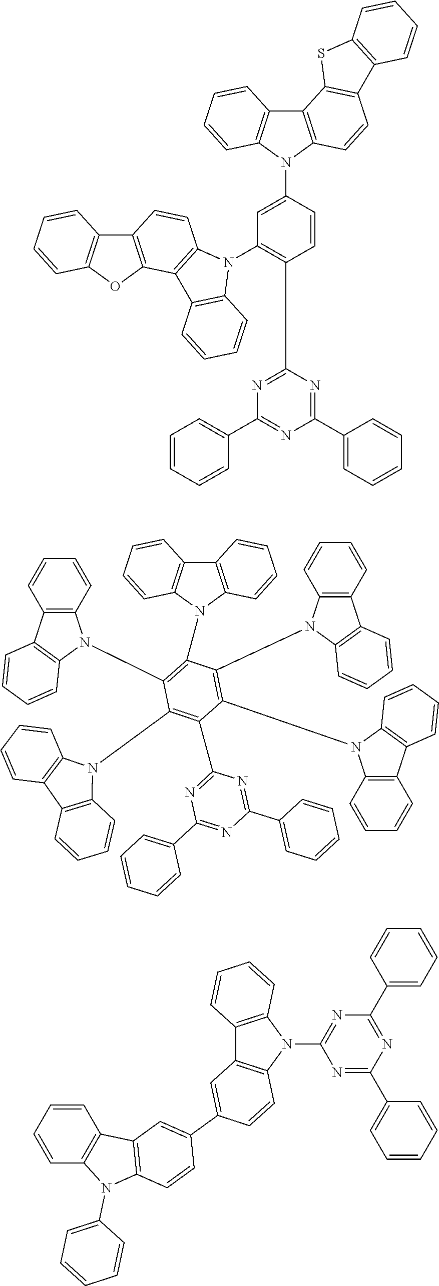 [SMILES: c1ccc(-c2nc(-c3ccccc3)nc(-c3ccc(-n4c5ccccc5c5c6sc7ccccc7c6ccc54)cc3-n3c4ccccc4c4c5oc6ccccc6c5ccc43)n2)cc1 ; c1ccc(-c2nc(-c3ccccc3)nc(-c3c(-n4c5ccccc5c5ccccc54)c(-n4c5ccccc5c5ccccc54)c(-n4c5ccccc5c5ccccc54)c(-n4c5ccccc5c5ccccc54)c3-n3c4ccccc4c4ccccc43)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

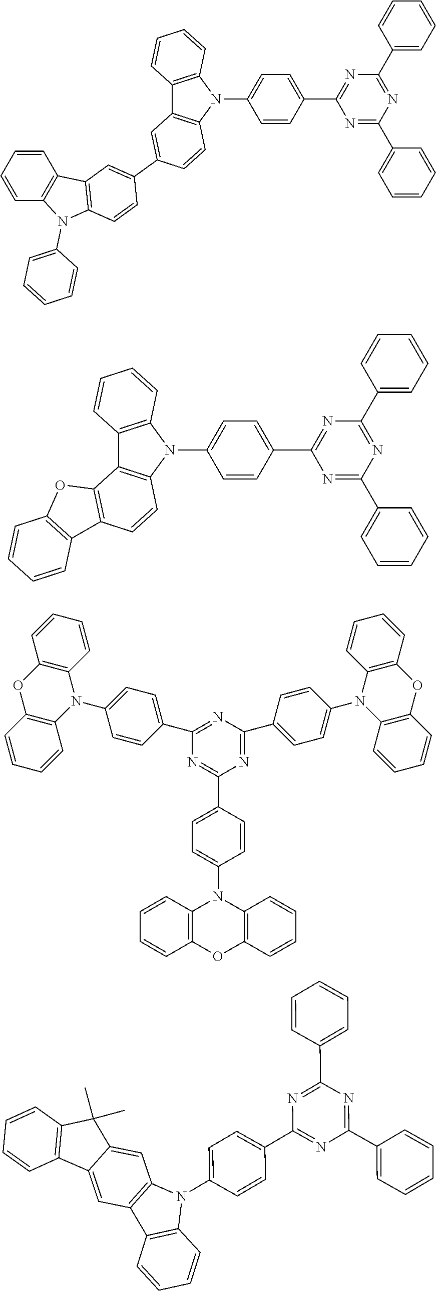

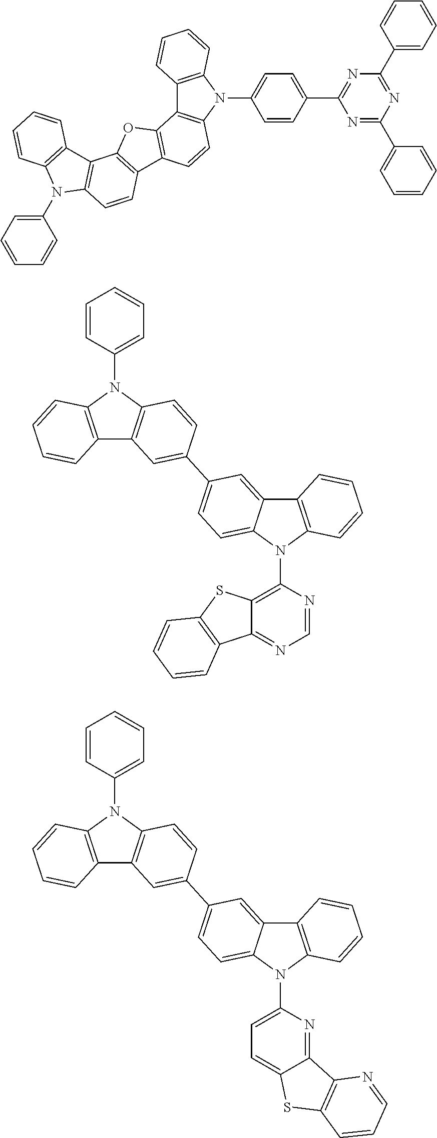 [SMILES: c1ccc(-c2nc(-c3ccccc3)nc(-c3ccc(-n4c5ccccc5c5c6oc7c(ccc8c7c7ccccc7n8-c7ccccc7)c6ccc54)cc3)n2)cc1 ; c1ccc(-n2c3ccccc3c3cc(-c4ccc5c(c4)c4ccccc4n5-c4ncnc5c4sc4ccccc45)ccc32)cc1 ; c1ccc(-n2c3ccccc3c3cc(-c4ccc5c(c4)c4ccccc4n5-c4ccc5sc6cccnc6c5n4)ccc32)cc1]

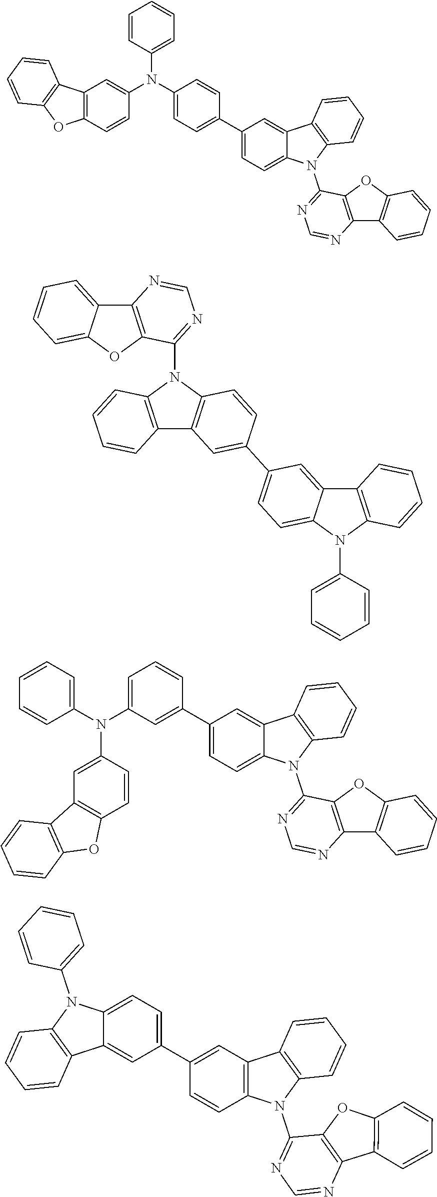

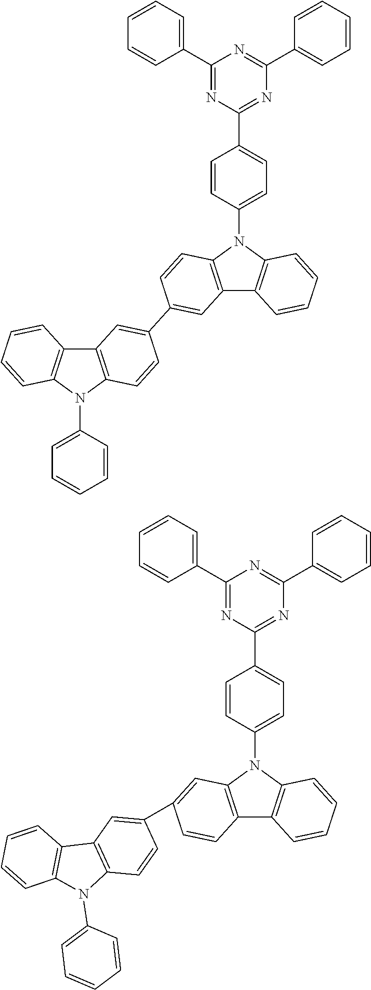 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1]

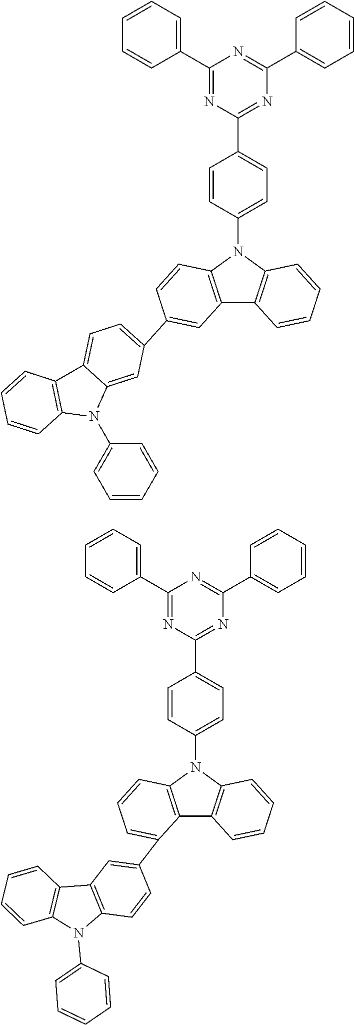

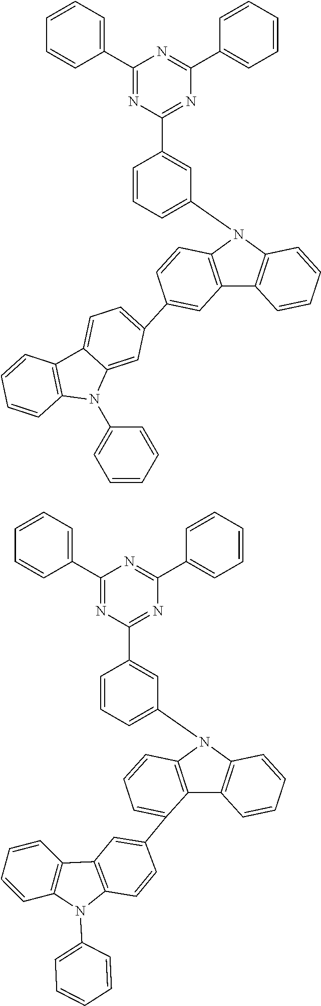 [SMILES: c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2nc(-c3ccccc3)nc(-c3cccc(-n4c5ccccc5c5c(-c6ccc7c(c6)c6ccccc6n7-c6ccccc6)cccc54)c3)n2)cc1]

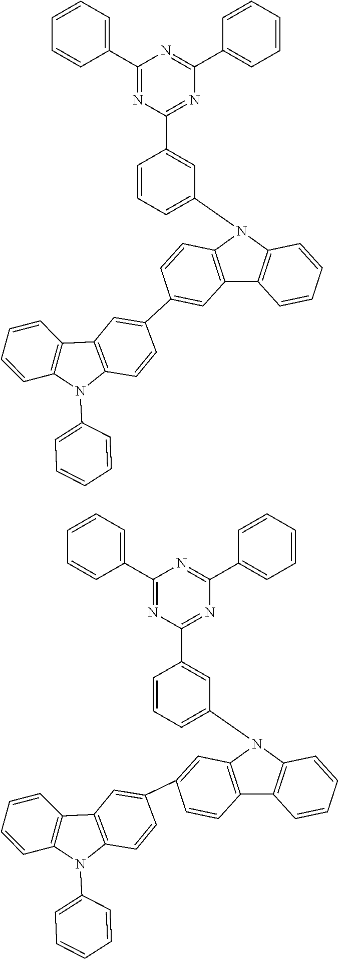

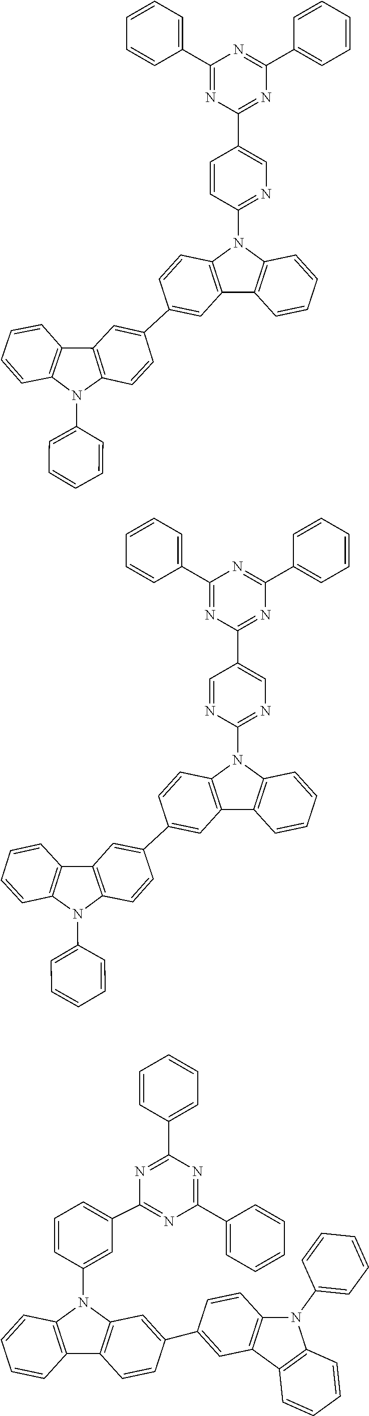 [SMILES: c1ccc(-c2nc(-c3ccccc3)nc(-c3ccc(-n4c5ccccc5c5cc(-c6ccc7c(c6)c6ccccc6n7-c6ccccc6)ccc54)nc3)n2)cc1 ; c1ccc(-c2nc(-c3ccccc3)nc(-c3cnc(-n4c5ccccc5c5cc(-c6ccc7c(c6)c6ccccc6n7-c6ccccc6)ccc54)nc3)n2)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

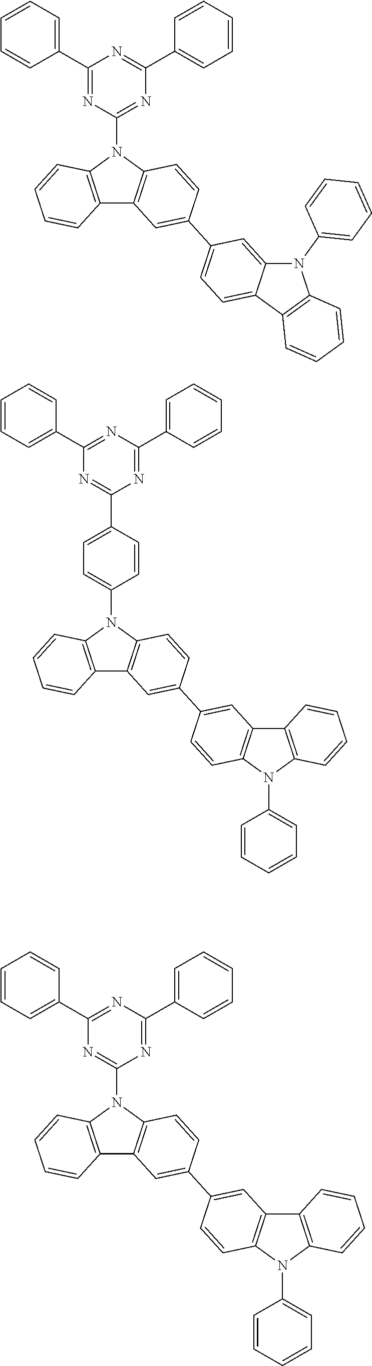 [SMILES: c1ccc(-c2nc(-c3ccccc3)nc(-n3c4ccccc4c4cc(-c5ccc6c7ccccc7n(-c7ccccc7)c6c5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

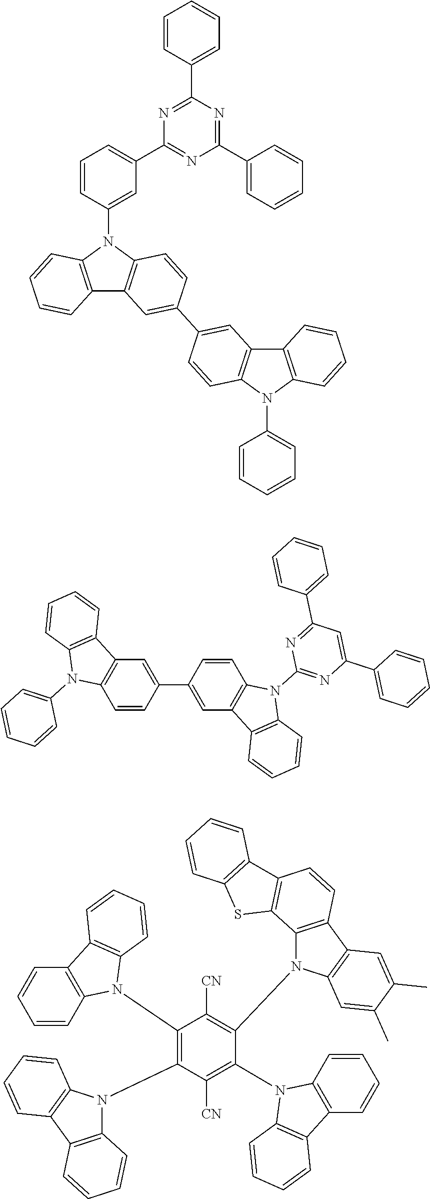 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2cc(-c3ccccc3)nc(-n3c4ccccc4c4cc(-c5ccc6c(c5)c5ccccc5n6-c5ccccc5)ccc43)n2)cc1 ; Cc1cc2c3ccc4c5ccccc5sc4c3n(-c3c(C#N)c(-n4c5ccccc5c5ccccc54)c(-n4c5ccccc5c5ccccc54)c(C#N)c3-n3c4ccccc4c4ccccc43)c2cc1C]

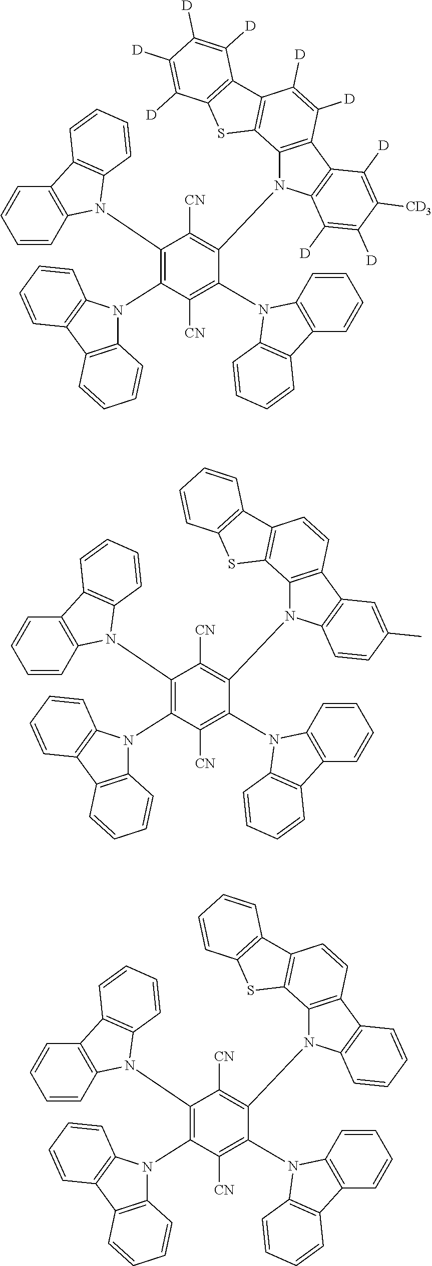 [SMILES: [2H]c1c([2H])c([2H])c2c(sc3c2c([2H])c([2H])c2c4c([2H])c(C([2H])([2H])[2H])c([2H])c([2H])c4n(-c4c(C#N)c(-n5c6ccccc6c6ccccc65)c(-n5c6ccccc6c6ccccc65)c(C#N)c4-n4c5ccccc5c5ccccc54)c32)c1[2H] ; Cc1ccc2c(c1)c1ccc3c4ccccc4sc3c1n2-c1c(C#N)c(-n2c3ccccc3c3ccccc32)c(-n2c3ccccc3c3ccccc32)c(C#N)c1-n1c2ccccc2c2ccccc21 ; N#Cc1c(-n2c3ccccc3c3ccccc32)c(-n2c3ccccc3c3ccccc32)c(C#N)c(-n2c3ccccc3c3ccc4c5ccccc5sc4c32)c1-n1c2ccccc2c2ccccc21]

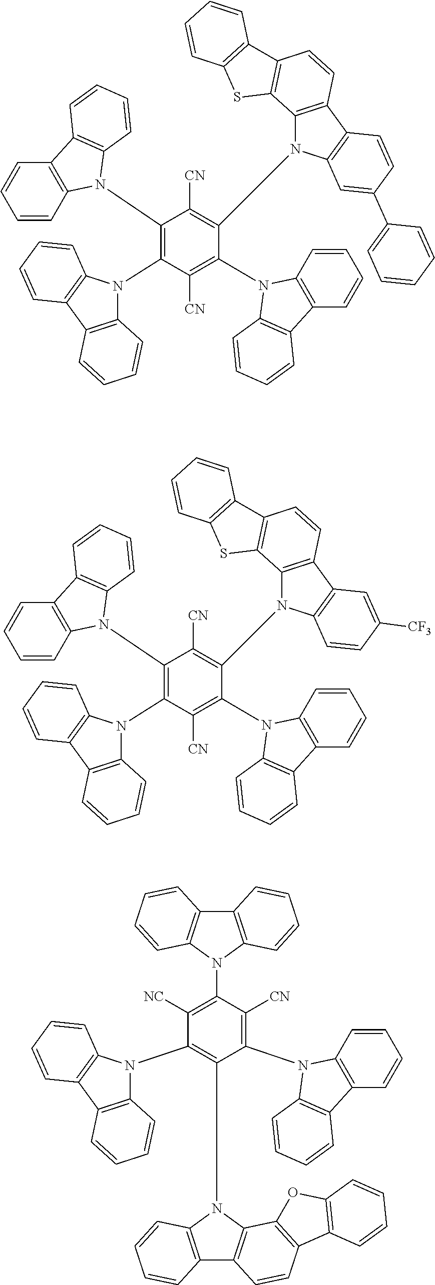 [SMILES: N#Cc1c(-n2c3ccccc3c3ccccc32)c(-n2c3ccccc3c3ccccc32)c(C#N)c(-n2c3cc(-c4ccccc4)ccc3c3ccc4c5ccccc5sc4c32)c1-n1c2ccccc2c2ccccc21 ; N#Cc1c(-n2c3ccccc3c3ccccc32)c(-n2c3ccccc3c3ccccc32)c(C#N)c(-n2c3ccc(C(F)(F)F)cc3c3ccc4c5ccccc5sc4c32)c1-n1c2ccccc2c2ccccc21 ; N#Cc1c(-n2c3ccccc3c3ccccc32)c(C#N)c(-n2c3ccccc3c3ccccc32)c(-n2c3ccccc3c3ccc4c5ccccc5oc4c32)c1-n1c2ccccc2c2ccccc21]

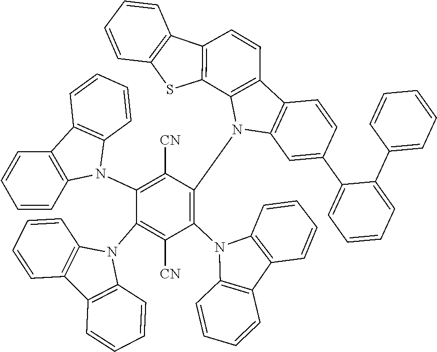 [SMILES: N#Cc1c(-n2c3ccccc3c3ccccc32)c(-n2c3ccccc3c3ccccc32)c(C#N)c(-n2c3cc(-c4ccccc4-c4ccccc4)ccc3c3ccc4c5ccccc5sc4c32)c1-n1c2ccccc2c2ccccc21]

### Compound M1

The compound M1 of the exemplary embodiment is not a phosphorescent metal complex. The compound M1 is preferably not a heavy metal complex. Further, the compound M1 is preferably not a metal complex.

Further, the compound M1 is preferably a compound exhibiting no thermally activated delayed fluorescence.

A fluorescent material is usable as the compound M1. Specific examples of the fluorescent material include a bisarylaminonaphthalene derivative, aryl-substituted naphthalene derivative, bisarylaminoanthracene derivative, aryl-substituted anthracene derivative, bisarylaminopyrene derivative, aryl-substituted pyrene derivative, bisarylamino chrysene derivative, aryl-substituted chrysene derivative, bisarylaminofluoranthene derivative, aryl-substituted fluoranthene derivative, indenoperylene derivative, acenaphthofluoranthene derivative, compound including a boron atom, pyromethene boron complex compound, compound having a pyromethene skeleton, metal complex of the compound having a pyrromethene skeleton, diketopyrrolopyrrole derivative, perylene derivative, and naphthacene derivative.

In the exemplary embodiment, the compound M1 that fluoresces is preferably a compound represented by a formula (20) below.

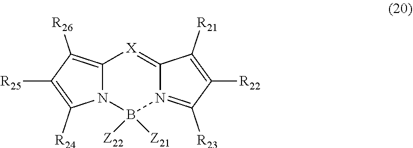

In the formula (20):

X is a nitrogen atom, or a carbon atom bonded to Y;

Y is a hydrogen atom or a substituent;

R₂₁ to R₂₆ are each independently a hydrogen atom or a substituent, or at least one of a combination of R₂₁ and R₂₂, a combination of R₂₂ and R₂₃, a combination of R₂₄ and R₂₅, or a combination of R₂₅ and R₂₆ are mutually bonded to form a ring;

Y and R₂₁ to R₂₆ as a substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;

Z₂₁ and Z₂₂ are each independently a substituent, or are mutually bonded to form a ring; and

Z₂₁ and Z₂₂ as a substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

In the exemplary embodiment, the fluorescent compound M1 is also preferably a compound represented by a formula (1) below.

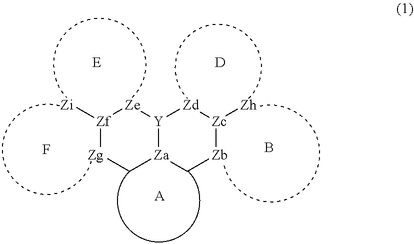

In the formula (1):

a ring A, ring B, ring D, ring E, and ring F are each independently a cyclic structure selected from the group consisting of a substituted or unsubstituted aryl ring having 6 to 30 ring carbon atoms and a substituted or unsubstituted heterocycle having 5 to 30 ring atoms;

one of the ring B and the ring D is present or both of the ring B and the ring D are present;

when both of the ring B and the ring D are present, the ring B and the ring D share a bond between Zc and Zh;

one of the ring E and the ring F is present or both of the ring E and the ring F are present;

when both of the ring E and the ring F are present, the ring E and the ring F share a bond between Zf and Zi;

Za is a nitrogen atom or a carbon atom;

Zb is a nitrogen atom or a carbon atom when the ring B is present;

Zb is an oxygen atom, a sulfur atom, NRb, C(Rb₁)(Rb₂), or Si(Rb₃)(Rb₄) when the ring B is not present;

Zc is a nitrogen atom or a carbon atom;

Zd is a nitrogen atom or a carbon atom when the ring D is present;

Zd is an oxygen atom, a sulfur atom, or NRd when the ring D is not present;

Ze is a nitrogen atom or a carbon atom when the ring E is present;

Ze is an oxygen atom, a sulfur atom, or NRe when the ring E is not present;

Zf is a nitrogen atom or a carbon atom;

Zg is a nitrogen atom or a carbon atom when the ring F is present;

Zg is an oxygen atom, a sulfur atom, NRg, C(Rg₁)(Rg₂), or Si(Rg₃)(Rg₄) when the ring F is not present;

Zh is a nitrogen atom or a carbon atom;

Zi is a nitrogen atom or a carbon atom;

Y is a boron atom, a phosphorus atom, SiRh, P═O or P═S;

Rb, Rb₁, Rb₂, Rb₃, Rb₄, Rd, Re, Rg, Rg₁, Rg₂, Rg₃, Rg₄, and Rh are each independently a hydrogen atom or a substituent;

Rb, Rb₁, Rb₂, Rb₃, Rb₄, Rd, Re, Rg, Rg₁, Rg₂, Rg₃, Rg₄, and Rh as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇);

a bond between Y and Za, a bond between Y and Zd, and a bond between Y and Ze are each a single bond; and

a bond between Y and Za, a bond between Y and Zd, and a bond between Y and Ze are each a single bond, where the single bond is not a coordinate bond but a covalent bond.

Herein, examples of a heterocycle include cyclic structures (heterocycles) excluding a bond from the examples of a “heterocyclic group” listed in the subtitle “Substituents Mentioned Herein.” These heterocycles may be substituted or unsubstituted.

Herein, examples of an aryl ring include cyclic structures (aryl rings) excluding a bond from the examples of an “aryl group” listed in the subtitle “Substituents Mentioned Herein.” These aryl rings may be substituted or unsubstituted.

In the exemplary embodiment, the compound M1 is also preferably a compound represented by a formula (11) below.

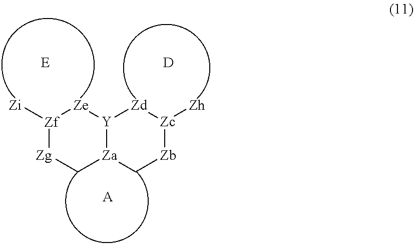

In the formula (11):

a ring A, ring D, and ring E are each independently a cyclic structure selected from the group consisting of a substituted or unsubstituted aryl ring having 6 to 30 ring carbon atoms and a substituted or unsubstituted heterocycle having 5 to 30 ring atoms;

Za is a nitrogen atom or a carbon atom;

Zb is an oxygen atom, a sulfur atom, NRb, C(Rb₁)(Rb₂), or Si(Rb₃)(Rb₄);

Zc is a nitrogen atom or a carbon atom;

Zd is a nitrogen atom or a carbon atom;

Ze is a nitrogen atom or a carbon atom;

Zf is a nitrogen atom or a carbon atom;

Zg is an oxygen atom, a sulfur atom, NRg, C(Rg₁)(Rg₂), or Si(Rg₃)(Rg₄);

Zh is a nitrogen atom or a carbon atom;

Zi is a nitrogen atom or a carbon atom;

Y is a boron atom, a phosphorus atom, SiRh, P═O or P═S;

Rb, Rb₁, Rb₂, Rb₃, Rb₄, Rg, Rg₁, Rg₂, Rg₃, Rg₄, and Rh each independently represent the same as Rb, Rb₁, Rb₂, Rb₃, Rb₄, Rg, Rg₁, Rg₂, Rg₃, Rg₄, and Rh in the formula (1).

In the exemplary embodiment, the compound M1 is also preferably a compound represented by a formula (16) below.

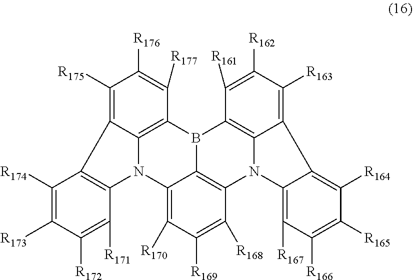

In the formula (16):

at least one combination of adjacent two or more of R₁₆₁ to R₁₇₇ are mutually bonded to form a substituted or unsubstituted monocyclic ring, mutually bonded to form a substituted or unsubstituted fused ring, or not mutually bonded; and

R₁₆₁ to R₁₇₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₆₁)(R₉₆₂)(R₉₆₃), a group represented by —O—(R₉₆₄), a group represented by —S—(R₉₆₅), a group represented by —N(R₉₆₆)(R₉₆₇), a group represented by —C(═O)R₉₆₈, a group represented by —COOR₉₆₉, a halogen atom, a cyano group, a nitro group, a substituted or unsubstituted aryl group having 6 to 50 ring carbon atoms, or a substituted or unsubstituted heterocyclic group having 5 to 50 ring atoms; and

R₉₆₁ to R₉₆₉ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;

when a plurality of R₉₆₁ are present, the plurality of R₉₆₁ are mutually the same or different;

when a plurality of R₉₆₂ are present, the plurality of R₉₆₂ are mutually the same or different;

when a plurality of R₉₆₃ are present, the plurality of R₉₆₃ are mutually the same or different;

when a plurality of R₉₆₄ are present, the plurality of R₉₆₄ are mutually the same or different;

when a plurality of R₉₆₅ are present, the plurality of R₉₆₅ are mutually the same or different;

when a plurality of R₉₆₆ are present, the plurality of R₉₆₆ are mutually the same or different;

when a plurality of R₉₆₇ are present, the plurality of R₉₆₇ are mutually the same or different;

when a plurality of R₉₆₈ are present, the plurality of R₉₆₈ are mutually the same or different; and

when a plurality of R₉₆₉ are present, the plurality of R₉₆₉ are mutually the same or different.

When the compound M1 is a fluorescent compound, the compound M1 preferably emits light having a maximum peak wavelength in a range from 400 nm to 700 nm.

Herein, the maximum peak wavelength means a peak wavelength of a fluorescence spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 manufactured by Hitachi High-Tech Science Corporation) is used as a measurement device.

The compound M1 preferably exhibits red or green light emission.

Herein, the red light emission refers to light emission whose maximum peak wavelength of fluorescence spectrum is in a range from 600 nm to 660 nm.

When the compound M1 is a red fluorescent compound, the maximum peak wavelength of the compound M1 is preferably in a range from 600 nm to 660 nm, more preferably in a range from 600 nm to 640 nm, further preferably in a range from 610 nm to 630 nm.

Herein, the green light emission refers to light emission whose maximum peak wavelength of fluorescence spectrum is in a range from 500 nm to 560 nm. When the compound M1 is a green fluorescent compound, the maximum peak wavelength of the compound M1 is preferably in a range from 500 nm to 560 nm, more preferably in a range from 500 nm to 540 nm, further preferably in a range from 510 nm to 540 nm.

Herein, the blue light emission refers to light emission whose maximum peak wavelength of fluorescence spectrum is in a range from 430 nm to 480 nm.

When the compound M1 is a blue fluorescent compound, the maximum peak wavelength of the compound M1 is preferably in a range from 430 nm to 480 nm, more preferably in a range from 440 nm to 480 nm.

In the exemplary embodiment, the emitting layer preferably contains an emitting compound that emits light having a maximum peak wavelength in a range from 600 nm to 660 nm.

The maximum peak wavelength of light emitted from the organic EL device is measured as follows.

Voltage is applied on the organic EL devices such that a current density becomes 10 mA/cm², where spectral radiance spectrum is measured by a spectroradiometer CS-2000 (manufactured by Konica Minolta, Inc.).

A peak wavelength of an emission spectrum, a luminous intensity of which is the maximum in the obtained spectral radiance spectrum, is measured and defined as the maximum peak wavelength (unit: nm).

### Manufacturing Method of Compound M1

The compound M1 can be manufactured by a known method.

Specific examples of the compound M1 are shown below. It should however be noted that the invention is not limited to the specific examples of the compound.

A coordinate bond between a boron atom and a nitrogen atom in a pyrromethene skeleton is shown by various means such as a solid line, a broken line, an arrow, and omission. Herein, the coordinate bond is shown by a solid line or a broken line, or the description of the coordinate bond is omitted.

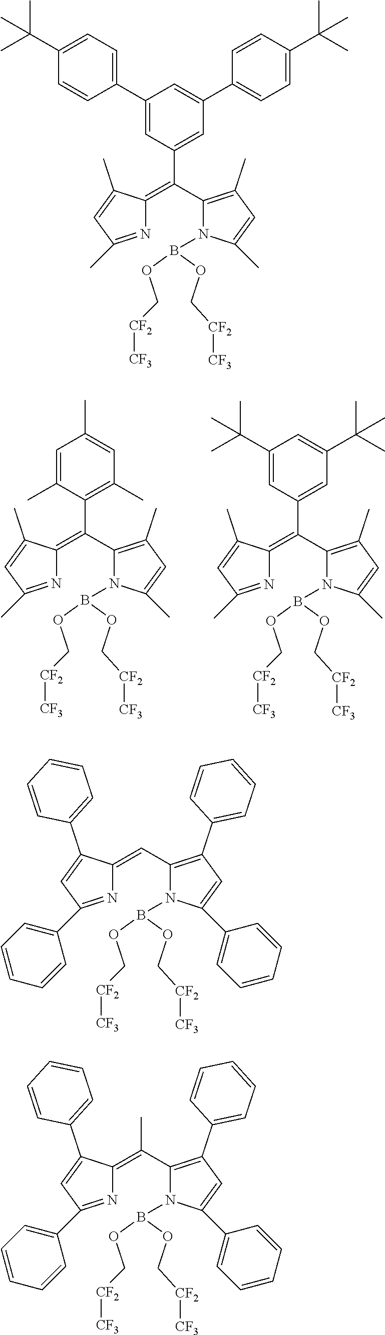

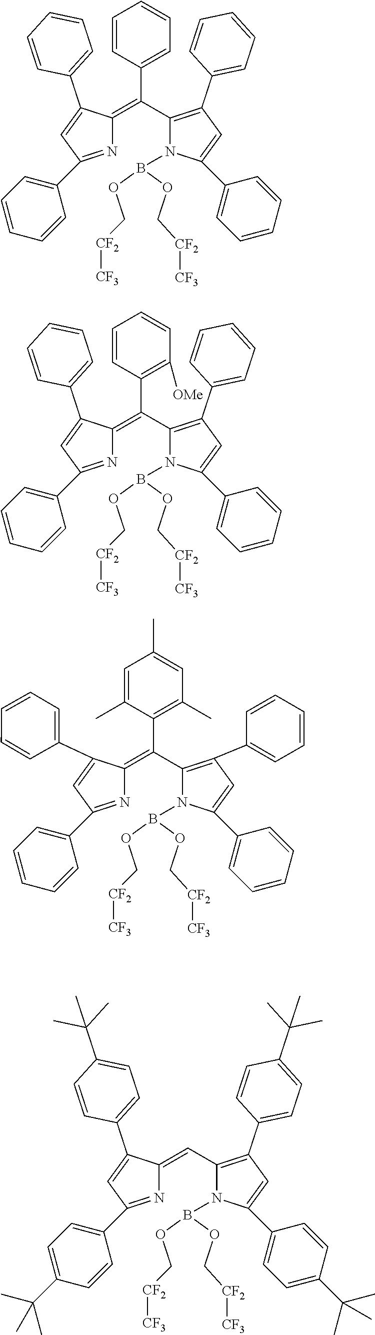

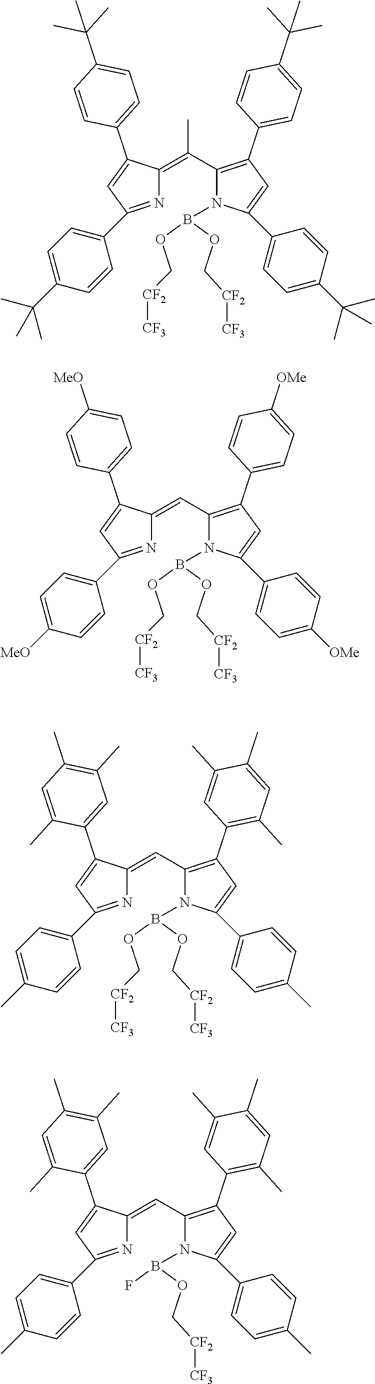

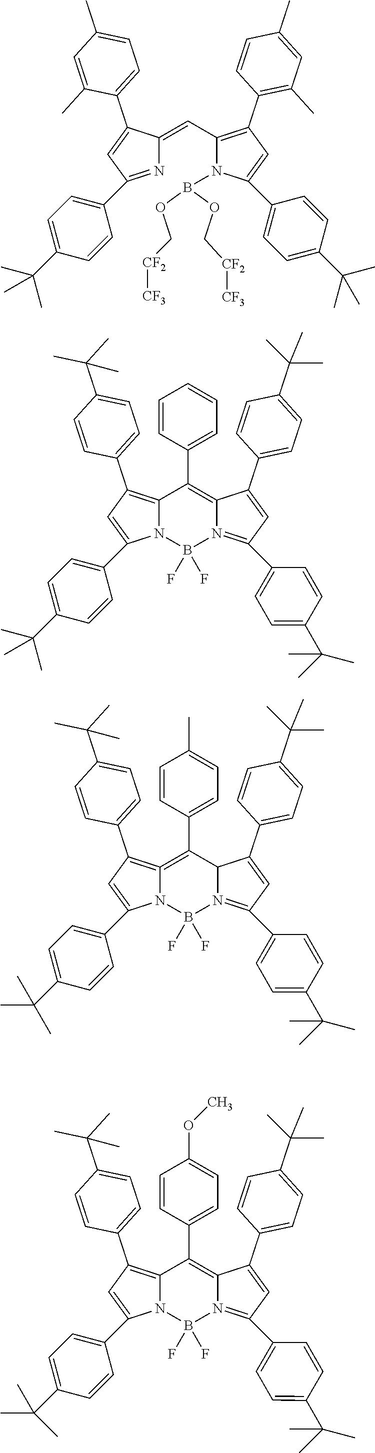

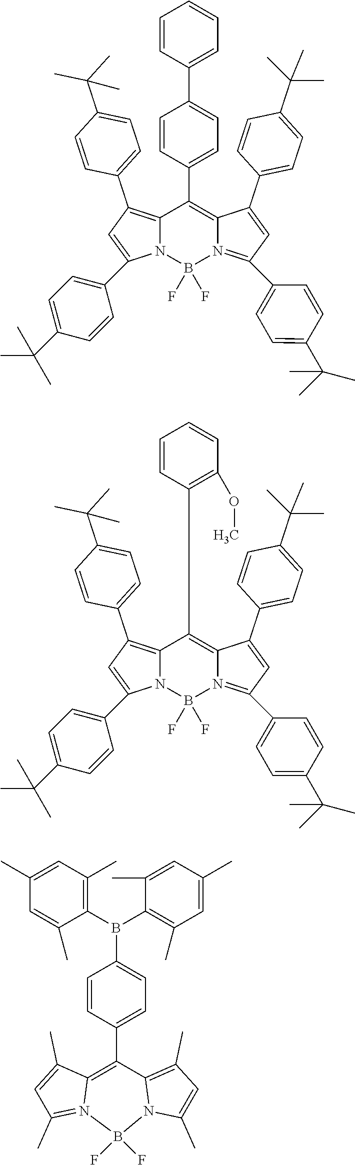

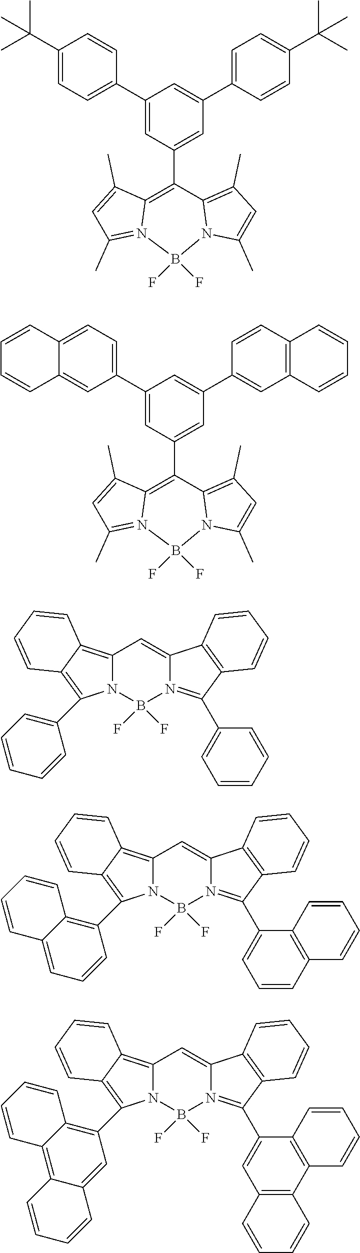

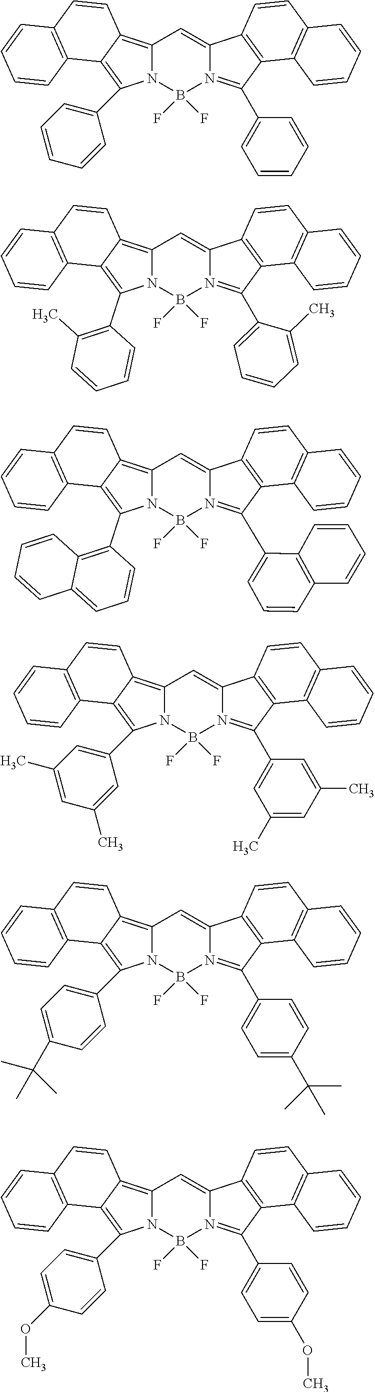

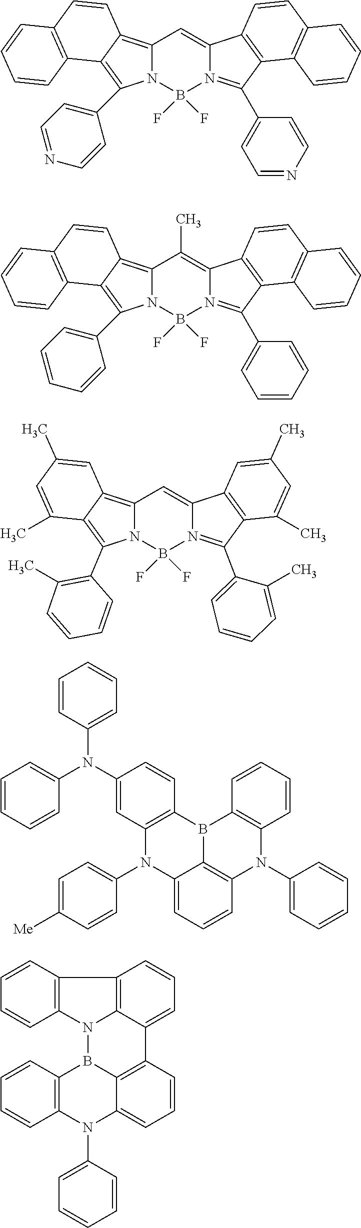

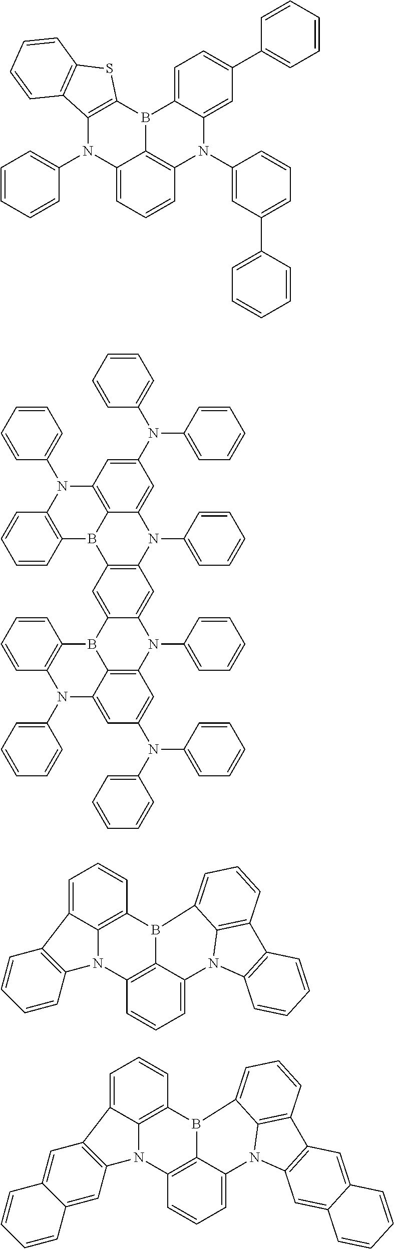

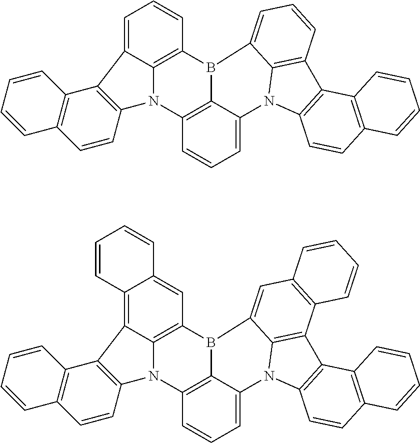 [SMILES: c1cc2c3c(c1)-n1c4ccc5ccccc5c4c4cccc(c41)B3c1cccc3c4c5ccccc5ccc4n-2c13 ; c1cc2c3c(c1)-n1c4ccc5ccccc5c4c4c5ccccc5cc(c41)B3c1cc3ccccc3c3c4c5ccccc5ccc4n-2c13]

### Relationship Between Compound M1 and Compound M2 in Emitting Layer

In the organic EL device of the exemplary embodiment, a singlet energy S₁(Mat2) of the compound M2 as a delayed fluorescent compound and a singlet energy S₁(Mat1) of the fluorescent compound M1 preferably satisfy a relationship of a numerical formula (Numerical Formula 3) below.

S₁(Mat2)>S₁(Mat1) (Numerical Formula 7)

An energy gap T_{77K}(Mat2) at 77K of the compound M2 and an energy gap T_{77K}(Mat1) at 77K of the compound M1 preferably satisfy a relationship of a numerical formula (Numerical Formula 7A) below.

T_{77K}(Mat2)>T_{77K}(Mat1) (Numerical Formula 7A)

It is preferable that, when the organic EL device of the exemplary embodiment emits light, the fluorescent compound M1 mainly emits light in the emitting layer.

### Relationship Between Triplet Energy and Energy Gap at 77K

Here, a relationship between a triplet energy and an energy gap at 77K will be described. In the exemplary embodiment, the energy gap at 77K is different from a typical triplet energy in some aspects.

The triplet energy is measured as follows. First, a solution in which a compound (measurement target) is dissolved in an appropriate solvent is encapsulated in a quartz glass tube to prepare a sample. A phosphorescent spectrum (ordinate axis: phosphorescent luminous intensity, abscissa axis: wavelength) of the sample is measured at a low temperature (77K). A tangent is drawn to the rise of the phosphorescent spectrum close to the short-wavelength region. The triplet energy is calculated by a predetermined conversion equation based on a wavelength value at an intersection of the tangent and the abscissa axis.

Here, the thermally activated delayed fluorescent compound among the compounds of the exemplary embodiment is preferably a compound having a small ΔST. When ΔST is small, intersystem crossing and inverse intersystem crossing are likely to occur even at a low temperature (77K), so that the singlet state and the triplet state coexist. As a result, the spectrum to be measured in the same manner as the above includes emission from both the singlet state and the triplet state. Although it is difficult to distinguish the emission from the singlet state from the emission from the triplet state, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is encapsulated in a quartz cell to provide a measurement sample. A phosphorescent spectrum (ordinate axis: phosphorescent luminous intensity, abscissa axis: wavelength) of the measurement sample is measured at a low temperature (77K). A tangent is drawn to the rise of the phosphorescent spectrum close to the short-wavelength region. An energy amount is calculated by a conversion equation (F1) below based on a wavelength value λ_{edge} [nm] at an intersection of the tangent and the abscissa axis and is defined as an energy gap T_{77K} at 77K.

T_{77K} [eV]=1239.85/λ_{edge} Conversion Equation (F1):

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased).

A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S1

A method of measuring the singlet energy S₁ with use of a solution (occasionally referred to as a solution method) is exemplified by a method below.

A toluene solution in which a measurement target compound is dissolved at a concentration of 10 μmol/L is prepared and is encapsulated in a quartz cell to provide a measurement sample. Absorption spectrum (ordinate axis: absorption intensity, abscissa axis: wavelength) of the sample is measured at normal temperature (300K). A tangent was drawn to the fall of the absorption spectrum close to the long-wavelength region, and a wavelength value λedge (nm) at an intersection of the tangent and the abscissa axis was assigned to a conversion equation (F2) below to calculate the singlet energy.

S₁ [eV]=1239.85/Δedge Conversion Equation (F2):

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the exemplary embodiment, a difference (S₁−T_{77K}) between the singlet energy S₁ and the energy gap T_{77K} at 77K is defined as ΔST.

In the exemplary embodiment, a difference ΔST(Mat2) between the singlet energy S₁ (Mat2) of the compound M2 and the energy gap T_{77K}(Mat2) at 77K of the compound M2 is preferably less than 0.3 eV, more preferably less than 0.2 eV, further preferably less than 0.1 eV, and still further preferably less than 0.01 eV. That is, ΔST(Mat2) preferably satisfies a relationship of one of numerical formulae (Numerical Formula 1A) to (Numerical Formula 1D) below.

ΔST(Mat2)=S₁(Mat2)−T_{77K}(Mat2)<0.3 eV (Numerical Formula 1A)

ΔST(Mat2)=S₁(Mat2)−T_{77K}(Mat2)<0.2 eV (Numerical Formula 1B)

ΔST(Mat2)=S₁(Mat2)−T_{77K}(Mat2)<0.1 eV (Numerical Formula 1C)

ΔST(Mat2)=S₁(Mat2)−T_{77K}(Mat2)<0.01 eV (Numerical Formula 1D)

The organic EL device of the exemplary embodiment preferably emits red light or green light.

When the organic EL device according to the exemplary embodiment emits green light, the maximum peak wavelength of the light emitted from the organic EL device is preferably in a range from 500 nm to 560 nm.

When the organic EL device according to the exemplary embodiment emits red light, the maximum peak wavelength of the light emitted from the organic EL device is preferably in a range from 600 nm to 660 nm.

When the organic EL device according to the exemplary embodiment emits blue light, the maximum peak wavelength of the light emitted from the organic EL device is preferably in a range from 430 nm to 480 nm.

The maximum peak wavelength of the light emitted from the organic EL device is measured as follows.

Voltage is applied on the organic EL devices such that a current density becomes 10 mA/cm², where spectral radiance spectrum is measured by a spectroradiometer CS-2000 (manufactured by Konica Minolta, Inc.).

A peak wavelength of an emission spectrum, a luminous intensity of which is the maximum in the obtained spectral radiance spectrum, is measured and defined as the maximum peak wavelength (unit: nm).

### Film Thickness of Emitting Layer

The film thickness of the emitting layer of the organic EL device in the exemplary embodiment is preferably in a range from 5 nm to 50 nm, more preferably in a range from 7 nm to 50 nm, and most preferably in a range from 10 nm to 50 nm. When the film thickness of the emitting layer is 5 nm or more, the formation of the emitting layer and the adjustment of the chromaticity are easy. When the film thickness of the emitting layer is 50 nm or less, an increase in the drive voltage is likely to be reducible.

### Content Ratios of Compounds in Emitting Layer

For instance, content ratios of the compound M2 and the compound M1 in the emitting layer preferably fall within ranges shown below.

The content ratio of the compound M2 is preferably in a range from 10 mass % to 80 mass %, more preferably in a range from 10 mass % to 60 mass %, and further preferably in a range from 20 mass % to 60 mass %.

The content ratio of the compound M1 is preferably in a range from 0.01 mass % to 10 mass %, more preferably in a range from 0.01 mass % to 5 mass %, and further preferably in a range from 0.01 mass % to 1 mass %.

It should be noted that the emitting layer according to the exemplary embodiment may contain a material other than the compound M2 and the compound M1.

The emitting layer may contain a single type of the compound M2 or may contain two or more types of the compound M2. The emitting layer may contain a single type of the compound M1 or may contain two or more types of the compound M1.

### TADF Mechanism

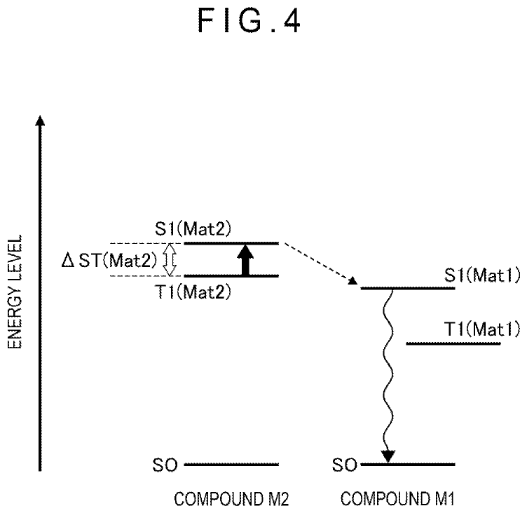

FIG. 4 shows an example of a relationship between energy levels of the compound M2 and the compound M1 in the emitting layer. In FIG. 4, S0 represents a ground state. S1(Mat2) represents a lowest singlet state of the compound M2. T1(Mat2) represents a lowest triplet state of the compound M2. S1(Mat1) represents a lowest singlet state of the compound M1. T1(Mat1) represents a lowest triplet state of the compound M1.

A dashed arrow directed from S1(Mat2) to S1(Mat1) in FIG. 4 represents Førster energy transfer from the lowest singlet state of the compound M2 to the lowest singlet state of the compound M1.

As shown in FIG. 4, when a compound having a small ΔST(Mat2) is used as the compound M2, inverse intersystem crossing from the lowest triplet state T1(Mat2) to the lowest singlet state S1(Mat2) can be caused by heat energy. Subsequently, Førster energy transfer from the lowest singlet state S1(Mat2) of the compound M2 to the compound M1 occurs to generate the lowest singlet state S1(Mat1). Consequently, fluorescence from the lowest singlet state S1(Mat1) of the compound M1 can be observed. It is inferred that the internal quantum efficiency can be theoretically raised up to 100% also by using delayed fluorescence by the TADF mechanism.

According to the first exemplary embodiment, an organic EL device that can achieve higher performance (especially, a decrease in voltage), specifically, both luminous efficiency and voltage suitable for practical use, can be provided.

The organic EL device according to the first exemplary embodiment is applicable to an organic electroluminescence display device (hereinafter, occasionally referred to as organic EL display device).

The organic EL device according to the first exemplary embodiment is applicable to an electronic device such as a display device and a light-emitting unit.

### Arrangement of Organic EL Device

An arrangement of the organic EL device 1 is further described below. It should be noted that the reference numerals are occasionally omitted below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

The elements belonging to the group 1 or 2 of the periodic table, which are a material having a small work function, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal are usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of materials for the cathode include elements belonging to the group 1 or 2 of the periodic table, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl(abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,34:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N, N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

When the hole transporting layer includes two or more layers, one of the layers with a larger energy gap is preferably provided closer to the emitting layer.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the present exemplary embodiment, a benzimidazole compound is preferably usable. The above-described substances mostly have an electron mobility of 10⁻⁶ cm²/(V·s) or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)] (abbreviation: PF-Py), poly[(9,9-dioctylfluorene-2,7-diyl)-co-(2,2′-bipyridine-6,6′-diyl)] (abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

The organic EL device 1 of the exemplary embodiment includes, between the anode 3 and the emitting layer 5, a hole transporting zone including at least one organic layer. The hole transporting zone shown in FIG. 1 is provided by the first layer 61 and the anode-side organic layer 63. The hole transporting zone preferably includes a plurality of organic layers.

### Layer Formation Method

A method for forming each layer of the organic EL device in the present exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

### Second Exemplary Embodiment

An arrangement of an organic EL device according to a second exemplary embodiment of the invention is described below. In the description of the second exemplary embodiment, the same components as those in the first exemplary embodiment are denoted by the same reference signs and names to simplify or omit an explanation of the components. In the second exemplary embodiment, the same materials and compounds as described in the first exemplary embodiment are usable, unless otherwise specified.

The organic EL device according to the second exemplary embodiment is different from the organic EL device according to an exemplary arrangement of the first exemplary embodiment in that the emitting layer contains a compound M3 in addition to the delayed fluorescent compound M2 and the fluorescent compound M1. Other components are the same as those in the first exemplary embodiment.

Specifically, in the organic EL device of the second exemplary embodiment, the emitting layer contains the delayed fluorescent compound M2, the fluorescent compound M1, and the compound M3; the first layer contains the first compound satisfying specific parameters (Numerical Formula 1 and Numerical Formula 2); the second layer contains the second compound satisfying a specific parameter (Numerical Formula 3); and the first layer has a film thickness of 15 nm or more.

In the second exemplary embodiment, the compound M2 in the emitting layer is preferably a host material, the compound M1 is preferably a dopant material, and the compound M3 is preferably a host material. One of the compound M2 and the compound M3 is occasionally referred to as a first host material, and the other is occasionally referred to as a second host material.

As the compound M2, the compound M2 described in the first exemplary embodiment is usable.

As the compound M1, the compound M1 described in the first exemplary embodiment is usable.

As the first compound, the first compound described in the first exemplary embodiment is usable.

As the second compound, the second compound described in the first exemplary embodiment is usable.

### Compound M3

The compound M3 may be a thermally activated delayed fluorescent compound or a compound exhibiting no thermally activated delayed fluorescence. However, the compound M3 is preferably a compound exhibiting no thermally activated delayed fluorescence.

In the exemplary embodiment, the compound M3 is preferably a compound represented by a formula (3X) or (3Y) below.

### Compound Represented by Formula (3X)

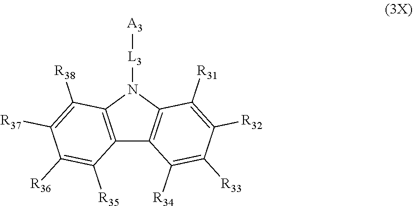

In the formula (3X):

A₃ is a substituted or unsubstituted aryl group having 6 to 50 ring carbon atoms, or a substituted or unsubstituted heterocyclic group having 5 to 50 ring atoms;

L₃ is a single bond, a substituted or unsubstituted arylene group having 6 to 50 ring carbon atoms, a substituted or unsubstituted divalent heterocyclic group having 5 to 50 ring atoms, a divalent group formed by bonding two groups selected from the group consisting of a substituted or unsubstituted arylene group having 6 to 50 ring carbon atoms and a substituted or unsubstituted divalent heterocyclic group having 5 to 50 ring atoms, or a divalent group formed by bonding three groups selected from the group consisting of a substituted or unsubstituted arylene group having 6 to 30 ring carbon atoms and a substituted or unsubstituted divalent heterocyclic group having 5 to 30 ring atoms;

at least one combination of adjacent two or more of R₃₁ to R₃₈ are mutually bonded to form a substituted or unsubstituted monocyclic ring, mutually bonded to form a substituted or unsubstituted fused ring, or not mutually bonded; and

R₃₁ to R₃₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, a substituted or unsubstituted heterocyclic group having 5 to 50 ring atoms, or a group represented by a formula (3A).

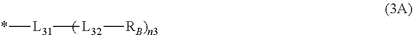

In the formula (3A):

R_{B} is a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms;

when a plurality of R_{B} are present, the plurality of R_{B} are mutually the same or different;

L₃₁ is: a single bond; a substituted or unsubstituted arylene group having 6 to 50 ring carbon atoms, or a trivalent, tetravalent, pentavalent, or hexavalent group derived from the arylene group; a substituted or unsubstituted divalent heterocyclic group having 5 to 50 ring atoms, or a trivalent, tetravalent, pentavalent, or hexavalent group derived from the heterocyclic group; or a divalent group formed by bonding two groups selected from the group consisting of a substituted or unsubstituted arylene group having 6 to 50 ring carbon atoms and a substituted or unsubstituted divalent heterocyclic group having 5 to 50 ring atoms, or a trivalent, tetravalent, pentavalent, or hexavalent group derived from the divalent group;

L₃₂ is a single bond, a substituted or unsubstituted arylene group having 6 to 50 ring carbon atoms, a substituted or unsubstituted divalent heterocyclic group having 5 to 50 ring atoms;

n₃ is 1, 2, 3, 4, or 5;

when L₃₁ is a single bond, n₃ is 1 and L₃₂ is bonded to a carbon atom in a six-membered ring in the formula (3X);

when a plurality of L₃₂ are present, the plurality of L₃₂ are mutually the same or different; and

* represents a bonding position to a carbon atom in a six-membered ring in the formula (3X).

In the compound M3:

R₉₀₁, R₉₀₂, R₉₀₃, R₉₀₄, R₉₀₅, R₉₀₆, R₉₀₇, R₉₀₈, R₉₀₉, R₉₃₁, R₉₃₂, R₉₃₃, R₉₃₄, R₉₃₅, R₉₃₆, and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₉₀₈ are present, the plurality of R₉₀₈ are mutually the same or different;

when a plurality of R₉₀₉ are present, the plurality of R₉₀₉ are mutually the same or different;

when a plurality of R₉₃₁ are present, the plurality of R₉₃₁ are mutually the same or different;

when a plurality of R₉₃₂ are present, the plurality of R₉₃₂ are mutually the same or different;

when a plurality of R₉₃₃ are present, the plurality of R₉₃₃ are mutually the same or different;

when a plurality of R₉₃₄ are present, the plurality of R₉₃₄ are mutually the same or different;

when a plurality of R₉₃₅ are present, the plurality of R₉₃₅ are mutually the same or different;

when a plurality of R₉₃₆ are present, the plurality of R₉₃₆ are mutually the same or different; and

when a plurality of R₉₃₇ are present, the plurality of R₉₃₇ are mutually the same or different.

The compound M3 is also preferably a compound represented by any of formulae (31) to (36) below.

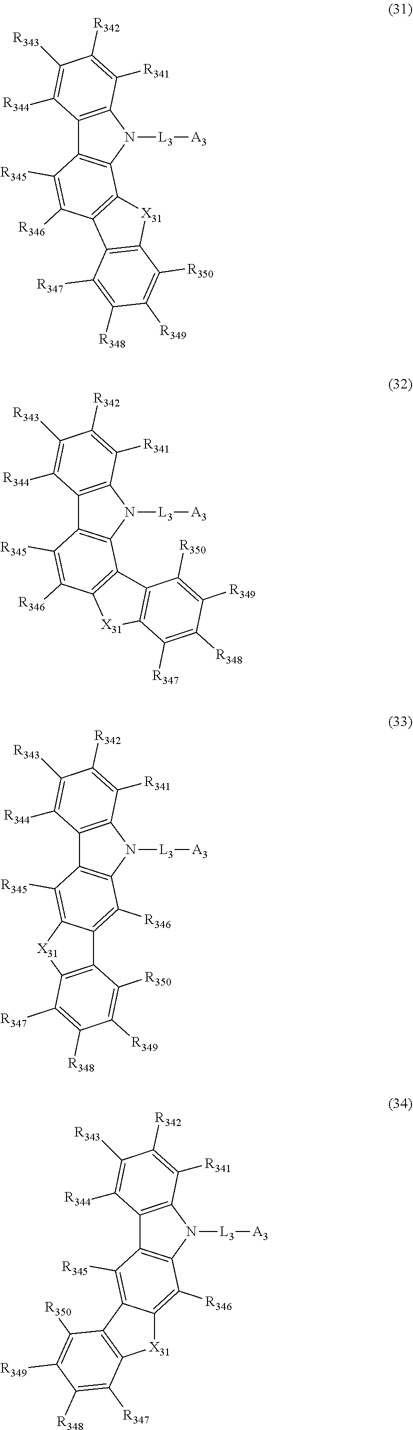

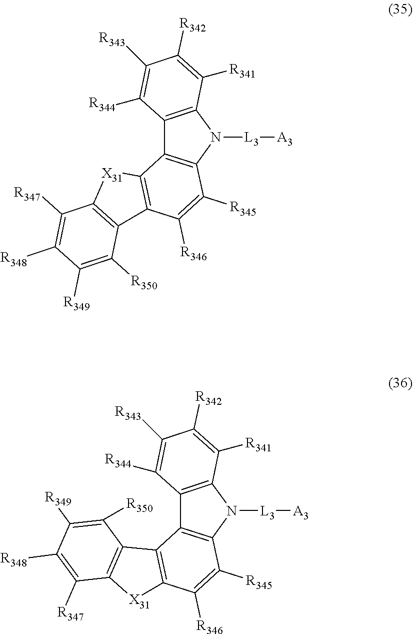

In the formulae (31) to (36):

A₃ and L₃ respectively represent the same as A₃ and L₃ in the formula (3X);

at least one combination of adjacent two or more of R₃₄₁ to R₃₅₀ are mutually bonded to form a substituted or unsubstituted monocyclic ring, mutually bonded to form a substituted or unsubstituted fused ring, or not mutually bonded;

X₃₁ is a sulfur atom, an oxygen atom, NR₃₅₂, or CR₃₅₃R₃₅₄;

a combination of R₃₅₃ and R₃₅₄ are mutually bonded to form a substituted or unsubstituted monocyclic ring, mutually bonded to form a substituted or unsubstituted fused ring, or not mutually bonded; and

R₃₄₁ to R₃₅₀ forming neither the substituted or unsubstituted monocyclic ring nor the substituted or unsubstituted fused ring, R₃₅₂, and R₃₅₃ and R₃₅₄ forming neither the substituted or unsubstituted monocyclic ring nor the substituted or unsubstituted fused ring each independently represent the same as R₃₁ to R₃₈ forming neither the substituted or unsubstituted monocyclic ring nor the substituted or unsubstituted fused ring.

In the compound M3, it is preferable that R₃₅₂ is a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M3, it is preferable that a combination of R₃₅₃ and R₃₅₄ are mutually bonded to form a substituted or unsubstituted monocyclic ring, mutually bonded to form a substituted or unsubstituted fused ring, or not mutually bonded; and R₃₅₃ and R₃₅₄ forming neither the substituted or unsubstituted monocyclic ring nor the substituted or unsubstituted fused ring are each independently a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M3, X₃₁ is preferably is a sulfur atom or an oxygen atom.

In the compound M3, A₃ is preferably a group represented by any of formulae (A31) to (A37) below.

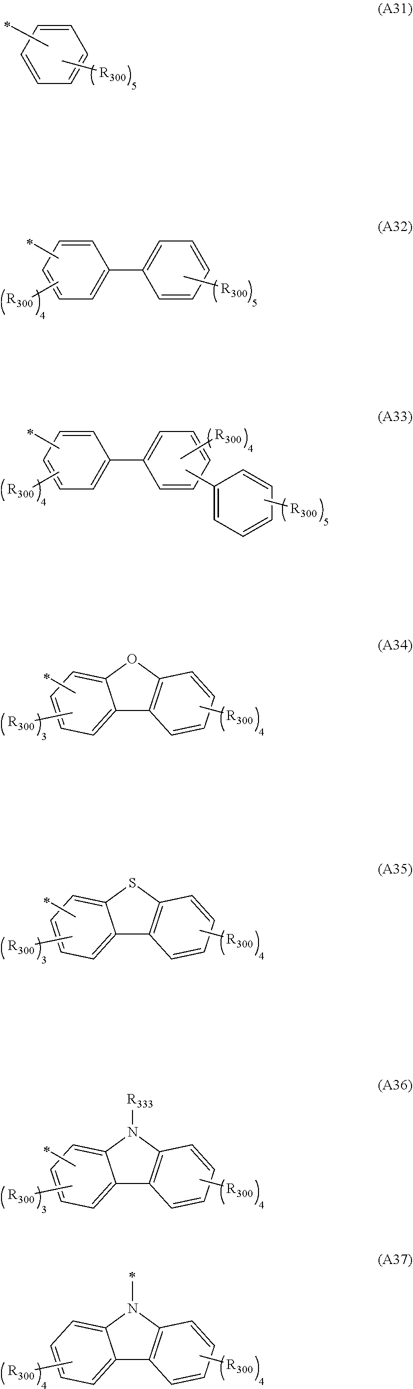

In the formulae (A31) to (A37):

at least one combination of adjacent two or more of a plurality of R₃₀₀ are mutually bonded to form a substituted or unsubstituted monocyclic ring, mutually bonded to form a substituted or unsubstituted fused ring, or not mutually bonded;

R₃₀₀ forming neither the substituted or unsubstituted monocyclic ring nor the substituted or unsubstituted fused ring, and R₃₃₃ each independently represent the same as R₃₁ to R₃₈ forming neither the substituted or unsubstituted monocyclic ring nor the substituted or unsubstituted fused ring; and

* in each of the formulae (A31) to (A37) represents a bonding position to L₃ of the compound M3.

In the compound M3, A₃ is also preferably a group represented by the formula (A34), (A35), or (A37).

The compound M3 is also preferably a compound represented by any of formulae (311) to (316) below.

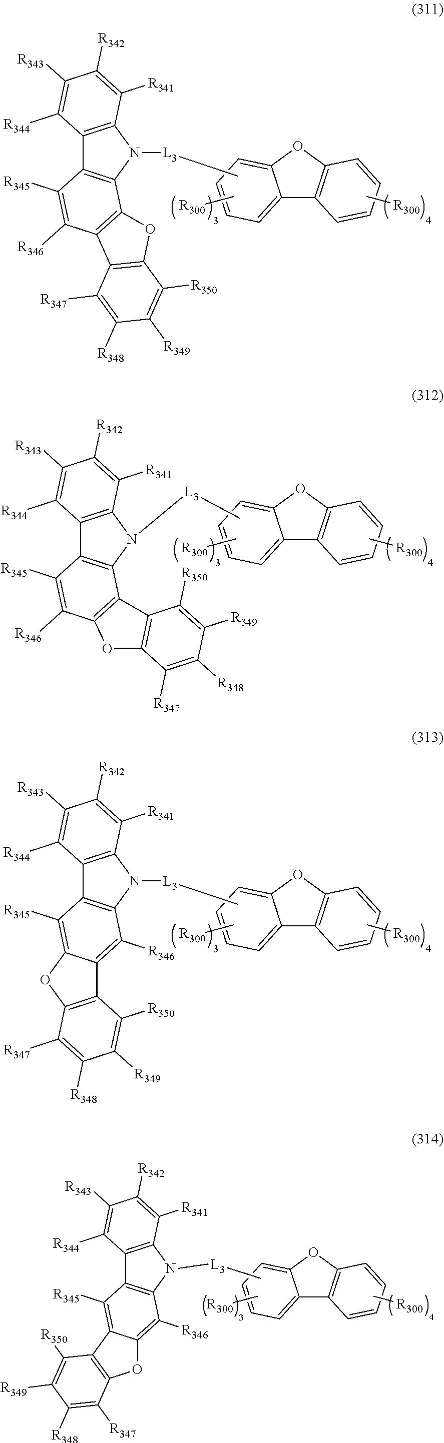

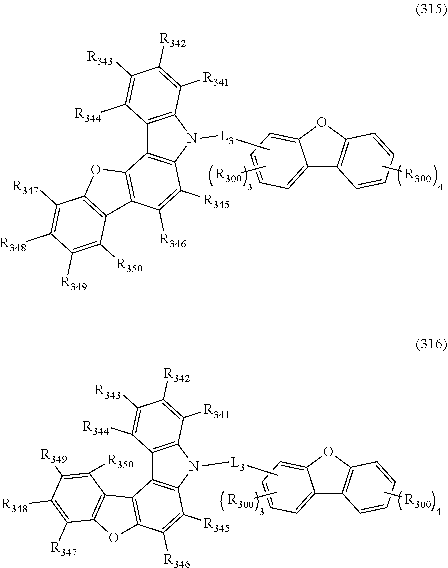

In the formulae (311) to (316):

L₃ represents the same as L₃ in the formula (3X);

at least one combination of adjacent two or more of a plurality of R₃₀₀ are mutually bonded to form a substituted or unsubstituted monocyclic ring, mutually bonded to form a substituted or unsubstituted fused ring, or not mutually bonded;

at least one combination of adjacent two or more of R₃₄₁ to R₃₅₀ are mutually bonded to form a substituted or unsubstituted monocyclic ring, mutually bonded to form a substituted or unsubstituted fused ring, or not mutually bonded; and

R₃₀₀ forming neither the substituted or unsubstituted monocyclic ring nor the substituted or unsubstituted fused ring, and R₃₄₁ to R₃₅₀ forming neither the substituted or unsubstituted monocyclic ring nor the substituted or unsubstituted fused ring each independently represent the same as R₃₁ to R₃₈ forming neither the substituted or unsubstituted monocyclic ring nor the substituted or unsubstituted fused ring.

The compound M3 is also preferably a compound represented by a formula (321) below.

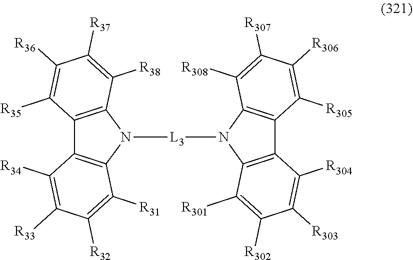

In the formula (321):

L₃ represents the same as L₃ in the formula (3X); and

R₃₁ to R₃₈, and R₃₀₁ to R₃₀₈ each independently represent the same as R₃₁ to R₃₈ forming neither the substituted or unsubstituted monocyclic ring nor the substituted or unsubstituted fused ring.

In the compound M3, L₃ is preferably a single bond, or a substituted or unsubstituted arylene group having 6 to 50 ring carbon atoms.

In the compound M3, L₃ is preferably a single bond, a substituted or unsubstituted phenylene group, a substituted or unsubstituted biphenylene group, or a substituted or unsubstituted terphenylene group.

In the compound M3, L₃ is preferably a group represented by a formula (317) below.

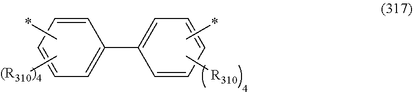

In the formula (317), each R₃₁₀ independently represents the same as R₃₁ to R₃₈ forming neither the substituted or unsubstituted monocyclic ring nor the substituted or unsubstituted fused ring, and each * independently represents a bonding position.

In the compound M3, L₃ also preferably contains a divalent group represented by a formula (318) or (319) below.

In the compound M3, L₃ is also preferably a divalent group represented by the formula (318) or (319).

The compound M3 is also preferably a compound represented by a formula (322) or (323) below.

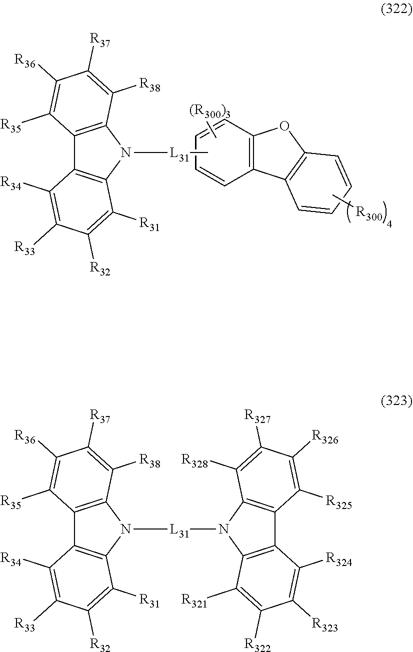

In the formulae (322) and (323):

L₃₁ is: a substituted or unsubstituted arylene group having 6 to 50 ring carbon atoms; a substituted or unsubstituted divalent heterocyclic group having 5 to 50 ring atoms; or a divalent group formed by bonding two groups selected from the group consisting of a substituted or unsubstituted arylene group having 6 to 50 ring carbon atoms and a substituted or unsubstituted divalent heterocyclic group having 5 to 50 ring atoms;

L₃₁ contains a divalent group represented by the formula (318) or (319); and

R₃₁ to R₃₈, R₃₀₀, and R₃₂₁ to R₃₂₈ each independently represent the same as R₃₁ to R₃₈ forming neither the substituted or unsubstituted monocyclic ring nor the substituted or unsubstituted fused ring.

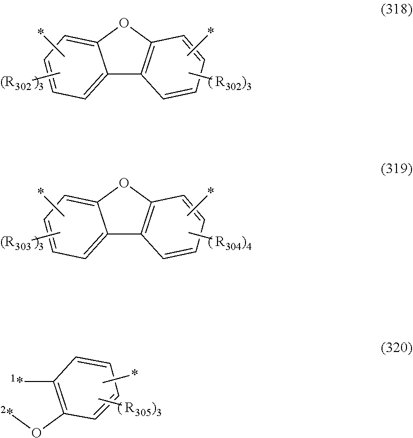

In the formula (319), a combination of adjacent two of a plurality of R₃₀₄ are mutually bonded to form a ring represented by the formula (320).

In the formula (320), 1* and 2* each independently represent a bonding position to a ring bonded to R₃₀₄.

R₃₀₂ in the formula (318), R₃₀₃ in the formula (318), R₃₀₃ in the formula (319), R₃₀₄ not forming the ring represented by the formula (320), and R₃₀₅ in the formula (320) each independently represent the same as R₃₁ to R₃₈ forming neither the substituted or unsubstituted monocyclic ring nor the substituted or unsubstituted fused ring.

* in each of the formulae (318) to (320) represents a bonding position.

In the compound M3, the group as L₃ or L₃₁ that is represented by the formula (319) is, for instance, a group represented by a formula (319A) below.

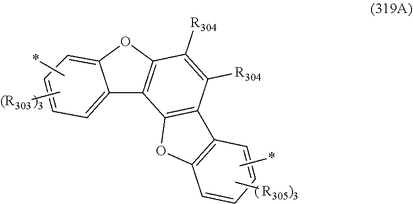

In the formula (319A), R₃₀₃, R₃₀₄, and R₃₀₅ each independently represent the same as R₃₁ to R₃₈ forming neither the substituted or unsubstituted monocyclic ring nor the substituted or unsubstituted fused ring, and each * in the formula (319A) represents a bonding position.

It is also preferable that the compound M3 is a compound represented by the formula (322) and L₃₁ is a group represented by the formula (318) below.

The compound M3 is also preferably a compound represented by a formula (324) below.

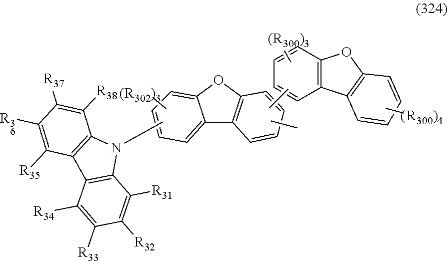

In the formula (324), R₃₁ to R₃₈, R₃₀₀, and R₃₀₂ each independently represent the same as R₃₁ to R₃₈ forming neither the substituted or unsubstituted monocyclic ring nor the substituted or unsubstituted fused ring.

It is preferable that R₃₁ to R₃₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted aryl group having 6 to 50 ring carbon atoms, a substituted or unsubstituted heterocyclic group having 5 to 50 ring atoms, or a group represented by the formula (3A); and R_{B} in the formula (3A) is a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

It is preferable that R₃₁ to R₃₈ forming neither the substituted or unsubstituted monocyclic ring nor the substituted or unsubstituted fused ring are each independently a hydrogen atom, a substituted or unsubstituted aryl group having 6 to 50 ring carbon atoms, or a group represented by the formula (3A); and R_{B} in the formula (3A) is a substituted or unsubstituted aryl group having 6 to 50 ring carbon atoms.

It is preferable that R₃₁ to R₃₈ forming neither the substituted or unsubstituted monocyclic ring nor the substituted or unsubstituted fused ring are each independently a hydrogen atom, a substituted or unsubstituted phenyl group, or a group represented by the formula (3A); and R_{B} in the formula (3A) is a substituted or unsubstituted phenyl group.

The compound M3 is also preferably a compound not having a pyridine ring, a pyrimidine ring, and a triazine ring.

### Compound Represented by Formula (3Y)

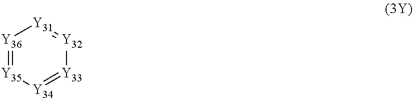

In the formula (3Y):

Y₃₁ to Y₃₆ are each independently CR₃ or a nitrogen atom;

two or more of Y₃₁ to Y₃₆ are each a nitrogen atom;

when a plurality of R₃ are present, at least one combination of adjacent two or more of the plurality of R₃ are mutually bonded to form a substituted or unsubstituted monocyclic ring, mutually bonded to form a substituted or unsubstituted fused ring, or not mutually bonded; and

each R₃ forming neither the substituted or unsubstituted monocyclic ring nor the substituted or unsubstituted fused ring is independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, a substituted or unsubstituted heterocyclic group having 5 to 50 ring atoms, or a group represented by a formula (3B) below.

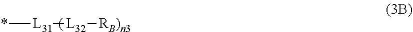

In the formula (3B): R_{B}, L₃₁, L₃₂ and n₃ each independently represent the same as R_{B}, L₃₁, L₃₂ and n₃ in the formula (3A);

when a plurality of R_{B} are present, the plurality of R_{B} are mutually the same or different;

when L₃₁ is a single bond, n₃ is 1 and L₃₂ is bonded to a carbon atom in a six-membered ring in the formula (3Y);

when a plurality of L₃₂ are present, the plurality of L₃₂ are mutually the same or different; and

* represents a bonding position to a carbon atom in a six-membered ring in the formula (3Y).

The compound M3 preferably does not include a pyridine ring in a molecule.

The compound M3 is also preferably a compound represented by a formula (31a) or (32a) below.

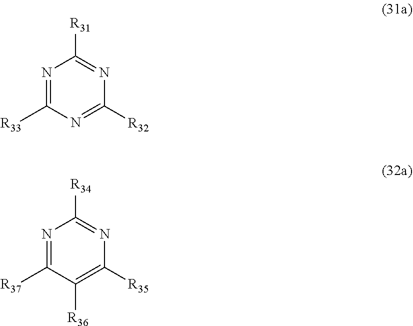

In the formula (32a):

at least one combination of adjacent two or more of R₃₅ to R₃₇ are mutually bonded to form a substituted or unsubstituted monocyclic ring, mutually bonded to form a substituted or unsubstituted fused ring, or not mutually bonded; and

R₃₁ to R₃₃ in the formula (31a), R₃₄ in the formula (32a), and R₃₅ to R₃₇ forming neither the substituted or unsubstituted monocyclic ring nor the substituted or unsubstituted fused ring each independently represent the same as R₃ in the formula (3Y).

The compound M3 is also preferably a compound represented by the formula (31a).

It is preferable that each R₃ in the formula (3Y) is independently a hydrogen atom, a substituted or unsubstituted alkyl group having 1 to 50 carbon atoms, a substituted or unsubstituted aryl group having 6 to 50 ring carbon atoms, a substituted or unsubstituted heterocyclic group having 5 to 50 ring atoms, or a group represented by the formula (3B).

It is preferable that each R₃ in the formula (3Y) is independently a hydrogen atom, a substituted or unsubstituted alkyl group having 1 to 50 carbon atoms, a substituted or unsubstituted aryl group having 6 to 50 ring carbon atoms, or a group represented by the formula (3B).

The compound M3 represented by the formula (3Y) preferably contains, in a molecule, at least one group selected from the group consisting of groups represented by formulae (B31) to (B44) below.

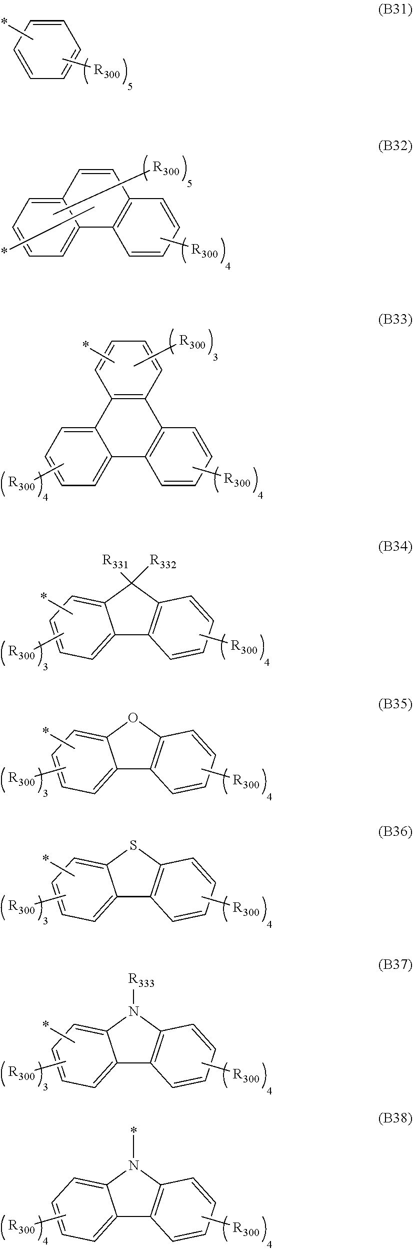

In the formulae (B31) to (B38):

at least one combination of adjacent two or more of a plurality of R₃₀₀ are mutually bonded to form a substituted or unsubstituted monocyclic ring, mutually bonded to form a substituted or unsubstituted fused ring, or not mutually bonded;

a combination of R₃₃₁ and R₃₃₂ are mutually bonded to form a substituted or unsubstituted monocyclic ring, mutually bonded to form a substituted or unsubstituted fused ring, or not mutually bonded;

R₃₀₀, R₃₃₁ and R₃₃₂ forming neither the substituted or unsubstituted monocyclic ring nor the substituted or unsubstituted fused ring, and R₃₃₃ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms; and

* in each of the formulae (B31) to (B38) represents a bonding position to any other atom in a molecule of the compound M3.

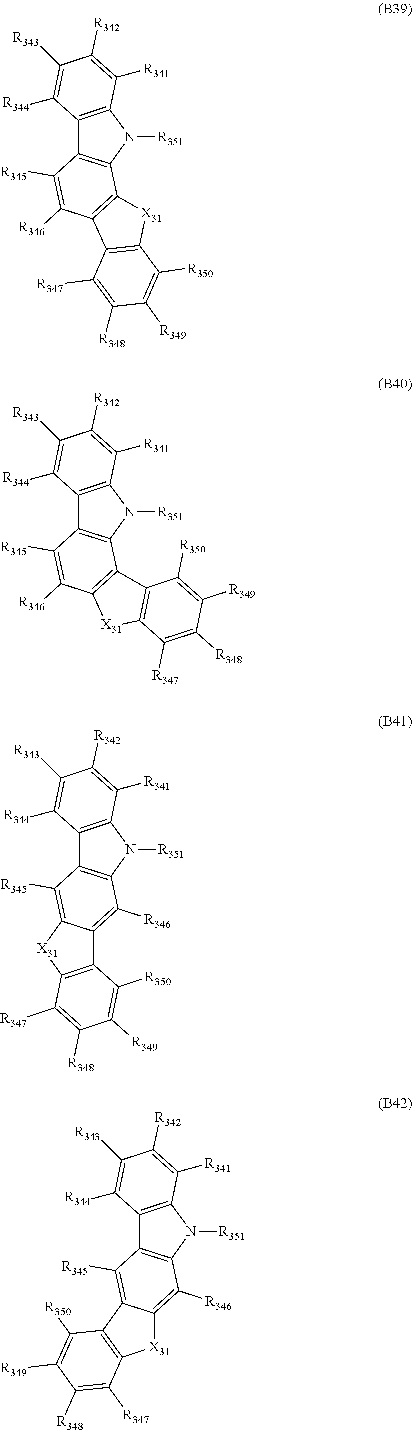

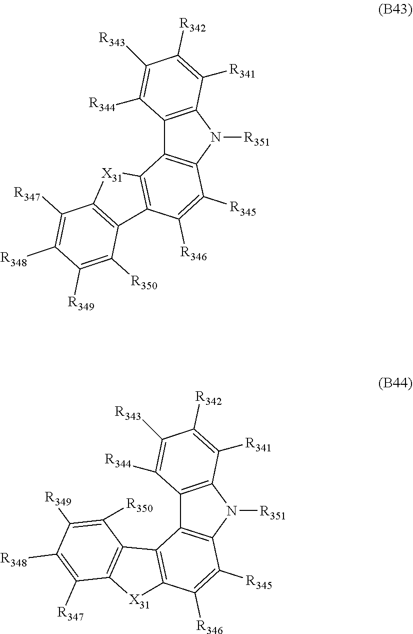

In the formulae (B39) to (B44):

at least one combination of adjacent two or more of R₃₄₁ to R₃₅₀ are mutually bonded to form a substituted or unsubstituted monocyclic ring, mutually bonded to form a substituted or unsubstituted fused ring, or not mutually bonded;

at least one of R₃₄₁ to R₃₅₁ represents a bonding position to any other atom in a molecule of the compound M3;

X₃₁ is a sulfur atom, an oxygen atom, NR₃₅₂, or CR₃₅₃R₃₅₄;
a combination of R₃₅₃ and R₃₅₄ are mutually bonded to form a substituted or unsubstituted monocyclic ring, mutually bonded to form a substituted or unsubstituted fused ring, or not mutually bonded; and

R₃₄₁ to R₃₅₁ not being the bonding position to any other atom in the molecule of the compound M3, not forming the substituted or unsubstituted monocyclic ring, and not forming the substituted or unsubstituted fused ring; R₃₅₂; and R₃₅₃ and R₃₅₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms.

The compound M3 represented by the formula (3Y) preferably contains, in a molecule, at least one group selected from the group consisting of groups represented by the formulae (B38) to (B44).

In the formula (3Y), it is preferable that at least one of Y₃₁ to Y₃₆ is CR₃, at least one R₃ is a group represented by the formula (3B), and R_{B} is a group represented by any of the formulae (B31) to (B44).

In the formula (3Y), it is preferable that at least one of Y₃₁ to Y₃₆ is CR₃, at least one R₃ is a group represented by the formula (3B), and R_{B} is a group represented by any of the formulae (B38) to (B44).

In the formulae (3A) and (3B), it is preferable that:

L₃₁ is: a single bond; a substituted or unsubstituted arylene group having 6 to 50 ring carbon atoms, or a trivalent, tetravalent, pentavalent, or hexavalent group derived from the arylene group; or a divalent group formed by bonding two groups selected from the group consisting of a substituted or unsubstituted arylene group having 6 to 50 ring carbon atoms, or a trivalent, tetravalent, pentavalent, or hexavalent group derived from the divalent group; and

each L₃₂ is independently a single bond, or a substituted or unsubstituted arylene group having 6 to 50 ring carbon atoms.

In the formulae (3A) and (3B), it is preferable that:

L₃₁ is a single bond, or a substituted or unsubstituted arylene group having 6 to 50 ring carbon atoms;

n₃ is 1; and

L₃₂ is a single bond, or a substituted or unsubstituted arylene group having 6 to 50 ring carbon atoms.

In the formulae (3A) and (3B), it is preferable that:

L₃₁ is: a single bond; a substituted or unsubstituted phenylene group; a substituted or unsubstituted biphenylene group; or a divalent group formed by bonding two groups selected from the group consisting of a substituted or unsubstituted phenylene group and a substituted or unsubstituted biphenylene group, or a trivalent, tetravalent, pentavalent, or hexavalent group derived from the divalent group;

n₃ is 1; and

L₃₂ is a single bond, a substituted or unsubstituted phenylene group, or a substituted or unsubstituted biphenylene group.

In the compounds represented by the formulae (3X) and (3Y), R₃₅₂ is preferably a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compounds represented by the formulae (3X) and (3Y), it is preferable that:

a combination of R₃₅₃ and R₃₅₄ are mutually bonded to form a substituted or unsubstituted monocyclic ring, mutually bonded to form a substituted or unsubstituted fused ring, or not mutually bonded; and

R₃₅₃ and R₃₅₄ forming neither the substituted or unsubstituted monocyclic ring nor the substituted or unsubstituted fused ring are each independently a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compounds represented by the formulae (3X) and (3Y), it is preferable that:

the substituent for the “substituted or unsubstituted” group is an unsubstituted alkyl group having 1 to 25 carbon atoms, an unsubstituted alkenyl group having 2 to 25 carbon atoms, an unsubstituted alkynyl group having 2 to 25 carbon atoms, an unsubstituted cycloalkyl group having 3 to 25 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), an unsubstituted aralkyl group having 7 to 50 carbon atoms, a group represented by —C(═O)R₉₀₈, a group represented by —COOR₉₀₉, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a group represented by —S(═O)₂R₉₃₈, a halogen atom, a cyano group, a nitro group, an unsubstituted aryl group having 6 to 25 ring carbon atoms, or an unsubstituted heterocyclic group having 5 to 25 ring atoms; and

R₉₀₁ to R₉₀₉ and R₉₃₁ to R₉₃₈ are each independently a hydrogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compounds represented by the formulae (3X) and (3Y), it is preferable that the substituent for the “substituted or unsubstituted” group is a halogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compounds represented by the formulae (3X) and (3Y), it is preferable that the substituent for the “substituted or unsubstituted” group is an unsubstituted alkyl group having 1 to 10 carbon atoms, an unsubstituted aryl group having 6 to 12 ring carbon atoms, or an unsubstituted heterocyclic group having 5 to 12 ring atoms.

In the compounds represented by the formulae (3X) and (3Y), it is also preferable that the groups specified to be “substituted or unsubstituted” are each an “unsubstituted” group.

### Manufacturing Method of Compound M3

The compound M3 according to the exemplary embodiment can be manufactured by a known method.

### Specific Examples of Compound M3

Specific examples of the compound M3 of the exemplary embodiment include compounds below. It should however be noted that the invention is not limited to the specific examples of the compound.

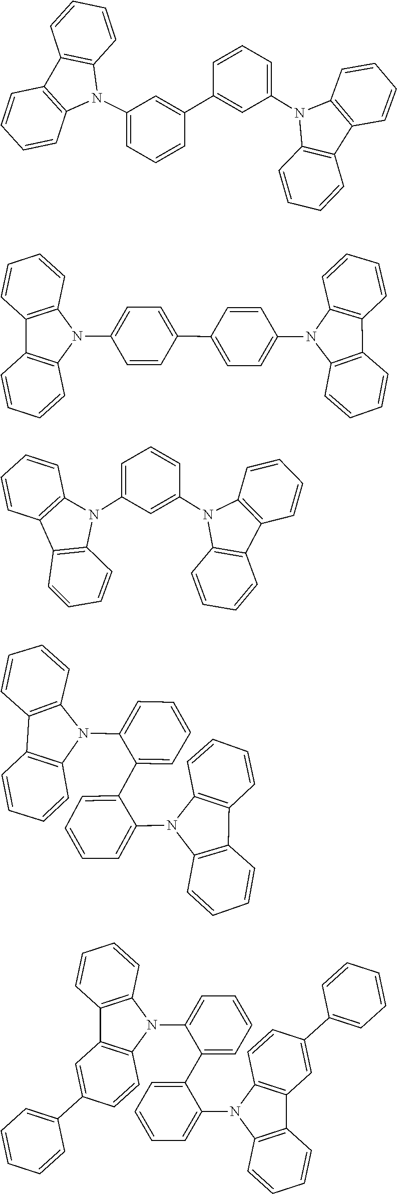

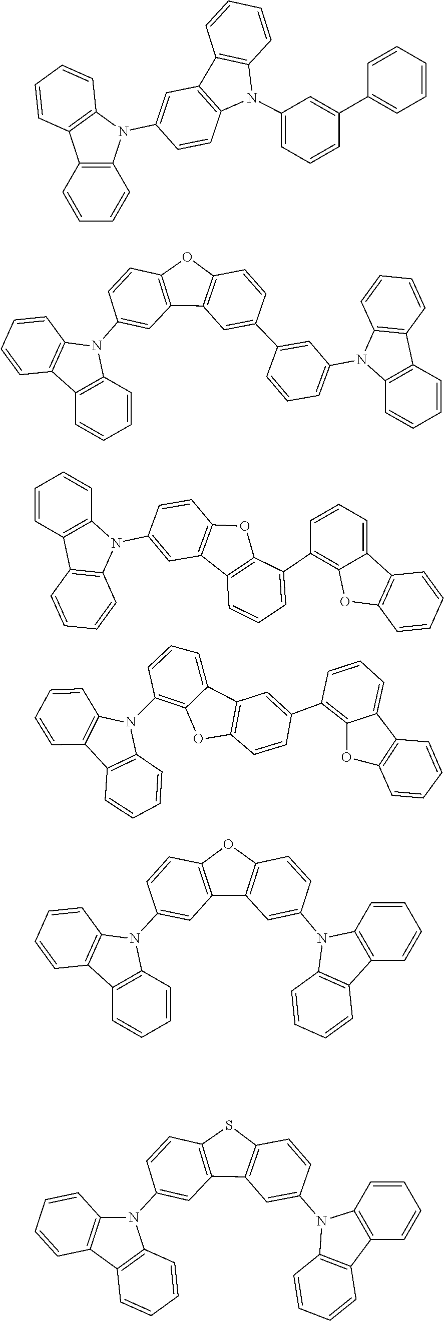 [SMILES: c1ccc(-c2cccc(-n3c4ccccc4c4cc(-n5c6ccccc6c6ccccc65)ccc43)c2)cc1 ; c1cc(-c2ccc3oc4ccc(-n5c6ccccc6c6ccccc65)cc4c3c2)cc(-n2c3ccccc3c3ccccc32)c1 ; c1ccc2c(c1)oc1c(-c3cccc4c3oc3ccc(-n5c6ccccc6c6ccccc65)cc34)cccc12 ; c1ccc2c(c1)oc1c(-c3ccc4oc5c(-n6c7ccccc7c7ccccc76)cccc5c4c3)cccc12 ; c1ccc2c(c1)c1ccccc1n2-c1ccc2oc3ccc(-n4c5ccccc5c5ccccc54)cc3c2c1 ; c1ccc2c(c1)c1ccccc1n2-c1ccc2sc3ccc(-n4c5ccccc5c5ccccc54)cc3c2c1]

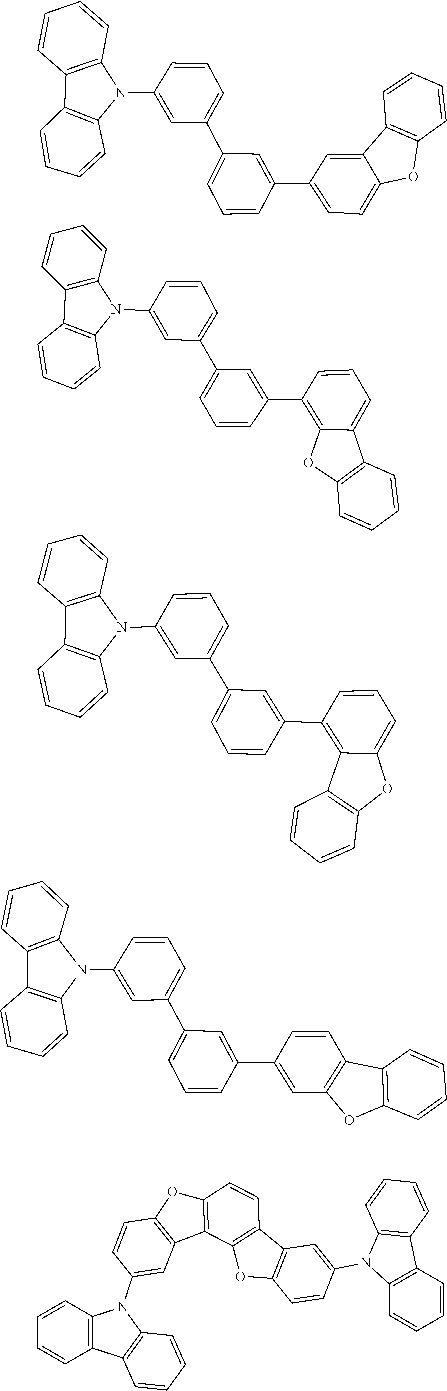 [SMILES: c1cc(-c2cccc(-n3c4ccccc4c4ccccc43)c2)cc(-c2ccc3oc4ccccc4c3c2)c1 ; c1cc(-c2cccc(-n3c4ccccc4c4ccccc43)c2)cc(-c2cccc3c2oc2ccccc23)c1 ; c1cc(-c2cccc(-n3c4ccccc4c4ccccc43)c2)cc(-c2cccc3oc4ccccc4c23)c1 ; c1cc(-c2cccc(-n3c4ccccc4c4ccccc43)c2)cc(-c2ccc3c(c2)oc2ccccc23)c1 ; c1ccc2c(c1)c1ccccc1n2-c1ccc2oc3c(ccc4oc5ccc(-n6c7ccccc7c7ccccc76)cc5c43)c2c1]

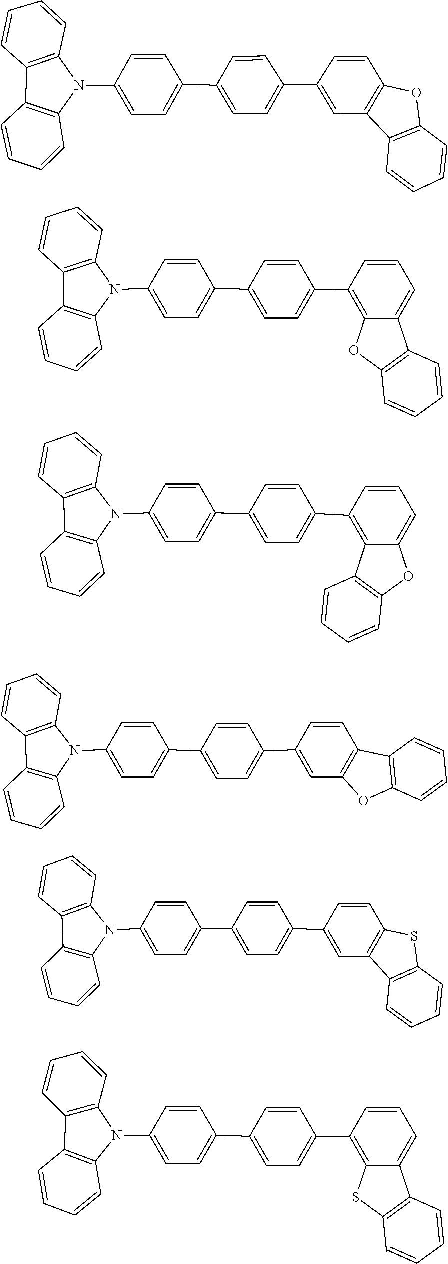

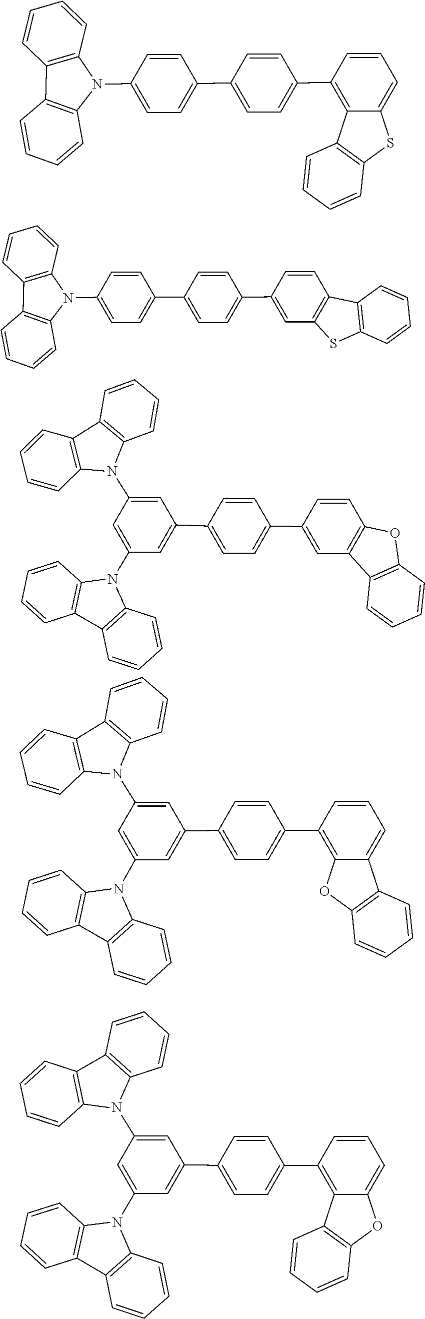

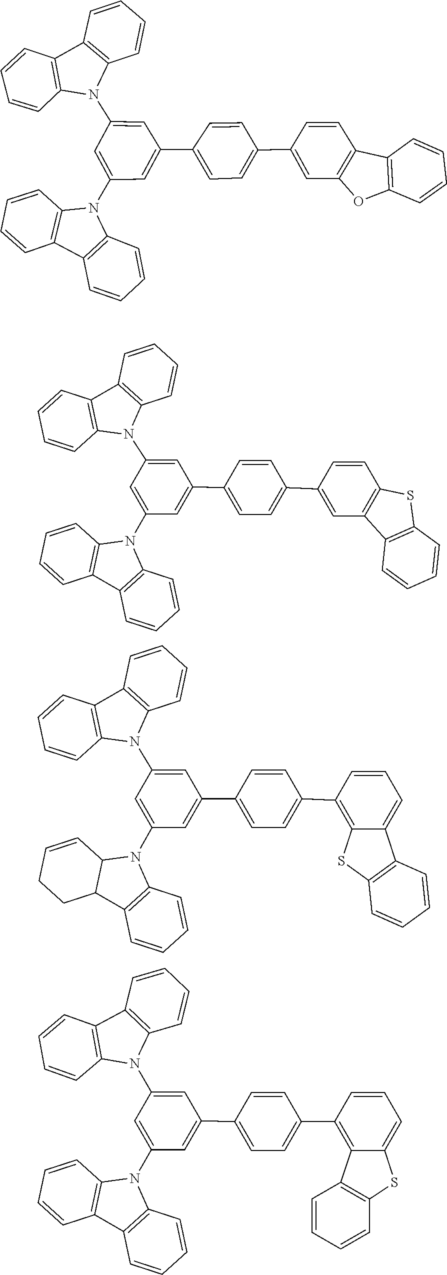

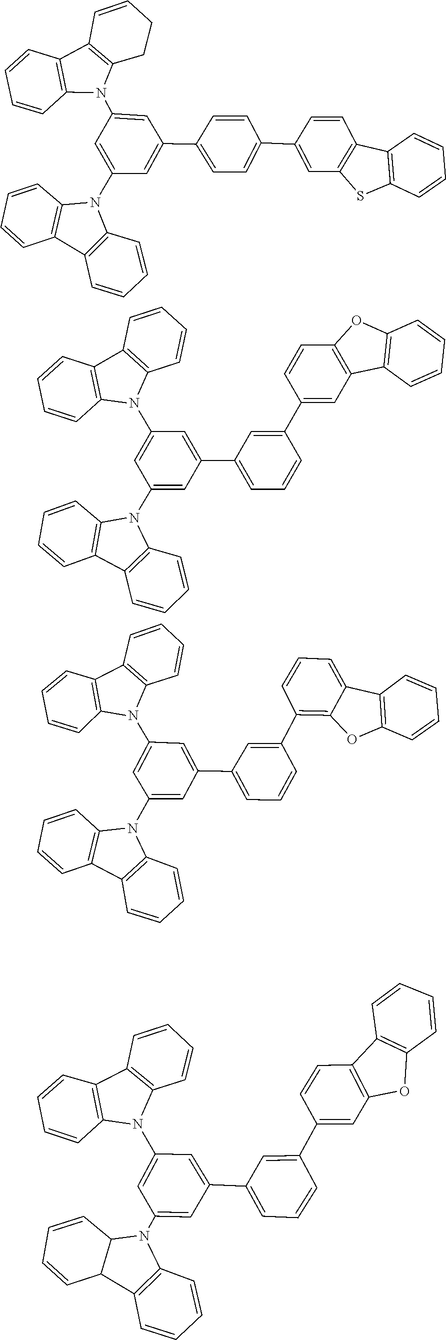

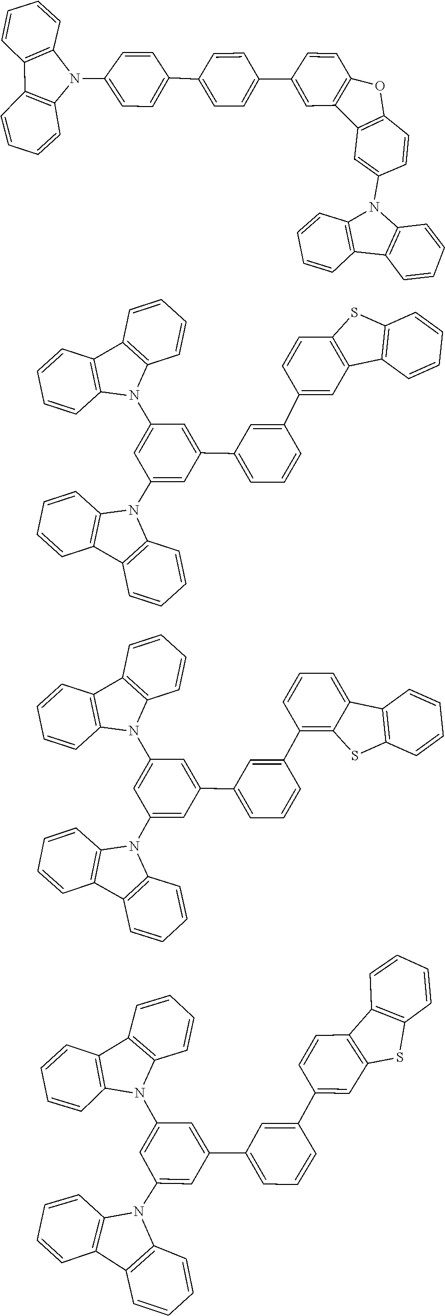

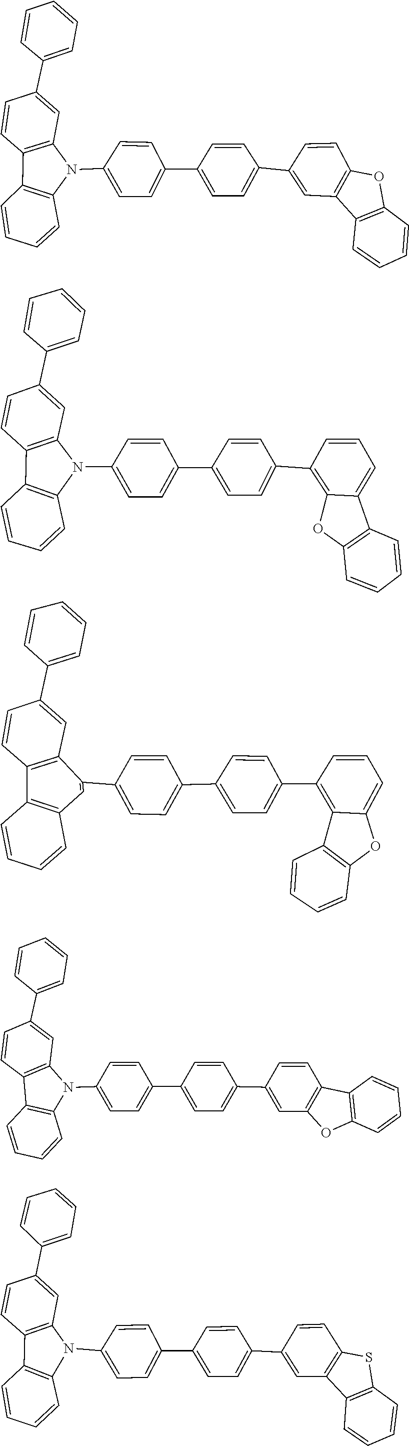

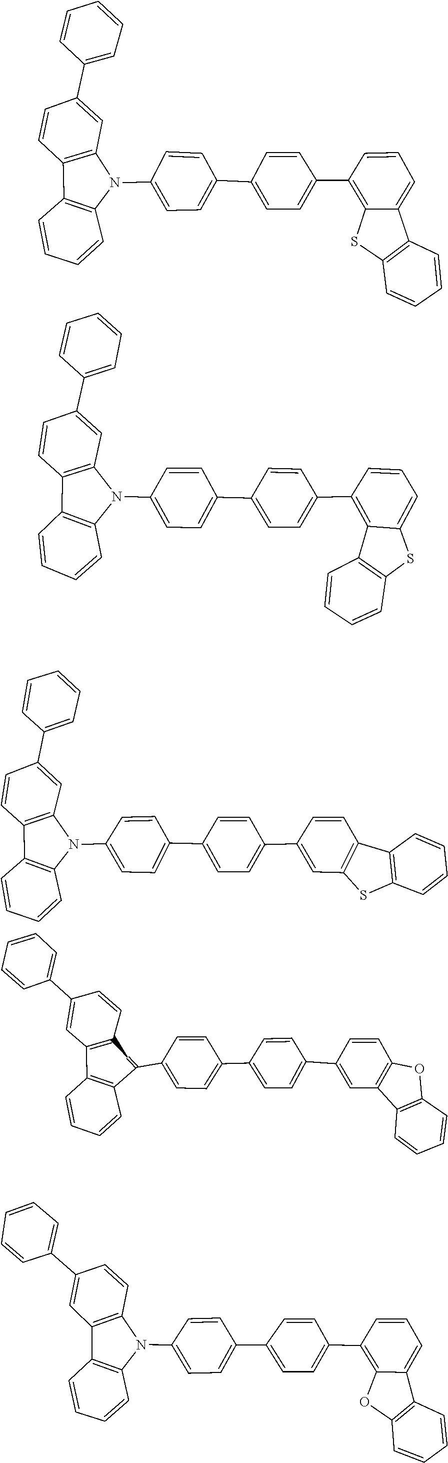

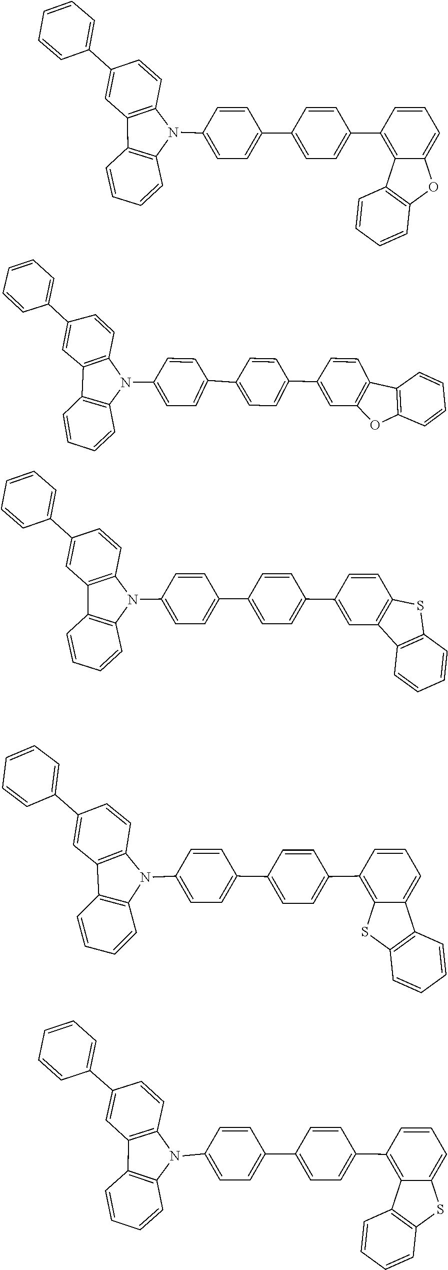 [SMILES: c1ccc(-c2ccc3c(c2)c2ccccc2n3-c2ccc(-c3ccc(-c4cccc5oc6ccccc6c45)cc3)cc2)cc1 ; c1ccc(-c2ccc3c(c2)c2ccccc2n3-c2ccc(-c3ccc(-c4ccc5c(c4)oc4ccccc45)cc3)cc2)cc1 ; c1ccc(-c2ccc3c(c2)c2ccccc2n3-c2ccc(-c3ccc(-c4ccc5sc6ccccc6c5c4)cc3)cc2)cc1 ; c1ccc(-c2ccc3c(c2)c2ccccc2n3-c2ccc(-c3ccc(-c4cccc5c4sc4ccccc45)cc3)cc2)cc1 ; c1ccc(-c2ccc3c(c2)c2ccccc2n3-c2ccc(-c3ccc(-c4cccc5sc6ccccc6c45)cc3)cc2)cc1]

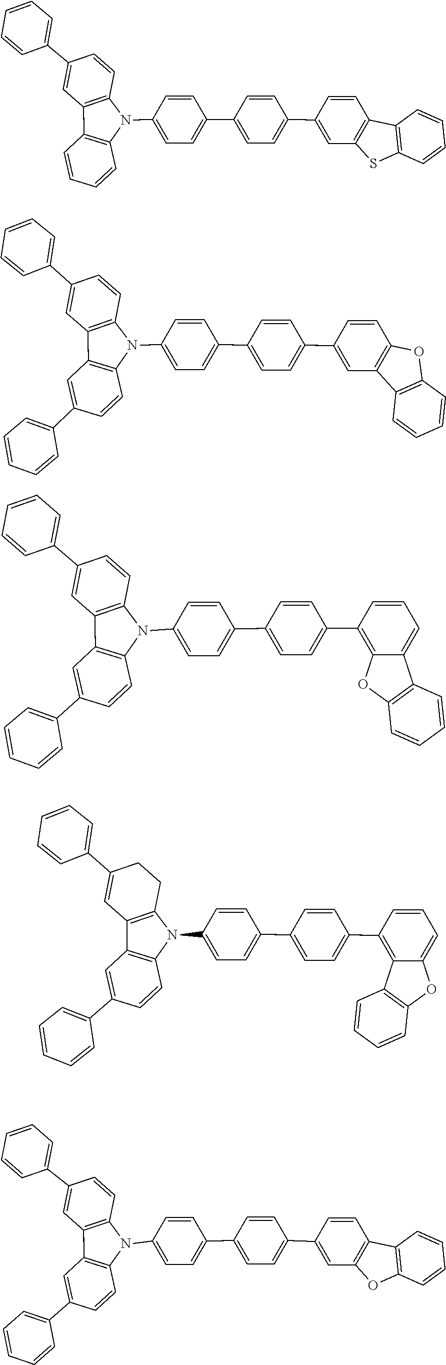

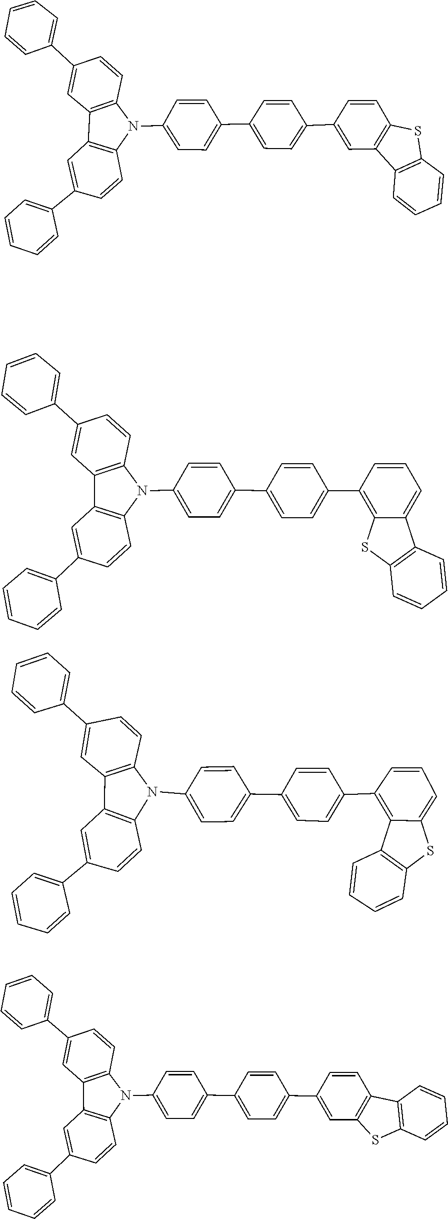

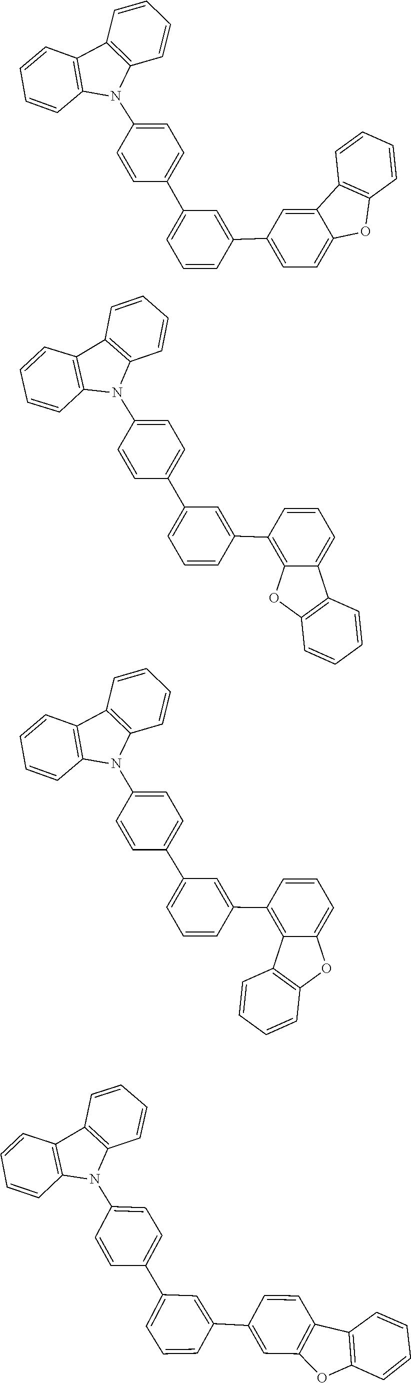 [SMILES: c1cc(-c2ccc(-n3c4ccccc4c4ccccc43)cc2)cc(-c2ccc3oc4ccccc4c3c2)c1 ; c1cc(-c2ccc(-n3c4ccccc4c4ccccc43)cc2)cc(-c2cccc3c2oc2ccccc23)c1 ; c1cc(-c2ccc(-n3c4ccccc4c4ccccc43)cc2)cc(-c2cccc3oc4ccccc4c23)c1 ; c1cc(-c2ccc(-n3c4ccccc4c4ccccc43)cc2)cc(-c2ccc3c(c2)oc2ccccc23)c1]

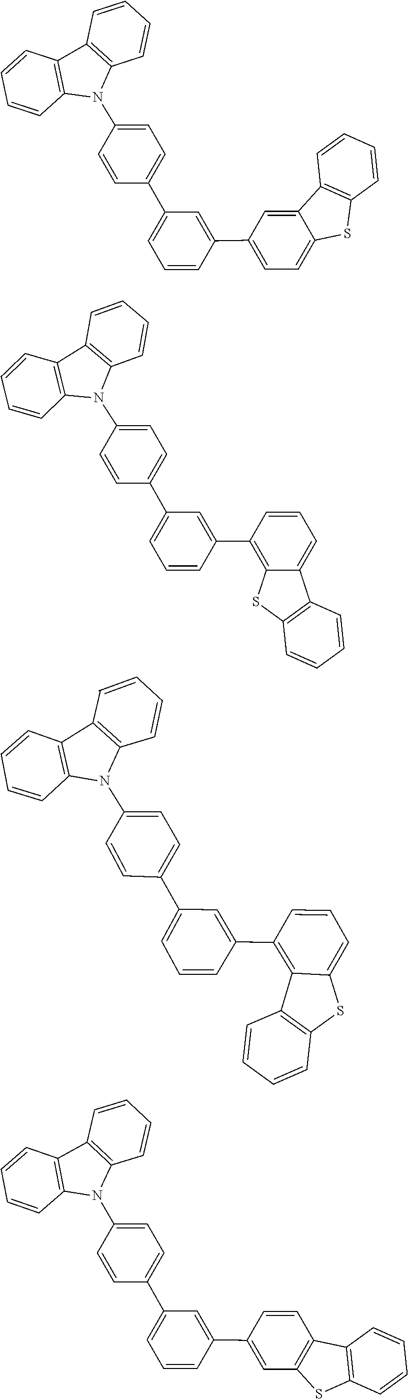 [SMILES: c1cc(-c2ccc(-n3c4ccccc4c4ccccc43)cc2)cc(-c2ccc3sc4ccccc4c3c2)c1 ; c1cc(-c2ccc(-n3c4ccccc4c4ccccc43)cc2)cc(-c2cccc3c2sc2ccccc23)c1 ; Cc1cccc2c1c1ccccc1n2-c1ccc(-c2cccc(-c3cccc4sc5ccccc5c34)c2)cc1 ; c1cc(-c2ccc(-n3c4ccccc4c4ccccc43)cc2)cc(-c2ccc3c(c2)sc2ccccc23)c1]

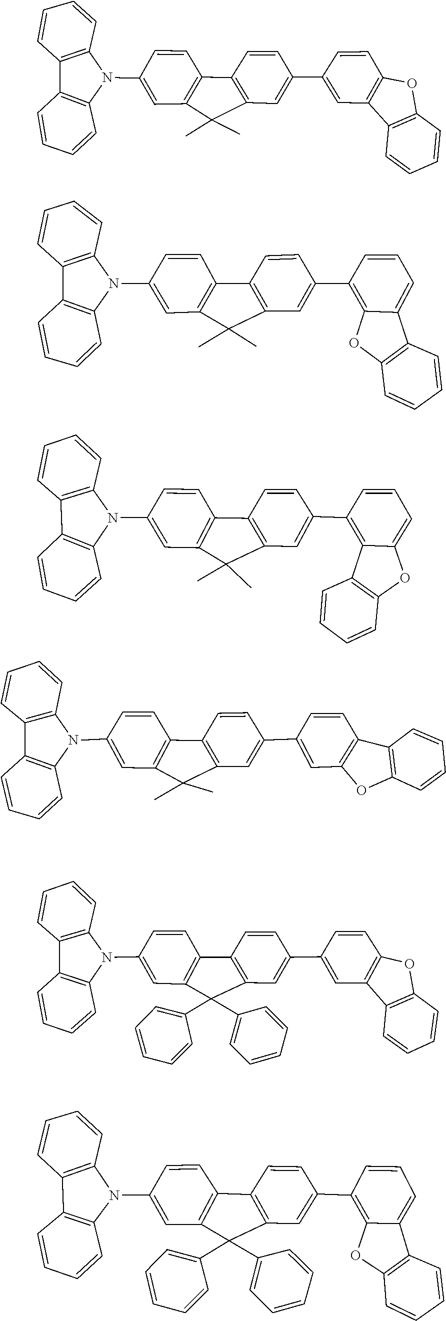 [SMILES: CC1(C)c2cc(-c3ccc4oc5ccccc5c4c3)ccc2-c2ccc(-n3c4ccccc4c4ccccc43)cc21 ; CC1(C)c2cc(-c3cccc4c3oc3ccccc34)ccc2-c2ccc(-n3c4ccccc4c4ccccc43)cc21 ; CC1(C)c2cc(-c3cccc4oc5ccccc5c34)ccc2-c2ccc(-n3c4ccccc4c4ccccc43)cc21 ; CC1(C)c2cc(-c3ccc4c(c3)oc3ccccc34)ccc2-c2ccc(-n3c4ccccc4c4ccccc43)cc21 ; c1ccc(C2(c3ccccc3)c3cc(-c4ccc5oc6ccccc6c5c4)ccc3-c3ccc(-n4c5ccccc5c5ccccc54)cc32)cc1 ; c1ccc(C2(c3ccccc3)c3cc(-c4cccc5c4oc4ccccc45)ccc3-c3ccc(-n4c5ccccc5c5ccccc54)cc32)cc1]

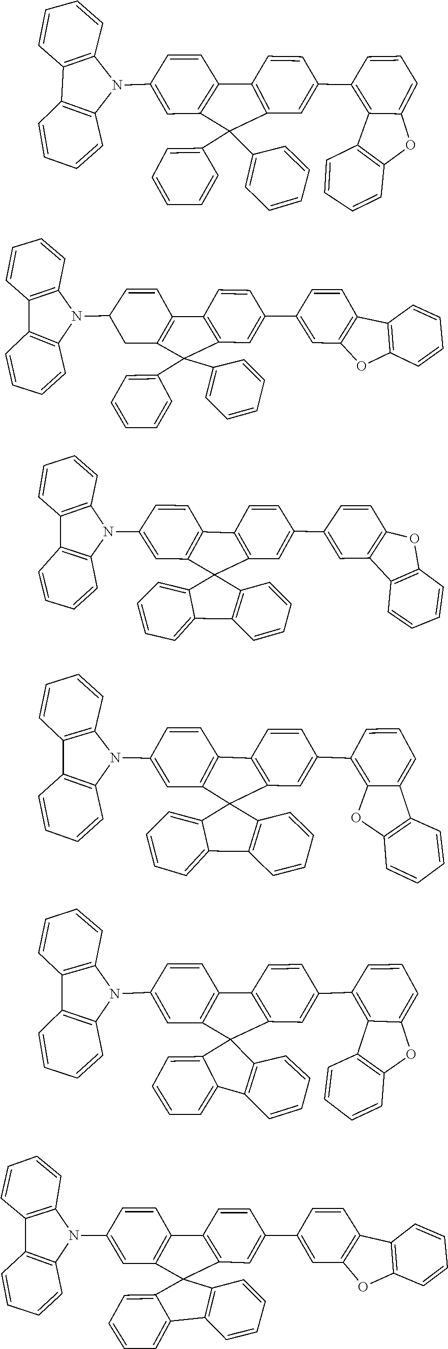

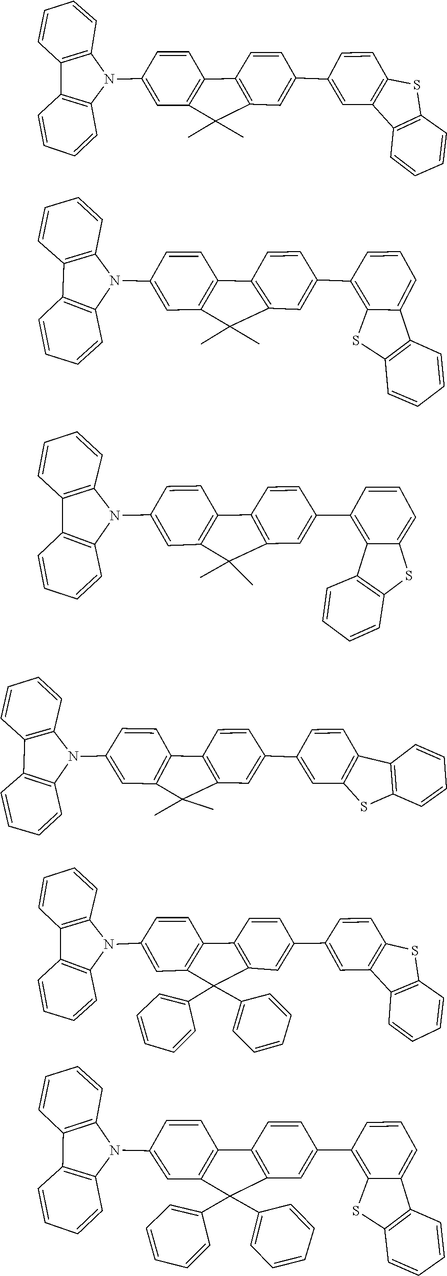

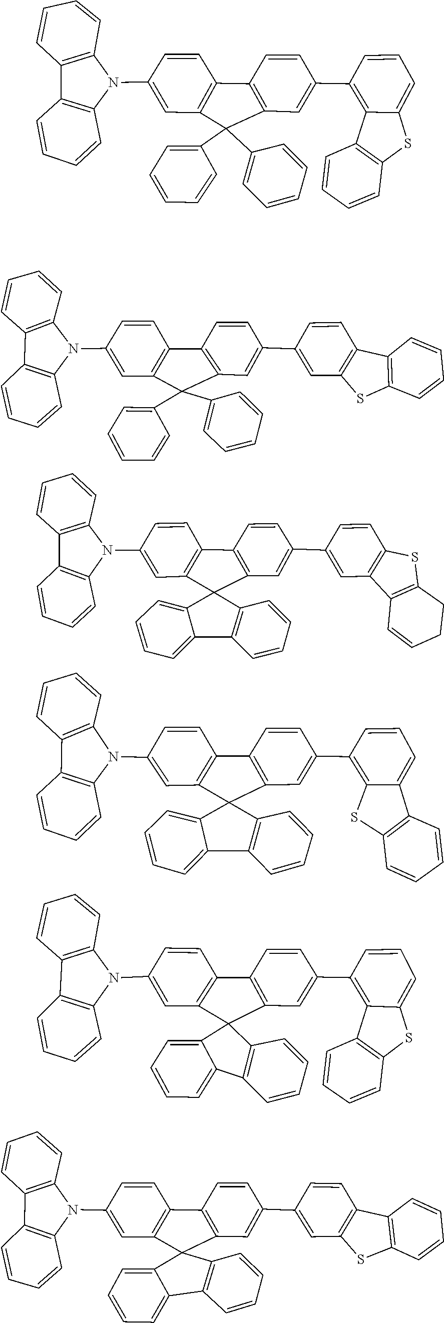

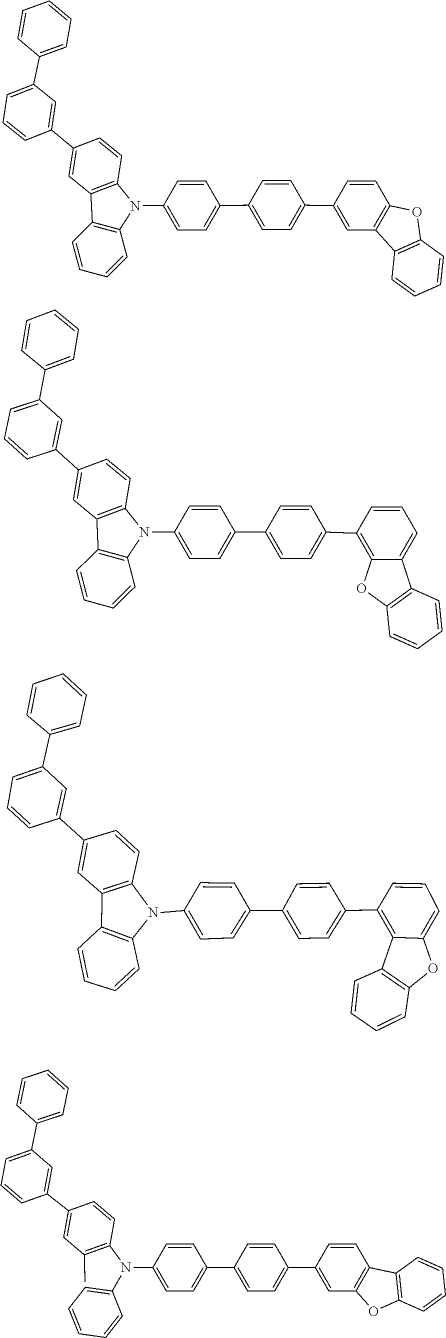 [SMILES: c1ccc(-c2cccc(-c3ccc4c(c3)c3ccccc3n4-c3ccc(-c4ccc(-c5ccc6oc7ccccc7c6c5)cc4)cc3)c2)cc1 ; c1ccc(-c2cccc(-c3ccc4c(c3)c3ccccc3n4-c3ccc(-c4ccc(-c5cccc6c5oc5ccccc56)cc4)cc3)c2)cc1 ; c1ccc(-c2cccc(-c3ccc4c(c3)c3ccccc3n4-c3ccc(-c4ccc(-c5cccc6oc7ccccc7c56)cc4)cc3)c2)cc1 ; Cc1cc(-c2cccc(-c3ccccc3)c2)ccc1N(c1ccccc1)c1ccc(-c2ccc(-c3ccc4c(c3)oc3ccccc34)cc2)cc1]

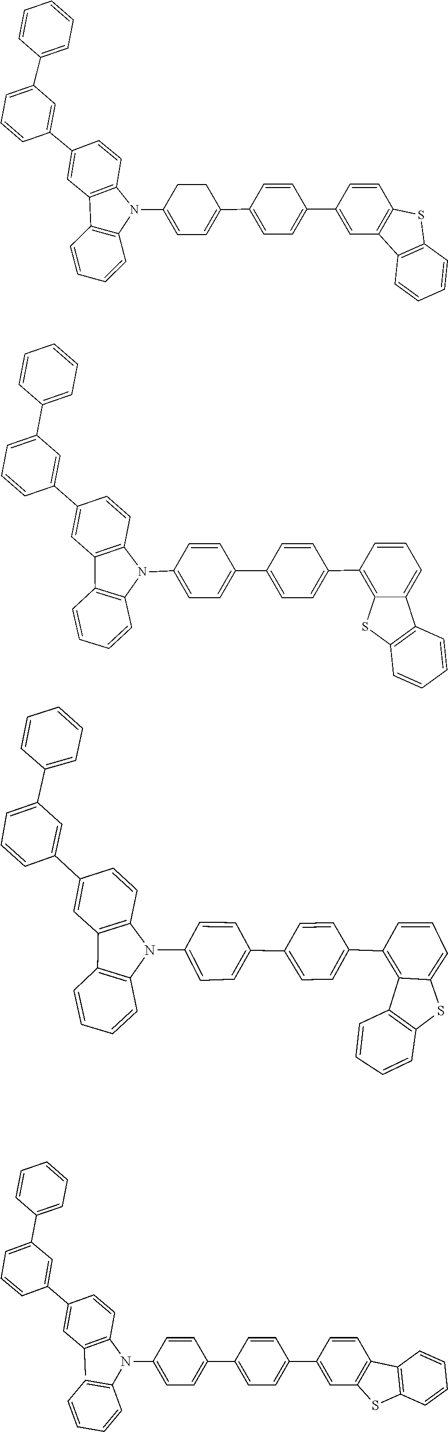 [SMILES: C1=C(c2ccc(-c3ccc4sc5ccccc5c4c3)cc2)CCC(n2c3ccccc3c3cc(-c4cccc(-c5ccccc5)c4)ccc32)=C1 ; c1ccc(-c2cccc(-c3ccc4c(c3)c3ccccc3n4-c3ccc(-c4ccc(-c5cccc6c5sc5ccccc56)cc4)cc3)c2)cc1 ; c1ccc(-c2cccc(-c3ccc4c(c3)c3ccccc3n4-c3ccc(-c4ccc(-c5cccc6sc7ccccc7c56)cc4)cc3)c2)cc1 ; Cc1cc(-c2cccc(-c3ccccc3)c2)ccc1N(c1ccccc1)c1ccc(-c2ccc(-c3ccc4c(c3)sc3ccccc34)cc2)cc1]

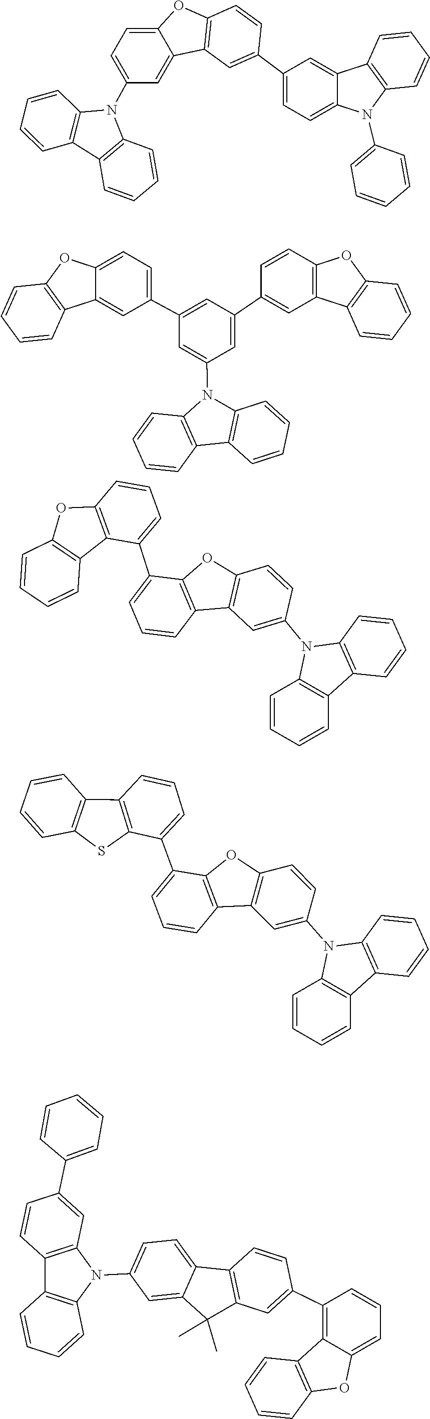

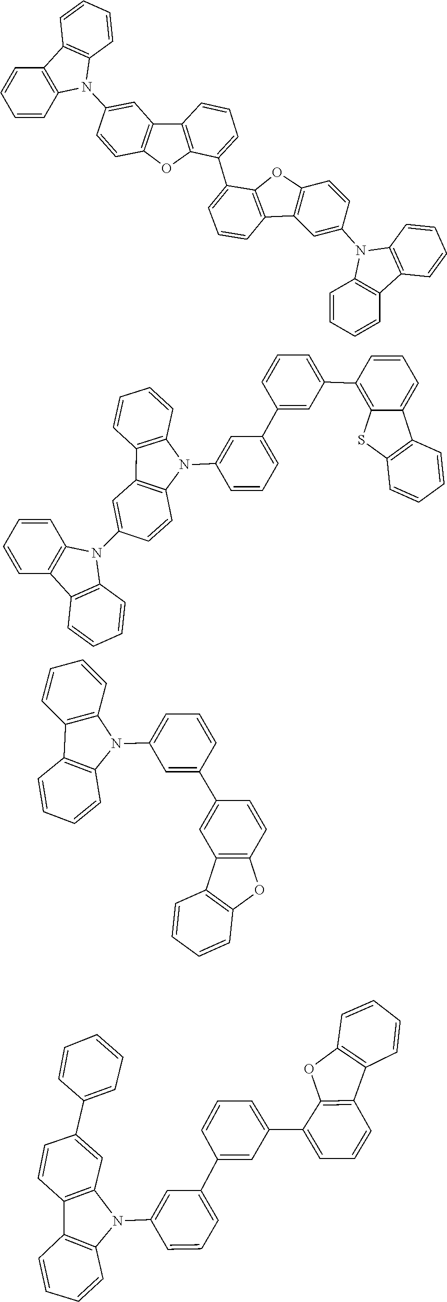

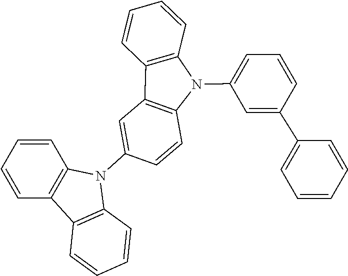 [SMILES: c1ccc(-c2cccc(-n3c4ccccc4c4cc(-n5c6ccccc6c6ccccc65)ccc43)c2)cc1]

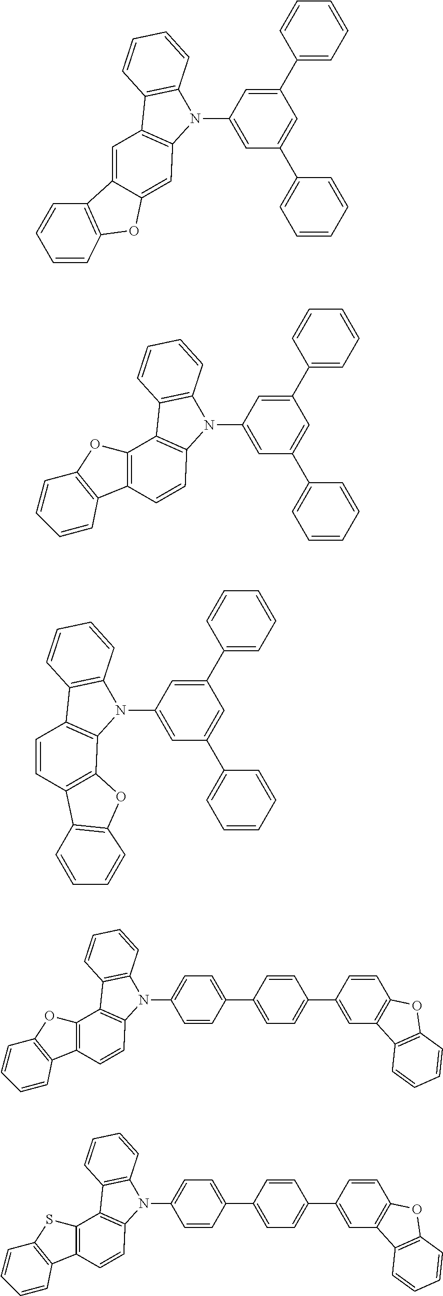 [SMILES: c1ccc(-c2cc(-c3ccccc3)cc(-n3c4ccccc4c4cc5c(cc43)oc3ccccc35)c2)cc1 ; c1ccc(-c2cc(-c3ccccc3)cc(-n3c4ccccc4c4c5oc6ccccc6c5ccc43)c2)cc1 ; c1ccc(-c2cc(-c3ccccc3)cc(-n3c4ccccc4c4ccc5c6ccccc6oc5c43)c2)cc1 ; c1ccc2c(c1)oc1ccc(-c3ccc(-c4ccc(-n5c6ccccc6c6c7oc8ccccc8c7ccc65)cc4)cc3)cc12 ; c1ccc2c(c1)oc1ccc(-c3ccc(-c4ccc(-n5c6ccccc6c6c7sc8ccccc8c7ccc65)cc4)cc3)cc12]

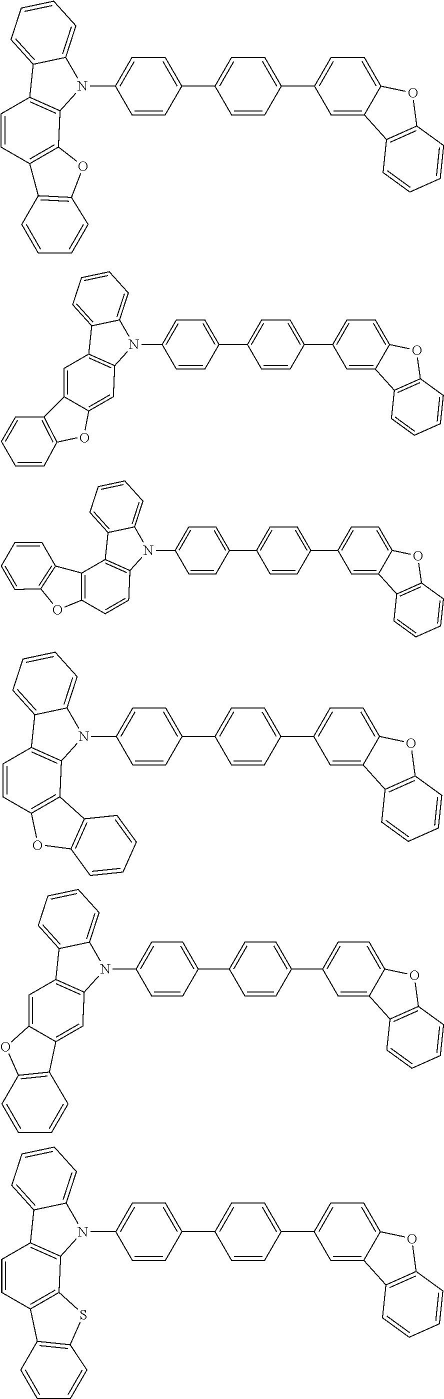

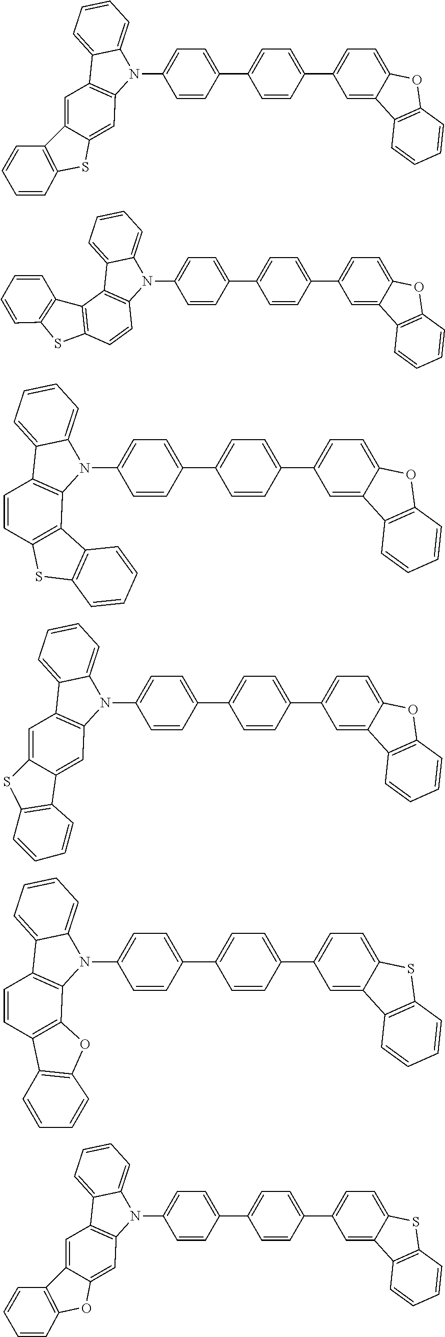

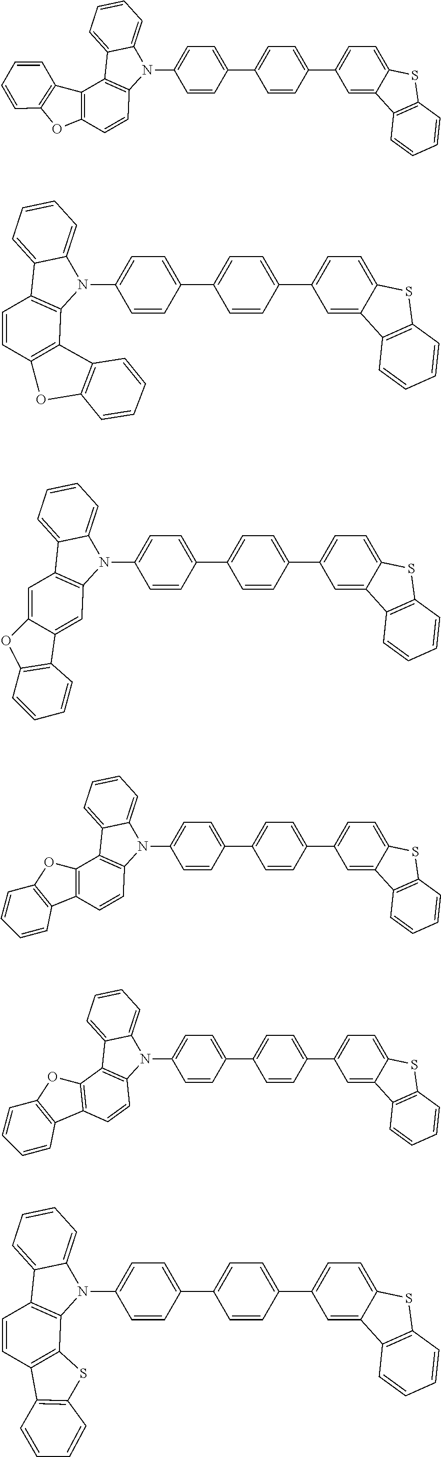 [SMILES: c1ccc2c(c1)oc1ccc3c(c4ccccc4n3-c3ccc(-c4ccc(-c5ccc6sc7ccccc7c6c5)cc4)cc3)c12 ; c1ccc2c(c1)oc1ccc3c4ccccc4n(-c4ccc(-c5ccc(-c6ccc7sc8ccccc8c7c6)cc5)cc4)c3c12 ; c1ccc2c(c1)oc1cc3c4ccccc4n(-c4ccc(-c5ccc(-c6ccc7sc8ccccc8c7c6)cc5)cc4)c3cc12 ; c1ccc2c(c1)oc1c2ccc2c1c1ccccc1n2-c1ccc(-c2ccc(-c3ccc4sc5ccccc5c4c3)cc2)cc1 ; c1ccc2c(c1)oc1c2ccc2c1c1ccccc1n2-c1ccc(-c2ccc(-c3ccc4sc5ccccc5c4c3)cc2)cc1 ; c1ccc2c(c1)sc1ccc(-c3ccc(-c4ccc(-n5c6ccccc6c6ccc7c8ccccc8sc7c65)cc4)cc3)cc12]

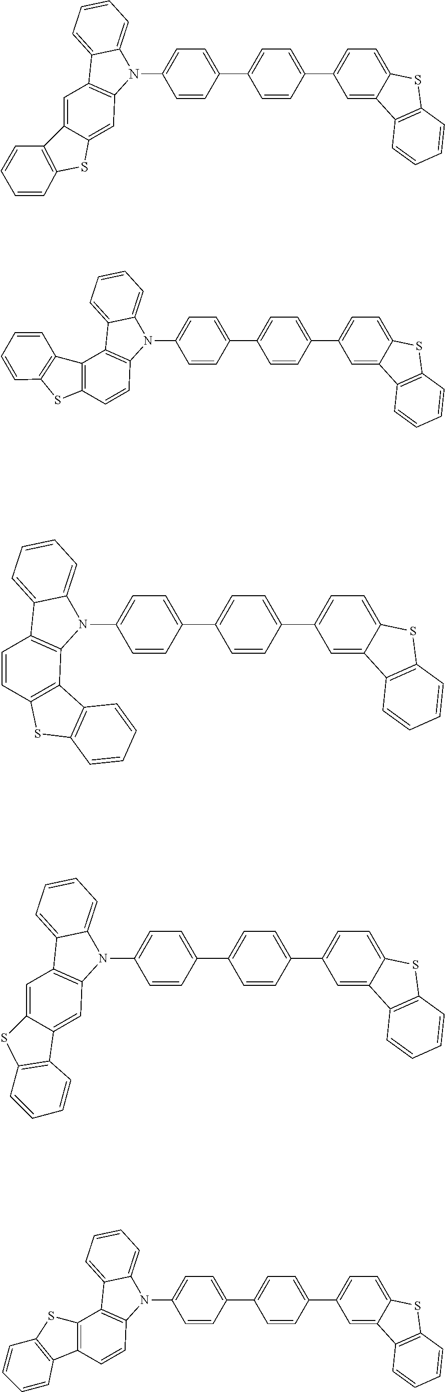 [SMILES: c1ccc2c(c1)sc1ccc(-c3ccc(-c4ccc(-n5c6ccccc6c6cc7c(cc65)sc5ccccc57)cc4)cc3)cc12 ; c1ccc2c(c1)sc1ccc(-c3ccc(-c4ccc(-n5c6ccccc6c6c7c(ccc65)sc5ccccc57)cc4)cc3)cc12 ; c1ccc2c(c1)sc1ccc(-c3ccc(-c4ccc(-n5c6ccccc6c6ccc7sc8ccccc8c7c65)cc4)cc3)cc12 ; c1ccc2c(c1)sc1ccc(-c3ccc(-c4ccc(-n5c6ccccc6c6cc7sc8ccccc8c7cc65)cc4)cc3)cc12 ; c1ccc2c(c1)sc1ccc(-c3ccc(-c4ccc(-n5c6ccccc6c6c7sc8ccccc8c7ccc65)cc4)cc3)cc12]

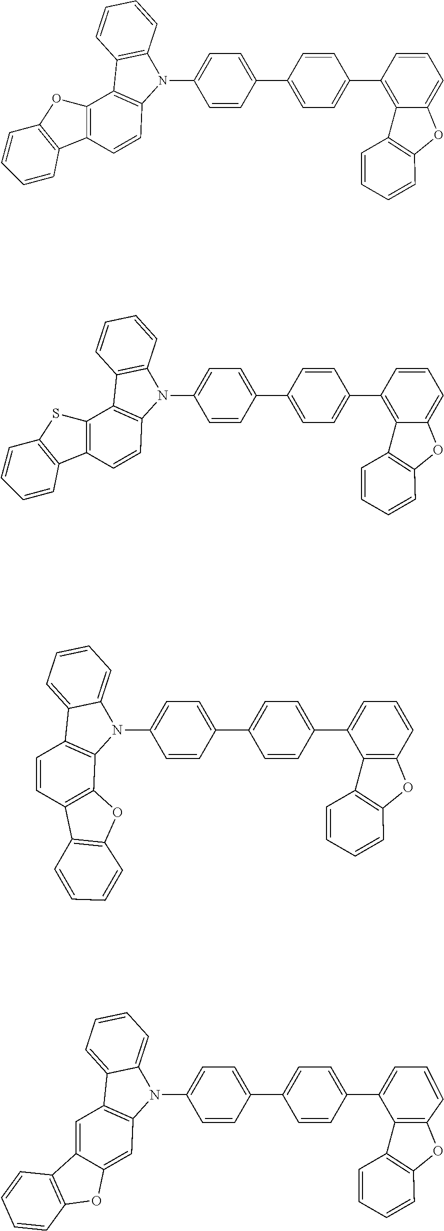 [SMILES: c1ccc2c(c1)oc1c2ccc2c1c1ccccc1n2-c1ccc(-c2ccc(-c3cccc4oc5ccccc5c34)cc2)cc1 ; c1ccc2c(c1)oc1cccc(-c3ccc(-c4ccc(-n5c6ccccc6c6c7sc8ccccc8c7ccc65)cc4)cc3)c12 ; c1ccc2c(c1)oc1c2ccc2c3ccccc3n(-c3ccc(-c4ccc(-c5cccc6oc7ccccc7c56)cc4)cc3)c21 ; c1ccc2c(c1)oc1cc3c(cc12)c1ccccc1n3-c1ccc(-c2ccc(-c3cccc4oc5ccccc5c34)cc2)cc1]

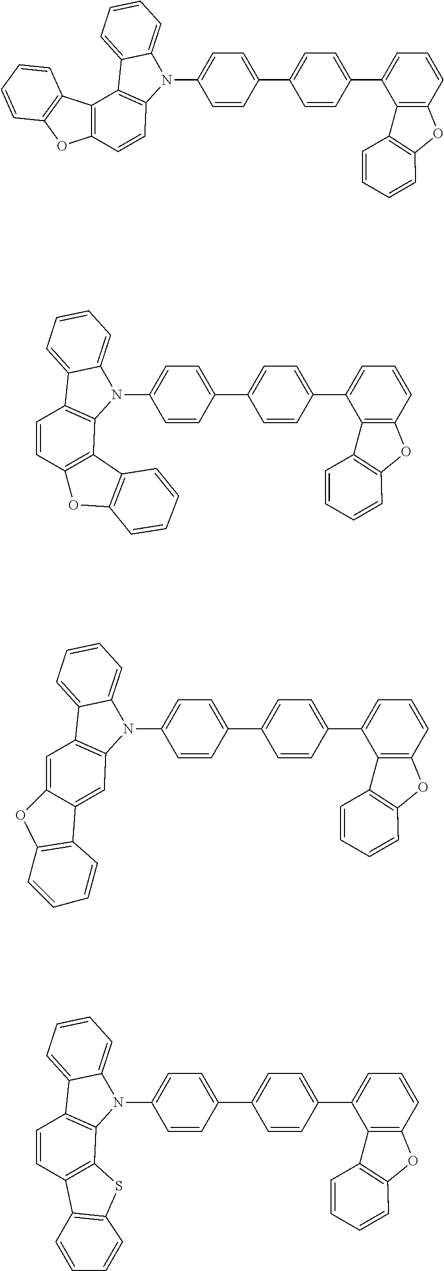 [SMILES: c1ccc2c(c1)oc1cccc(-c3ccc(-c4ccc(-n5c6ccccc6c6c7c(ccc65)oc5ccccc57)cc4)cc3)c12 ; c1ccc2c(c1)oc1cccc(-c3ccc(-c4ccc(-n5c6ccccc6c6ccc7oc8ccccc8c7c65)cc4)cc3)c12 ; c1ccc2c(c1)oc1cc3c4ccccc4n(-c4ccc(-c5ccc(-c6cccc7oc8ccccc8c67)cc5)cc4)c3cc12 ; c1ccc2c(c1)oc1cccc(-c3ccc(-c4ccc(-n5c6ccccc6c6ccc7c8ccccc8sc7c65)cc4)cc3)c12]

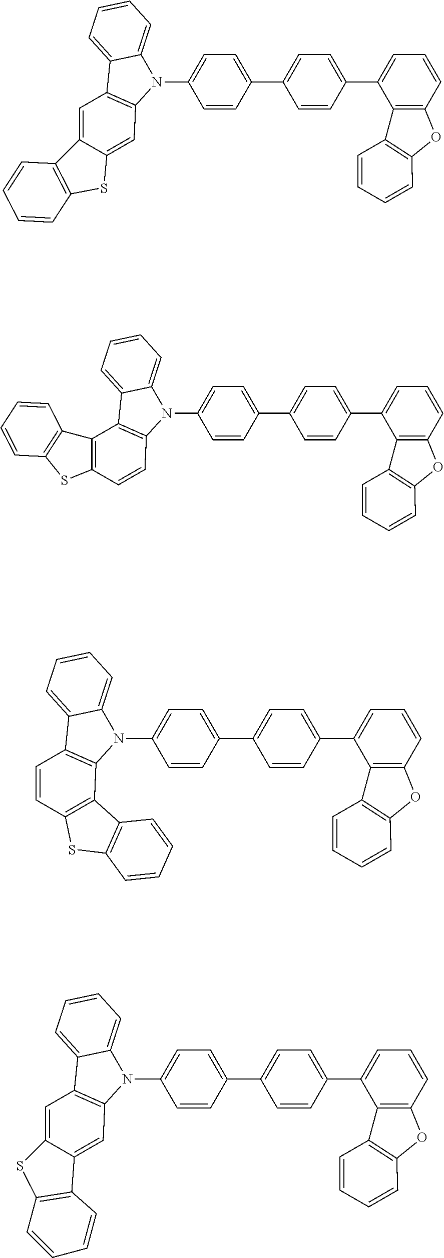 [SMILES: c1ccc2c(c1)oc1cccc(-c3ccc(-c4ccc(-n5c6ccccc6c6cc7c(cc65)sc5ccccc57)cc4)cc3)c12 ; c1ccc2c(c1)oc1cccc(-c3ccc(-c4ccc(-n5c6ccccc6c6c7c(ccc65)sc5ccccc57)cc4)cc3)c12 ; c1ccc2c(c1)oc1cccc(-c3ccc(-c4ccc(-n5c6ccccc6c6ccc7sc8ccccc8c7c65)cc4)cc3)c12 ; c1ccc2c(c1)oc1cccc(-c3ccc(-c4ccc(-n5c6ccccc6c6cc7sc8ccccc8c7cc65)cc4)cc3)c12]

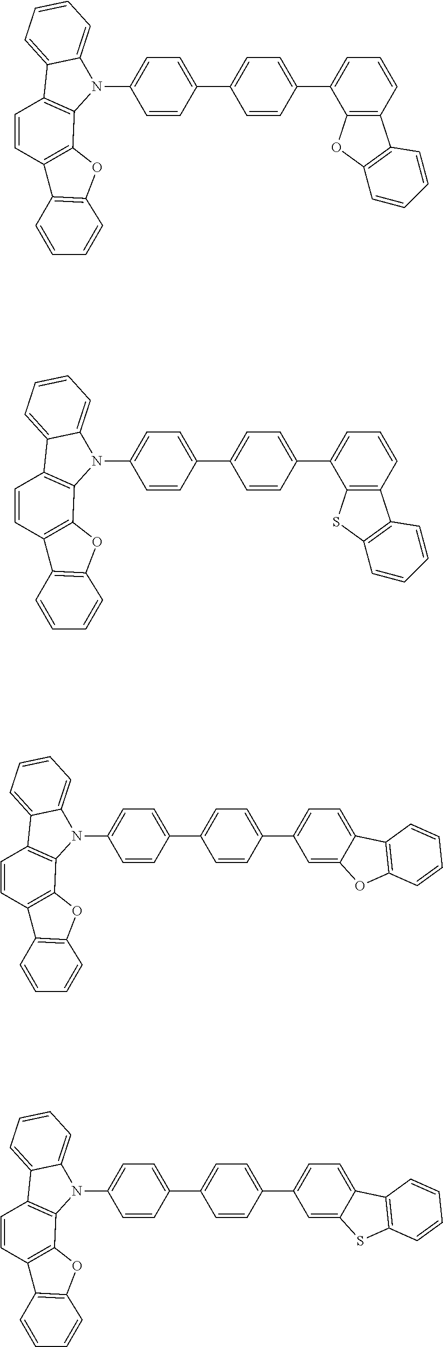 [SMILES: c1ccc2c(c1)oc1c(-c3ccc(-c4ccc(-n5c6ccccc6c6ccc7c8ccccc8oc7c65)cc4)cc3)cccc12 ; c1ccc2c(c1)oc1c2ccc2c3ccccc3n(-c3ccc(-c4ccc(-c5cccc6c5sc5ccccc56)cc4)cc3)c21 ; c1ccc2c(c1)oc1cc(-c3ccc(-c4ccc(-n5c6ccccc6c6ccc7c8ccccc8oc7c65)cc4)cc3)ccc12 ; c1ccc2c(c1)oc1c2ccc2c3ccccc3n(-c3ccc(-c4ccc(-c5ccc6c(c5)sc5ccccc56)cc4)cc3)c21]

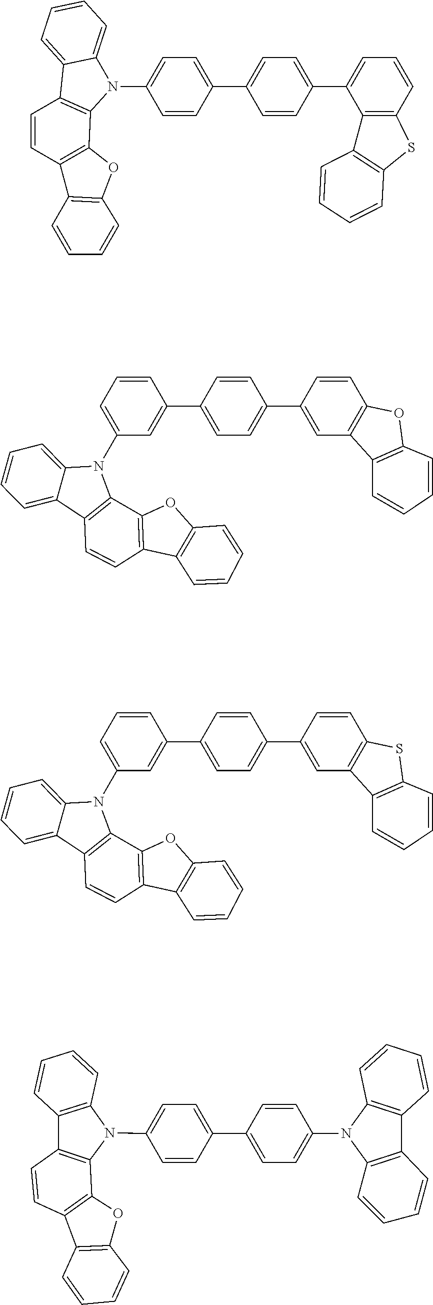 [SMILES: c1ccc2c(c1)oc1c2ccc2c3ccccc3n(-c3ccc(-c4ccc(-c5cccc6sc7ccccc7c56)cc4)cc3)c21 ; c1cc(-c2ccc(-c3ccc4oc5ccccc5c4c3)cc2)cc(-n2c3ccccc3c3ccc4c5ccccc5oc4c32)c1 ; c1cc(-c2ccc(-c3ccc4sc5ccccc5c4c3)cc2)cc(-n2c3ccccc3c3ccc4c5ccccc5oc4c32)c1 ; c1ccc2c(c1)oc1c2ccc2c3ccccc3n(-c3ccc(-c4ccc(-n5c6ccccc6c6ccccc65)cc4)cc3)c21]

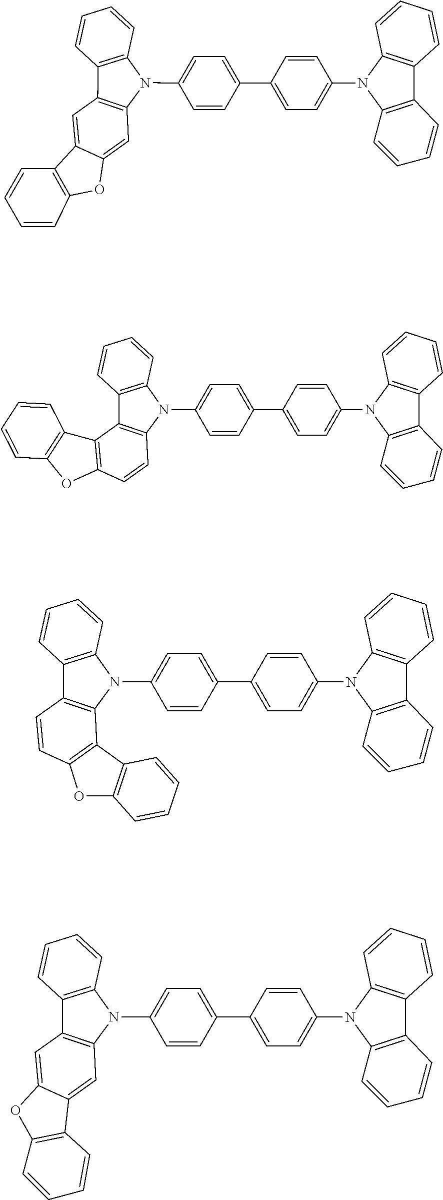 [SMILES: c1ccc2c(c1)oc1cc3c(cc12)c1ccccc1n3-c1ccc(-c2ccc(-n3c4ccccc4c4ccccc43)cc2)cc1 ; c1ccc2c(c1)oc1ccc3c(c4ccccc4n3-c3ccc(-c4ccc(-n5c6ccccc6c6ccccc65)cc4)cc3)c12 ; c1ccc2c(c1)oc1ccc3c4ccccc4n(-c4ccc(-c5ccc(-n6c7ccccc7c7ccccc76)cc5)cc4)c3c12 ; c1ccc2c(c1)oc1cc3c4ccccc4n(-c4ccc(-c5ccc(-n6c7ccccc7c7ccccc76)cc5)cc4)c3cc12]

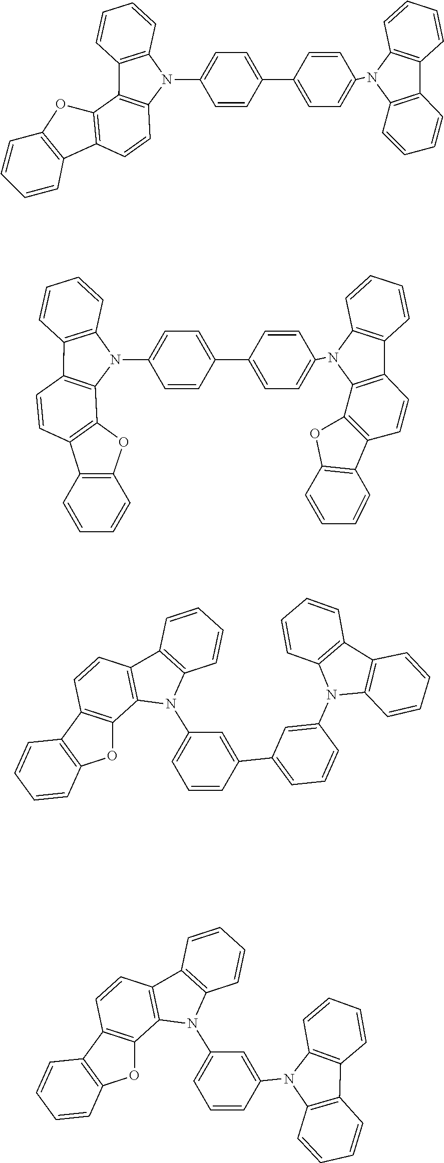 [SMILES: c1ccc2c(c1)oc1c2ccc2c1c1ccccc1n2-c1ccc(-c2ccc(-n3c4ccccc4c4ccccc43)cc2)cc1 ; c1ccc2c(c1)oc1c2ccc2c3ccccc3n(-c3ccc(-c4ccc(-n5c6ccccc6c6ccc7c8ccccc8oc7c65)cc4)cc3)c21 ; c1cc(-c2cccc(-n3c4ccccc4c4ccc5c6ccccc6oc5c43)c2)cc(-n2c3ccccc3c3ccccc32)c1 ; c1cc(-n2c3ccccc3c3ccccc32)cc(-n2c3ccccc3c3ccc4c5ccccc5oc4c32)c1]

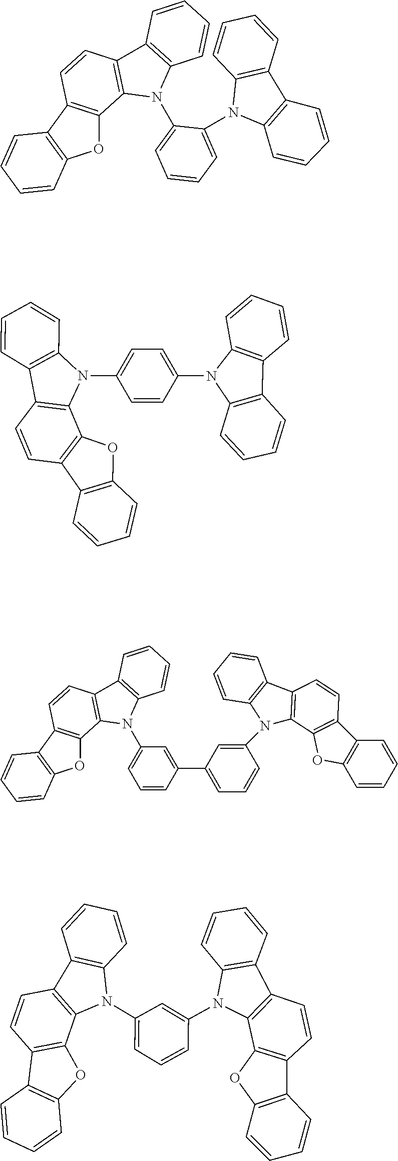 [SMILES: c1ccc(-n2c3ccccc3c3ccc4c5ccccc5oc4c32)c(-n2c3ccccc3c3ccccc32)c1 ; c1ccc2c(c1)oc1c2ccc2c3ccccc3n(-c3ccc(-n4c5ccccc5c5ccccc54)cc3)c21 ; c1cc(-c2cccc(-n3c4ccccc4c4ccc5c6ccccc6oc5c43)c2)cc(-n2c3ccccc3c3ccc4c5ccccc5oc4c32)c1 ; c1cc(-n2c3ccccc3c3ccc4c5ccccc5oc4c32)cc(-n2c3ccccc3c3ccc4c5ccccc5oc4c32)c1]

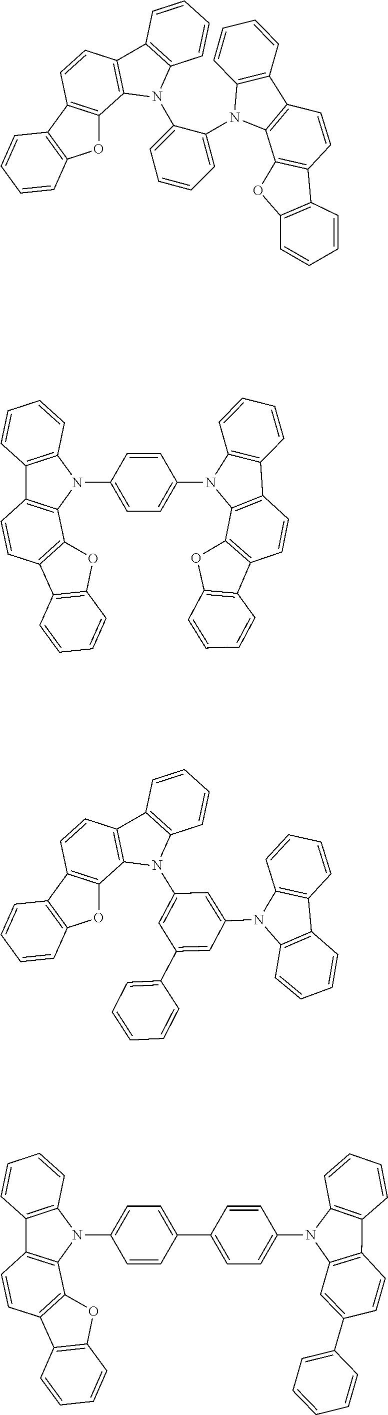 [SMILES: c1ccc(-n2c3ccccc3c3ccc4c5ccccc5oc4c32)c(-n2c3ccccc3c3ccc4c5ccccc5oc4c32)c1 ; c1ccc2c(c1)oc1c2ccc2c3ccccc3n(-c3ccc(-n4c5ccccc5c5ccc6c7ccccc7oc6c54)cc3)c21 ; c1ccc(-c2cc(-n3c4ccccc4c4ccccc43)cc(-n3c4ccccc4c4ccc5c6ccccc6oc5c43)c2)cc1 ; c1ccc(-c2ccc3c4ccccc4n(-c4ccc(-c5ccc(-n6c7ccccc7c7ccc8c9ccccc9oc8c76)cc5)cc4)c3c2)cc1]

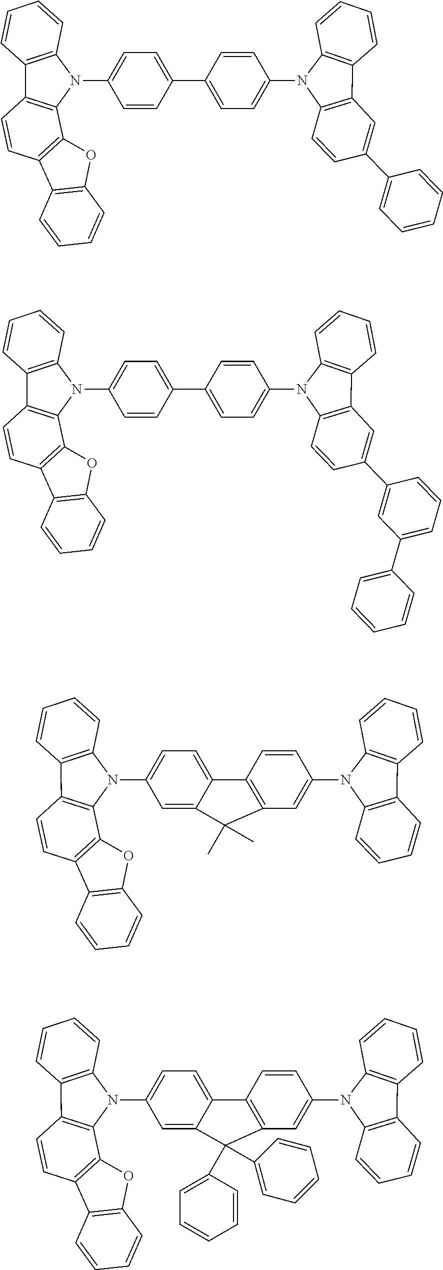 [SMILES: c1ccc(-c2ccc3c(c2)c2ccccc2n3-c2ccc(-c3ccc(-n4c5ccccc5c5ccc6c7ccccc7oc6c54)cc3)cc2)cc1 ; c1ccc(-c2cccc(-c3ccc4c(c3)c3ccccc3n4-c3ccc(-c4ccc(-n5c6ccccc6c6ccc7c8ccccc8oc7c65)cc4)cc3)c2)cc1 ; CC1(C)c2cc(-n3c4ccccc4c4ccccc43)ccc2-c2ccc(-n3c4ccccc4c4ccc5c6ccccc6oc5c43)cc21 ; c1ccc(C2(c3ccccc3)c3cc(-n4c5ccccc5c5ccccc54)ccc3-c3ccc(-n4c5ccccc5c5ccc6c7ccccc7oc6c54)cc32)cc1]

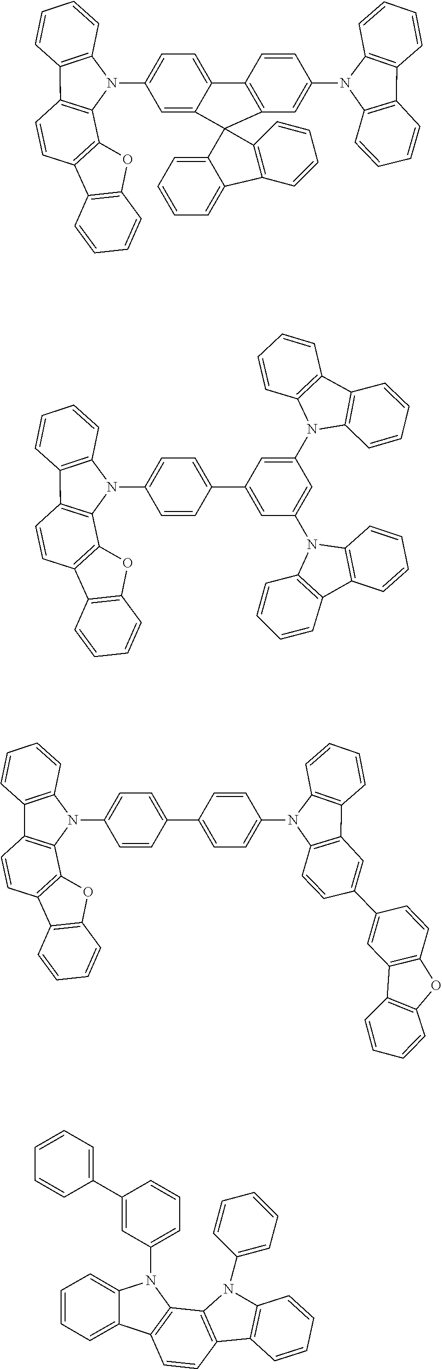 [SMILES: c1ccc2c(c1)-c1ccccc1C21c2cc(-n3c4ccccc4c4ccccc43)ccc2-c2ccc(-n3c4ccccc4c4ccc5c6ccccc6oc5c43)cc21 ; c1ccc2c(c1)oc1c2ccc2c3ccccc3n(-c3ccc(-c4cc(-n5c6ccccc6c6ccccc65)cc(-n5c6ccccc6c6ccccc65)c4)cc3)c21 ; c1ccc2c(c1)oc1ccc(-c3ccc4c(c3)c3ccccc3n4-c3ccc(-c4ccc(-n5c6ccccc6c6ccc7c8ccccc8oc7c65)cc4)cc3)cc12 ; c1ccc(-c2cccc(-n3c4ccccc4c4ccc5c6ccccc6n(-c6ccccc6)c5c43)c2)cc1]

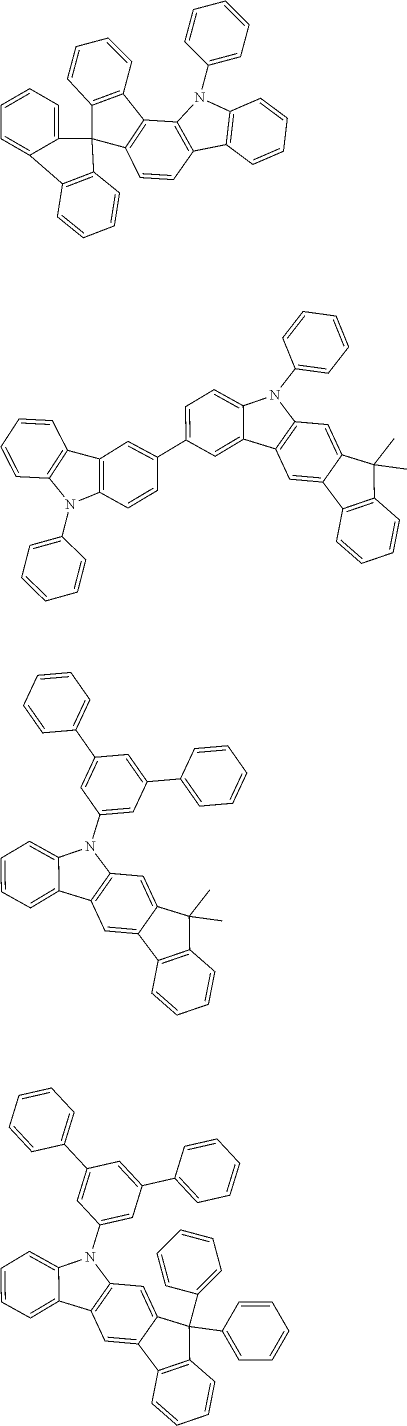 [SMILES: c1ccc(-n2c3ccccc3c3ccc4c(c32)-c2ccccc2C42c3ccccc3-c3ccccc32)cc1 ; CC1(C)c2ccccc2-c2cc3c4cc(-c5ccc6c(c5)c5ccccc5n6-c5ccccc5)ccc4n(-c4ccccc4)c3cc21 ; CC1(C)c2ccccc2-c2cc3c4ccccc4n(-c4cc(-c5ccccc5)cc(-c5ccccc5)c4)c3cc21 ; c1ccc(-c2cc(-c3ccccc3)cc(-n3c4ccccc4c4cc5c(cc43)C(c3ccccc3)(c3ccccc3)c3ccccc3-5)c2)cc1]

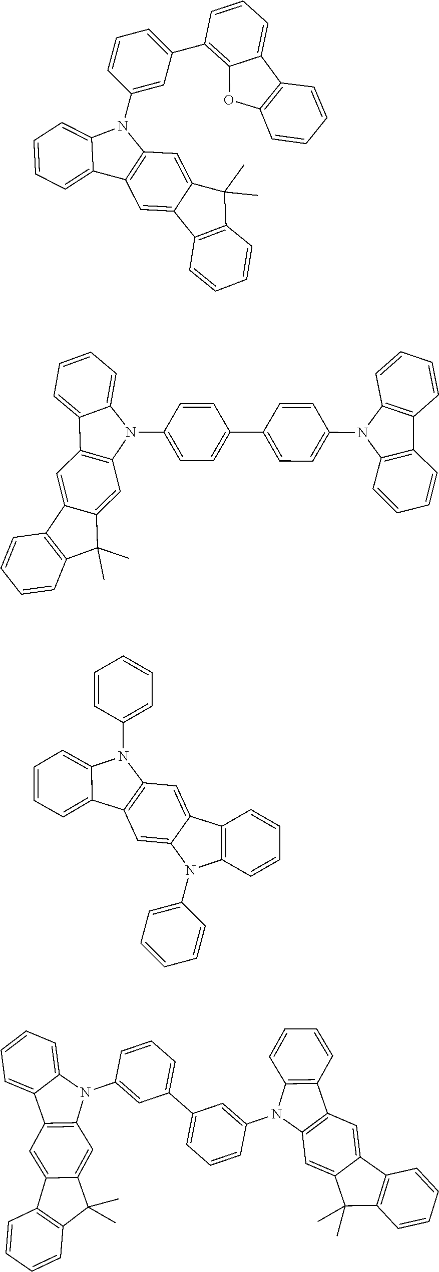 [SMILES: CC1(C)c2ccccc2-c2cc3c4ccccc4n(-c4cccc(-c5cccc6c5oc5ccccc56)c4)c3cc21 ; CC1(C)c2ccccc2-c2cc3c4ccccc4n(-c4ccc(-c5ccc(-n6c7ccccc7c7ccccc76)cc5)cc4)c3cc21 ; c1ccc(-n2c3ccccc3c3cc4c(cc32)c2ccccc2n4-c2ccccc2)cc1 ; CC1(C)c2ccccc2-c2cc3c4ccccc4n(-c4cccc(-c5cccc(-n6c7ccccc7c7cc8c(cc76)C(C)(C)c6ccccc6-8)c5)c4)c3cc21]

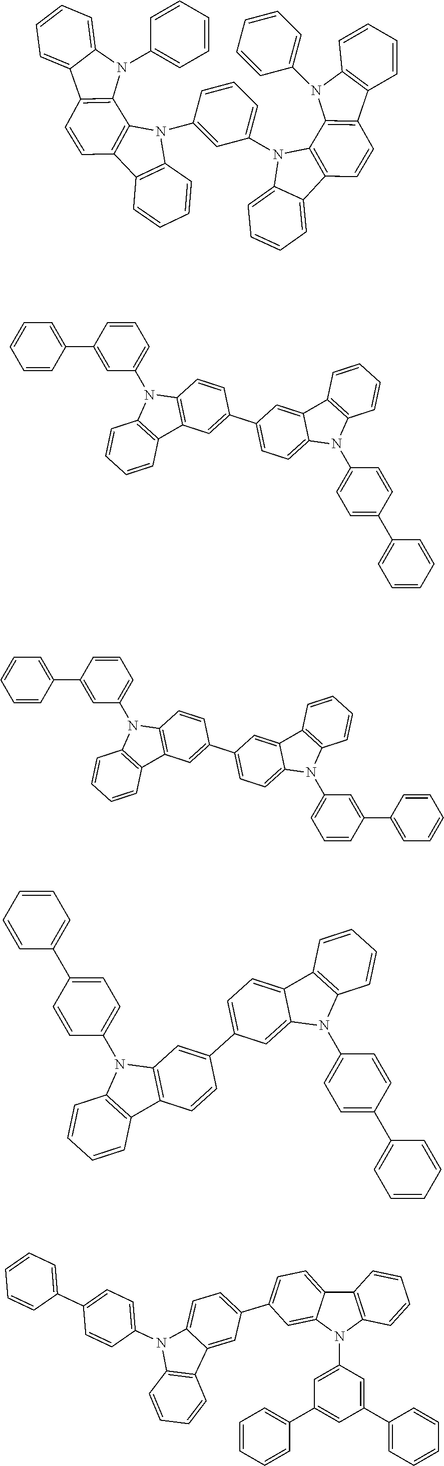 [SMILES: c1ccc(-n2c3ccccc3c3ccc4c5ccccc5n(-c5cccc(-n6c7ccccc7c7ccc8c9ccccc9n(-c9ccccc9)c8c76)c5)c4c32)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-n3c4ccccc4c4ccc(-c5ccc6c7ccccc7n(-c7ccc(-c8ccccc8)cc7)c6c5)cc43)cc2)cc1 ; c1ccc(-c2ccc(-n3c4ccccc4c4cc(-c5ccc6c7ccccc7n(-c7cc(-c8ccccc8)cc(-c8ccccc8)c7)c6c5)ccc43)cc2)cc1]

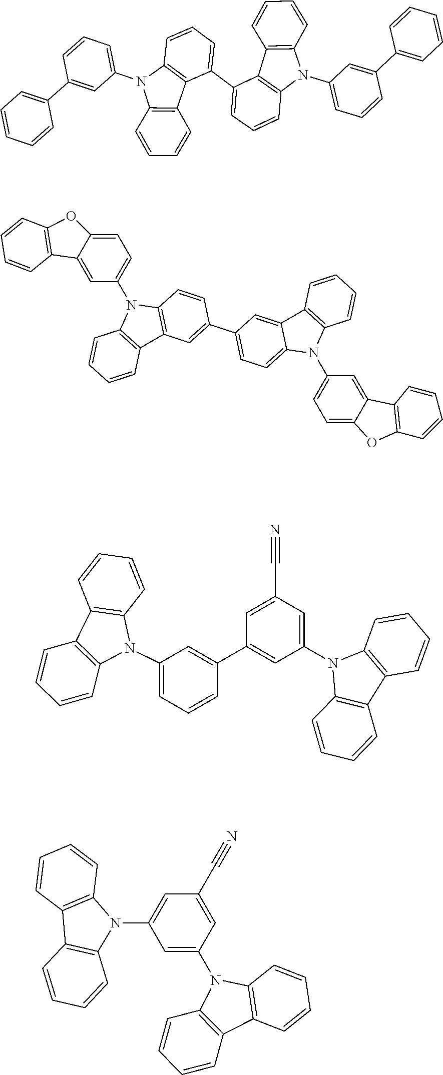 [SMILES: c1ccc(-c2cccc(-n3c4ccccc4c4c(-c5cccc6c5c5ccccc5n6-c5cccc(-c6ccccc6)c5)cccc43)c2)cc1 ; c1ccc2c(c1)oc1ccc(-n3c4ccccc4c4cc(-c5ccc6c(c5)c5ccccc5n6-c5ccc6oc7ccccc7c6c5)ccc43)cc12 ; N#Cc1cc(-c2cccc(-n3c4ccccc4c4ccccc43)c2)cc(-n2c3ccccc3c3ccccc32)c1 ; N#Cc1cc(-n2c3ccccc3c3ccccc32)cc(-n2c3ccccc3c3ccccc32)c1]

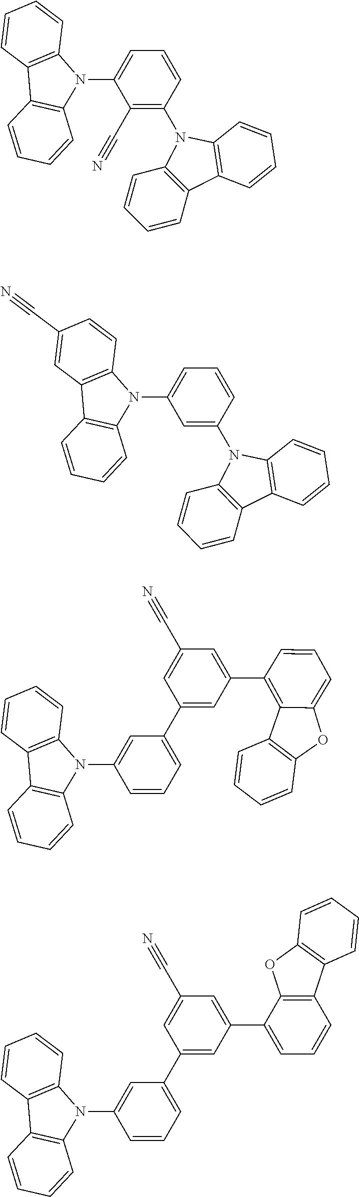 [SMILES: N#Cc1c(-n2c3ccccc3c3ccccc32)cccc1-n1c2ccccc2c2ccccc21 ; N#Cc1ccc2c(c1)c1ccccc1n2-c1cccc(-n2c3ccccc3c3ccccc32)c1 ; N#Cc1cc(-c2cccc(-n3c4ccccc4c4ccccc43)c2)cc(-c2cccc3oc4ccccc4c23)c1 ; N#Cc1cc(-c2cccc(-n3c4ccccc4c4ccccc43)c2)cc(-c2cccc3c2oc2ccccc23)c1]

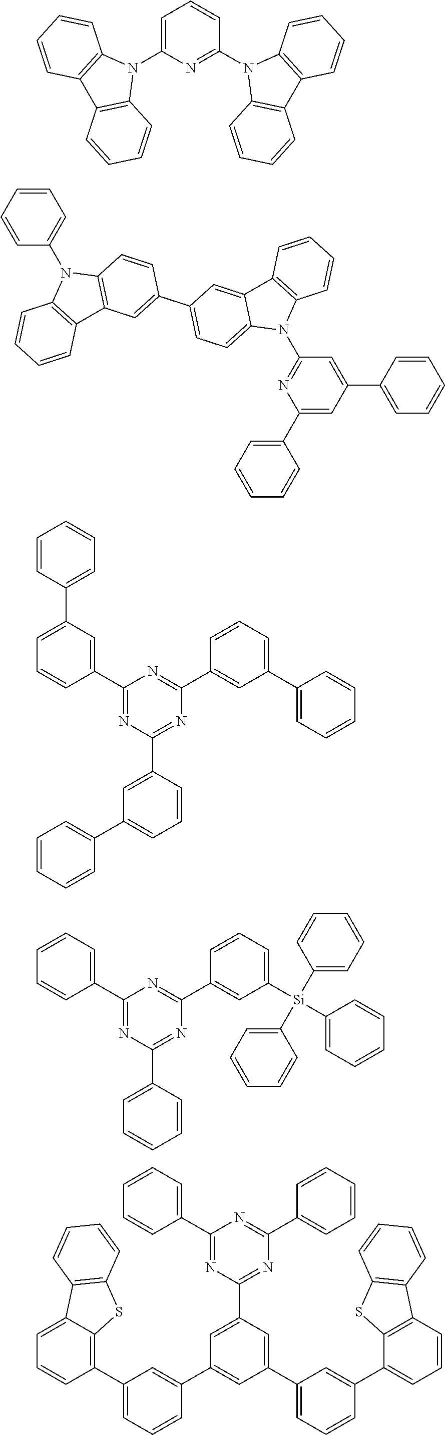

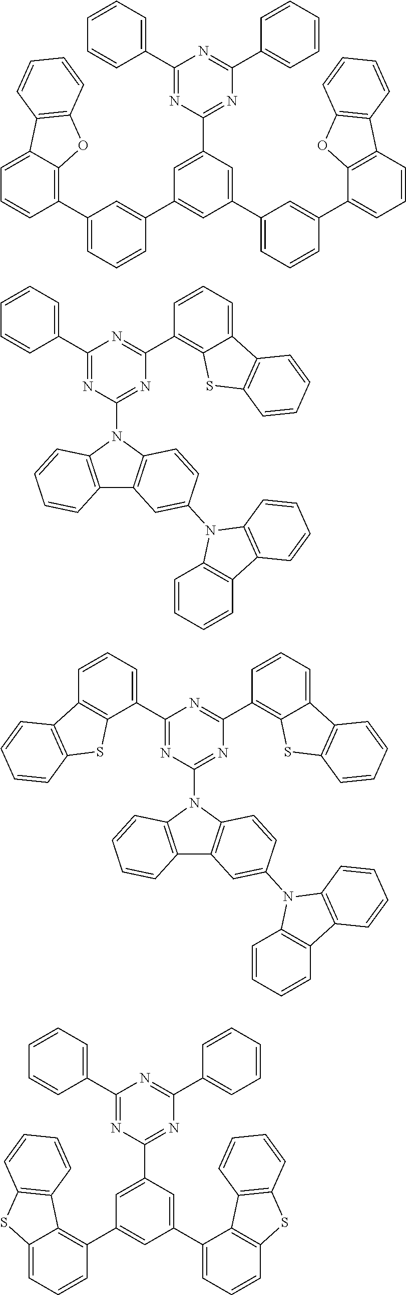 [SMILES: c1ccc(-c2nc(-c3ccccc3)nc(-c3cc(-c4cccc(-c5cccc6c5oc5ccccc56)c4)cc(-c4cccc(-c5cccc6c5oc5ccccc56)c4)c3)n2)cc1 ; c1ccc(-c2nc(-c3cccc4c3sc3ccccc34)nc(-n3c4ccccc4c4cc(-n5c6ccccc6c6ccccc65)ccc43)n2)cc1 ; c1ccc2c(c1)sc1c(-c3nc(-c4cccc5c4sc4ccccc45)nc(-n4c5ccccc5c5cc(-n6c7ccccc7c7ccccc76)ccc54)n3)cccc12 ; c1ccc(-c2nc(-c3ccccc3)nc(-c3cc(-c4cccc5sc6ccccc6c45)cc(-c4cccc5sc6ccccc6c45)c3)n2)cc1]

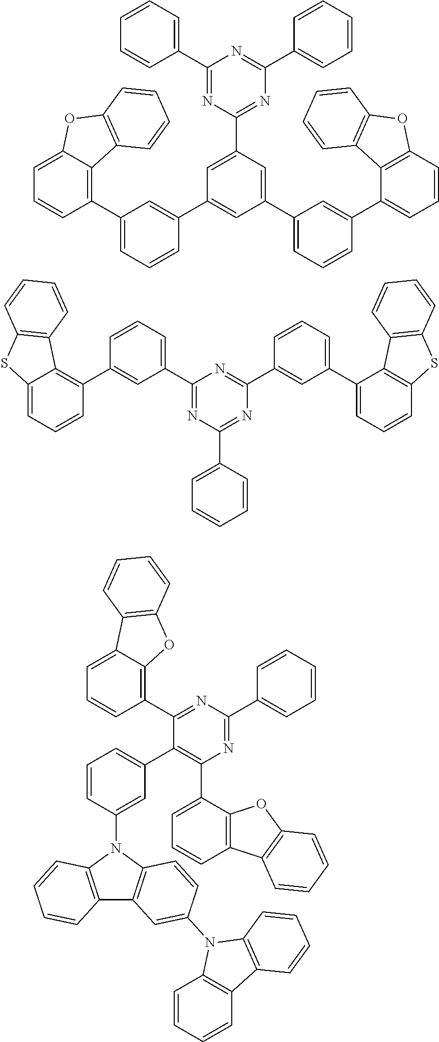

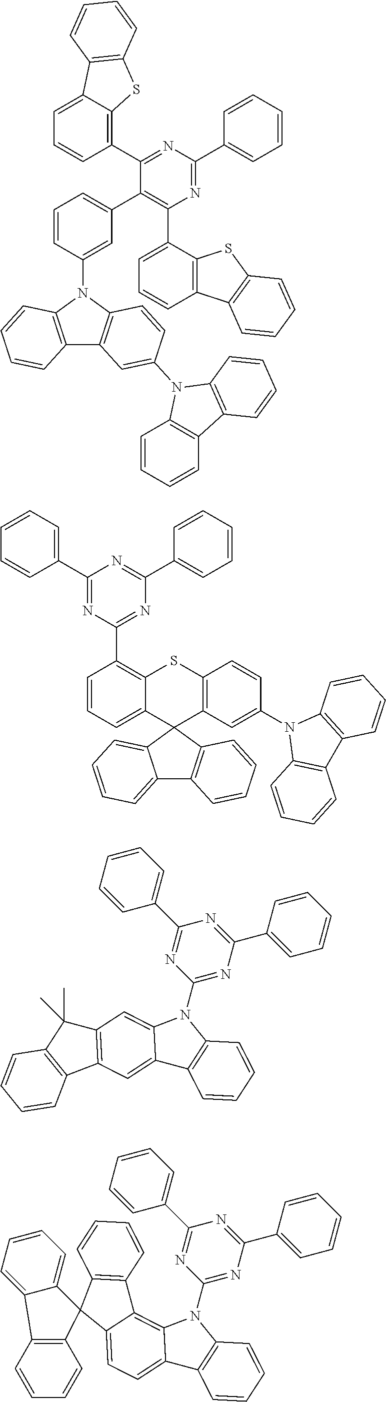

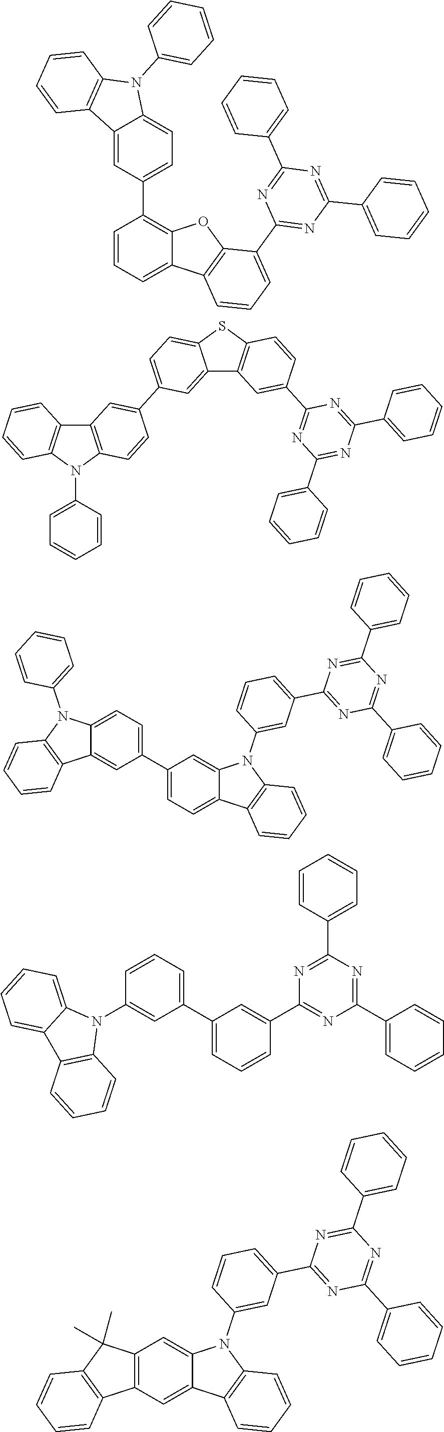

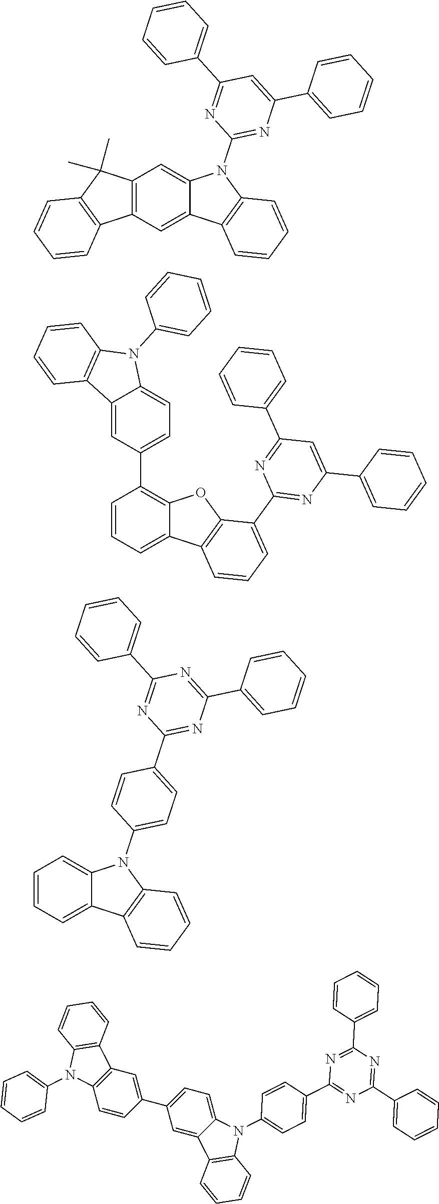

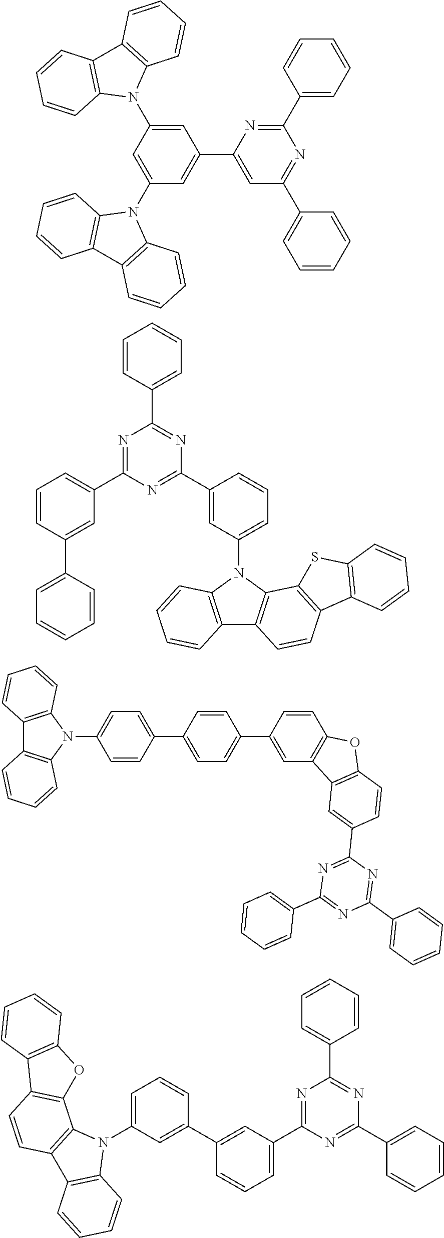

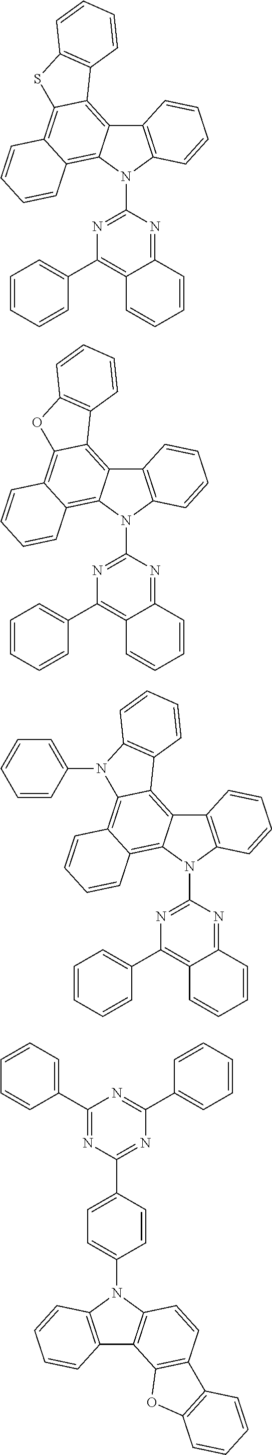

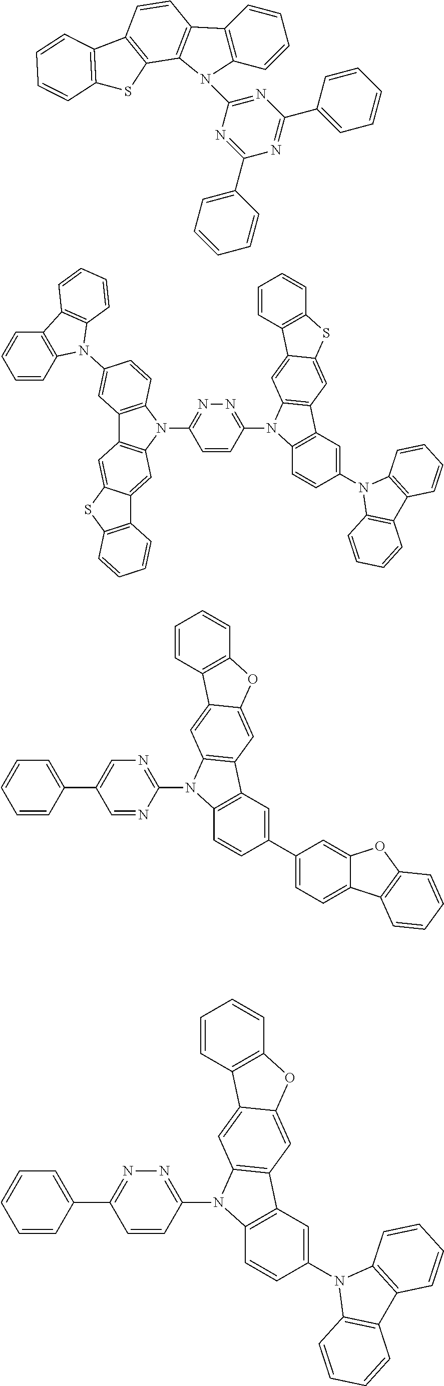

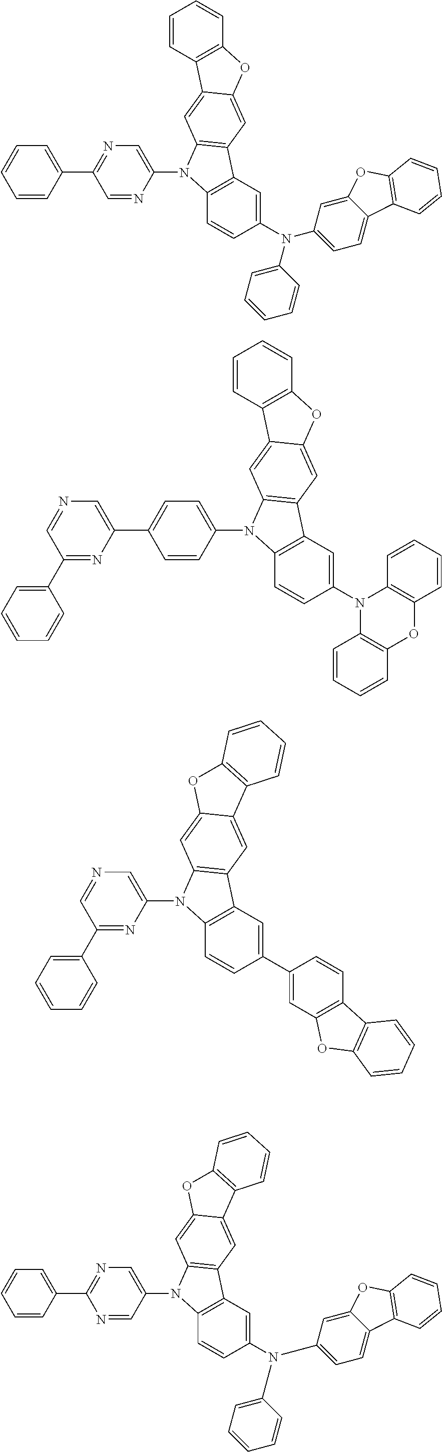

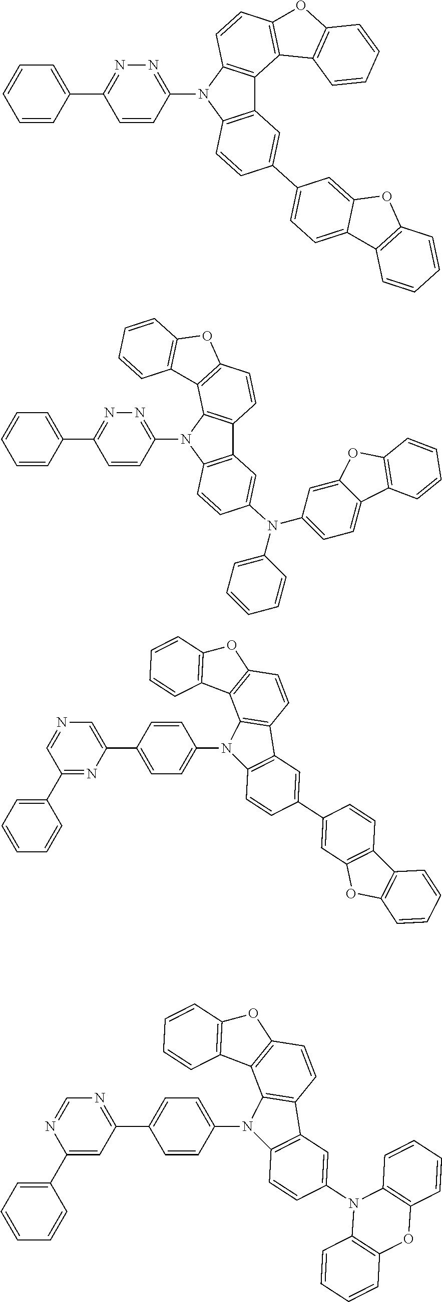

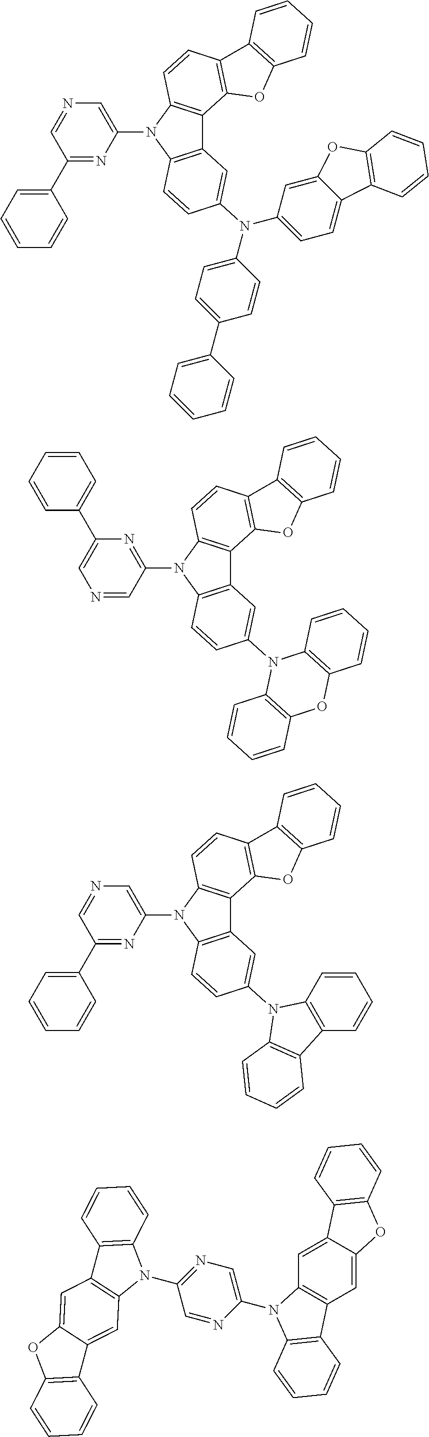

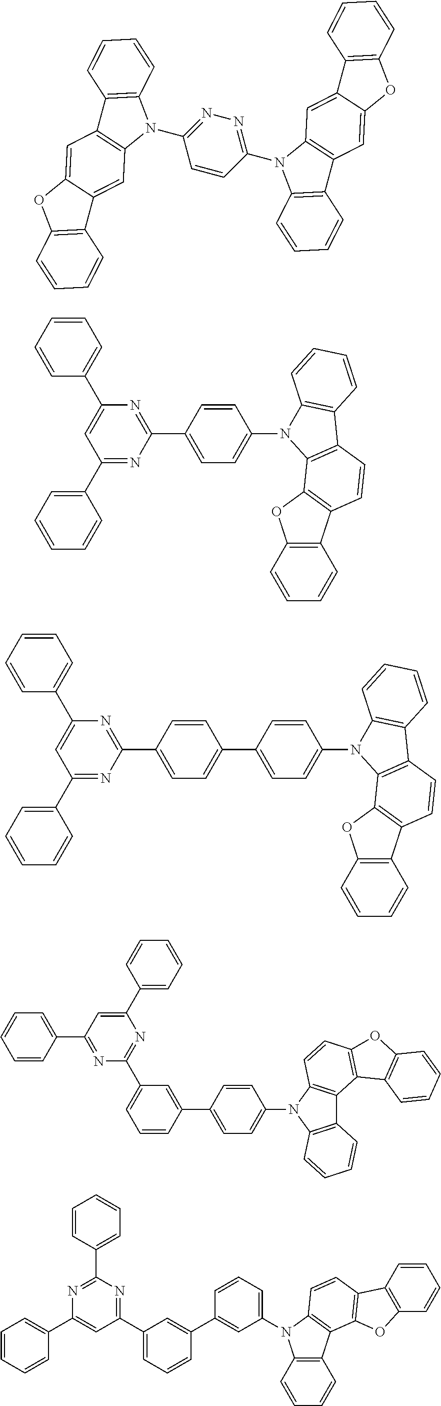

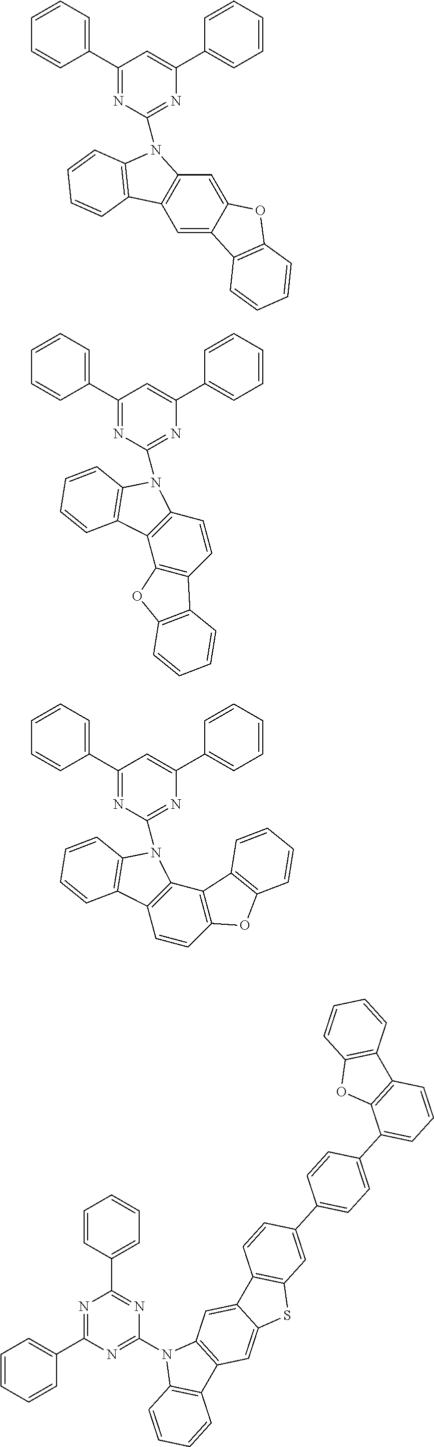

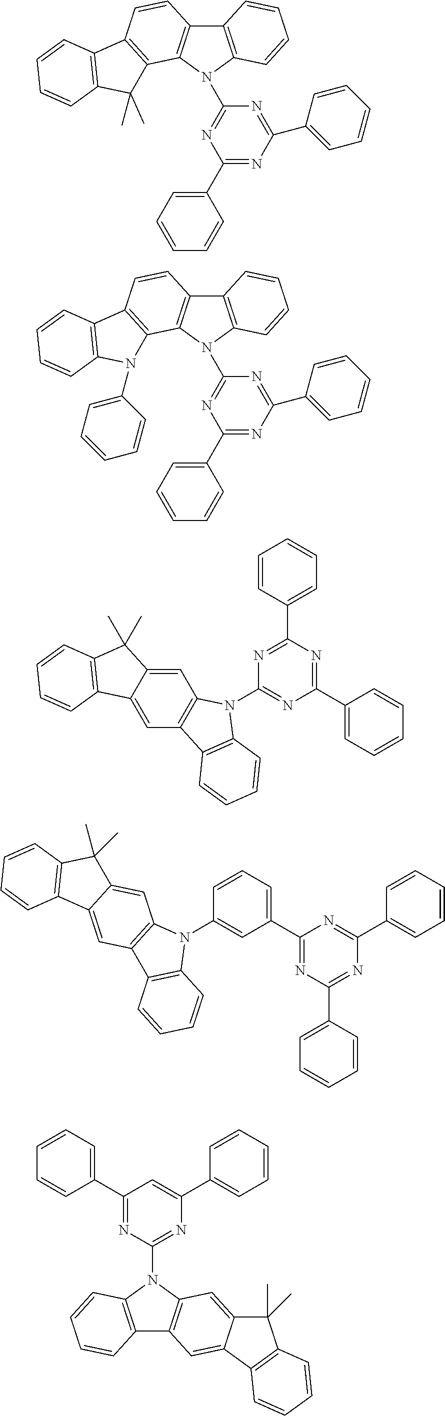

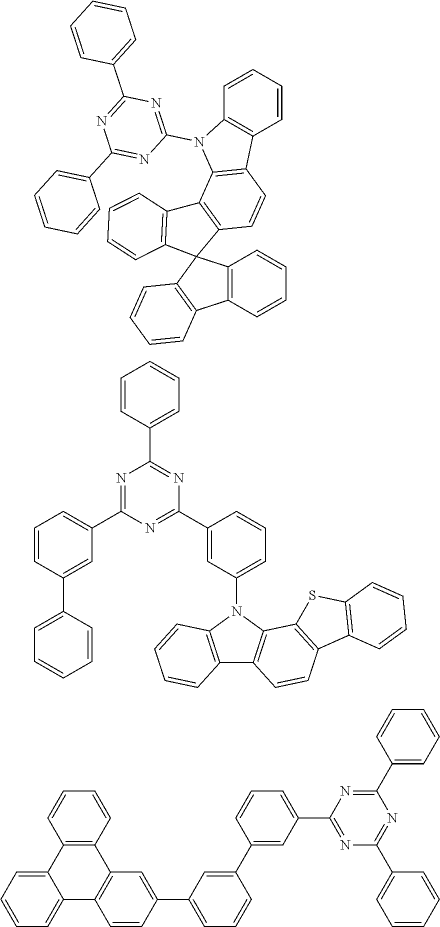

Relationship between Compound M1, Compound M2, and Compound M3 in Emitting Layer

In the organic EL device according to the exemplary embodiment, the singlet energy S₁ (Mat2) of the compound M2 and a singlet energy S₁ (Mat3) of the compound M3 preferably satisfy a relationship of a numerical formula (Numerical Formula 4) below.

S₁(Mat3)>S₁(Mat2) (Numerical Formula 4)

An energy gap T_{77K}(Mat3) at 77K of the compound M3 is preferably larger than the energy gap T_{77K}(Mat2) at 77K of the compound M2.

The energy gap T_{77K}(Mat3) at 77K of the compound M3 is preferably larger than the energy gap T_{77K}(Mat1) at 77K of the compound M1.

In the organic EL device according to the exemplary embodiment, the singlet energy S₁ (Mat2) of the compound M2, the singlet energy S₁ (Mat1) of the compound M1, and the singlet energy S₁(Mat3) of the compound M3 preferably satisfy a relationship of a numerical formula (Numerical Formula 5) below.

S₁(Mat3)>S₁(Mat2)>S₁(Mat1) (Numerical Formula 5)

In the organic EL device according to the exemplary embodiment, the energy gap T_{77K}(Mat2) at 77K of the compound M2, the energy gap T_{77K}(Mat1) at 77K of the compound M1, and the energy gap T_{77K}(Mat3) at 77K of the compound M3 preferably satisfy a relationship of a numerical formula (Numerical Formula 5A) below.

T_{77K}(Mat3)>T_{77K}(Mat2)>T_{77K}(Mat1) (Numerical Formula 5A)

It is preferable that, when the organic EL device of the exemplary embodiment emits light, the fluorescent compound M1 mainly emits light in the emitting layer.

The organic EL device of the exemplary embodiment preferably emits red light or green light.

The maximum peak wavelength of light emitted from the organic EL device can be measured by the same method as that for the organic EL device of the first exemplary embodiment.

### Content Ratios of Compounds in Emitting Layer

For instance, content ratios of the compound M1, the compound M2, and the compound M3 in the emitting layer preferably fall within ranges shown below.

The content ratio of the compound M1 is preferably in a range from 0.01 mass % to 10 mass %, more preferably in a range from 0.01 mass % to 5 mass %, further preferably in a range from 0.01 mass % to 1 mass %.

The content ratio of the compound M2 is preferably in a range from 10 mass % to 80 mass %, more preferably in a range from 10 mass % to 60 mass %, further preferably in a range from 20 mass % to 60 mass %.

The content ratio of the compound M3 is preferably in a range from 10 mass % to 80 mass %.

The upper limit of the total of the content ratios of the compound M1, the compound M2, and the compound M3 in the emitting layer is 100 mass %. It should be noted that the emitting layer according to the exemplary embodiment may contain a material other than the compound M1, the compound M2, and the compound M3.

The emitting layer may contain a single type of the compound M1 or may contain two or more types of the compound M1. The emitting layer may contain a single type of the compound M2 or may contain two or more types of the compound M2. The emitting layer may contain a single type of the compound M3 or may contain two or more types of the compound M3.

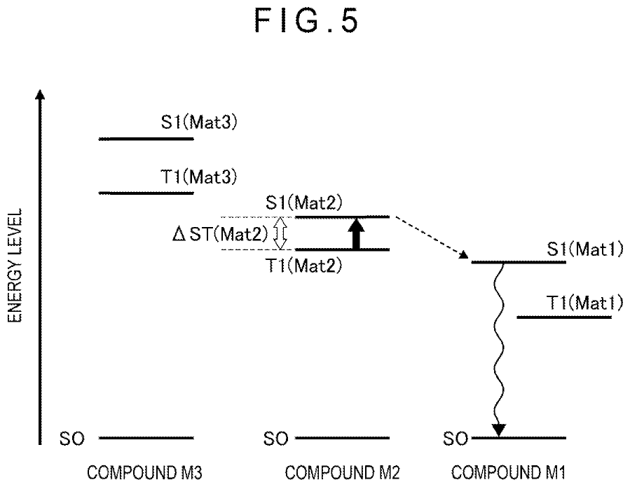

FIG. 5 shows an example of a relationship between energy levels of the compound M1, the compound M2 and the compound M3 in the emitting layer. In FIG. 5, S0 represents a ground state. S1(Mat1) represents a lowest singlet state of the compound M1, and T1(Mat1) represents a lowest triplet state of the compound M1. S1(Mat2) represents a lowest singlet state of the compound M2, and T1(Mat2) represents a lowest triplet state of the compound M2. S1(Mat3) represents a lowest singlet state of the compound M3, and T1(Mat3) represents a lowest triplet state of the compound M3. A dashed arrow directed from S1(Mat2) to S1(Mat1) in FIG. 5 represents Førster energy transfer from the lowest singlet state of the compound M2 to the lowest singlet state of the compound M1.

As shown in FIG. 5, when a compound having a small ΔST(Mat2) is used as the compound M2, inverse intersystem crossing from the lowest triplet state T1(Mat2) to the lowest singlet state S1(Mat2) can be caused by heat energy. Subsequently, Førster energy transfer from the lowest singlet state S1(Mat2) of the compound M2 to the compound M1 occurs to generate the lowest singlet state S1(Mat1). Consequently, fluorescence from the lowest singlet state S1(Mat2) of the compound M1 can be observed. It is inferred that the internal quantum efficiency can be theoretically raised up to 100% also by using delayed fluorescence by the TADF mechanism.

According to the second exemplary embodiment, an organic EL device that can achieve higher performance (especially, a decrease in voltage), specifically, both luminous efficiency and voltage suitable for practical use, can be provided.

The organic EL device according to the second exemplary embodiment is applicable to an organic EL display device.

The organic EL device according to the second exemplary embodiment is applicable to an electronic device such as a display device and a light-emitting unit.

### Third Exemplary Embodiment

An arrangement of an organic EL device according to a third exemplary embodiment of the invention is described below. In the description of the third exemplary embodiment, the same components as those in the first and second exemplary embodiments are denoted by the same reference signs and names to simplify or omit an explanation of the components. In the third exemplary embodiment, any materials and compounds that are not specified may be the same as those in the first and second exemplary embodiments.

The organic EL device according to the third exemplary embodiment is different from the organic EL device according to an exemplary arrangement of the first exemplary embodiment in that the emitting layer contains the delayed fluorescent compound M2 and a compound M4. Other components are the same as those in the first exemplary embodiment.

Specifically, in the organic EL device of the third exemplary embodiment, the emitting layer contains the delayed fluorescent compound M2 and the compound M4; the first layer contains the first compound satisfying specific parameters (Numerical

Formula 1 and Numerical Formula 2); the second layer contains the second compound satisfying a specific parameter (Numerical Formula 3); and the first layer has a film thickness of 15 nm or more.

In the third exemplary embodiment, the compound M2 in the emitting layer is preferably a dopant material, and the compound M4 is preferably a host material.

The compound M4 may be a delayed fluorescent compound or a compound exhibiting no delayed fluorescence.

The compound M4 is not particularly limited, and the compound M3 described in the second exemplary embodiment is usable as the compound M4.

As the compound M2, the compound M2 described in the first exemplary embodiment is usable.

As the first compound, the first compound described in the first exemplary embodiment is usable.

As the second compound, the second compound described in the first exemplary embodiment is usable.

Relationship between Compound M2 and Compound M4 in Emitting Layer In the organic EL device according to the exemplary embodiment, the singlet energy S₁ (Mat2) of the compound M2 and a singlet energy S₁ (Mat4) of the compound M4 preferably satisfy a relationship of a numerical formula (Numerical Formula 6) below.

S₁(Mat4)>S₁(Mat2) (Numerical Formula 6)

An energy gap T_{77K}(Mat4) at 77K of the compound M4 is preferably larger than the energy gap T_{77K}(Mat2) at 77K of the compound M2.

It is preferable that, when the organic EL device of the exemplary embodiment emits light, the compound M2 mainly emits light in the emitting layer.

### Content Ratios of Compounds in Emitting Layer

For instance, content ratios of the compound M2 and the compound M4 in the emitting layer preferably fall within ranges shown below.

The content ratio of the compound M2 is preferably in a range from 10 mass % to 80 mass %, more preferably in a range from 10 mass % to 60 mass %, and further preferably in a range from 20 mass % to 60 mass %.

The content ratio of the compound M4 is preferably in a range from 20 mass % to 90 mass %, more preferably in a range from 40 mass % to 90 mass %, and further preferably in a range from 40 mass % to 80 mass %.

It should be noted that the emitting layer according to the exemplary embodiment may contain a material other than the compound M2 and the compound M4.

The emitting layer may contain a single type of the compound M2 or may contain two or more types of the compound M2. The emitting layer may contain a single type of the fourth compound or may contain two or more types of the fourth compound.

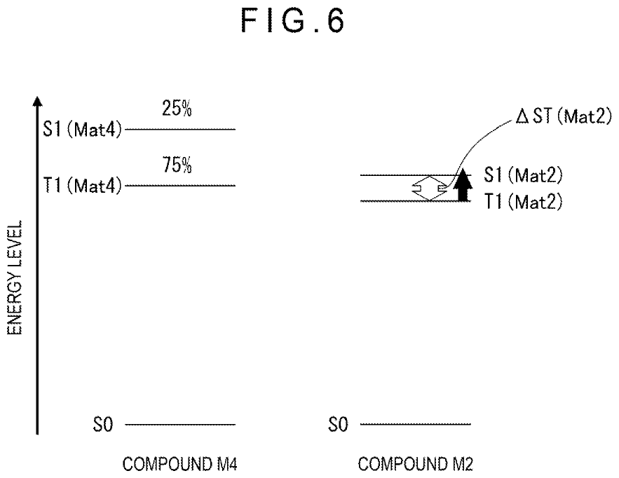

FIG. 6 shows an example of a relationship between energy levels of the compound M2 and the compound M4 in the emitting layer. In FIG. 6, 50 represents a ground state. S1(Mat2) represents a lowest singlet state of the compound M2, and T1(Mat2) represents a lowest triplet state of the compound M2. S1(Mat4) represents a lowest singlet state of the compound M4, and T1(Mat4) represents a lowest triplet state of the compound M4. As shown in FIG. 6, when a material having a small ΔST(Mat2) is used as the compound M2, inverse intersystem crossing from the lowest triplet state T1 to the lowest singlet state S1 in the compound M2 can be caused by heat energy.

The inverse intersystem crossing caused in the compound M2 enables light emission from the lowest singlet state S1(Mat2) of the compound M2 to be observed when the emitting layer does not contain a fluorescent dopant with the lowest singlet state S1 smaller than the lowest singlet state S1(Mat2) of the compound M2. It is inferred that the internal quantum efficiency can be theoretically raised up to 100% also by using delayed fluorescence by the TADF mechanism.

According to the third exemplary embodiment, an organic EL device that can achieve higher performance (especially, a decrease in voltage), specifically, both luminous efficiency and voltage suitable for practical use, can be provided.

The organic EL device according to the third exemplary embodiment is applicable to an organic EL display device.

The organic EL device according to the third exemplary embodiment is applicable to an electronic device such as a display device and a light-emitting unit.

### Fourth Exemplary Embodiment

### Organic Electroluminescence Display Device

An organic EL display device of a fourth exemplary embodiment is an organic electroluminescence display device including: an anode and a cathode arranged to face each other; a blue-emitting organic EL device as a blue pixel; a green-emitting organic EL device as a green pixel; and a red-emitting organic EL device as a red pixel, in which:

the red pixel includes the organic EL device according to any of the first to third exemplary embodiments as the red-emitting organic EL device;

the red-emitting organic EL device includes a red emitting layer as the emitting layer, the first layer provided between the red emitting layer and the anode, and the second layer provided between the first layer and the anode;

the blue-emitting organic EL device includes a blue emitting layer provided between the anode and the cathode;

the green-emitting organic EL device includes a green emitting layer provided between the anode and the cathode; and

the second layer is provided between the anode and each of the blue emitting layer, the green emitting layer, and the first layer in a shared manner across the blue-emitting organic EL device, the green-emitting organic EL device, and the red-emitting organic EL device.

In the organic EL display device of the fourth exemplary embodiment, the red-emitting organic EL device included in the red pixel is an organic EL device that emits light using the TADF mechanism, and the red-emitting organic EL device is the organic EL device according to any of the first to third exemplary embodiments.

Specifically, the organic EL display device of the fourth exemplary embodiment includes the red-emitting organic EL device in which the first layer provided between the red emitting layer and the anode contains the first compound satisfying specific parameters (Numerical Formula 1 and Numerical Formula 2); and the second layer provided between the first layer and the anode contains the second compound satisfying a specific parameter (Numerical Formula 3); and the first layer has a film thickness of 15 nm or more. Even when the red-emitting organic EL device includes the first layer with an increased film thickness due to the same reasons as in the first exemplary embodiment, both luminous efficiency and voltage suitable for practical use can be achieved.

Thus, in the organic EL display device of the fourth exemplary embodiment, cavity adjustment can be easily performed, for instance, by simply increasing the film thickness of the first layer of the red-emitting organic EL device.

Since the organic EL display device of the fourth exemplary embodiment includes the red-emitting organic EL device that can achieve higher performance (especially, a decrease in voltage), specifically, both luminous efficiency and voltage suitable for practical use, the organic EL display device of the fourth exemplary embodiment can achieve higher performance.

Herein, “blue”, “green”, or “red” used for each element, such as “pixel”, “emitting layer”, “organic layer”, or “material”, is used to distinguish one from another. Although “blue”, “green”, or “red” may represent a color of light emitted from “pixel”, “emitting layer”, “organic layer”, or “material”, “blue”, “green”, or “red” does not mean the color of appearance of each element.

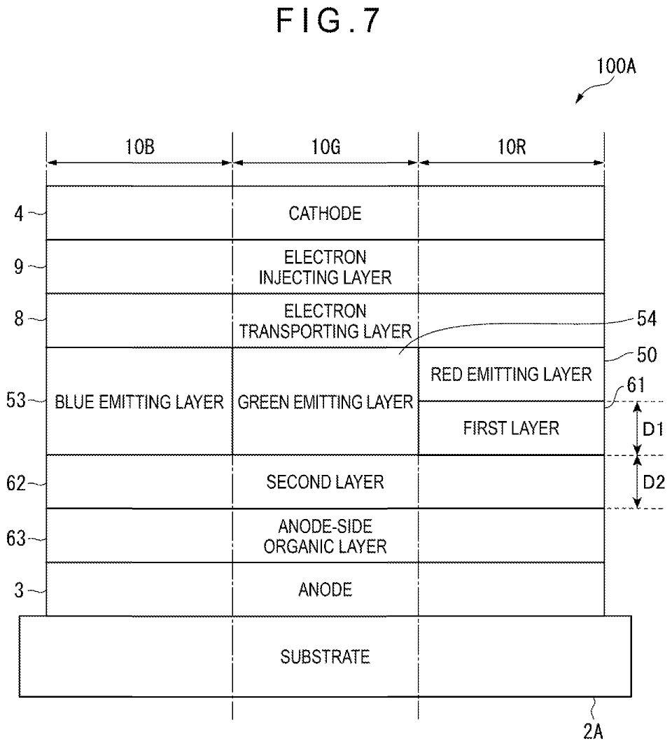

Referring to FIG. 7, an arrangement of an organic EL display device according to the fourth exemplary embodiment of the invention is described below.

FIG. 7 shows an organic EL display device 100A according to an exemplary embodiment.

The organic EL display device 100A includes electrodes and organic layers supported by a substrate 2A.

The organic EL display device 100A includes an anode 3 and a cathode 4 arranged to face each other.

The organic EL display device 100A includes a blue-emitting organic EL device 10B as a blue pixel, a green-emitting organic EL device 10G as a green pixel, and a red-emitting organic EL device 10R as a red pixel.

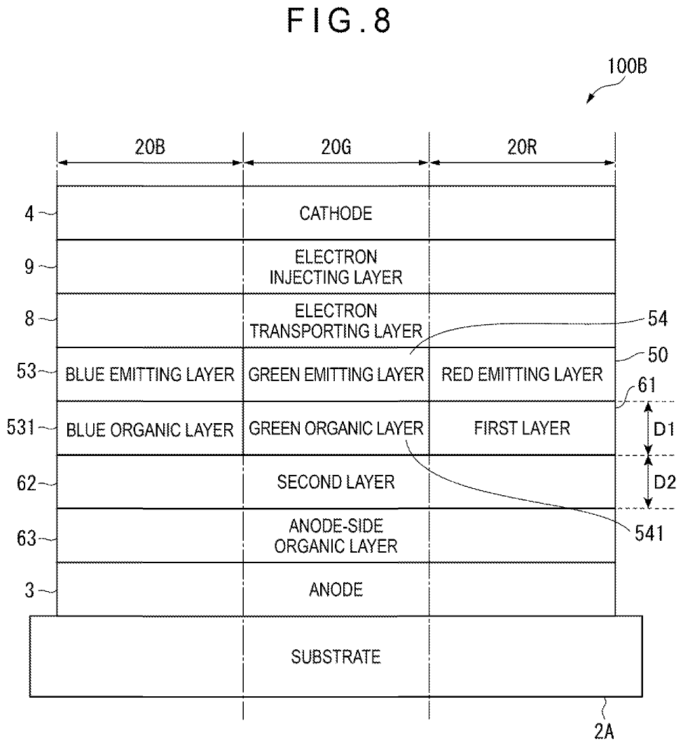

FIG. 7, which schematically shows the organic EL display device 100A, does not limit a size of the device 100A, a thickness of each layer of the device 100A, and the like. For instance, although a blue emitting layer 53 and a green emitting layer 54 in FIG. 7 have the same thickness, these layers may have different thicknesses in an actual organic EL display device. The same applies to an organic EL display device shown in FIG. 8.

In the blue-emitting organic EL device 10B, the green-emitting organic EL device 10G, and the red-emitting organic EL device 10R of the organic EL display device 100A, the anode-side organic layer 63 and the second layer 62 are provided between the anode 3 and each of the blue emitting layer 53, the green emitting layer 54, and the first layer 61.

The anode-side organic layer 63 and the second layer 62 are arranged in a shared manner across the blue-emitting organic EL device 10B, the green-emitting organic EL device 10G, and the red-emitting organic EL device 10R.

In the blue-emitting organic EL device 10B, the green-emitting organic EL device 10G, and the red-emitting organic EL device 10R of the organic EL display device 100A, the electron transporting layer 8 and the electron injecting layer 9 are provided between the cathode 4 and each of the blue emitting layer 53, the green emitting layer 54, and a red emitting layer 50.

The electron transporting layer 8 and the electron injecting layer 9 are arranged in a shared manner across the blue-emitting organic EL device 10B, the green-emitting organic EL device 10G, and the red-emitting organic EL device 10R.

In the red-emitting organic EL device 10R, the first layer 61 as the non-common layer is provided between the red emitting layer 50 and the second layer 62. The red emitting layer 50 corresponds to the emitting layer according to any of the first exemplary embodiment, the second exemplary embodiment, and the third exemplary embodiment. The first layer 61 corresponds to the first layer according to any of the first exemplary embodiment, the second exemplary embodiment, and the third exemplary embodiment. The second layer 62 corresponds to the second layer according to any of the first exemplary embodiment, the second exemplary embodiment, and the third exemplary embodiment. In FIG. 7, D1 represents a film thickness of the first layer 61. D1 (the film thickness of the first layer 61) is 15 nm or more. D2 represents a film thickness of the second layer 62. D2 (the film thickness of the second layer 62) is preferably in a range from 80 nm to 140 nm.

In FIG. 7, the first layer 61 is in direct contact with the red emitting layer 50 and the second layer 62. The first layer 61 is preferably an electron blocking layer.

The anode 3 is independently provided for each of the blue-emitting organic EL device 10B, the green-emitting organic EL device 10G, and the red-emitting organic EL device 10R. Thus, the blue-emitting organic EL device 10B, the green-emitting organic EL device 10G, and the red-emitting organic EL device 10R can be individually driven in the organic EL display device 100A. The respective anodes of the organic EL devices 10B,10G, and 10R are insulated from each other by an insulation material (not shown). The cathode 4 is provided in a shared manner across the blue-emitting organic EL device 10B, the green-emitting organic EL device 10G, and the red-emitting organic EL device 10R.

In an exemplary embodiment, the blue-emitting organic EL device 10B, the green-emitting organic EL device 10G, and the red-emitting organic EL device 10R as pixels are arranged in parallel with each other on the substrate 2A.

In the organic EL display device 100A according to the fourth exemplary embodiment, the second layer 62 is preferably in direct contact with each of the blue emitting layer 53, the green emitting layer 54, and the first layer 61.

The invention is not limited to the arrangement of the organic EL display device shown in FIG. 7.

For instance, in an exemplary arrangement of the organic EL display device of the exemplary embodiment, the blue-emitting organic EL device, the green-emitting organic EL device, and the red-emitting organic EL device may each independently further include a layer different from the layers shown in FIG. 7. For instance, a hole blocking layer may be provided as the common layer between the electron transporting layer and each of the blue emitting layer, the green emitting layer, and the red emitting layer.

For instance, in an exemplary arrangement of the organic EL display device of the exemplary embodiment, the blue-emitting organic EL device and the green-emitting organic EL device may be each independently a device that fluoresces or a device that phosphoresces. The red-emitting organic EL device is preferably a device that fluoresces.

The blue-emitting organic EL device and the green-emitting organic EL device are explained. The organic EL device according to any of the first to third exemplary embodiments is applicable to the red-emitting organic EL device.

In an exemplary arrangement of the organic EL display device of the exemplary embodiment, the blue emitting layer contains a host material. For instance, the blue emitting layer contains 50 mass % or more of the host material with respect to a total mass of the blue emitting layer.

In an exemplary arrangement of the organic EL display device of the exemplary embodiment, the blue emitting layer of the blue-emitting organic EL device contains a blue emitting compound that emits light having a maximum peak wavelength in a range from 430 nm to 500 nm. The blue emitting compound is, for instance, a fluorescent compound that emits fluorescence having a maximum peak wavelength in a range from 430 nm to 500 nm. Further, the blue emitting compound is, for instance, a phosphorescent compound that exhibits phosphorescence having a maximum peak wavelength in a range from 430 nm to 500 nm. Herein, the blue light emission refers to a light emission in which the maximum peak wavelength of emission spectrum is in a range from 430 nm to 500 nm.

The fluorescent compound is a compound capable of emitting in a singlet state. The phosphorescent compound is a compound capable of emitting in a triplet state.

Examples of a blue fluorescent compound usable for the blue emitting layer include a pyrene derivative, a styrylamine derivative, a chrysene derivative, a fluoranthene derivative, a fluorene derivative, a diamine derivative, and a triarylamine derivative. Specific examples thereof include N,N′-bis[4-(9H-carbazole-9-yl)phenyl]-N,N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

Examples of a blue phosphorescent compound usable for the blue emitting layer include metal complexes such as an iridium complex, an osmium complex, and a platinum complex. Specific examples thereof include bis[2-(4′,6′-difluorophenyl)pyridinato-N,C2′]iridium(III)tetrakis(1-pyrazolyl)borate (abbreviation: Flr6), bis[2-(4′,6′-difluorophenyl)pyridinato-N, C2′]iridium(III)picolinate (abbreviation: Flrpic), bis[2-(3′,5′bistrifluoromethylphenyl)pyridinato-N,C2′]iridium(III)picolinate (abbreviation: Ir(CF₃ppy)₂(pic)), and bis[2-(4′,6′-difluorophenyl)pyridinato-N,C2′]iridium(III)acetylacetonato (abbreviation: Flracac).

### Maximum Phosphorescence Peak Wavelength (PH-peak)

A maximum peak wavelength (maximum phosphorescence peak wavelength) of a phosphorescent compound is measurable by the following method.

A measurement target compound is dissolved in EPA (diethylether:isopentane:ethanol=5:5:2 in volume ratio) so as to fall within a range from 10⁻⁵ mol/L to 10⁻⁴ mol/L, and the obtained EPA solution is encapsulated in a quartz cell to provide a measurement sample. A phosphorescence spectrum (ordinate axis: phosphorescent luminous intensity, abscissa axis: wavelength) of the measurement sample is measured at a low temperature (77K). The local maximum value closest to the short-wavelength region among the local maximum values of the phosphorescence spectrum is defined as the maximum phosphorescence peak wavelength. A spectrophotofluorometer F-7000 manufactured by Hitachi High-Tech Science Corporation can be used to measure phosphorescence. Any device for phosphorescence measurement is usable. A combination of a cooling unit, a low temperature container, an excitation light source and a light-receiving unit may be used for phosphorescence measurement. Herein, the maximum peak wavelength of phosphorescence is occasionally referred to as the maximum phosphorescence peak wavelength (PH-peak).

In an exemplary arrangement of the organic EL display device of the exemplary embodiment, the green emitting layer contains a host material. For instance, the green emitting layer contains 50 mass % or more of the host material with respect to a total mass of the green emitting layer.

In an exemplary arrangement of the organic EL display device of the exemplary embodiment, the green emitting layer of the green-emitting organic EL device contains a green emitting compound that emits light having a maximum peak wavelength in a range from 500 nm to 550 nm. The green emitting compound is, for instance, a fluorescent compound that emits fluorescence having a maximum peak wavelength in a range from 500 nm to 550 nm. Further, the green emitting compound is, for instance, a phosphorescent compound that exhibits phosphorescence having a maximum peak wavelength in a range from 500 nm to 550 nm. Herein, the green light emission refers to a light emission in which a maximum peak wavelength of emission spectrum is in a range from 500 nm to 550 nm.

The fluorescent compound is a compound capable of emitting in a singlet state. The phosphorescent compound is a compound capable of emitting in a triplet state.

Examples of a green fluorescent compound usable for the green emitting layer include an aromatic amine derivative. Examples of a green phosphorescent compound usable for the green emitting layer include an iridium complex.

In an exemplary arrangement of the organic EL display device of the exemplary embodiment, the host material in the blue emitting layer and the host material in the green emitting layer are, for instance, a compound for dispersing a highly emittable substance (dopant material) in the emitting layers. As the host material in the blue emitting layer and the host material in the green emitting layer, it is possible to use, for instance, a substance having a higher Lowest Unoccupied Molecular Orbital (LUMO) level and a lower Highest Occupied Molecular Orbital (HOMO) level than the highly emittable substance.

For instance, the following compounds (1) to (4) are each independently usable as the host material in the blue emitting layer and the host material in the green emitting layer.

(1) a metal complex such as an aluminum complex, a beryllium complex, or a zinc complex
(2) a heterocyclic compound such as an oxadiazole derivative, a benzimidazole derivative, or a phenanthroline derivative
(3) a fused aromatic compound such as a carbazole derivative, an anthracene derivative, a phenanthrene derivative, a pyrene derivative or a chrysene derivative
(4) an aromatic amine compound such as a triarylamine derivative or a fused polycyclic aromatic amine derivative

Referring to FIG. 7, the organic EL display device according to the exemplary embodiment is further explained. Descriptions on the same arrangements as in the first to third exemplary embodiments are simplified or omitted.

### Anode

In an exemplary embodiment, the anode 3 is disposed to face the cathode 4.

In an exemplary embodiment, the anode 3 is typically the non-common layer. In an exemplary embodiment, for instance, when the anode 3 is the non-common layer, the respective anodes in the blue-emitting organic EL device 10B, the green-emitting organic EL device 10G and the red-emitting organic EL device 10R are physically separated from each other, and specifically, may be insulated from each other by an insulation material (not shown) or the like.

### Cathode

In an exemplary embodiment, the cathode 4 is disposed to face the anode 3.

In an exemplary embodiment, the cathode 4 may be the common layer or the non-common layer.

In an exemplary embodiment, the cathode 4 is preferably the common layer provided in a shared manner across the blue-emitting organic EL device 10B, the green-emitting organic EL device 10G, and the red-emitting organic EL device 10R.

In an exemplary embodiment, the cathode 4 is in direct contact with the electron injecting layer 9.

In an exemplary embodiment, when the cathode 4 is the common layer, the film thickness of the cathode 4 is constant over the blue-emitting organic EL device 10B, the green-emitting organic EL device 10G, and the red-emitting organic EL device 10R. When the cathode 4 is the common layer, the cathode 4 provided for the blue-emitting organic EL device 10B, the green-emitting organic EL device 10G, and the red-emitting organic EL device 10R can be produced without changing a mask or the like. The organic EL display device 100A thus has enhanced productivity.

### Electron Transporting Layer

In an exemplary embodiment, the electron transporting layer 8 is the common layer provided in a shared manner across the blue-emitting organic EL device 10B, the green-emitting organic EL device 10G, and the red-emitting organic EL device 10R.

In an exemplary embodiment, the electron transporting layer 8 is provided between the electron injecting layer 9 and the emitting layers of the blue-emitting organic EL device 10B, the green-emitting organic EL device 10G, and the red-emitting organic EL device 10R.

In an exemplary embodiment, the side of the electron transporting layer 8 close to the anode 3 is in direct contact with the blue emitting layer 53, the green emitting layer 54, and the red emitting layer 50.

The side of the electron transporting layer 8 close to the cathode 4 is in direct contact with the electron injecting layer 9.

In an exemplary embodiment, the electron transporting layer 8 is the common layer. In this case, the film thickness of the electron transporting layer 8 is constant over the blue-emitting organic EL device 10B, the green-emitting organic EL device 10G, and the red-emitting organic EL device 10R. When the electron transporting layer 8 is the common layer, the electron transporting layer 8 provided for the blue-emitting organic EL device 10B, the green-emitting organic EL device 10G, and the red-emitting organic EL device 10R can be produced without changing a mask or the like. The organic EL display device 100A thus has enhanced productivity.

### Electron Injecting Layer

In an exemplary embodiment, the electron injecting layer 9 is the common layer provided in a shared manner across the blue-emitting organic EL device 10B, the green-emitting organic EL device 10G, and the red-emitting organic EL device 10R.

In an exemplary embodiment, the electron injecting layer 9 is disposed between the electron transporting layer 8 and the cathode 4.

In an exemplary embodiment, the electron injecting layer 9 is in direct contact with the electron transporting layer 8.

In an exemplary embodiment, the electron injecting layer 9 is the common layer. In this case, the film thickness of the electron injecting layer 9 is constant over the blue-emitting organic EL device 10B, the green-emitting organic EL device 10G, and the red-emitting organic EL device 10R. When the electron injecting layer 9 is the common layer, the electron injecting layer 9 provided for the blue-emitting organic EL device 10B, the green-emitting organic EL device 10G, and the red-emitting organic EL device 10R can be produced without changing a mask or the like. The organic EL display device 100A thus has enhanced productivity.

### Fifth Exemplary Embodiment

An arrangement of an organic EL display device according to a fifth exemplary embodiment of the invention is described below. In the description of the fifth exemplary embodiment, the same components as those in the first to fourth exemplary embodiments are denoted by the same reference signs and names to simplify or omit an explanation of the components. In the fifth exemplary embodiment, any materials and compounds that are not specified may be the same as those in the first to fourth exemplary embodiments.

The organic EL display device according to the fifth exemplary embodiment is different from the organic EL display device according to the fourth exemplary embodiment in that the blue-emitting organic EL device includes a blue organic layer provided between the blue emitting layer and the anode and the green-emitting organic EL device includes a green organic layer provided between the green emitting layer and the anode. Other components are the same as those in the organic EL display device of the fourth exemplary embodiment.

In the organic EL display device of the fifth exemplary embodiment, similar to the fourth exemplary embodiment, the red-emitting organic EL device included in the red pixel is an organic EL device that emits light using the TADF mechanism, and the red-emitting organic EL device is the organic EL device according to any of the first to third exemplary embodiments. Even when the red-emitting organic EL device includes the first layer with an increased film thickness, both luminous efficiency and voltage suitable for practical use can be achieved.

Thus, in the organic EL display device of the fifth exemplary embodiment, cavity adjustment can be easily performed, for instance, by simply increasing the film thickness of the first layer of the red-emitting organic EL device.

Since the organic EL display device of the fifth exemplary embodiment includes the red-emitting organic EL device that can achieve higher performance (especially, a decrease in voltage), specifically, both luminous efficiency and voltage suitable for practical use, the organic EL display device of the fifth exemplary embodiment can achieve higher performance.

Further, in the organic EL display device of the fifth exemplary embodiment, an emission position in the blue-emitting organic EL device is easily adjustable by providing the blue organic layer in the blue-emitting organic EL device. Further, an emission position in the green-emitting organic EL device is easily adjustable by providing the green organic layer in the green-emitting organic EL device.

FIG. 8 schematically shows an arrangement of the organic EL display device according to the fifth exemplary embodiment.

An organic EL display device 100B shown in FIG. 8 is configured the same as the organic EL display device 100A shown in FIG. 7 except for a blue-emitting organic EL device 20B as a blue pixel and a green-emitting organic EL device 20G as a green pixel. Thus, only the differences from the organic EL display device 100A are described below.

The blue-emitting organic EL device 20B includes a blue organic layer 531 as the non-common layer between the blue emitting layer 53 and the second layer 62. In FIG. 8, the blue organic layer 531 is in direct contact with the blue emitting layer 53 and the second layer 62. The blue organic layer 531 is preferably an electron blocking layer.

The green-emitting organic EL device 20G includes a green organic layer 541 as the non-common layer between the green emitting layer 54 and the second layer 62. In FIG. 8, the green organic layer 541 is in direct contact with the green emitting layer 54 and the second layer 62. The green organic layer 541 is preferably an electron blocking layer.

The second layer 62 as the common layer is preferably in direct contact with each of the blue organic layer 531, the green organic layer 541, and the first layer 61.

The second layer 62 is preferably in direct contact with the anode-side organic layer 63. The anode-side organic layer 63 is preferably in direct contact with the second layer 62 and the anode 3.

The invention is not limited to the arrangement of the organic EL display device shown in FIG. 8.

The blue organic layer and the green organic layer are explained.

The first layer described in the first exemplary embodiment is applicable to the first layer included in the red-emitting organic EL device. The second layer described in the first exemplary embodiment is applicable to the second layer provided in a shared manner across the blue-emitting organic EL device, the green-emitting organic EL device, and the red-emitting organic EL device.

The blue organic layer contains a blue organic material. As the blue organic material, for instance, it is possible to use a material usable for the hole transporting layer (e.g., an aromatic amine compound, a carbazole derivative, and an anthracene derivative) described in the above Arrangement of Organic EL Device.

Although the blue organic material and the second compound contained in the second layer may be the same compound or different compounds, the blue organic material is preferably different from the second compound.

The blue organic material is a compound different from the host material and the blue emitting compound contained in the blue emitting layer.

The green organic layer contains a green organic material. As the green organic material, for instance, it is possible to use a material usable for the hole transporting layer (e.g., an aromatic amine compound, a carbazole derivative, and an anthracene derivative) described in the above Arrangement of Organic EL Device.

Although the green organic material and the second compound contained in the second layer may be the same compound or different compounds, the green organic material is preferably different from the second compound.

The green organic material is a compound different from the host material and the red emitting compound contained in the green emitting layer.

Although the green organic material contained in the green organic layer of the green-emitting organic EL device and the blue organic material contained in the blue organic layer of the blue-emitting organic EL device may be the same compound or different compounds, the green organic material is preferably different from the blue organic material.

### Manufacturing Method of Organic EL Display Device

As an exemplary manufacturing method of the organic EL display device of the fourth exemplary embodiment, explanation is made about a manufacturing method of the organic EL display device 100A shown in FIG. 8.

First, the anode 3 is formed on the substrate 2A.

Subsequently, the anode-side organic layer 63 as the common layer is formed on the anode 3. The anode-side organic layer 63 provided for the blue-emitting organic EL device 10B, the green-emitting organic EL device 10G, and the red-emitting organic EL device 10R is formed to have a constant film thickness.

Next, the second layer 62 is formed on the anode-side organic layer 63 in a region corresponding to the anode 3 of the blue-emitting organic EL device, the green-emitting organic EL device, and the red-emitting organic EL device. The second layer 62 provided for the blue-emitting organic EL device 10B, the green-emitting organic EL device 10G, and the red-emitting organic EL device 10R is formed to have a constant film thickness.

Next, the blue emitting layer 53 is formed on the second layer 62 in a region corresponding to the anode 3 of the blue-emitting organic EL device 10B using a predetermined film-forming mask (mask for the blue-emitting organic EL device).

Next, the green emitting layer 54 is formed on the second layer 62 in a region corresponding to the anode 3 of the green-emitting organic EL device 10G using a predetermined film-forming mask (mask for the green-emitting organic EL device).

Next, the first layer 61 is formed on the second layer 62 in a region corresponding to the anode 3 of the red-emitting organic EL device 10R using a predetermined film-forming mask (mask for the red-emitting organic EL device). After forming the first layer 61, the red emitting layer 50 is formed on the first layer 61.

The blue emitting layer 53, the green emitting layer 54, and the red emitting layer 50 are formed from mutually different materials.

After the formation of the second layer 62, the order of forming the non-common layers of the blue-emitting organic EL device 10B, the green-emitting organic EL device 10G, and the red-emitting organic EL device 10R is not particularly limited.

For instance, after forming the second layer 62, the green emitting layer 54 of the green-emitting organic EL device 10G may be formed, then the blue emitting layer 53 of the blue-emitting organic EL device 10B may be formed, and then the first layer 61 and the red emitting layer 50 of the red-emitting organic EL device 10R may be formed.

Alternatively, for instance, after forming the second layer 62, the first layer 61 and the red emitting layer 50 of the red-emitting organic EL device 10R may be formed, then the green emitting layer 54 of the green-emitting organic EL device 10G may be formed, and then the blue emitting layer 53 of the blue-emitting organic EL device 10B may be formed.

Subsequently, the electron transporting layer 8 as the common layer is formed on the blue emitting layer 53, the green emitting layer 54, and the red emitting layer 50 to extend thereover. The electron transporting layer 8 of the blue-emitting organic EL device 10B, the green-emitting organic EL device 10G, and the red-emitting organic EL device 10R is formed to have a constant film thickness using the same material.

Subsequently, the electron injecting layer 9 as the common layer is formed on the electron transporting layer 8. The electron injecting layer 9 of the blue-emitting organic EL device 10B, the green-emitting organic EL device 10G, and the red-emitting organic EL device 10R is formed to have a constant film thickness using the same material.

Subsequently, the cathode 4 as the common layer is formed on the electron injecting layer 9. The cathode 4 of the blue-emitting organic EL device 10B, the green-emitting organic EL device 10G, and the red-emitting organic EL device 10R is formed to have a constant film thickness using the same material.

The organic EL display device 100A shown in FIG. 7 is produced as described above.

The organic EL display device 100B shown in FIG. 8 is different from the organic EL display device 100A shown in FIG. 7 in that the blue organic layer 531 and the green organic layer 541 are provided. In manufacture of the organic EL display device 100B shown in FIG. 8, the blue organic layer 531 is formed on the second layer 62 in a region corresponding to the anode 3 of the blue-emitting organic EL device 20B using a predetermined film-forming mask (mask for the blue-emitting organic EL device). Next, the blue emitting layer 53 is formed on the blue organic layer 531.

Next, the green organic layer 541 is formed on the second layer 62 in a region corresponding to the anode 3 of the green-emitting organic EL device 20G using a predetermined film-forming mask (mask for the green-emitting organic EL device). Next, the green emitting layer 54 is formed on the green organic layer 541.

Any other manufacturing steps of the organic EL display device 100B are similar to those of the organic EL display device 100A.

In an exemplary embodiment of the organic EL display device shown in FIG. 7, any other layer than the blue emitting layer 53, the green emitting layer 54, the red emitting layer 50, and the first layer 61 is preferably provided in a shared manner across the blue-emitting organic EL device 10B, the green-emitting organic EL device 10G, and the red-emitting organic EL device 10R. Reducing the number of the non-common layers in the organic EL display device improves productivity of the device.

In an exemplary embodiment of the organic EL display device shown in FIG. 8, any other layer than the blue emitting layer 53, the green emitting layer 54, the red emitting layer 50, the blue organic layer 531, the first layer 61, and the green organic layer 541 is preferably provided in a shared manner across the blue-emitting organic EL device 20B, the green-emitting organic EL device 20G, and the red-emitting organic EL device 20R. Reducing the number of the non-common layers in the organic EL display device improves productivity of the device.

### Sixth Exemplary Embodiment

### Compound

A compound according to a sixth exemplary embodiment is a compound represented by a formula (10) below.

The compound according to the sixth exemplary embodiment is an exemplary arrangement of the first compound usable for the organic EL devices according to the first to third exemplary embodiments, and an exemplary arrangement of the first compound usable for the organic EL display devices according to the fourth and fifth exemplary embodiments.

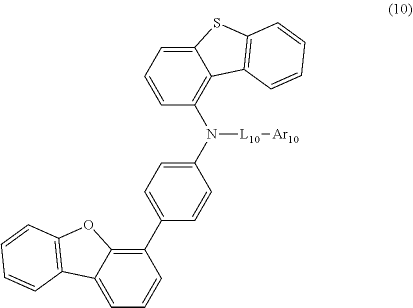

In the formula (10):

L₁₀ is a single bond, or a substituted or unsubstituted arylene group having 6 to 12 ring carbon atoms;

a substituent, if present, for L₁₀ is an unsubstituted phenyl group;

Ar₁₀ is a substituted or unsubstituted aryl group having 6 to 18 ring carbon atoms; and

a substituent, if present, for Ar₁₀ is an unsubstituted phenyl group or an unsubstituted naphthyl group.

In the compound according to the sixth exemplary embodiment, L₁₀ is preferably a single bond, a substituted or unsubstituted phenylene group, a substituted or unsubstituted biphenylene group, or a substituted or unsubstituted naphthylene group.

In the compound according to the sixth exemplary embodiment, L₁₀ is preferably a single bond, or a substituted or unsubstituted phenylene group.

In the compound according to the sixth exemplary embodiment, Ar₁₀ is preferably a substituted or unsubstituted phenyl group, a substituted or unsubstituted biphenyl group, a substituted or unsubstituted terphenyl group, a substituted or unsubstituted naphthyl group, or a substituted or unsubstituted phenanthryl group.

In the compound according to the sixth exemplary embodiment, Ar₁₀ is preferably a group represented by any of formulae (10a) to (27a) below, more preferably a group represented by any of the formulae (10a) to (14a), (17a), (18a), and (26a). In the formulae below, * represents a bonding position.

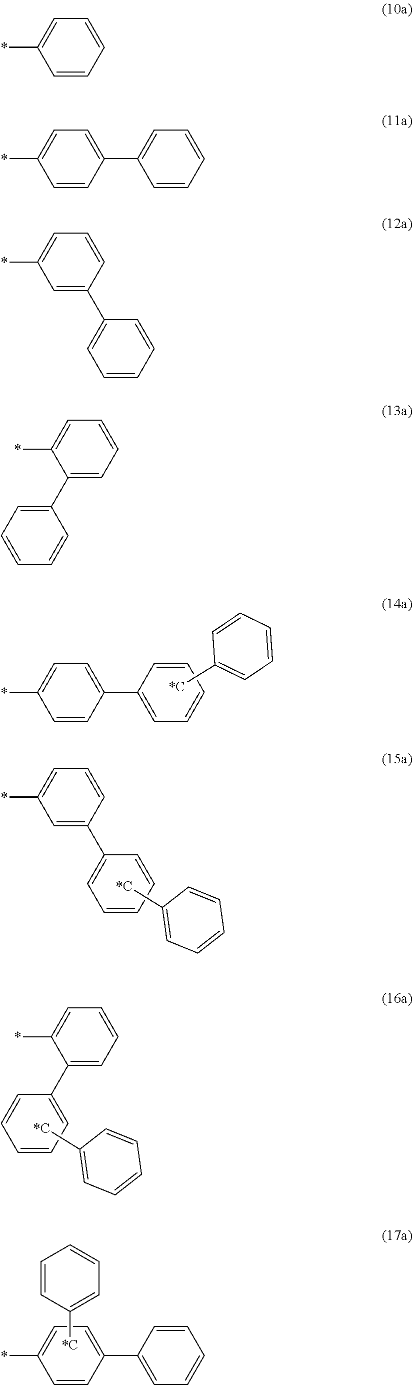

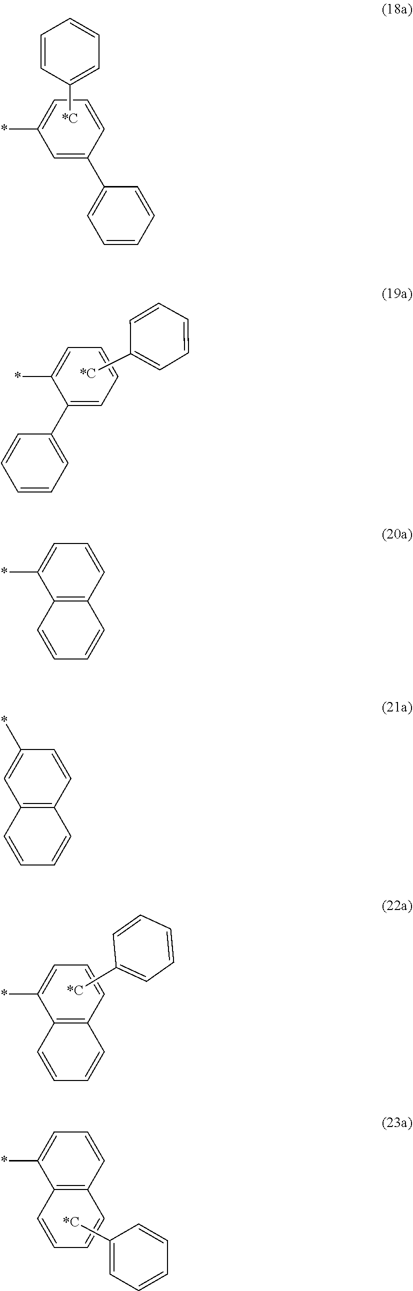

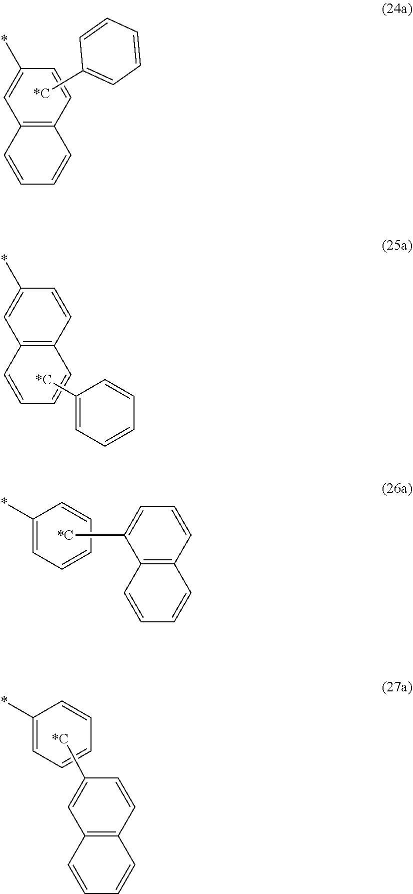

In the formulae (14a) to (19a) and (22a) to (27a), *C represents a bonding position to a carbon atom on a benzene ring.

In the compound according to the sixth exemplary embodiment, Ar₁₀ is further preferably a group represented by any of formulae (10a) to (13a), (14a-1) to (18a-1), (20a) to (21a), and (26a-1) below. In the formulae below, * represents a bonding position.

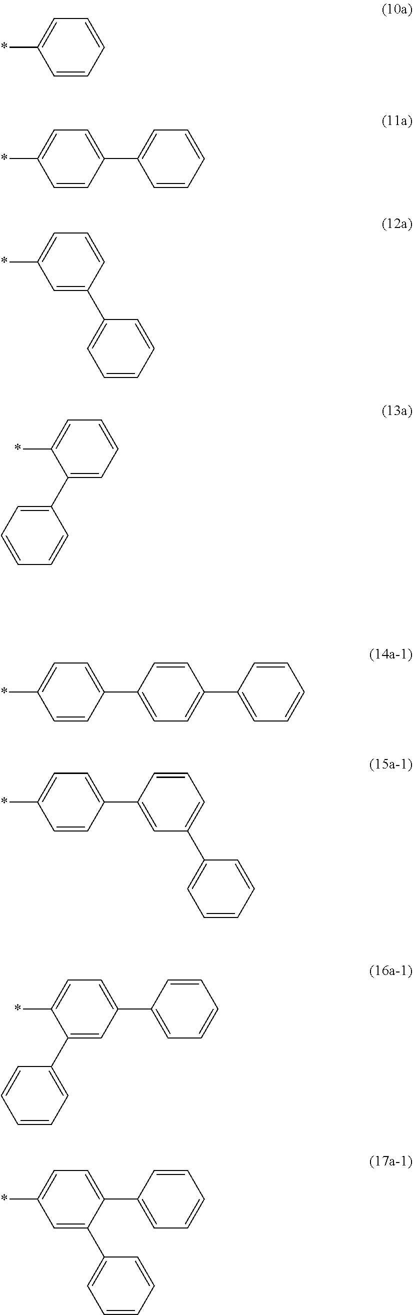

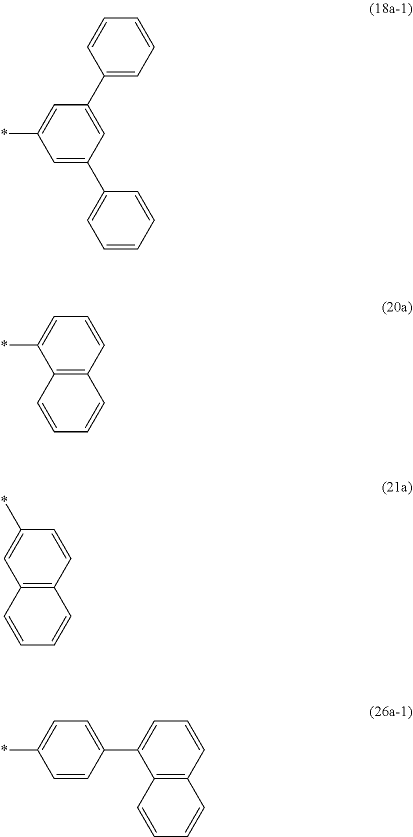 [SMILES: *c1cc(-c2ccccc2)cc(-c2ccccc2)c1 ; *c1cccc2ccccc12 ; *c1ccc2ccccc2c1 ; *c1ccc(-c2cccc3ccccc23)cc1]

### Organic EL Device

In an organic EL device in an exemplary arrangement of the sixth exemplary embodiment, the first compound used in the organic EL device according to the first exemplary embodiment, the second exemplary embodiment, or the third exemplary embodiment is replaced with the compound according to the sixth exemplary embodiment (the compound represented by the formula (10)).

The compound according to the sixth exemplary embodiment allows the organic EL device to achieve higher performance (especially, a decrease in voltage), specifically, both luminous efficiency and voltage suitable for practical use.

Thus, the organic EL device in an exemplary arrangement of the sixth exemplary embodiment also achieves higher performance.

### Organic EL Display Device

In an organic EL display device in an exemplary arrangement of the sixth exemplary embodiment, the first compound used in the organic EL display device according to the fourth exemplary embodiment or the fifth exemplary embodiment is replaced with the compound according to the sixth exemplary embodiment (the compound represented by the formula (10)).

The compound according to the sixth exemplary embodiment allows the organic EL display device to achieve higher performance (especially, a decrease in voltage), specifically, both luminous efficiency and voltage suitable for practical use.

Thus, the organic EL display device in an exemplary arrangement of the sixth exemplary embodiment also achieves higher performance.

### Seventh Exemplary Embodiment

### Electronic Device

An electronic device according to a seventh exemplary embodiment is installed with one of the organic EL devices according to the above exemplary embodiments or one of the organic EL display devices according to the above exemplary embodiments. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited to the above-described exemplary embodiments but includes any modification and improvement as long as such modification and improvement are compatible with the invention.

For instance, the emitting layer is not limited to a single layer, but may be provided by layering two emitting layers or multiple layers exceeding two. For instance, in some embodiments, the rest of the emitting layers is a fluorescent emitting layer or a phosphorescent emitting layer with use of emission caused by electron transfer from the triplet excited state directly to the ground state. When the organic EL device includes a plurality of emitting layers, these emitting layers may be in direct contact with each other, or may form a so-called tandem organic EL device, in which a plurality of emitting units are layered via an intermediate layer (occasionally also referred to as a charge generating layer).

Further, for instance, a blocking layer is optionally provided adjacent to a side of the emitting layer close to the cathode. The blocking layer provided in direct contact with the side of the emitting layer close to the cathode preferably blocks at least one of holes or excitons.

For instance, when the blocking layer is provided in contact with the side of the emitting layer close to the cathode, the blocking layer permits transport of electrons and blocks holes from reaching a layer provided closer to the cathode (e.g., the electron transporting layer) beyond the blocking layer. When the organic EL device includes the electron transporting layer, the blocking layer may be disposed between the emitting layer and the electron transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the like) closer to the electrode(s) than the blocking layer. The emitting layer is preferably in direct contact with the blocking layer.

Specific structure, shape and the like of the components in the invention may be designed in any manner as long as an object of the invention can be achieved.

### EXAMPLES

Examples of the invention are described below. The invention, however, is not limited to Examples.

### Compounds

Structures of the first compound used for manufacturing the organic EL devices in Examples are shown below.

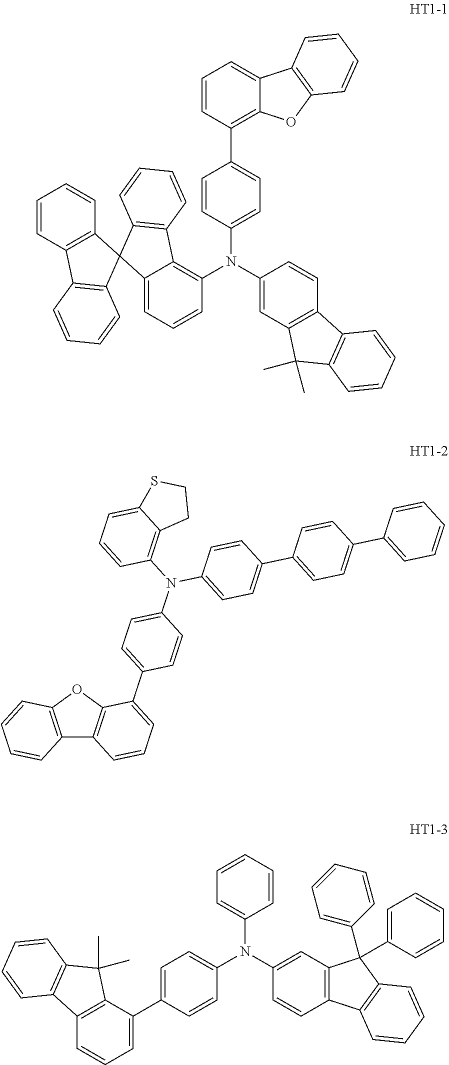 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21 ; c1ccc(-c2ccc(-c3ccc(N(c4ccc(-c5cccc6c5oc5ccccc56)cc4)c4cccc5c4CCS5)cc3)cc2)cc1 ; CC1(C)c2ccccc2-c2cccc(-c3ccc(N(c4ccccc4)c4ccc5c(c4)C(c4ccccc4)(c4ccccc4)c4ccccc4-5)cc3)c21]

Structures of the second compound used for manufacturing the organic EL devices in Examples are shown below.

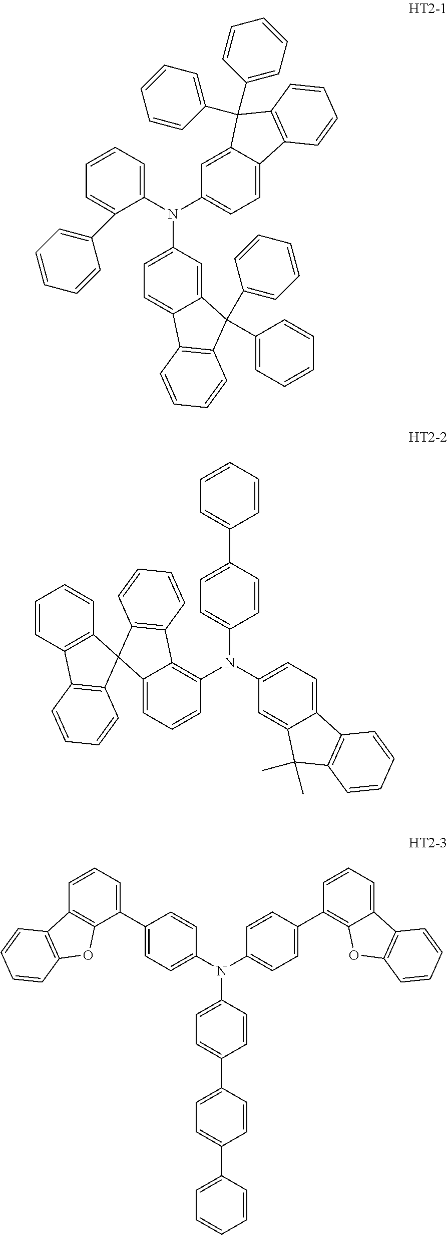 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; CC1(C)c2ccccc2-c2ccc(N(c3ccc(-c4ccccc4)cc3)c3cccc4c3-c3ccccc3C43c4ccccc4-c4ccccc43)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

Structures of comparative compounds used for manufacturing organic EL devices in Comparatives are shown below.

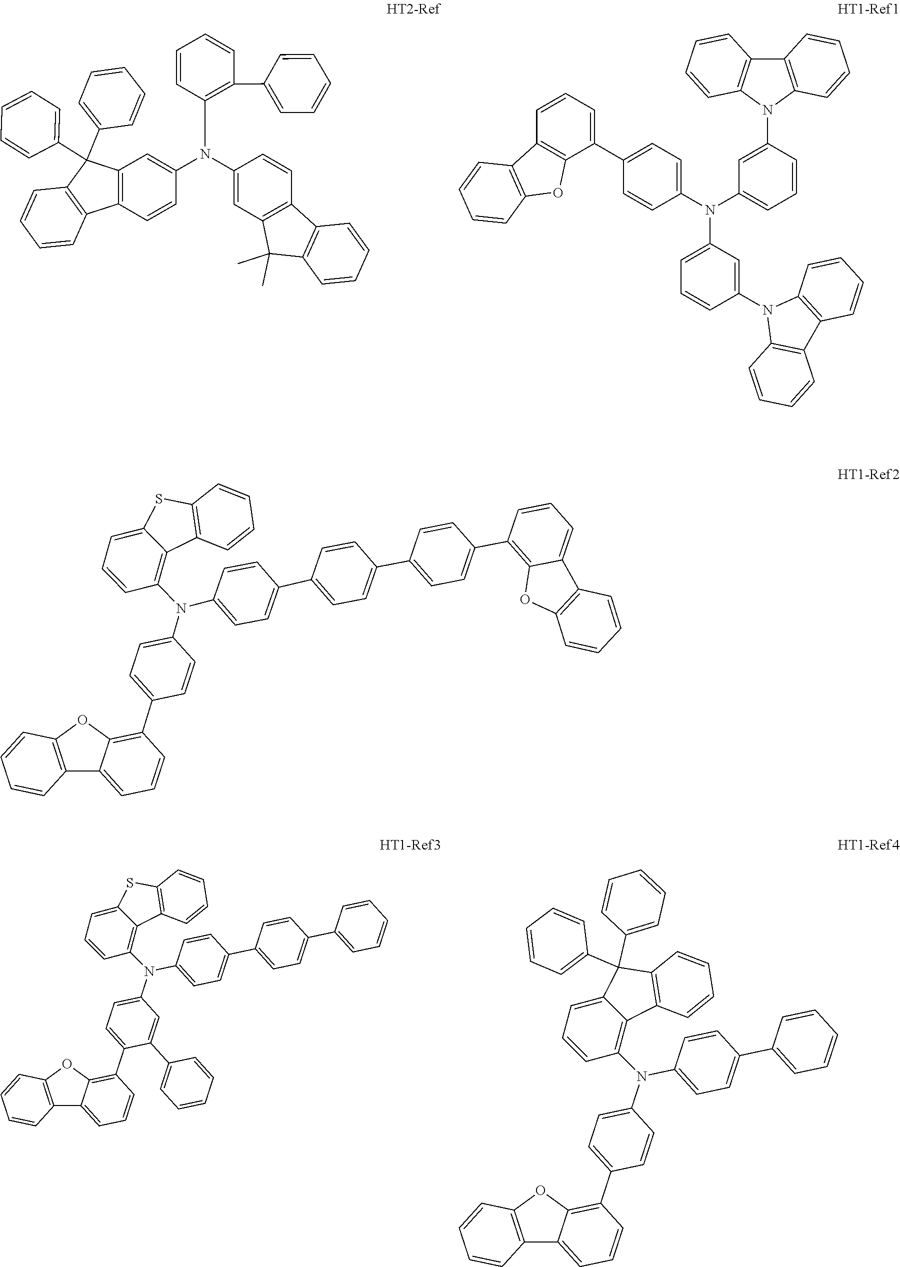 [SMILES: CC1(C)c2ccccc2-c2ccc(N(c3ccc4c(c3)C(c3ccccc3)(c3ccccc3)c3ccccc3-4)c3ccccc3-c3ccccc3)cc21 ; c1cc(N(c2ccc(-c3cccc4c3oc3ccccc34)cc2)c2cccc(-n3c4ccccc4c4ccccc43)c2)cc(-n2c3ccccc3c3ccccc32)c1 ; c1ccc2c(c1)oc1c(-c3ccc(-c4ccc(-c5ccc(N(c6ccc(-c7cccc8c7oc7ccccc78)cc6)c6cccc7sc8ccccc8c67)cc5)cc4)cc3)cccc12 ; c1ccc(-c2ccc(-c3ccc(N(c4ccc(-c5cccc6c5oc5ccccc56)c(-c5ccccc5)c4)c4cccc5sc6ccccc6c45)cc3)cc2)cc1 ; c1ccc(-c2ccc(N(c3ccc(-c4cccc5c4oc4ccccc45)cc3)c3cccc4c3-c3ccccc3C4(c3ccccc3)c3ccccc3)cc2)cc1]

Structures of other compounds used for manufacturing organic EL devices in Examples and Comparatives are shown below.

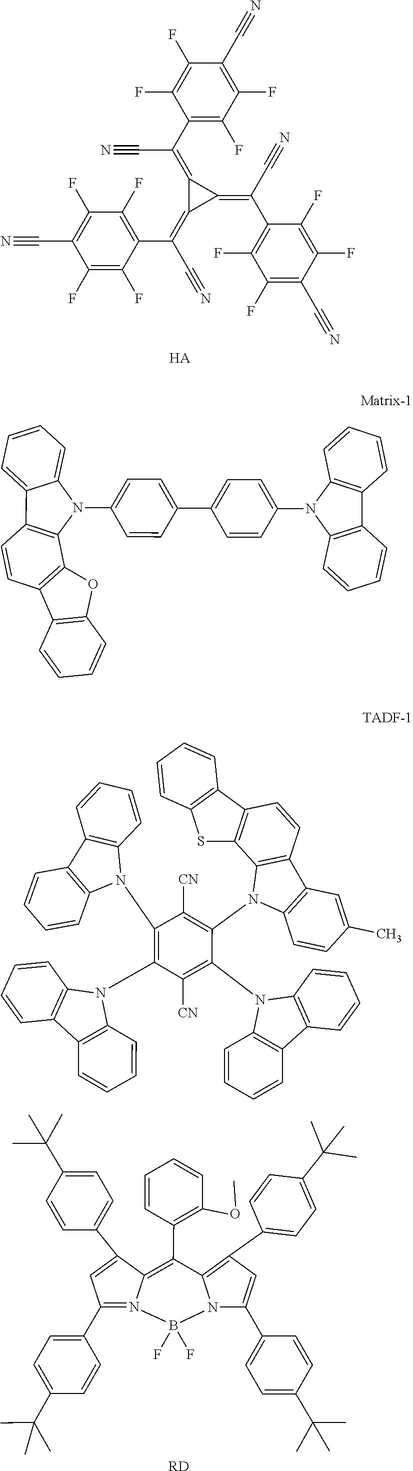

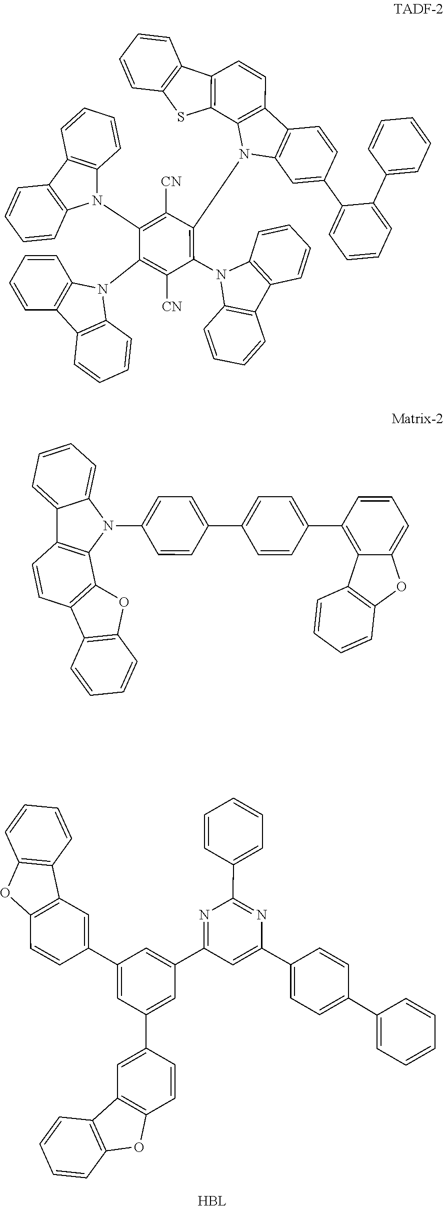 [SMILES: N#Cc1c(-n2c3ccccc3c3ccccc32)c(-n2c3ccccc3c3ccccc32)c(C#N)c(-n2c3cc(-c4ccccc4-c4ccccc4)ccc3c3ccc4c5ccccc5sc4c32)c1-n1c2ccccc2c2ccccc21 ; c1ccc2c(c1)oc1c2ccc2c3ccccc3n(-c3ccc(-c4ccc(-c5cccc6oc7ccccc7c56)cc4)cc3)c21 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

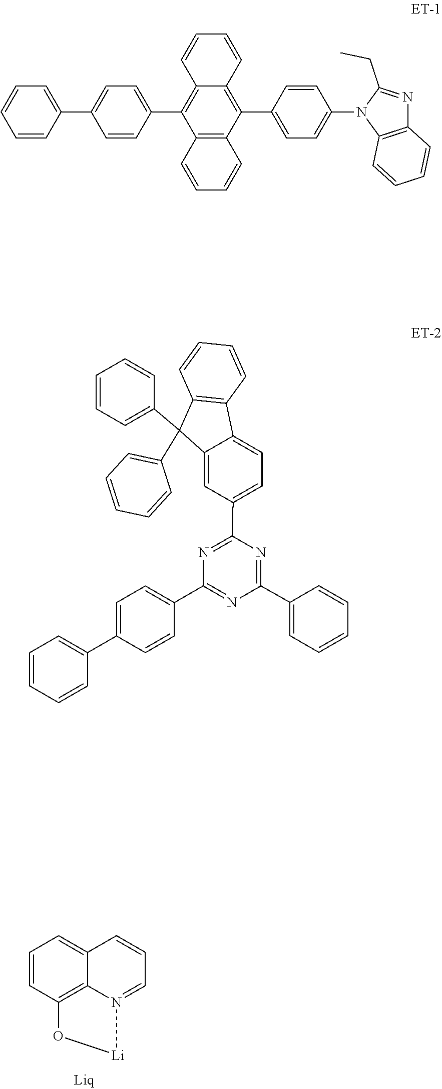

### Manufacture 1 of Organic EL Device

The organic EL devices were prepared and evaluated as follows.

### Example 1-1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for one minute. A film of ITO was 130 nm thick.

After the glass substrate having the transparent electrode line was cleaned, the glass substrate was mounted on a substrate holder of a vacuum evaporation apparatus. First, a compound HT2-1 and a compound HA were co-deposited on a surface of the glass substrate, where the transparent electrode line was provided, to cover the transparent electrode, thereby forming a 10-nm-thick hole injecting layer. The concentrations of the compound HT2-1 and the compound HA in the hole injecting layer were 97 mass % and 3 mass %, respectively.

Next, the compound HT2-1 as the second compound was vapor-deposited on the hole injecting layer to form a 130-nm-thick second layer (occasionally also referred to as a first hole transporting layer).

Next, a compound HT1-1 as the first compound was vapor-deposited on the second layer to form an 80-nm-thick first layer (occasionally also referred to as a second hole transporting layer or an electron blocking layer).

Next, a compound Matrix-1 as the compound M3, a compound TADF-1 as the compound M2, and a compound RD as the compound M1 were co-deposited on the first layer to form a 25-nm-thick emitting layer. The concentrations of the compound Matrix-1, the compound TADF-1, and the compound RD in the emitting layer were 74 mass %, 25 mass %, and 1 mass % respectively.

Next, a compound HBL was vapor-deposited on the emitting layer to form a 10-nm-thick hole blocking layer.

Next, the compound ET-1 was vapor-deposited on the hole blocking layer to form a 30-nm-thick electron transporting layer.

Next, LiF was vapor-deposited on the electron transporting layer to form a 1-nm-thick electron injecting layer.

Next, metal aluminum (Al) was vapor-deposited on the electron injecting layer to form an 80-nm-thick metal Al cathode.

A device arrangement of the organic EL device of Example 1-1 is roughly shown as follows.

ITO(130)/HT2-1:HA(10.97%:0:3%)/HT2-1(130)/HT1-1(80)/Matrix-1:TADF-1:RD(25.74%:25%:1%)/HBL(10)/ET-1(30)/LiF(1)/Al(80)

Numerals in parentheses represent a film thickness (unit: nm).

The numerals (97%:3%) represented by percentage in the same parentheses indicate a ratio (mass %) between the compound HT2-1 and the compound HA in the hole injecting layer. The numerals (74%:25%:1%) represented by percentage in the same parentheses indicate a ratio (mass %) between the compound Matrix-1, the compound TADF-1, and the compound RD in the emitting layer.

### Examples 1-2 to 1-3 and Comparative 1-1

The organic EL devices of Examples 1-2 to 1-3 and Comparative 1-1 were manufactured in the same manner as that of Example 1-1 except that the first compound and the second compound used in Example 1-1 were replaced with the compounds listed in Table 1.

### Comparatives 1-2 to 1-3

The organic EL devices of Comparatives 1-2 to 1-3 were manufactured in the same manner as that of Example 1-1 except that the first compound used in Example 1-1 was replaced with the compounds listed in Table 1.

### Evaluation of Organic EL Device

The manufactured organic EL devices were evaluated as follows. Table 1 shows the results.

### Maximum Peak Wavelength λp

Voltage was applied on the organic EL devices such that a current density was 10 mA/cm², where spectral radiance spectrum was measured by a spectroradiometer CS-2000 (manufactured by Konica Minolta, Inc.). The maximum peak wavelength λp (unit: nm) was obtained from the measured spectral radiance spectrum.

### Drive Voltage

A voltage (unit: V) was measured when current was applied between the anode and the cathode such that a current density was 10 mA/cm².

### External Quantum Efficiency EQE

Voltage was applied on the organic EL devices such that a current density was 10 mA/cm², where spectral radiance spectrum was measured by a spectroradiometer CS-2000 (manufactured by Konica Minolta, Inc.). The external quantum efficiency EQE (unit: %) was calculated based on the obtained spectral radiance spectra, assuming that the spectra was provided under a Lambertian radiation.

TABLE 1

| | Second Layer | | | First Layer | | | | | | | | | |
|---|---|---|---|---|---|---|---|---|---|---|---|---|---|
| | Second Compound | | Film | First Compound | | | Film | Emitting Layer | | | Device Evaluation | | |
| | | Ip | Thick- | | Ip | μh | Thick- | Compound | Compound | Compound | λp | Voltage | EQE |
| | Name | [eV] | ness | Name | [eV] | [cm²/Vs] | ness | M3 | M2 | M1 | [nm] | [V] | [%] |
| Ex. 1-1 | HT2-1 | 5.61 | 130 | HT1-1 | 5.70 | 8.76 × 10⁻⁵ | 80 | Matrix-1 | TADF-1 | RD | 623 | 4.80 | 16.6 |
| Ex. 1-2 | HT2-1 | 5.61 | 130 | HT1-2 | 5.70 | 1.25 × 10⁻⁴ | 80 | Matrix-1 | TADF-1 | RD | 623 | 4.73 | 17.2 |
| Ex. 1-3 | HT2-2 | 5.69 | 130 | HT1-2 | 5.70 | 1.25 × 10⁻⁴ | 80 | Matrix-1 | TADF-1 | RD | 623 | 4.61 | 17.2 |
| Comp. | HT2- | 5.56 | 130 | HT1- | 5.86 | 5.18 × 10⁻¹⁰ | 80 | Matrix-1 | TADF-1 | RD | 623 | 9.37 | 18.8 |
| 1-1 | Ref | | | Ref1 | | | | | | | | | |
| Comp. | HT2-1 | 5.61 | 130 | HT1- | 5.67 | 1.12 × 10⁻⁴ | 80 | Matrix-1 | TADF-1 | RD | 623 | 4.75 | 14.3 |
| 1-2 | | | | Ref2 | | | | | | | | | |
| Comp. | HT2-1 | 5.61 | 130 | HT1- | 5.70 | 1.31 × 10⁻⁶ | 80 | Matrix-1 | TADF-1 | RD | 623 | 6.32 | 17.3 |
| 1-3 | | | | Ref3 | | | | | | | | | |

In the organic EL devices of Examples 1-1 to 1-3, the first layer contains the first compound satisfying Numerical Formula 1 and Numerical Formula 2, the second layer contains the second compound satisfying Numerical Formula 3, and the film thickness of the first layer is thickened (80 nm).

In the organic EL device of Comparative 1-1, the first layer contains a compound not satisfying Numerical Formula 2, the second layer contains a compound not satisfying Numerical Formula 3, and the film thickness of the first layer is thickened (80 nm).

In the organic EL device of Comparative 1-2, the first layer contains a compound not satisfying Numerical Formula 1 and the film thickness of the first layer is thickened (80 nm).

In the organic EL device of Comparative 1-3, the first layer contains a compound not satisfying Numerical Formula 2 and the film thickness of the first layer is thickened (80 nm).

The organic EL devices of Examples 1-1 to 1-3 emitted light while achieving both drive voltage (low voltage) and luminous efficiency suitable for practical use. For the organic EL devices of Comparatives 1-1 and 1-3, the luminous efficiency was suitable for practical use, but the drive voltage was too high to be practically used. For the organic EL device of Comparative 1-2, the drive voltage was suitable for practical use, but the luminous efficiency was low.

### Manufacture 2 of Organic EL Device

The organic EL devices were prepared and evaluated as follows.

### Example 2-1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for one minute. A film of ITO was 130 nm thick.

After the glass substrate having the transparent electrode line was cleaned, the glass substrate was mounted on a substrate holder of a vacuum evaporation apparatus. First, the compound HT2-1 and the compound HA were co-deposited on a surface of the glass substrate, where the transparent electrode line was provided, to cover the transparent electrode, thereby forming a 10-nm-thick hole injecting layer. The concentrations of the compound HT2-1 and the compound HA in the hole injecting layer were 97 mass % and 3 mass %, respectively.

Next, the compound HT2-1 as the second compound was vapor-deposited on the hole injecting layer to form a 130-nm-thick second layer (occasionally also referred to as the first hole transporting layer).

Next, the compound HT1-1 as the first compound was vapor-deposited on the second layer to form an 80-nm-thick first layer (occasionally also referred to as the second hole transporting layer or the electron blocking layer).

Next, the compound Matrix-1 as the compound M3, a compound TADF-2 as the compound M2, and the compound RD as the compound M1 were co-deposited on the first layer to form a 25-nm-thick emitting layer. The concentrations of the compound Matrix-1, the compound TADF-2, and the compound RD in the emitting layer were 74 mass %, 25 mass %, and 1 mass % respectively.

Next, the compound HBL was vapor-deposited on the emitting layer to form a 10-nm-thick hole blocking layer.

Next, a compound ET-2 and a compound Liq were co-deposited on the hole blocking layer to form a 30-nm-thick electron transporting layer. The concentrations of the compound ET-2 and the compound Liq in the electron transporting layer were 50 mass % and 50 mass %, respectively.

Next, Yb was vapor-deposited on the electron transporting layer to form a 1-nm-thick electron injecting layer.

Next, metal aluminum (Al) was vapor-deposited on the electron injecting layer to form a 50-nm-thick metal Al cathode.

A device arrangement of the organic EL device of Example 2-1 is roughly shown as follows.

ITO(130)/HT2-1:HA(10.97%0:3%)/HT2-1(130)/HT1-1(80)/Matrix-1:TADF-2:RD(25.74%:25%:1%)/HBL(10)/ET-2:Liq(30.50%:50%)/Yb(1)/Al(50)

Numerals in parentheses represent a film thickness (unit: nm).

The numerals (97%:3%) represented by percentage in the same parentheses indicate a ratio (mass %) between the compound HT2-1 and the compound HA in the hole injecting layer. The numerals (74%:25%:1%) represented by percentage in the same parentheses indicate a ratio (mass %) between the compound Matrix-1, the compound TADF-2, and the compound RD in the emitting layer. The numerals (50%:50%) represented by percentage in the same parentheses indicate a ratio (mass %) between the compound ET-2 and the compound Liq in the electron transporting layer.

### Examples 2-2 to 2-3

The organic EL devices of Examples 2-2 to 2-3 were manufactured in the same manner as that of Example 2-1 except that the first compound used in Example 2-1 was replaced with the compounds listed in Table 2.

### Examples 2-4 and 2-7

The organic EL devices of Examples 2-4 and 2-7 were manufactured in the same manner as that of Example 2-1 except that the second compound used in Example 2-1 was replaced with the compounds listed in Table 2.

### Examples 2-5 to 2-6, Examples 2-8 to 2-9, and Comparatives 2-1 to 2-2

The organic EL devices of Examples 2-5 to 2-6, Examples 2-8 to 2-9, and Comparatives 2-1 to 2-2 were manufactured in the same manner as that of Example 2-1 except that the first compound and the second compound used in Example 2-1 were replaced with the compounds listed in Table 2.

### Example 3-1

The organic EL device of Example 3-1 was manufactured in the same manner as that of Example 2-1 except that the compound Matrix-1 used in Example 2-1 was replaced with the compound listed in Table 3.

### Examples 3-2 to 3-3

The organic EL devices of Examples 3-2 to 3-3 were manufactured in the same manner as that of Example 3-1 except that the first compound used in Example 3-1 was replaced with the compounds listed in Table 3.

### Example 3-4

The organic EL device of Example 3-4 was manufactured in the same manner as that of Example 3-1 except that the second compound used in Example 3-1 was replaced with the compound listed in Table 3.

### Examples 3-5 to 3-6 and Comparatives 3-1 to 3-2

The organic EL devices of Examples 3-5 to 3-6 and Comparatives 3-1 to 3-2 were manufactured in the same manner as that of Example 3-1 except that the first compound and the second compound used in Example 3-1 were replaced with the compounds listed in Table 3.

### Evaluation of Organic EL Device

The manufactured organic EL devices were evaluated in the same manner as in Example 1-1. Tables 2 and 3 show the evaluation results.

TABLE 2

| | Second Layer | | | First Layer | | | | | | | | | |
|---|---|---|---|---|---|---|---|---|---|---|---|---|---|
| | Second Compound | | Film | First Compound | | | Film | Emitting Layer | | | Device Evaluation | | |
| | | Ip | Thick- | | Ip | μh | Thick- | Compound | Compound | Compound | λp | Voltage | EQE |
| | Name | [eV] | ness | Name | [eV] | [cm²/Vs] | ness | M3 | M2 | M1 | [nm] | [V] | [%] |
| Ex. 2-1 | HT2-1 | 5.61 | 130 | HT1-1 | 5.70 | 8.76 × 10⁻⁵ | 80 | Matrix-1 | TADF-2 | RD | 622 | 4.80 | 15.9 |
| Ex. 2-2 | HT2-1 | 5.61 | 130 | HT1-2 | 5.70 | 1.25 × 10⁻⁴ | 80 | Matrix-1 | TADF-2 | RD | 622 | 4.66 | 16.3 |
| Ex. 2-3 | HT2-1 | 5.61 | 130 | HT1-3 | 5.73 | 7.00 × 10⁻⁵ | 80 | Matrix-1 | TADF-2 | RD | 622 | 4.57 | 17.6 |
| Ex. 2-4 | HT2-2 | 5.69 | 130 | HT1-1 | 5.70 | 8.76 × 10⁻⁵ | 80 | Matrix-1 | TADF-2 | RD | 623 | 5.63 | 16.5 |
| Ex. 2-5 | HT2-2 | 5.69 | 130 | HT1-2 | 5.70 | 1.25 × 10⁻⁴ | 80 | Matrix-1 | TADF-2 | RD | 623 | 5.73 | 16.6 |
| Ex. 2-6 | HT2-2 | 5.69 | 130 | HT1-3 | 5.73 | 7.00 × 10⁻⁵ | 80 | Matrix-1 | TADF-2 | RD | 622 | 5.60 | 17.8 |
| Ex. 2-7 | HT2-3 | 5.66 | 130 | HT1-1 | 5.70 | 8.76 × 10⁻⁵ | 80 | Matrix-1 | TADF-2 | RD | 623 | 5.16 | 16.8 |
| Ex. 2-8 | HT2-3 | 5.66 | 130 | HT1-2 | 5.70 | 1.25 × 10⁻⁴ | 80 | Matrix-1 | TADF-2 | RD | 623 | 5.09 | 17.7 |
| Ex. 2-9 | HT2-3 | 5.66 | 130 | HT1-3 | 5.73 | 7.00 × 10⁻⁵ | 80 | Matrix-1 | TADF-2 | RD | 623 | 4.87 | 18.9 |
| Comp. | HT2- | 5.56 | 130 | HT1- | 5.86 | 5.18 × 10⁻¹⁰ | 80 | Matrix-1 | TADF-2 | RD | 623 | 8.49 | 18.2 |
| 2-1 | Ref | | | Ref1 | | | | | | | | | |
| Comp. | HT2- | 5.56 | 130 | HT1- | 5.78 | 2.00 × 10⁻⁶ | 80 | Matrix-1 | TADF-2 | RD | 622 | 6.25 | 18.1 |
| 2-2 | Ref | | | Ref4 | | | | | | | | | |

In the organic EL devices of Examples 2-1 to 2-9, the first layer contains the first compound satisfying Numerical Formula 1 and Numerical Formula 2, the second layer contains the second compound satisfying Numerical Formula 3, and the film thickness of the first layer is thickened (80 nm).

In the organic EL devices of Comparatives 2-1 to 2-2, the first layer contains a compound not satisfying Numerical Formula 2, the second layer contains a compound not satisfying Numerical Formula 3, and the film thickness of the first layer is thickened (80 nm).

The organic EL devices of Examples 2-1 to 2-9 emitted light while achieving both drive voltage (low voltage) and luminous efficiency suitable for practical use. For the organic EL devices of Comparatives 2-1 to 2-2, the luminous efficiency was suitable for practical use, but voltage was too high to be practically used.

TABLE 3

| | Second Layer | | | First Layer | | | | | | | | | |
|---|---|---|---|---|---|---|---|---|---|---|---|---|---|
| | Second Compound | | Film | First Compound | | | Film | Emitting Layer | | | Device Evaluation | | |
| | | Ip | Thick- | | Ip | μh | Thick- | Compound | Compound | Compound | λp | Voltage | EQE |
| | Name | [eV] | ness | Name | [eV] | [cm²/Vs] | ness | M3 | M2 | M1 | [nm] | [V] | [%] |
| Ex. 3-1 | HT2-1 | 5.61 | 130 | HT1-1 | 5.70 | 8.76 × 10⁻⁵ | 80 | Matrix-2 | TADF-2 | RD | 621 | 5.04 | 14.2 |
| Ex. 3-2 | HT2-1 | 5.61 | 130 | HT1-2 | 5.70 | 1.25 × 10⁻⁴ | 80 | Matrix-2 | TADF-2 | RD | 622 | 5.13 | 15.5 |
| Ex. 3-3 | HT2-1 | 5.61 | 130 | HT1-3 | 5.73 | 7.00 × 10⁻⁵ | 80 | Matrix-2 | TADF-2 | RD | 622 | 5.02 | 16.6 |
| Ex. 3-4 | HT2-3 | 5.66 | 130 | HT1-1 | 5.70 | 8.76 × 10⁻⁵ | 80 | Matrix-2 | TADF-2 | RD | 622 | 5.42 | 15.7 |
| Ex. 3-5 | HT2-3 | 5.66 | 130 | HT1-2 | 5.70 | 1.25 × 10⁻⁴ | 80 | Matrix-2 | TADF-2 | RD | 622 | 5.43 | 17.4 |
| Ex. 3-6 | HT2-3 | 5.66 | 130 | HT1-3 | 5.73 | 7.00 × 10⁻⁵ | 80 | Matrix-2 | TADF-2 | RD | 622 | 5.18 | 18.5 |
| Comp. | HT2- | 5.56 | 130 | HT1- | 5.86 | 5.18 × 10⁻¹⁰ | 80 | Matrix-2 | TADF-2 | RD | 622 | 8.53 | 19.0 |
| 3-1 | Ref | | | Ref1 | | | | | | | | | |
| Comp. | HT2- | 5.56 | 130 | HT1- | 5.78 | 2.00 × 10⁻⁶ | 80 | Matrix-2 | TADF-2 | RD | 622 | 6.40 | 18.4 |
| 3-2 | Ref | | | Ref4 | | | | | | | | | |

In the organic EL devices of Examples 3-1 to 3-6, the first layer contains the first compound satisfying Numerical Formula 1 and Numerical Formula 2, the second layer contains the second compound satisfying Numerical Formula 3, and the film thickness of the first layer is thickened (80 nm).

In the organic EL devices of Comparatives 3-1 to 3-2, the first layer contains a compound not satisfying Numerical Formula 2, the second layer contains a compound not satisfying Numerical Formula 3, and the film thickness of the first layer is thickened (80 nm).

The organic EL devices of Examples 3-1 to 3-6 emitted light while achieving both drive voltage (low voltage) and luminous efficiency suitable for practical use. For the organic EL devices of Comparatives 3-1 to 3-2, the luminous efficiency was suitable for practical use, but voltage was too high to be practically used.

### Evaluation of Compounds

Values of physical properties of the compounds shown in Tables 1 to 3 were measured by the following method. Tables 1 to 4 show the results.

### Thermally Activated Delayed Fluorescence

### Delayed Fluorescence of Compound TADF-1

Delayed fluorescence properties were checked by measuring transient photoluminescence (PL) using a device shown in FIG. 2. The compound TADF-1 was dissolved in toluene to prepare a dilute solution with an absorbance of 0.05 or less at the excitation wavelength to eliminate the contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the above sample solution was measured with a spectrofluorometer FP-8600 (manufactured by JASCO Corporation), and the fluorescence spectrum of a 9,10-diphenylanthracene ethanol solution was measured under the same conditions. Using the fluorescence area intensities of both spectra, the total fluorescence quantum yield is calculated by an equation (1) in Morris et al. J. Phys. Chem. 80 (1976) 969.

Prompt emission was observed immediately when the excited state was achieved by exciting the compound TADF-1 with a pulse beam (i.e., a beam emitted from a pulse laser) having a wavelength to be absorbed by the compound TADF-1, and Delay emission was observed not immediately when the excited state was achieved but after the excited state was achieved. The delayed fluorescence in Examples means that an amount of Delay Emission is 5% or more with respect to an amount of Prompt Emission. Specifically, provided that the amount of Prompt emission is denoted by X_{P} and the amount of Delay emission is denoted by X_{D}, the delayed fluorescence means that a value of X_{D}/X_{P} is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

Measurement for the compound TADF-2 was performed in the same manner as the compound TADF-1. It was confirmed that the amount of Delay Emission was 5% or more with respect to the amount of Prompt Emission in the compounds TADF-1 and TADF-2. Specifically, the value of X_{D}/X_{P} was 0.05 or more in the compounds TADF-1 and TADF-2.

### Singlet Energy S₁

The singlet energy S₁ of each measurement target compound was measured according to the above-described solution method.

### Energy Gap T_{77K}

T_{77K} of each measurement target compound was measured. T_{77K} was measured by the measurement method of the energy gap T_{77K} described in “Relationship between Triplet Energy and Energy Gap at 77K.”

T_{77K} of the compound Matrix-1 was 2.89 eV.

T_{77K} of the compound Matrix-2 was 2.79 eV.

### ΔST

ΔST was calculated based on the measured lowest singlet energy S₁ and energy gap T_{77K} at 77K. In Tables, the notation “<0.01” indicates that ΔST was less than 0.01 eV.

### Maximum Peak Wavelength A of Compounds

A toluene solution of each measurement target compound at a concentration of 5 μmol/L was prepared and put in a quartz cell. A fluorescence spectrum (ordinate axis: fluorescence intensity, abscissa axis: wavelength) of each sample was measured at a normal temperature (300K). In Examples, a fluorescence spectrum was measured with a spectrophotofluorometer (manufactured by Hitachi High-Tech Science Corporation: F-7000). It should be noted that the fluorescence spectrum measuring device may be different from the above device. A peak wavelength of a fluorescence spectrum, a luminous intensity of which is the maximum in the fluorescence spectrum, was defined as the maximum peak wavelength A.

TABLE 4

| | | S₁ | ΔST | λ |
|---|---|---|---|---|
| | | [eV] | [eV] | [nm] |
| Compound M1 | RD | 2.02 | — | 609 |
| Compound M2 | TADF-1 | 2.32 | <0.01 | 545 |
| | TADF-2 | 2.34 | <0.01 | 539 |
| Compound M3 | matrix-1 | 3.42 | 0.53 | — |
| | matrix-2 | 3.42 | 0.63 | — |

### Ionization Potential Ip

The ionization potential Ip of each compound was measured under atmosphere using a photoelectron spectroscope (“AC-3” manufactured by RIKEN KEIKI Co., Ltd.). Specifically, the material was irradiated with light and the amount of electrons generated by charge separation was measured to measure the ionization potential of the compound. The ionization potential is occasionally referred to as Ip.

### Hole Mobility μh

The hole mobility μh was measured using a mobility evaluation device manufactured by the following steps.

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV-ozone-cleaned for 30 minutes. A film of ITO was 130 nm thick.

After the glass substrate was cleaned, the glass substrate was mounted on a substrate holder of a vacuum evaporation apparatus. First, the compound HA-2 was vapor-deposited on a surface of the glass substrate, where the transparent electrode line was provided, to cover the transparent electrode, thereby forming a 5-nm-thick hole injecting layer.

A compound HT-A was vapor-deposited on the formed hole injecting layer to form a 10-nm-thick hole transporting layer.

Subsequently, a compound Target to be measured for the hole mobility μh was vapor-deposited to form a 200-nm-thick measurement target layer.

Metal aluminum (Al) was vapor-deposited on this measurement target layer to form an 80-nm-thick metal cathode.

An arrangement of the mobility evaluation device above is roughly shown as follows.

ITO(130)/HA-2(5)/HT-A(10)/Target(200)/Al(80)

Numerals in parentheses represent a film thickness (nm).

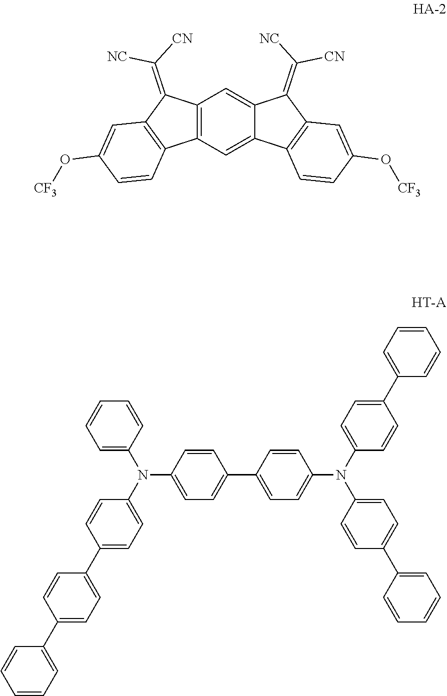 [SMILES: N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

Subsequently, the hole mobility is measured by the following steps using the mobility evaluation device manufactured as described above.

The mobility evaluation device was set in an impedance measurement device to perform an impedance measurement.

In the impedance measurement, a measurement frequency was swept from 1 Hz to 1 MHz. At this time, an alternating current amplitude of 0.1 V and a direct current voltage V were applied to the device.

A modulus M was calculated from a measured impedance Z using a relationship of a calculation formula (C1) below.

M=jωZ Calculation Formula (C1):

In the calculation formula (C1), j is an imaginary unit whose square is −1 and ω is an angular frequency [rad/s].

In a bode plot in which an imaginary part of the modulus M is represented by an ordinate axis and the frequency [Hz] is represented by an abscissa axis, an electrical time constant τ of the mobility evaluation device was obtained from a frequency fmax showing a peak using a calculation formula (C2) below.

τ=1/(2πf max) Calculation Formula (C2):

π in the calculation formula (C2) is a symbol representing a circumference ratio.

The hole mobility μh was calculated from a relationship of a calculation formula (C3) below using T.

μh=d²/(Vτ) Calculation Formula (C3):

d in the calculation formula (C3) is a total film thickness of organic thin film(s) forming the device. As in the arrangement of the mobility evaluation device, d=215 [nm] is satisfied.

The mobility herein is a value obtained when a square root of an electric field intensity meets E^{1/2}=500 [V^{1/2}/cm^{1/2}]. The square root of the electric field intensity, E^{1/2}, can be calculated from a relationship of a calculation formula (C4) below.

E^{1/2}=V^{1/2}/d^{1/2} Calculation Formula (C4):

For the impedance measurement in Examples, a 1260 type by Solartron Analytical was used as the impedance measurement device, and a 1296 type dielectric constant measurement interface by Solartron Analytical was used together therewith to enhance measurement accuracy.

### Synthesis of Compound

### Synthesis Example 1

As the compound represented by the formula (10), a compound HT1-2 was synthesized.

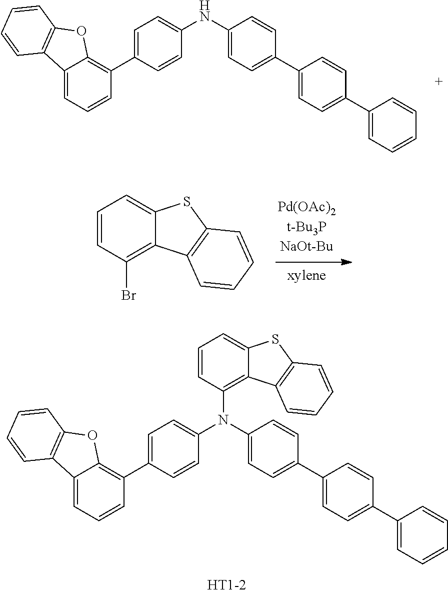 [SMILES: c1ccc(-c2ccc(-c3ccc(Nc4ccc(-c5cccc6c5oc5ccccc56)cc4)cc3)cc2)cc1 ; Brc1cccc2sc3ccccc3c12 ; c1ccc(-c2ccc(-c3ccc(N(c4ccc(-c5cccc6c5oc5ccccc56)cc4)c4cccc5sc6ccccc6c45)cc3)cc2)cc1]

Under argon atmosphere, a mixture of N-[4-(dibenzo[b,d]furan-4-yl)phenyl][1,1′:4′,1″-terphenyl]-4-amine (59.0 g, 121 mmol), 1-bromodibenzo[b,d]thiophene (35.0 g, 133 mmol), palladium acetate (0.540 g, 2.42 mmol), tri-tert-butylphosphine (0.980 g, 4.84 mmol), sodium-t-butoxide (17.4 g, 182 mmol), and xylene (940 mL) was refluxed at 100 degrees C. for 10 hours. The reaction solution was cooled to room temperature, which was then concentrated under reduced pressure. The obtained residue was refined by column chromatography and recrystallization to obtain 42.1 g of a white solid. The yield was 52%.

As a result of mass spectrum analysis, this white solid was the compound HT1-2. m/e was equal to 670 while a calculated molecular weight was 669.84.

1: An organic electroluminescence device comprising:
an anode;
a cathode;
an emitting layer provided between the anode and the cathode;
a first layer provided between the anode and the emitting layer; and
a second layer provided between the anode and the first layer, wherein
the emitting layer comprises a delayed fluorescent compound,
the first layer comprises a first compound,
the second layer comprises a second compound,
an ionization potential Ip(HT1) of the first compound satisfies Numerical Formula 1,
a hole mobility μh(HT1) of the first compound satisfies Numerical Formula 2,
an ionization potential Ip(HT2) of the second compound satisfies Numerical Formula 3, and
the first layer has a film thickness of 15 nm or more,
Ip(HT1)≥5.69 eV (Numerical Formula 1)
μh(HT1)≥1.00×10⁻⁵ cm²/Vs (Numerical Formula 2)
Ip(HT2)≥5.60 eV (Numerical Formula 3).

2: The organic electroluminescence device according to claim 1, wherein
the ionization potential IP(HT1) of the first compound and the ionization potential Ip(HT2) of the second compound satisfy Numerical Formula 10,
Ip(HT1)>Ip(HT2) (Numerical Formula 10).

3: The organic electroluminescence device according to claim 1, wherein
the ionization potential Ip(HT1) of the first compound satisfies Numerical Formula 11,
Ip(HT1)≥5.70 eV (Numerical Formula 11).

4: The organic electroluminescence device according to claim 1, wherein
the emitting layer comprises an emitting compound that emits light having a maximum peak wavelength in a range from 600 nm to 660 nm.

5: The organic electroluminescence device according to claim 1, wherein
the first layer is in direct contact with the emitting layer.

6: The organic electroluminescence device according to claim 1, wherein
the first layer is in direct contact with the second layer.

7: The organic electroluminescence device according to claim 1, wherein
the emitting layer comprises a compound M2 as the delayed fluorescent compound and a fluorescent compound M1, and
a singlet energy S₁(Mat2) of the compound M2 and a singlet energy S₁(Mat1) of the compound M1 satisfy Numerical Formula 7,
S₁(Mat2)>S₁(Mat1) (Numerical Formula 7).

8: The organic electroluminescence device according to claim 1, wherein
the emitting layer comprises a compound M2 as the delayed fluorescent compound and a compound M3, and
a singlet energy S₁(Mat2) of the compound M2 and a singlet energy S₁(Mat3) of the compound M3 satisfy Numerical Formula 4,
S₁(Mat3)>S₁(Mat2) (Numerical Formula 4).

9: The organic electroluminescence device according to claim 1, wherein
the first layer has a film thickness of 45 nm or more.

10: The organic electroluminescence device according to claim 1, wherein
the first layer has a film thickness of 55 nm or more.

11: The organic electroluminescence device according to claim 1, wherein
the ionization potential Ip(HT2) of the second compound satisfies Numerical Formula 31,
Ip(HT2)≥5.65 eV (Numerical Formula 31).

12: The organic electroluminescence device according to claim 1, wherein
the second layer has a film thickness in a range from 80 nm to 140 nm.

13: The organic electroluminescence device according to claim 1, wherein
the emitting layer does not comprise a metal complex.

14: The organic electroluminescence device according to claim 1, wherein
the first compound is a compound of formula (10), 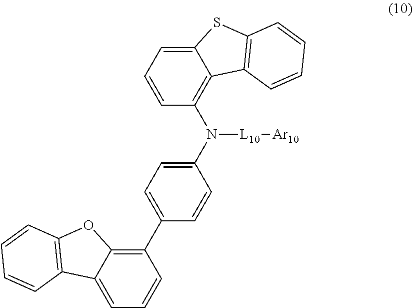
wherein:
L₁₀ is a single bond, or a substituted or unsubstituted arylene group having 6 to 12 ring carbon atoms;
a substituent, if present, for L₁₀ is an unsubstituted phenyl group;
Ar₁₀ is a substituted or unsubstituted aryl group having 6 to 18 ring carbon atoms; and
a substituent, if present, for Ar₁₀ is an unsubstituted phenyl group or an unsubstituted naphthyl group.

15: The organic electroluminescence device according to claim 14, wherein
L₁₀ is a single bond, or a substituted or unsubstituted phenylene group.

16: The organic electroluminescence device according to claim 14, wherein
Ar₁₀ is a group represented by any of formulae (10a) to (27a), 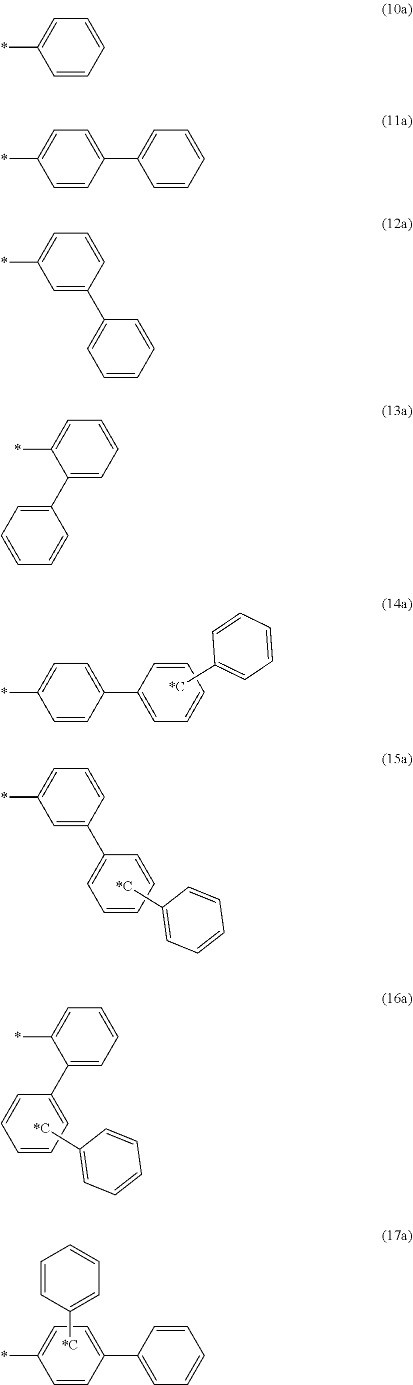 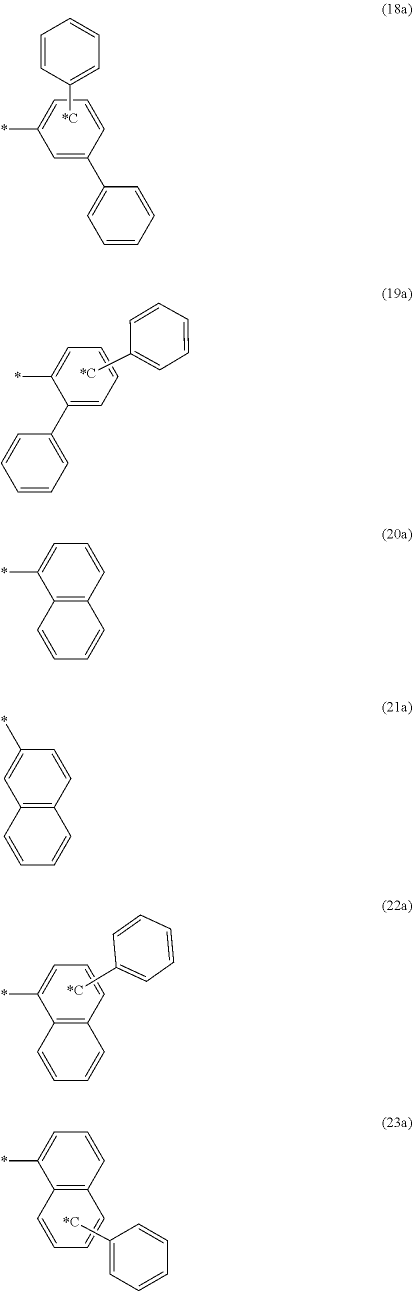 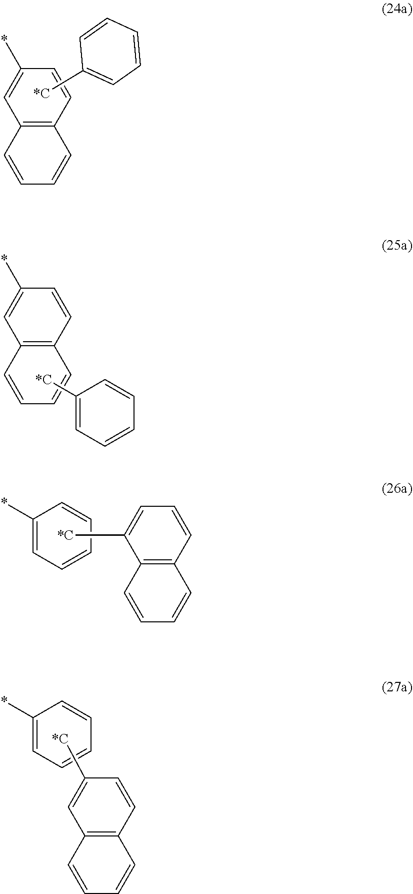
where, in the formulae (14a) to (19a) and (22a) to (27a), *C represents a bonding position to a carbon atom on a benzene ring.

17: The organic electroluminescence device according to claim 16, wherein
Ar₁₀ is a group represented by any of the formulae (10a) to (14a), (17a), (18a), and (26a).

18: An electronic device comprising the organic electroluminescence device according to claim 1.

19: An organic electroluminescence display device, comprising:
an anode and a cathode arranged to face each other;
a blue-emitting organic EL device as a blue pixel; a green-emitting organic EL device as a green pixel; and a red-emitting organic EL device as a red pixel, wherein
the red pixel comprises the organic electroluminescence device according to claim 1 as the red-emitting organic EL device,
the red-emitting organic EL device comprises: a red emitting layer as the emitting layer; the first layer provided between the red emitting layer and the anode; and the second layer provided between the first layer and the anode,
the blue-emitting organic EL device comprises a blue emitting layer provided between the anode and the cathode,
the green-emitting organic EL device comprises a green emitting layer provided between the anode and the cathode, and
the second layer is provided between the anode and each of the blue emitting layer, the green emitting layer, and the first layer in a shared manner across the blue-emitting organic EL device, the green-emitting organic EL device, and the red-emitting organic EL device.

20: The organic electroluminescence display device according to claim 19, wherein
the second layer is in direct contact with each of the blue emitting layer, the green emitting layer, and the first layer.

21: An electronic device comprising the organic electroluminescence display device according to claim 19.

22: A compound represented by a formula (10), 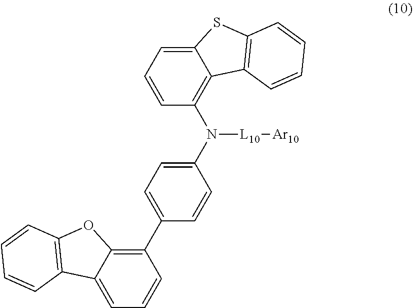
wherein
L₁₀ is a single bond, or a substituted or unsubstituted arylene group having 6 to 12 ring carbon atoms;
a substituent, if present, for L₁₀ is an unsubstituted phenyl group;
Ar₁₀ is a substituted or unsubstituted aryl group having 6 to 18 ring carbon atoms; and
a substituent, if present, for Ar₁₀ is an unsubstituted phenyl group or an unsubstituted naphthyl group.

23: The compound according to claim 22, wherein
L₁₀ is a single bond, or a substituted or unsubstituted phenylene group.

24: The compound according to claim 22, wherein
Ar₁₀ is a group represented by any of formulae (10a) to (27a), 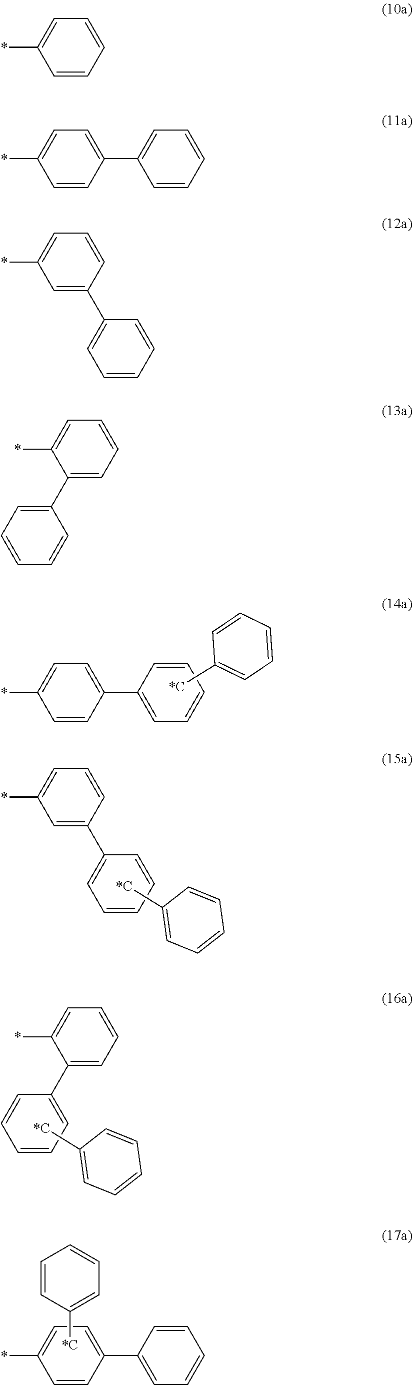 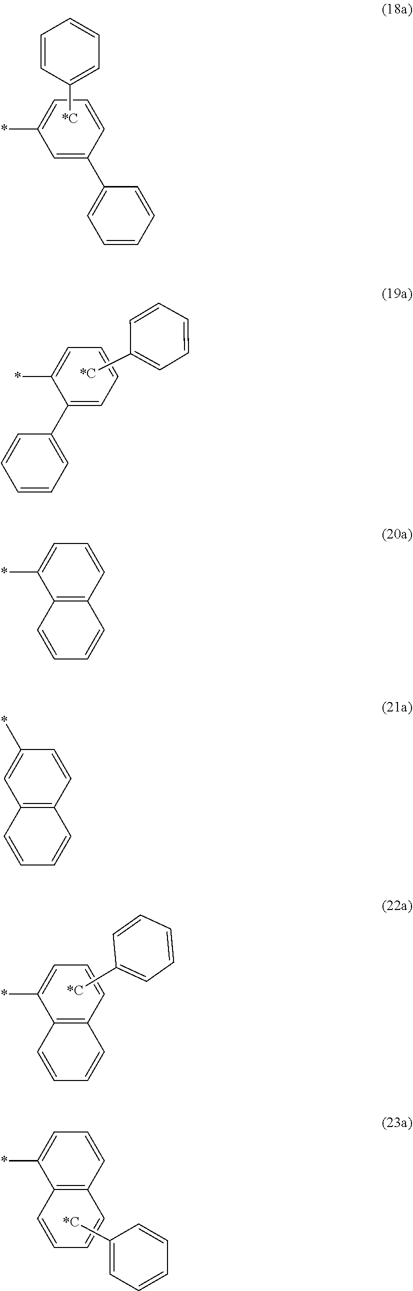 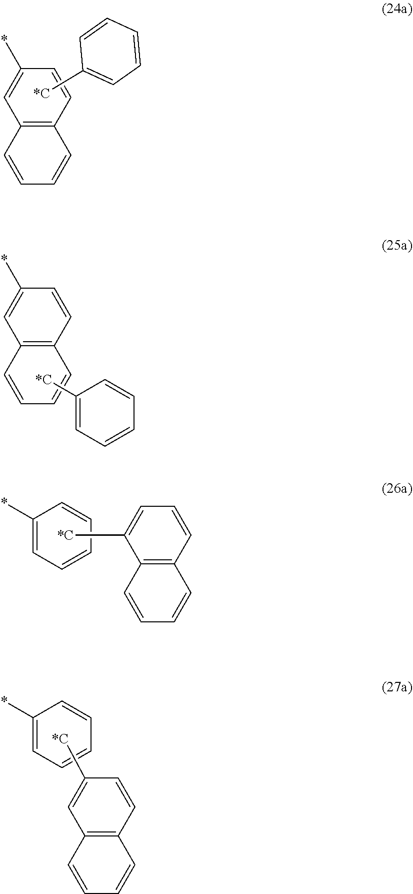
wherein, in the formulae (14a) to (19a) and (22a) to (27a), *C represents a bonding position to a carbon atom on a benzene ring.

25: The compound according to claim 24, wherein
Ar₁₀ is a group represented by any of formulae (10a) to (14a), (17a), (18a), and (26a).
